(19) United States
(12) Patent Application Publication
(10) Pub. No.: US 2025/0109186 A1
(43) Pub. Date: Apr. 3, 2025

(54) SARS-CoV-2 BINDING PROTEINS AND METHODS OF USE THEREOF

(71) Applicant: Regents of the University of Minnesota, Minneapolis, MN (US)

(72) Inventors: Frank Jonathan Lee, Minneapolis, MN (US); Gang Ye, Minneapolis, MN (US); Bin Liu, Minneapolis, MN (US); Fan Bu, Minneapolis, MN (US)

(21) Appl. No.: 18/889,190

(22) Filed: Sep. 18, 2024

Related U.S. Application Data
(60) Provisional application No. 63/541,208, filed on Sep. 28, 2023.
(60) Provisional application No. 63/682,259, filed on Aug. 12, 2024.

(51) Int. Cl. C07K 16/10; A61K 39/00; A61P 31/14; G01N 33/569

(52) U.S. Cl. CPC C07K 16/1003; A61P 31/14; G01N 33/56983; A61K 2039/505; C07K 2317/22; C07K 2317/52; C07K 2317/569; C07K 2317/76; C07K 2317/92; C07K 2317/94; G01N 2333/165

(57) ABSTRACT

Certain embodiments of the invention provide isolated anti-SARS-CoV-2 sdAbs, as well as polypeptides and protein molecules comprising such sdAbs. Certain embodiments of the invention also provide methods of using these sdAbs, polypeptides and protein molecules for treating or preventing a SARS-CoV-2 infection.

### CROSS-REFERENCE TO RELATED APPLICATION

This application claims priority to U.S. Provisional Application No. 63/541,208 that was filed on Sep. 28, 2023, and U.S. Provisional Application No. 63/682,259 that was filed on Aug. 12, 2024. The entire content of the applications referenced above is hereby incorporated by reference herein.

### GOVERNMENT FUNDING

This invention was made with government support under AI157975, AI171954, All 10700, and AI089728 awarded by the National Institutes of Health. The government has certain rights in the invention.

### SEQUENCE LISTING

The instant application contains a Sequence Listing which has been submitted electronically in XML file format via Patent Center and is hereby incorporated by reference in its entirety. Said XML copy, created on Nov. 4, 2024, is named 09531_570US1_SL.xml and is 207,575 bytes in size.

### BACKGROUND OF THE INVENTION

The COVID-19 pandemic has exposed the serious limitations of conventional antibodies as therapeutics, such as their limited potency, ineffectiveness against new variants of severe acute respiratory syndrome-associated coronavirus (SARS-CoV), high cost, and injection-only administration route. Thus, there is a need for highly potent, wide-spectrum, cost effective, and easily administered anti-SARS-CoV-2 therapeutics.

### SUMMARY OF THE INVENTION

Certain embodiments of the invention provide an isolated anti-SARS-CoV-2 binder protein that comprises one or more complementarity determining regions (CDRs) selected from the group consisting of:
(a) a CDR1 comprising an amino acid sequence having at least 75% sequence identity to an amino acid sequence of FNFETSTV (SEQ ID NO: 2);
(b) a CDR2 comprising an amino acid sequence having at least 75% sequence identity to an amino acid sequence of CINKGYEDTN (SEQ ID NO: 3); and
(c) a CDR3 comprising an amino acid sequence having at least 75% sequence identity to an amino acid sequence of AAHNEPYFCDYSGRFRWNEYSY (SEQ ID NO: 4).

Certain embodiments of the invention provide an isolated anti-SARS-CoV-2 binder protein that comprises one or more complementarity determining regions (CDRs) selected from the group consisting of:
(a) a CDR1 comprising an amino acid sequence having at least 75% sequence identity to an amino acid sequence of SIFSPNTM (SEQ ID NO: 8) or STSASNSM (SEQ ID NO: 34);
(b) a CDR2 comprising an amino acid sequence having at least 75% sequence identity to an amino acid sequence of VISSIASTQ (SEQ ID NO: 9), FISSIASTS (SEQ ID NO: 157), or TAANGDIRS (SEQ ID NO: 35); and
(c) a CDR3 comprising an amino acid sequence having at least 75% sequence identity to an amino acid sequence of YAVDKSQDY (SEQ ID NO: 10) or YSVDSYRDY (SEQ ID NO: 36).

Certain embodiments of the invention provide an isolated anti-SARS-CoV-2 binder protein that comprises one or more complementarity determining regions (CDRs) selected from the group consisting of:
(a) a CDR1 comprising an amino acid sequence having at least 75% sequence identity to an amino acid sequence of FTLDYYAI (SEQ ID NO: 14), FILDFYAI (SEQ ID NO: 47), TTLDHYAI (SEQ ID NO: 65), FTVNSHAI (SEQ ID NO: 70), or FALDYYAI (SEQ ID NO: 76);
(b) a CDR2 comprising an amino acid sequence having at least 75% sequence identity to an amino acid sequence of CISSSGGRTN (SEQ ID NO: 15), CISSSGGSTN (SEQ ID NO: 66), or CISISGGSTN (SEQ ID NO: 77); and
(c) a CDR3 comprising an amino acid sequence having at least 75% sequence identity to an amino acid sequence of AAWEASRWYCPLQFSADFSS (SEQ ID NO: 16) AAWEGSTRYCPIQTSADFVS (SEQ ID NO: 48), AAWEGSSEYCPLQFSADFAS (SEQ ID NO: 67) or AAWEGSSEYCPLQYSADFDS (SEQ ID NO: 73).

Certain embodiments of the invention provide an isolated anti-SARS-CoV-2 binder protein (e.g., Class 4, including Nanosota-5, as described in Table 4) that comprises one or more complementarity determining regions (CDRs) selected from the group consisting of:
(a) a CDR1 comprising an amino acid sequence having at least 75% sequence identity to an amino acid sequence of SIFRFEAV (SEQ ID NO: 90), SIFRMDVV (SEQ ID NO: 95), or SIFRMELM (SEQ ID NO: 102);
(b) a CDR2 comprising an amino acid sequence having at least 75% sequence identity to an amino acid sequence of TVARDGTTN (SEQ ID NO: 91), SITRSGSTN (SEQ ID NO: 96), or TINRCGSTN (SEQ ID NO: 103), or TITRSGSTN (SEQ ID NO: 163); and
(c) a CDR3 comprising an amino acid sequence having at least 75% sequence identity to an amino acid sequence of NARWWTNF (SEQ ID NO: 92), HARTWTSY (SEQ ID NO: 97), or HARTWTSS (SEQ ID NO: 104).

Certain embodiments of the invention provide an isolated anti-SARS-CoV-2 binder protein that comprises one or more complementarity determining regions (CDRs) selected from the group consisting of:
(a) a CDR1 comprising an amino acid sequence having at least 75% sequence identity to an amino acid sequence of SVTFNSM (SEQ ID NO: 135);
(b) a CDR2 comprising an amino acid sequence having at least 75% sequence identity to an amino acid sequence of QITAGGDTH (SEQ ID NO: 136); and
(c) a CDR3 comprising an amino acid sequence having at least 75% sequence identity to an amino acid sequence of HLQVPFLGGGYDY (SEQ ID NO: 137).

Certain embodiments of the invention provide an isolated anti-SARS-CoV-2 binder protein that comprises one or more complementarity determining regions (CDRs) selected from the group consisting of:
(a) a CDR1 comprising an amino acid sequence having at least 75% sequence identity to an amino acid sequence of SIFSINAM (SEQ ID NO: 144);
(b) a CDR2 comprising an amino acid sequence having at least 75% sequence identity to an amino acid sequence of GITDDGSTN (SEQ ID NO: 145); and
(c) a CDR3 comprising an amino acid sequence having at least 75% sequence identity to an amino acid sequence of NAKIHTPYNY (SEQ ID NO: 146).

Certain embodiments of the invention provide a sdAb-Fc fusion protein comprising an isolated anti-SARS-CoV-2 sdAb as described herein operably linked to an Fc domain amino acid sequence.

Certain embodiments of the invention provide an isolated anti-SARS-CoV-2 binder protein comprising two independently selected sbAb-Fc fusion proteins as described herein, wherein the two Fc polypeptides are linked to form a dimer.

Certain embodiments of the invention provide a composition comprising isolated anti-SARS-CoV-2 binder protein as described herein, and a carrier.

Certain embodiments of the invention provide an isolated polynucleotide comprising a nucleotide sequence encoding an isolated anti-SARS-CoV-2 binder protein as described herein.

Certain embodiments of the invention provide a vector comprising the polynucleotide as described herein.

Certain embodiments of the invention provide a cell comprising the polynucleotide as described herein or the vector as described herein.

Certain embodiments of the invention provide broad-spectrum SARS-CoV binder protein(s), or mixture thereof.

Certain embodiments of the invention provide a method of inhibiting the activity of SARS-CoV (e.g., SARS-CoV-1, or SARS-CoV-2 or variant thereof, such as Omicron), comprising contacting SARS-CoV with an isolated anti-SARS-CoV-2 binder protein as described herein.

Certain embodiments of the invention provide a method for treating or preventing a SARS-CoV (e.g., SARS-CoV-1, or SARS-CoV-2 or variant thereof, such as Omicron) infection in a mammal, comprising administering an effective amount of an isolated anti-SARS-CoV-2 binder protein as described herein, to the mammal.

Certain embodiments of the invention provide an isolated anti-SARS-CoV-2 binder protein as described herein, for the prophylactic or therapeutic treatment of a SARS-CoV-2 infection (e.g., SARS-CoV-1, or SARS-CoV-2 or variant thereof, such as Omicron).

Certain embodiments of the invention provide an isolated anti-SARS-CoV-2 binder protein as described herein, for use in medical therapy.

Certain embodiments of the invention provide a kit comprising:
1) an isolated anti-SARS-CoV-2 binder protein as described herein;
2) packaging material; and
3) instructions for detecting the presence of SARS-CoV-2 or for administering the binder protein to a mammal to treat or prevent a SARS-CoV-2 infection (e.g., Omicron variant).

Certain embodiments of the invention provide a method of detecting the presence of SARS-CoV-2 in a biological sample, the method comprising contacting the biological sample with an isolated anti-SARS-CoV-2 binder protein as described herein, and detecting whether a complex is formed between 1) the binder protein; and 2) SARS-CoV-2.

The invention also provides processes and intermediates disclosed herein that are useful for preparing anti-SARS-CoV-2 binder proteins as described herein (e.g., sdAbs, and polypeptides), as well as compositions described herein. For example, certain embodiments provide a method as described herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Construction and screening of induced nanobody phage display library for anti-SARS-CoV-2 nanobodies. An alpaca was immunized with recombinant prototypic SARS-CoV-2 spike protein and the peripheral blood mononuclear cells were used to construct an induced nanobody phage display library. Panning against the library using the spike protein was performed to identify strong binders, which were then subjected to pseudovirus entry assay for selection of neutralizing nanobodies. Three neutralizing nanobodies (Nanosota-2A, Nanosota-3A and Nanosota-4A) were identified and they all bind to the RBD. Fc-tagged nanobodies were constructed and prepared; their binding kinetics against prototypic SARS-CoV-2 RBD were determined using surface plasmon resonance (SPR).

FIGS. 2A-2B. Nanosota-2, -3 and -4 demonstrate super potency and wide spectrum against SARS-CoV-2 in vitro. (FIG. 2A) Efficacy of Fc-tagged nanobodies in neutralizing SARS-CoV-2 and SARS-CoV-1 pseudoviruses. Retroviruses pseudotyped with coronavirus spike protein entered human ACE2-expressing cells in the presence of one of the nanobodies at different concentrations. Entry efficiency was characterized as luciferase signal accompanying entry. The efficacy of each nanobody was expressed as the concentration capable of neutralizing pseudovirus entry by 50% (i.e., IC₅₀). Bat SARS2 refers to strain BANAL236 strain; Bat SARS1 refers to strain Rs3367. N/A means no obvious neutralization. Each experiment was repeated at least twice. (FIG. 2B) Efficacy of Fc-tagged nanobodies in neutralizing live infectious SARS-CoV-2 in vitro. Prototypic SARS-CoV-2 or omicron variant infected Vero cells in the presence of Nanosota-2A-Fc or Nanosota-3A-Fc, respectively, at different concentrations. Infection was characterized as the number of virus plaques formed in overlaid cells. Data are the mean±SEM (n=3). Nonlinear regression was performed using a log (inhibitor) versus normalized response curve. The efficacy of each nanobody was expressed as the concentration capable of reducing the relative percentage of infection by 50% (i.e., IC₅₀). Each experiment was repeated at least twice. The potency metrics of Nanosota-2A-Fc in inhibiting SARS-CoV-2 in vitro are among the best of all known anti-SARS-CoV-2 entry inhibitors.

FIGS. 3A-3B. Nanosota-2 demonstrates super potency against prototypic SARS-CoV-2 in mouse model. The efficacy of Nanosota-2A-Fc was evaluated for treating the infection of prototypic SARS-CoV-2 in mice. (FIG. 3A) Nanosota-2A-Fc was administered at 4 hours post-challenge and a dosage of 10 mg/Kg weight. Human ACE2-transgenic mice were challenged via intranasal inoculation of prototypic SARS-CoV-2. In the treatment group (n=5), mice received Nanosota-2A-Fc via intraperitoneal injection. In the control group (n=4), mice were administered PBS buffer. Body weight changes up to day 5 post-challenge were recorded and lung virus titers on day 2 post-challenge were measured. (FIG. 3B) Nanosota-2A-Fc was administered at a delayed time point (18 hours post-challenge at 16 mg/Kg weight) or a lowered dosage (4 mg/Kg weight at 4 hours post-challenge). n=5 for both the treatment and control groups. Comparisons of lung virus titers between the control and treatment groups were performed using unpaired two-tailed Student's t-test. Error bars represent SEM. *p<0.05; **p<0.01; ***p<0.001. The potency metrics of Nanosota-2A-Fc in inhibiting SARS-CoV-2 in vivo are among the best of all known anti-SARS-CoV-2 entry inhibitors.

FIGS. 4A-4C. Nanosota-3 demonstrates high potency against SARS-CoV-2 omicron BA.1 in mouse models via different administration routes. The efficacy of Nanosota-3A-Fc was evaluated for treating the infection of SARS-CoV-2 omicron variant in mouse models and via different administration routes: (FIG. 4A) human ACE2-transgenic mice (n=6) and intraperitoneal delivery; (FIG. 4B) Balb/c mice (n=5) and intraperitoneal delivery; (FIG. 4C) Balb/c mice (n=5) and intranasal delivery. Nanosota-3A-Fc was administered at 4 hours post-challenge and a dosage of 10 mg/Kg weight. In the control groups, mice were administered PBS buffer. Lung virus titers on day 3 post-challenge (for intraperitoneal delivery) or day 2 post-challenge (for intranasal delivery) were measured. Comparisons of lung virus titers between the control and treatment groups were performed using unpaired two-tailed Student's t-test. Error bars represent SEM. **p<0.01; ***p<0.001.

FIGS. 5A-5C. Structural basis for anti-SARS-CoV-2 potency and spectrum of Nanosota-2, -3 and -4. The cryo-EM structures of prototypic SARS-CoV-2 spike complexed with each of the three nanobodies were determined. Atomic models were built for the spike protein in all of the three structures and for Nanosota-3A, whereas local docking models were generated for Nanosota-2A and Nanosota-4A. (FIG. 5A) Superimposition of spike-bound Nanosota-2A, -3A and -4A onto the structure of the same spike protein (with 1 RBD up). Spike trimer is colored in gray, with three RBDs (1 up and two down) colored in cyan. Nanosota-2A, -3A and -4A are colored in red, blue and brown, respectively. Nanosota-3A and Nanosota-4A bind to both standing-up and lying-down RBDs, whereas Nanosota-2A only binds to the standing-up RBD. (FIG. 5B) Superimposition of RBD-bound Nanosota-2A, -3A, -4A, and human ACE2 onto the structure of the same RBD. The three nanobodies are colored the same as in (FIG. 5A). The core structure of the RBD is colored in cyan and the receptor-binding motif (RBM) is colored in magenta. ACE2 (PDB 6MOJ) is colored in green. The clashes between each nanobody and ACE2 are labeled. (FIG. 5C) Superimposition of the footprints of the nanobodies and the mutations of viral variants onto the RBD. The footprints of Nanosota-2A and Nanosota-4A (from docking models) are shown as red and brown transparent ovals, respectively, whereas the footprint of Nanosota-3A (from atomic model) is shown as blue surface presentation. Mutations (i.e., residue changes) in different SARS-CoV-2 variants and SARS-CoV-1 are mapped to the prototypic SARS-CoV-2 RBD in different colors: mutations appearing in multiple viral variants are all colored black, whereas unique mutations for a specific viral variant are colored uniquely as labeled. Mutations overlapping with the footprints of nanobodies are labeled with the specific residue changes, whereas mutations outside the footprints are only labeled with the viral strains in which they appeared.

FIGS. 6A-6D. Binding kinetics between Fc-tagged nanobodies and prototypic SARS-CoV-2 RBD as measured by surface plasmon resonance. (FIG. 6A) Nanosota-2A-Fc. (FIG. 6B) Nanosota-3A-Fc. (FIG. 6C) Nanosota-4A-Fc. Each of the Fc-tagged nanobodies was immobilized to a protein A sensor chip. Prototypic SARS-CoV-2 RBD was injected at different concentrations (2.5 nM-80 nM). The resulting data were fitted to a 1:1 binding model using Biacore Evaluation Software. (FIG. 6D) Competition SPR analysis. His-tagged RBD was immobilized onto four CM5 sensor chips. Each of the Fc-tagged nanobodies was injected to one of the first three chips. In contrast, the fourth chip received only the running buffer. Following this, after the nanobodies had bound to the RBD, a mixture of recombinant human ACE2 and the corresponding nanobody was added to the first three chips. The fourth chip received only ACE2. Sensorgrams from all chips were then overlaid for comparison. No change in the resonance signal from nanobody-bound RBD indicated that ACE2 could not displace nanobody from binding to the RBD, as in the case for Nanosota-2A-Fc and Nanosota-3A-Fc. A slight increase in the resonance signal from nanobody-bound RBD suggested ACE2 largely could not displace nanobody from binding to the RBD, as in the case for Nanosota-4A-Fc.

FIGS. 7A-7B. Pathological analysis of lung tissues in SARS-CoV-2-challenged mice. (FIG. 7A) Representative hematoxylin and eosin (HE) staining of lung tissues harvested on day 5 post-challenge from mice in FIG. 3A. Inflammation (arrows, top row) and edema in airspaces (asterisks, bottom row) were much more evident in the control group (two left panels) compared to the treatment group (two right panels). (FIG. 7B) Representative HE staining of lung tissues harvested on day 5 post-challenge from mice in FIG. 3B. The control group (two left panels) shows much more evident cellular perivascular cellular aggregates (arrows) and smaller airspaces compared to the treatment groups (two right panels).

FIGS. 8A-8D. Structures of the complexes of prototypic SARS-CoV-2 spike and each of Nanosota-2A, -3A and -4A. (FIG. 8A) SARS-CoV-2 spike (with one RBD up) complexed with Nanosota-2A. The spike trimer is colored by different subunits: subunit A in cyan, subunit B in green and subunit C in magenta. Nanosota-2A is colored in red. (FIG. 8B) SARS-CoV-2 spike (with one RBD up) complexed with two Nanosota-3A molecules. (FIG. 8C) SARS-CoV-2 spike (with two RBDs up) complexed with three Nanosota-3A molecules. (FIG. 8D) SARS-CoV-2 spike (with two RBDs up) complexed with three Nanosota-4A molecules.

FIG. 9. Flow chart of cryo-EM image processing and 3D reconstruction for prototypic SARS-CoV-2 spike/Nanosota-2A complex. Representative raw cryo-EM image and 2D classes are presented. 3D refinements using all the particles from good 3D classes generated a 2.1 Å map. Two rounds of masked 3D classification and further local refinement visualized the density for the bound nanobody. Angular distribution plot, the final maps, half-map FSC curves and accompanying local resolution illustrations (also see the heat map bar showing resolution scale) are enclosed in the dashed black boxes.

FIG. 10. Flow chart of cryo-EM image processing and 3D reconstruction for prototypic SARS-CoV-2 spike/Nanosota-3A complex. Representative raw cryo-EM image and 2D classes are presented. 3D refinements using the particles from individual good 3D classes generated 2.5 Å maps. Further local refinements improved the density for the bound nanobodies. The final maps, half-map FSC curves, angular distribution plot and accompanying local resolution illustrations (also see the heat map bar showing resolution scale) are enclosed in the dashed black boxes.

FIG. 11. Flow chart of cryo-EM image processing and 3D reconstruction for prototypic SARS-CoV-2 spike/Nanosota-4A complex. Representative raw cryo-EM image and 2D classes are presented. 3D refinements using the particles in the good 3D class generated a 3.4 Å map. Further local refinements improved the density for the bound nanobodies. The final maps, half-map FSC curves, angular distribution plot and accompanying local resolution illustrations (also see the heat map bar showing resolution scale) are enclosed in the dashed black boxes.

FIGS. 12A-12D. Structural interfaces of spike/Nanosota-3A and spike/Nanosota-4A. (FIG. 12A) Cryo-EM densities (after local refinement) of the interface between the lying-down RBD and Nanosota-3A. RBD is colored in cyan, Nanosota-3A is colored in blue, and cryo-EM densities are colored in magenta. (FIG. 12B) Structural details of the spike/Nanosota-3A interface involving the L452R mutation that appeared in SARS-CoV-2 delta variant. RBM is colored in magenta and Nanosota-3A is colored in blue. (FIG. 12C) Nanosota-4A (heavy chain only antibody) binds to the lying-down RBD by fitting into a cavity in the trimeric spike. Only the three receptor-binding subunits are shown. RBD is colored in cyan. Nanosota-4A is colored in brown. (FIG. 12D) The antigen-binding region (both heavy and light chains) of a conventional antibody (PDB 7DEO) would not fit into the cavity and hence would not bind to the lying-down RBD in the same way as Nanosota-4A does. The clash of the antibody and the spike protein is labeled.

FIGS. 13A-13C. Structure of SARS-CoV-2 spike complexed with all of the spike-bound nanobodies. (FIG. 13A) Schematic drawing of SARS-CoV-2 spike ectodomain (prototypic strain). S1: receptor-binding subunit. S2: membrane-fusion subunit. NTD: N-terminal domain of S1. RBD: receptor-binding domain of S1. RBM: receptor-binding motif of RBD. SD1: subdomain 1 of S1. SD2: subdomain 2 of S1. Furin site: cleavage site for the furin protease. (FIG. 13B) Cryo-EM structure of trimeric SARS-CoV-2 spike ectodomain complexed with all of the spike-bound nanobodies. Nine nanobodies (colored in blue) bound to four unique epitopes on the trimeric spike. These epitopes represent dominant epitopes recognized by high-affinity and prevalent nanobodies. The spike domains are colored in the same way as (A). Among the three spike subunits, one contains a standing-up RBD (top right; i.e., open subunit) and the other two lying-down RBDs (i.e., closed subunits). 57 glycans are shown as red balls. (FIG. 13C) Structure of monomeric spike subunit (open) complexed with all of the spike-bound nanobodies. Nanosota-4, which bound to the RBD of a closed subunit, was modeled to the equivalent site on the RBD of this open subunit.

FIGS. 14A-14D. Structures of SARS-CoV-2 spike complexed with individual spike-binding nanobodies. (FIG. 14A) Overlay of the cryo-EM structures of SARS-CoV-2 monomeric spike subunit complexed with individual nanobodies (Nanosota-2, -3, -4, -5, -6). The binding site for Nanosota-7 could not be identified. The six nanobodies were discovered individually from top spike-binding phages. The numbers in parenthesis represent the prevalence of each nanobody in the mixture of top spike-binding nanobodies. The spike domains, nanobodies, and glycans are colored in the same way as FIG. 13. (FIG. 14B) Overlay of the structures of the RBD complexed with Nanosota-2, -3, -4 and ACE2. ACE2 is in green. Clashes between the nanobodies and ACE2 are indicated. (FIG. 14C) Structure of Nanosota-5 bound to the NTD and SD2 from the same spike subunit. (FIG. 14D) Structure of Nanosota-6 bound to the NTD and SD1 from two different spike subunits.

FIGS. 15A-15D. Functions of spike-binding nanobodies in SARS-CoV-2 pseudovirus entry. Retroviruses pseudotyped with prototypic SARS-CoV-2 spike (i.e., SARS-CoV-2 pseudoviruses) entered ACE2-expressing cells in the presence of one of the nanobodies at different concentrations. Entry efficiency was characterized as luciferase signal accompanying entry. The efficacy of each nanobody in neutralizing pseudovirus entry was expressed as the concentration capable of neutralizing pseudovirus entry by 50% (i.e., IC₅₀). (FIG. 15A) Activities of three Fc-tagged RBD-targeting nanobodies: they all potently neutralized SARS-CoV-2 pseudovirus entry. Error bars represent SEM (n=3). (FIG. 15B) Activities of three Fc-tagged non-RBD-targeting nanobodies: Nanosota-5-Fc enhances pseudovirus entry, Nanosota-6-Fc enhances pseudovirus entry at low and high concentrations, respectively, and Nanosota-7-C neutralizes pseudovirus entry at high concentrations. Error bars represent SEM (n=4). (FIG. 15C) Activities of three His-tagged non-RBD-targeting nanobodies: Nanosota-5-His and Nanosota-6-His enhances pseudovirus entry using an Fc-independent mechanism. Error bars represent SEM (n=4). (FIG. 15D) Summary of the functions of the six spike-binding nanobodies. NA: not available.

FIGS. 16A-16C. Further investigation of the functions of spike-binding nanobodies using depletion assay. (FIG. 16A) RBD-targeting nanobodies were depleted from the mixture of spike-binding nanobodies using RBD-conjugated beads. All nanobodies contain an Fc tag. (FIG. 16B) ELISA was performed to detect RBD binding by nanobody mixtures before and after depletion. The result confirmed the effectiveness of the depletion experiment. (FIG. 16C) SARS-CoV-2 pseudovirus entry was carried out to further investigate the functions of spike-binding nanobodies. The result confirmed that RBD-targeting nanobodies account for neutralization, whereas non-RBD-targeting nanobodies account for ADE. Error bars represent SEM (n=4).

FIGS. 17A-17B. Non-RBD-targeting nanobody causes antibody-dependent enhancement of live SARS-CoV-2 infection in cultured cells. Recombinant SARS-CoV-2-Venus virus was used to infect ACE2-expressing cells in the presence of different concentrations of Nanosota-5-Fc. Absence of Nanosota-5-Fc was used as a negative control. Infection efficiency was characterized as percentage of infected cells as detected by flow cytometry. Two different virus titers were used. MOI stands for multiplicity of infection. Nanosota-5-Fc caused more prominent ADE at the lower virus titer (FIG. 17A) than at the higher virus titer (FIG. 17B). Comparisons of viral infections between the negative control and different concentrations of Nanosota-5-Fc were performed using unpaired two-tailed Student's t-test. Error bars represent SEM (n=3). ***p<0.001; **p<0.01.

FIG. 18. Construction and screening of induced nanobody phage display library for SARS-CoV-2 spike-targeting nanobodies. An alpaca was immunized with purified prototypic SARS-CoV-2 spike ectodomain and its peripheral blood mononuclear cells were used to construct an induced nanobody phage display library. Bio-panning against the library using the purified spike was performed to identify spike-binding phages. The collection of spike-binding phages expressed a mixture of spike-binding nanobodies. The complex of the spike and all the spike-bound nanobodies was purified on gel filtration chromatography and subjected to cryo-EM analysis.

FIG. 19. Flow chart of cryo-EM image processing and 3D reconstruction for SARS-CoV-2 spike/nanobody mixture complex. Representative raw cryo-EM image and 2D classes are presented. 3D refinements using the good particles generated an overall 3.4 Å map. Further local refinement improved the densities of the bound nanobodies. Angular distribution plots, the final maps, half-map FSC curves and accompanying local resolution illustrations are enclosed in the dashed black boxes.

FIGS. 20A-20B. Classification of SARS-CoV-2 spike-binding nanobodies. (FIG. 20A) Screening of top spike-binding phages. From the mixture of spike-binding phages in FIG. 1, 96 phages were randomly picked and assayed for their spike-binding affinity using ELISA. The nanobody genes from the top 48 spike-binding phages were subjected to sequencing. (FIG. 20B) Classification of top spike-binding nanobodies. The top 48 spike-binding nanobodies were classified into six classes based on their CDR3 region. The prevalence of each nanobody class is shown in parentheses. FIG. 20B discloses SEQ ID NOS: 179-226, respectively, in order of appearance.

FIG. 21. Flow chart of cryo-EM image processing and 3D reconstruction for SARS-CoV-2 spike/Nanosota-2 complex. Representative raw cryo-EM image and 2D classes are presented. 3D refinements using all the particles from good 3D classes generated a 2.1 Å map. Two rounds of masked 3D classification and further local refinement visualized the density for the bound nanobody. Angular distribution plot, the final maps, half-map FSC curves and accompanying local resolution illustrations are enclosed in the dashed black boxes.

FIG. 22. Flow chart of cryo-EM image processing and 3D reconstruction for SARS-CoV-2 spike/Nanosota-3 complex. Representative raw cryo-EM image and 2D classes are presented. 3D refinements using the particles from individual good 3D classes generated 2.5 Å maps. Further local refinements improved the density for the bound nanobodies. The final maps, half-map FSC curves, angular distribution plot and accompanying local resolution illustrations are enclosed in the dashed black boxes.

FIG. 23. Flow chart of cryo-EM image processing and 3D reconstruction for SARS-CoV-2 spike/Nanosota-4 complex. Representative raw cryo-EM image and 2D classes are presented. 3D refinements using the particles in the good 3D class generated a 3.4 Å map. Further local refinements improved the density for the bound nanobodies. The final maps, half-map FSC curves, angular distribution plot and accompanying local resolution illustrations are enclosed in the dashed black boxes.

FIG. 24. Flow chart of cryo-EM image processing and 3D reconstruction for SARS-CoV-2 spike/Nanosota-5 complex. Representative raw cryo-EM image and 2D classes are presented. 3D refinements using the good particles generated an overall 3.8 Å maps with C3 symmetry. The final map, half-map FSC curves, angular distribution plot and accompanying local resolution illustration are enclosed in the dashed black boxes.

FIG. 25. Flow chart of cryo-EM image processing and 3D reconstruction for SARS-CoV-2 spike/Nanosota-6 complex. Representative raw cryo-EM image and 2D classes are presented. 3D refinements using the good particles generated an overall 2.8 Å maps with C3 symmetry. The final map, half-map FSC curves, angular distribution plot and accompanying local resolution illustration are enclosed in the dashed black boxes.

FIGS. 26A-26D. Structures of the complexes of prototypic SARS-CoV-2 spike and each of the RBD-targeting nanobodies. (FIG. 26A) SARS-CoV-2 spike (with one RBD up) complexed with one Nanosota-2 molecule. The trimeric spike is colored in the same way as in FIG. 14. (FIG. 26B) SARS-CoV-2 spike (with one RBD up) complexed with two Nanosota-3 molecules. (FIG. 26C) SARS-CoV-2 spike (with two RBDs up) complexed with three Nanosota-3 molecules. (FIG. 26D) SARS-CoV-2 spike (with two RBDs up) complexed with three Nanosota-4 molecules.

FIGS. 27A-27C. Structures of the complexes of prototypic SARS-CoV-2 spike and each of non-RBD-targeting nanobodies. (FIG. 27A) SARS-CoV-2 spike (with three down RBDs) complexed with three Nanosota-5 molecules. The trimeric spike is colored in the same way as in FIG. 14. (FIG. 27B) SARS-CoV-2 spike (with three down RBDs) complexed with three Nanosota-6 molecules. (FIG. 27C) Nanosota-7 binds to a non-RBD epitope in S1. The binding interactions between nanobodies (columns shown from left to right as Nanosota-4, -5, and -7) and SARS-CoV-2 spike domains (RBD, S1, and spike ectodomain) were examined using ELISA. PBS buffer was used as a negative control. Nanosota-4 binds to the RBD, whereas Nanosota-5 and -7 both bind to non-RBD regions in S1. Comparisons of target binding between the negative control and nanobodies were performed using unpaired two-tailed Student's t-test. Error bars represent SEM (n=3). ***p<0.001.

FIGS. 28A-28B. Comparison of nanobody epitopes discovered in this study and human antibody epitopes deposited in the PDB. Numerous spike-targeting human antibodies have been discovered individually from many COVID-19 patients. They bind to eight epitopes on the spike: five on the RBD and three on the NTD. Only one representative human antibody for each of the eight epitopes is shown. The SARS-CoV-2 spike domains are colored in the same way as in FIG. 13. Nanobodies are colored in blue. (FIG. 28A) Overlay of nanobody epitopes and human antibody epitopes on SARS-CoV-2 RBD. The PBD IDs for the five representative human antibodies are: 6XC2 (in red) whose epitope overlaps with the Nanosota-2 epitope, 7SN2 (in green) whose epitope overlaps with the Nanosota-3 epitope, 7BWJ (in yellow) whose epitope overlaps with the Nanosota-4 epitope, 7AKD (in orange), and 7EAM (in brown). (FIG. 28B) Overlay of nanobody epitopes and human antibody epitopes on SARS-CoV-2 NTD. The PBD IDs for the three representative human antibodies are: 7DZX (in red) whose epitope overlaps partially with Nanosota-5 epitope, 7RW2 (in yellow) whose epitope overlaps with Nanosota-6 epitope, and 7LY3 (in cyan).

FIGS. 29A-29C. Coronavirus entry pathways through viral receptor or Fc receptor (FcR). (FIG. 29A) SARS-CoV-2 entry through viral receptor ACE2. (FIG. 29B) Coronavirus entry through RBD-targeting antibodies and FcR, which is a molecular mechanism that we identified previously for antibody-dependent enhance (ADE) of coronavirus entry (see main text). (FIG. 29C) SARS-CoV-2 entry through viral receptor ACE2 and enhanced by non-RBD targeting nanobodies, which is a novel molecular mechanism that the current study identifies for ADE of coronavirus entry.

FIGS. 30A-30B. Structure-guided in vitro affinity maturation of Nanosota-3. (FIG. 30A) Binding between Nanosota-3 and the spike proteins of different SARS-CoV-2 variants (from left to right: prototypic, BA.1, BA.5, XBB.1.5) using ELISA. Nanosota-3A is the original version of Nanosota-3, which binds to the spikes of the prototypic strain and omicron BA.1 subvariant, but not the spike of the currently circulating XBB.1.5. Structure-guided in vitro affinity maturation of Nanosota-3A using phage display generated Nanosota-3B, which binds the XBB.1.5 spike. Comparisons of the ELISA results between Nanosota-3A and Nanosota-3B were performed using unpaired two-tailed Student's t-test. Error bars represent SEM. **p<0.01; ***p<0.001. (FIG. 30B) Detailed structure at the interface between prototypic RBD and Nanosota-3A. The RBD is colored in magenta and Nanosota-3A in blue. RBD residue 490 mutated from a phenylalanine in the prototypic strain (also the BA.1 subvariant) to a serine in XBB.1.5 subvariant. Three Nanosota-3A residues (Met47, Val50 and Asn58) surrounding RBD residue 490 were subjected to random mutagenesis in in vitro affinity maturation to generate Nanosota-3B. Compared to Nanosota-3A, Nanosota-3B contains two mutations, V50F and Q58S.

FIGS. 31A-31E. Structures of prototypic SARS-CoV-2 spike complexed with individual non-RBD-targeting nanobodies. (FIG. 31A) Schematic drawing of SARS-CoV-2 spike ectodomain (prototypic variant). S1: receptor-binding subunit. S2: membrane-fusion subunit. NTD: N-terminal domain of S1. RBD: receptor-binding domain of S1. RBM: receptor-binding motif of RBD. SD1: subdomain 1 of S1. SD2: subdomain 2 of S1. Furin site: cleavage site for the furin protease. (FIG. 31B) Structure of prototypic SARS-CoV-2 spike complexed with Nanosota-5. Three bound Nanosota-5 molecules are colored in blue. The spike domains are colored in the same way as (FIG. 31A). The furin cleavage site is labeled. (FIG. 31C) The binding site of Nanosota-5 on prototypic SARS-CoV-2 spike. A glycan N-linked to Asn61 on the spike is shown as red balls. NTD and SD2 bound by Nanosota-5 are from the same spike protomer. (FIG. 31D) Structure of prototypic SARS-CoV-2 spike complexed with Nanosota-6. Three bound Nanosota-6 molecules are colored in blue. (FIG. 31E) The binding site of Nanosota-6 on prototypic SARS-CoV-2 spike. NTD and SD1 bound by Nanosota-6 are from two spike protomers.

FIGS. 32A-32C. A non-RBD epitope enhances cell infection by live prototypic SARS-CoV-2. Recombinant SARS-CoV-2-Venus (prototypic variant) was used to infect Vero E6 cells in the presence of various concentrations of Nanosota-5-Fc. PBS buffer was used as a negative control. (FIG. 32A) Nanosota-5-Fc enhances cell infection by live prototypic SARS-CoV-2 at a high virus titer. (FIG. 32B) Nanosota-5-Fc enhances cell infection by live prototypic SARS-CoV-2 at a low virus titer. (FIG. 32C) Nanosota-3-Fc, an RBD-targeting neutralizing nanobody, was used for comparison to Nanosota-5-Fc. Infection efficiency was characterized as the percentage of infected cells detected by flow cytometry. MOI stands for multiplicity of infection. Comparisons of viral infections between the negative control and various concentrations of Nanosota-5-Fc or Nanosota-3-Fc were performed using an unpaired two-tailed Student's t-test. Error bars represent SEM (n=3). ***p<0.001; **p<0.01.

FIGS. 33A-33C. A non-RBD epitope neutralizes the cell entry of SARS-CoV-2 Omicron variant. (FIG. 33A) Pseudovirus entry assay shows that Nanosota-5-Fc neutralizes the cell entry of three Omicron subvariants. Error bars represent SEM (n=4). IC₅₀ values were calculated for each of the three Omicron subvariants. (FIG. 33B) Cell-cell fusion assay shows that Nanosota-5-Fc decreases XBB.1.5-spike-mediated cell-cell fusion (left), but increases prototypic-spike-mediated cell-cell fusion (right). Spike-expressing cells and ACE2-expressing cells were incubated together for fusion in the presence of various concentrations of Nanosota-5-Fc. Comparisons of cell-cell fusion between the negative control (PBS buffer) and various concentrations of Nanosota-5-Fc were performed using an unpaired two-tailed Student's t-test. Error bars represent SEM (n=4). ***p<0.001; **p<0.01. **p<0.05. NS: not statistically significant. (FIG. 33C) Nanosota-5-Fc neutralizes cell infection by live Omicron subvariants. The efficacy of Nanosota-5-Fc against Omicron subvariants was calculated and expressed as the concentration capable of maintaining the cell viability by 50% (IC₅₀) compared to control virus. Error bars represent SEM (n=4).

FIGS. 34A-34D. Structure of XBB.1.5 spike complexed with non-RBD-targeting nanobody. (FIG. 34A) Structure of XBB.1.5 spike complexed with Nanosota-5. Three bound Nanosota-5 molecules are colored in blue. The spike domains are colored as in FIG. 31A. (FIG. 34B) Interactions between Nanosota-5 and the NTD of XBB.1.5 spike. Three complementarity-determining regions (CDRs) and part of a framework region (FR) of Nanosota-5 (shown as ribbons) are directly involved in binding the NTD residues (shown as sticks).

FIG. 34B discloses “FFSN” as SEQ ID NO: 227 and “LDPL” as SEQ ID NO: 228. (FIG. 34C) Interactions between Nanosota-5 and the SD2 of XBB.1.5 spike. CDR1 and CDR3 of Nanosota-5 (shown as ribbons) are directly involved in binding the SD2 residues (shown as sticks). All Nanosota-5-contacting residues in the NTD and SD2 are conserved from the prototypic to Omicron spikes (see FIG. 41). FIG. 34C discloses “WRVYST” as SEQ ID NO: 229 and “NQVAV” as SEQ ID NO: 230. (FIG. 34D) ELISA comparing the binding of Nanosota-5-His to the spike ectodomains from prototypic SARS-CoV-2, Omicron BA. 1, Omicron BA.5, and Omicron XBB.1.5.

FIGS. 35A-35D. Mechanism for non-RBD epitopes exhibiting opposite functions across different SARS-CoV-2 variants. Flow cytometry assay shows that Nanosota-5-Fc increases ACE2 binding by prototypic spike (FIG. 35A) but decreases ACE2 binding by XBB.1.5 spike (FIG. 35B). A mixture of recombinant ACE2 ectodomain and recombinant Nanosota-5-Fc was incubated with spike-expressing cells, and the binding between the recombinant ACE2 ectodomain and the cell-surface spike was measured using flow cytometry. Nanosota-2, which, like ACE2, only binds to the standing up RBD, replaced the ACE2 ectodomain in (FIG. 35C). PBS buffer was used as a negative control. Comparisons between the negative control and Nanosota-5-Fc for their impact on spike/ACE2 binding were performed using an unpaired two-tailed Student's t-test. Error bars represent SEM (n=3). ***p<0.001, **p<0.01, *p<0.05. (FIG. 35D) Post-attachment pseudovirus entry. Pseudoviruses were incubated with cells before adding Nanosota-5-Fc, allowing pseudovirus entry to occur.

FIG. 36. Flow chart of cryo-EM image processing and 3D reconstruction for the complex of prototypic SARS-CoV-2 spike and Nanosota-5. Representative raw cryo-EM images and 2D classes are presented. 3D refinements using the good particles generated an overall 3.8 Å map with C3 symmetry. The final map, half-map FSC curves, angular distribution plot, and accompanying local resolution illustration are enclosed in the dashed black boxes.

FIG. 37. Flow chart of cryo-EM image processing and 3D reconstruction for the complex of prototypic SARS-CoV-2 spike and Nanosota-6. Representative raw cryo-EM images and 2D classes are presented. 3D refinements using the good particles generated an overall 2.8 Å map with C3 symmetry. The final map, half-map FSC curves, angular distribution plot, and accompanying local resolution illustration are enclosed in the dashed black boxes.

FIG. 38. Nanosota-7 binds to a non-RBD epitope in S1. The binding interactions between nanobodies (columns from left to right: Nanosota-4, -5, and -7) and SARS-CoV-2 spike domains (RBD, S1, and spike ectodomain) were examined using ELISA. PBS buffer was used as a negative control. Nanosota-4 binds to the RBD, whereas Nanosota-5 and -7 both bind to non-RBD regions in S1. Comparisons of target binding between the negative control and nanobodies were performed using an unpaired two-tailed Student's t-test. Error bars represent SEM (n=3). ***p<0.001.

FIGS. 39A-39B. Additional data on Nanosota-5's enhancement of the cell entry of pre-Omicron SARS-CoV-2 pseudoviruses. (FIG. 39A) Even at very high concentrations (e.g., 0.4 mg/ml), Nanosota-5-Fc continued to enhance the cell entry of prototypic SARS-CoV-2 pseudoviruses. (FIG. 39B) Nanosota-5-Fc enhanced the cell entry of both the alpha and delta variants of SARS-CoV-2 pseudoviruses. Comparisons of pseudovirus entry between conditions with and without Nanosota-5-Fc (i.e., 100% of pseudovirus entry) were performed using an unpaired two-tailed Student's t-test. Error bars represent SEM (n=3). Statistical significance is indicated as follows: ***p<0.001; **p<0.01; *p<0.05.

FIG. 40. Flow chart of cryo-EM image processing and 3D reconstruction for the complex of SARS-CoV-2 XBB.1.5 spike and Nanosota-5. Representative raw cryo-EM images and 2D classes are presented. 3D refinements using the good particles generated an overall 3.49 Å map with C3 symmetry. The final map, half-map FSC curves, angular distribution plot, and accompanying local resolution illustration are enclosed in the dashed black boxes.

FIG. 41. Comparison of Nanosota-5-contacting residues on prototypic and omicron spikes. The Nanosota-5-contacting residues on prototypic and XBB.1.5 spikes were identified using cryo-EM structures of the respective spike/Nanosota-5 complexes analyzed by PDBePISA (https://www.ebi.ac.uk/pdbe/pisa/). Additionally, the spikes from two other Omicron subvariants, BA.1 and BA.5, were included in the sequence comparisons. #Due to deletions, the residue numbering in the Omicron spikes is three units lower than their corresponding residues in the prototypic spike. For clarity, this difference is not depicted in the figure. FIG. 41 discloses SEQ ID NOS: 231, 228, 231, 228, 231, 228, 231, 228, 232, 229, 232, 229, 232, 229, 232 and 229, respectively, in order of appearance.

FIGS. 42A-42B. Comparison of surface electrostatic potentials in the Nanosota-5 epitope and adjacent regions. The residues near but outside the Nanosota-5 binding site were analyzed, revealing several differences between the prototypic spike (FIG. 42A) and the XBB.1.5 spike (FIG. 42B). This figure was created using PyMol v2.5.2.

FIGS. 43A-43C. Impact of cellular proteases on Nanosota-5's activities. (FIG. 43A) ELISA comparing the binding affinity of Nanosota-5-His for the recombinant prototypic SARS-CoV-2 spike ectodomain, with or without the furin cleavage site. (FIG. 43B) Western blot analysis of cells co-expressing Nanosota-5-Fc and either the prototypic or XBB.1.5 spike. The amounts of Nanosota-5-Fc-expressing plasmid used for co-transfection with the spike-expressing plasmid are indicated. The cleavage state of the cell-surface-expressed spike was detected by Western blot using anti-C9 antibodies targeting the C-terminal C9 tag of the spikes. (FIG. 43C) Pseudovirus entry into cells co-expressing human ACE2 and TMPRSS2.

FIGS. 44A-44C. Coronavirus entry pathways via viral receptor or Fc receptor (FcR). (FIG. 44A) SARS-CoV-2 entry through the viral receptor ACE2. (FIG. 44B) Coronavirus entry facilitated by RBD-targeting antibodies and FcR, a molecular mechanism previously identified for antibody-dependent enhancement (ADE) of coronavirus entry (see main text). (FIG. 44C) SARS-CoV-2 entry through the viral receptor ACE2, enhanced by non-RBD targeting nanobodies, a molecular mechanism identified in the current study.

FIG. 45. Comparison of the entry-enhancing epitope identified in the current study with the entry-enhancing human antibody epitope in the PDB. The PDB ID for the entry-enhancing human antibody epitope is 7DZX (shown in dark gray). The entry-enhancing epitope identified in the current study (shown in blue) partially overlaps with the entry-enhancing human antibody epitope.

FIGS. 46A-46B. Efficacy of Nanosota-5-Fc against live SARS-CoV-2 in mice. (FIG. 46A) Nanosota-5-Fc had no impact on prototypic SARS-CoV-2 in mice. It was administered at dosages of 2 or 10 mg/kg body weight, 4 hours post-challenge. Mice were challenged via intranasal inoculation with the prototypic SARS-CoV-2 (mouse-adapted prototypic variant M15, containing mutations in the RBD but not in the Nanosota-5 epitope). (FIG. 46B) Nanosota-5-Fc effectively neutralized different Omicron subvariants in mice. It was administered at a dosage of 10 mg/kg body weight, 4 hours post-challenge. Mice were challenged via intranasal inoculation with each of the indicated Omicron subvariants. For both (FIG. 46A) and (FIG. 46B), the (−) control groups received PBS buffer, and virus titers in the mouse lungs were measured on day 2 post-challenge. Comparisons of lung virus titers between the control and treatment groups were performed using an unpaired two-tailed Student's t-test. Error bars represent SEM. ***p<0.001. n.s. not significant.

FIGS. 47A-47C. Structure-guided in vitro evolution of nanobodies targeting new viral variants. (FIG. 47A) Structure of the prototypic SARS-CoV-2 RBD complexed with Nanosota-3A (PDB: 8G73). The core structure and the receptor-binding motif (RBM) of the RBD are shown in cyan and magenta, respectively. Two RBM residues that underwent mutations from prototypic SARS-CoV-2 to Omicron XBB.1.5 within the Nanosota-3-binding epitope are depicted as spheres. (FIG. 47B) Six nanobody residues adjacent to the two RBM residues that were selected for in vitro evolution. All eight residues are shown as sticks. The dotted line indicates a hydrogen bond, and double-arrowed lines indicate hydrophobic interactions. (FIG. 47C) Flow chart of the structure-guided in vitro evolution procedure.

FIGS. 48A-48B. Screening for Nanosota-3 variants that bind to XBB.1.5 spike with high affinity. (FIG. 48A) Summary of the mutations in Nanosota-3 selected through the structure-guided in vitro evolution process, enabling Nanosota-3 to overcome the two mutations in the XBB.1.5 RBD. (FIG. 48B) ELISA between Nanosota-3 variants and the XBB.1.5 spike ectodomain. His-tagged XBB.1.5 spike ectodomain was coated on ELISA plates, followed by the addition of HA- and His-double-tagged Nanosota-3 (Nanosota-3A, -3B, or one of the Nanosota-3C variants). Binding was detected using anti-HA-tag antibodies. Error bars represent SEM (n=3). A Student's two-tailed t-test was performed to analyze the statistical differences between the indicated groups, with results labeled above each bar. *** P<0.001.

FIGS. 49A-49B. Functional characterization of Nanosota-3's binding affinity for XBB.1.5 spike. (FIG. 49A) Binding interactions between Nanosota-3 variants and Omicron spikes were evaluated using ELISA. His-tagged Omicron spike ectodomain (from the BA.1 or XBB.1.5 subvariant) was coated on ELISA plates, followed by the addition of HA- and His-double-tagged nanobodies (Nanosota-3A, -3B, or -3C). Binding was detected using anti-HA-tag antibodies. Error bars represent SEM (n=3). (FIG. 49B) Binding kinetics between Nanosota-3 variants and Omicron RBDs were determined using surface plasmon resonance (SPR). His-tagged nanobodies (Nanosota-3A or -3C) were immobilized onto a CM5 chip through chemical crosslinking. His-tagged Omicron RBDs (from the BA.1 or XBB.1.5 subvariant) were then injected at various concentrations. The resulting data (red curves) were fitted to a 1:1 binding model (black curves) using Biacore Evaluation Software.

FIGS. 50A-50B. Functional characterization of Nanosota-3's neutralizing capability against XBB.1.5 entry. (FIG. 50A) Efficacy of Nanosota-3A and -3C in neutralizing Omicron pseudoviruses. Retroviruses pseudotyped with Omicron spike (from either the BA.1 or XBB.1.5 subvariant) were used to transduce human ACE2-expressing cells in the presence of Nanosota-3A-Fc or -3C-Fc at various concentrations. Entry efficiency was measured by the accompanying luciferase signal. The efficacy of Nanosota-3A-Fc or -3C-Fc against each pseudovirus type was expressed as the concentration capable of neutralizing pseudovirus entry by 50% (i.e., IC₅₀). Error bars represent SEM (n=3). Each experiment was repeated at least three times, with similar results obtained. (FIG. 50B) Efficacy of Nanosota-3C in neutralizing live XBB.1.5 virus in a mouse model. Nanosota-3C-Fc was administered at a dosage of 10 mg/kg body weight either 24 hours pre-challenge or 4 hours post-challenge. C57BL/6 mice were challenged via intranasal inoculation with XBB.1.5 virus. In the treatment group (n=5), mice received Nanosota-3C-Fc via intraperitoneal delivery. In the control group (n=5), mice were administered PBS buffer. Lung virus titers on day 2 post-challenge were measured. Comparisons of lung virus titers between the control and treatment groups were performed using an unpaired two-tailed Student's t-test. Error bars represent SEM (n=5). *** p<0.001.

FIGS. 51A-51C. Structure of XBB.1.5 spike complexed with Nanosota-3C. (FIG. 51A) The cryo-EM structure of the XBB.1.5 spike ectodomain complexed with Nanosota-3C was determined. XBB.1.5 spike is shown in gray, with the XBB.1.5 RBD in cyan. Among the three copies of the XBB.1.5 RBD, two are in the standing-up conformation, and the third is in the lying-down conformation. Nanosota-3C binds to all three copies of the RBD. (FIG. 51B) Superposition of the structures of the prototypic RBD/Nanosota-3A complex and the XBB.1.5 RBD/Nanosota-3C complex in the loop regions where mutations occurred in the XBB.1.5 RBD and Nanosota-3C. (FIG. 51C) Detailed structure of the XBB.1.5 RBD/Nanosota-3C interface. Mutations in the XBB.1.5 RBD and Nanosota-3C are shown as sticks. The dotted line indicates a hydrogen bond, and double-arrowed lines indicate hydrophobic interactions. *To clarify the representation in panel C, we have maintained the labeling of residues 484 and 490 from the prototypic RBD, which correspond to residues 480 and 486 in the XBB.1.5 RBD, respectively. This labeling choice was made to avoid confusion and ensure consistency in our comparisons.

FIG. 52. Flow chart of cryo-EM image processing and 3D reconstruction for the XBB.1.5 spike/Nanosota-3C complex. Representative raw cryo-EM images and 2D classes are shown. 3D refinements using all the particles from the good 3D classes produced a 3.19 Å map. Further local refinement improved the density for the bound nanobody. The angular distribution plot, final maps, half-map FSC curves, and accompanying local resolution illustrations are enclosed in the dashed black boxes.

FIGS. 53A-53C. Cryo-EM densities of the XBB.1.5 spike/Nanosota-3C interface. The XBB.1.5 chain (FIG. 53B) is shown in magenta, and the Nanosota-3C chain (FIG. 53A) is shown in blue. An interface is shown (FIG. 53C). All residues are represented as gray sticks.

FIGS. 54A-54B. Contribution of individual mutations in Nanosota-3C to the protein's target-binding affinity. (FIG. 54A) The binding interactions between the XBB.1.5 spike ectodomain and Nanosota-3A containing one of the six mutations evolved in Nanosota-3C were evaluated using ELISA at different nanobody concentrations. Nanosota-3A and -3C were used as controls. Error bars represent SEM (n=3). (FIG. 54B) ELISA data at the highest concentration of the nanobodies. A Student's two-tailed t-test was performed to analyze the statistical differences between Nanosota-3A and each of the other nanobodies; the results are indicated above each bar. *** p<0.001. * p<0.05. n.s. not statistically significant.

FIG. 55. Thermal stabilities of Nanosota-3A-Fc, -3B-Fc, and -3C-Fc. The thermal stabilities of the three nanobody variants were measured using a differential scanning fluorimetry (DSF) assay. Protein stability was assessed by monitoring the fluorescence signal during protein denaturation at increasing temperatures. The negative first derivative of the fluorescence signal was plotted against temperature, with the peak indicating the melting temperature (Tm). RFU: relative fluorescence units.

### DETAILED DESCRIPTION

Single-domain antibodies (sdAbs; also referenced herein as nanobodies) are much smaller than traditional antibodies (e.g., about 15 kDa vs 150 kDa), which provides certain advantages as therapeutics. For example, sdAbs are highly stable, expressed at high yields, easy to store and transport, highly cost effective, capable of accessing/recognizing less exposed epitopes, and are able to penetrate tissues and barriers in the human body efficiently. Moreover, like traditional antibodies, sdAbs can bind antigens with high affinity and specificity. While sdAbs can be cleared by the kidneys due to their small size, the pharmacokinetics and/or effector function of a sdAb can be modulated by constructing sdAb-Fc proteins.

As described herein, sdAbs were generated, which showed superior therapeutic properties. For example, three exemplary anti-SARS-CoV-2 sdAb clones, named Nanosota-2, -3 and -4, were identified. Nanosota-2 inhibits the infection of prototypic SARS-CoV-2 in vitro at low IC₅₀ (2 pM or 0.16 ng/ml against live virus; 6.2 pM or 0.5 ng/ml against pseudovirus) and in mice at low dosage (4 mg/Kg) or late administration time (18 hours post-challenge); these potency metrics are among the best of known anti-SARS-CoV-2 entry inhibitors. Nanosota-3 potently inhibits the infection of the omicron variant in vitro and in mice. Nanosota-4 uniquely inhibits both SARS-CoV-1 and SARS-CoV-2. Cryo-EM data (also see Example 1) revealed that the three sdAbs bind to functionally critical and non-overlapping regions in the spike protein. The combined antiviral spectrum of the three sdAbs covers SARS-CoV-2 or its major variants (e.g., alpha, delta, omicron), and SARS-CoV-1. Given their cost-effectiveness, ease to adapt to new viral variants through phage display and potential to being administered as inhalers, the Nanosota series are powerful therapeutic tools against coronavirus pandemics.

On the other hand, to date there is a lack of an overview of the dominant epitopes of the SARS-CoV-2 spike protein or other viral glycoproteins. Here using sdAbs (single-domain antibodies) isolated from a spike-immunized alpaca, all of the dominant epitopes on SARS-CoV-2 spike (e.g., also see Example 2) were mapped. Cryo-EM data in Example 2 revealed two neutralizing epitopes on the receptor-binding domain (RBD) and two epitopes outside the RBD that induce antibody-dependent enhancement (ADE) of viral entry for prototypic SARS-CoV-2. Different from previously known ADE that depends on Fc receptors, the ADE observed in Example 2 depends on the viral receptor, not Fc receptors. At lower virus titers, ADE becomes more prominent (viral infection in cultured cells increased by -five fold). Depletion assay confirmed the functions of RBD epitopes and non-RBD epitopes.

However, in vitro studies in Example 3 suggested that one of the conserved non-RBD epitope (bound by Nanosota-5, also see Example 3) appears to be a unique dual-role epitope that shows opposing effects on viral entry among different variants, which enhances viral entry for pre-Omicron variants such as prototypic SARS-CoV-2, Alpha, or Delta, but neutralizes viral entry for Omicron variant.

The terms “nanobody” and “single-domain antibody” are used interchangeably herein. As used herein, the term “nanobody” or “single-domain antibody” refers to a single monomeric variable antibody domain comprising three complementarity-determining regions (CDRs including CDR1, CDR2, CDR3) and four framework regions (FRs including FR1, FR2, FR3, FR4), such as a VHH, a humanized VHH or a camelized VH (such as a camelized human VH) or generally a sequence optimized VHH (such as e.g., optimized for chemical stability and/or solubility, maximum overlap with known human framework regions and maximum expression), which is capable of binding to a specific antigen. The terms “nanobody” and “single-domain antibody” are used herein in its broadest form to include variants that may have various amino acid substitutions (e.g., conservative substitutions) and also functional “fragment” or “antigen binding fragment” of the sdAb as long as the fragment retains binding to the specific antigen.

As used herein, the term “anti-SARS-CoV-2 binder protein” refers to a protein having binding affinity for a SARS-CoV-2 viral antigen (e.g., SARS-CoV-2 spike protein). Such a protein may comprise or consist of a sdAb as described herein, or an antigen binding fragment thereof. In certain embodiments, an anti-SARS-CoV2 binder protein comprises a sdAb as described herein. In certain embodiments, an anti-SARS-CoV2 binder protein consists of a sdAb as described herein. In certain embodiments, an anti-SARS-CoV2 binder protein comprises an antigen binding fragment of a sdAb as described herein. In certain embodiments, an anti-SARS-CoV2 binder protein consists of an antigen binding fragment of a sdAb as described herein.

Accordingly, certain embodiments of the invention provide an anti-SARS-CoV-2 binder protein (e.g., targeting a SARS-CoV-2 spike protein) as described herein. In certain embodiments, the anti-SARS-CoV-2 binder protein comprises a sdAb, and optionally, one or more polypeptide tags, wherein the sdAb is operably linked to the one or more polypeptide tags (e.g., a monomeric sdAb with an optional His tag and/or HA tag). In certain embodiments, the anti-SARS-CoV-2 binder protein comprises two sdAb-Fc fusion proteins that are dimerized via the Fc domain. In certain embodiments, an anti-SARS-CoV-2 binder protein comprises or consists of one or more polypeptide as described in Examples 1-3, or in Table A, Table 1 (e.g., Nanosota-2), Table 2 (e.g., Nanosota-3), Table 3 (e.g., Nanosota-4), Table 4 (e.g., Nanosota-5 from Class 4), Table 5 (e.g., Nanosota-6), or Table 6 (Nanosota-7).

In some embodiments, the binder protein (e.g., an anti-SARS-CoV-2 sdAb or sdAb-Fc fusion protein(s)) comprises: (1) one or more complementarity determining region (CDR) sequences as described herein; and/or (2) a heavy chain variable region (VHH) sequence as described herein (e.g., as described in Table A, or Tables 1-6 below).

In some embodiments, the binder protein comprises one, two, or three CDRs selected from one table as described herein (e.g., from one table of Tables 1-6 below). In some embodiments, the binder protein comprises two, or three CDRs selected from one table as described herein (e.g., from one table of Tables 1-6 below). In some embodiments, the binder protein comprises three CDRs (CDR1, CDR2, and CDR3) selected from one table as described herein (e.g., from one table of Tables 1-6 below). Accordingly, certain embodiments provide a binder protein comprising:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of a CDR1 as described herein (e.g., a table described herein, such as in one of Tables 1-6);
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of a CDR2 as described herein (e.g., a table described herein, such as in one of Tables 1-6); and/or
(c) a CDR3 comprising an amino acid sequence having at least 75% (e.g., 76%, 77%, 78%7, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of a CDR3 as described herein (e.g., a table described herein, such as in one of Tables 1-6).

In some embodiments, the binder protein comprises one, two, or three CDRs of a clone as described herein (e.g., a clone as described in Table A, or Tables 1-6 below). In some embodiments, the binder protein comprises two, or three CDRs of a clone as described herein (e.g., a clone as described in Table A, or Tables 1-6 below). In some embodiments, the binder protein comprises three CDRs (CDR1, CDR2, and CDR3) of a clone as described herein (e.g., a clone as described in Table A, or Tables 1-6 below). Accordingly, in some embodiments, the binder protein comprises:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of the CDR1 of a clone described herein;
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of the CDR2 of a clone described herein; and/or
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of the CDR3 of a clone described herein.

For example, in some embodiments, an anti-SARS-CoV-2 binder protein comprises one, two, or three CDRs of a clone as described in Table A. In certain embodiments, the clone is nanosota-2A. In certain embodiments, the clone is nanosota-3A. In certain embodiments, the clone is nanosota-3B, or nanosota-3C. In certain embodiments, the clone is nanosota-4A.

In some embodiments, an anti-SARS-CoV-2 binder protein comprises one, two, or three CDRs of a clone as described in Table 1. In certain embodiments, the clone is H6 (i.e., nanosota-2). In certain embodiments, the clone is D4.

In some embodiments, an anti-SARS-CoV-2 binder protein comprises one, two, or three CDRs of a clone as described in Table 2. In certain embodiments, the clone is A3 (i.e., nanosota-3). In certain embodiments, the clone is B3. In certain embodiments, the clone is E2. In certain embodiments, the clone is D3. In certain embodiments, the clone is E5. In certain embodiments, the clone is A12.

In some embodiments, an anti-SARS-CoV-2 binder protein comprises one, two, or three CDRs of a clone as described in Table 3. In certain embodiments, the clone is E8 (i.e., nanosota-4A). In certain embodiments, the clone is A6. In certain embodiments, the clone is A7. In certain embodiments, the clone is C9. In certain embodiments, the clone is D5. In certain embodiments, the clone is F10. In certain embodiments, the clone is F11. In certain embodiments, the clone is G4. In certain embodiments, the clone is D7. In certain embodiments, the clone is G8. In certain embodiments, the clone is G3. In certain embodiments, the clone is F9. In certain embodiments, the clone is C10. In certain embodiments, the clone is C12. In certain embodiments, the clone is H11. In certain embodiments, the clone is C8. In certain embodiments, the clone is F2. In certain embodiments, the clone is E12.

In some embodiments, an anti-SARS-CoV-2 binder protein comprises one, two, or three CDRs of a clone as described in Table 4. In certain embodiments, the clone is A2. In certain embodiments, the clone is E6. In certain embodiments, the clone is F6. In certain embodiments, the clone is E10 (i.e., nanosota-5). In certain embodiments, the clone is D6. In certain embodiments, the clone is C2. In certain embodiments, the clone is D1. In certain embodiments, the clone is E1. In certain embodiments, the clone is E4. In certain embodiments, the clone is H4. In certain embodiments, the clone is A5. In certain embodiments, the clone is B1. In certain embodiments, the clone is B6. In certain embodiments, the clone is B7. In certain embodiments, the clone is F1. In certain embodiments, the clone is F3. In certain embodiments, the clone is F4. In certain embodiments, the clone is H5.

In some embodiments, an anti-SARS-CoV-2 binder protein comprises one, two, or three CDRs of a clone as described in Table 5. In certain embodiments, the clone is H1 (i.e., nanosota-6). In certain embodiments, the clone is H3. In certain embodiments, the clone is D2.

In some embodiments, an anti-SARS-CoV-2 binder protein comprises one, two, or three CDRs of a clone as described in Table 6. In certain embodiments, the clone is B6. In certain embodiments, the clone is C3 (i.e., nanosota-7).

In certain embodiments, an anti-SARS-CoV-2 binder protein (e.g., Class 1 such as Nanosota-2) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of FNFETSTV (SEQ ID NO: 2);
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of CINKGYEDTN (SEQ ID NO: 3); and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of AAHNEPYFCDYSGRFRWNEYSY (SEQ ID NO: 4).

In certain embodiments, an anti-SARS-CoV-2 binder protein (e.g., Class 2) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SIFSPNTM (SEQ ID NO: 8) or STSASNSM (SEQ ID NO: 34);
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of VISSIASTQ (SEQ ID NO: 9), FISSIASTS (SEQ ID NO: 157), or TAANGDIRS (SEQ ID NO: 35) or a CDR2 comprising an amino acid sequence of (V/F)ISSIAST(Q/S) (SEQ ID NO: 159); and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of YAVDKSQDY (SEQ ID NO: 10) or YSVDSYRDY (SEQ ID NO: 36), or a CDR3 comprising an amino acid sequence of Y(A/S)VD(K/S)(S/Y)(Q/R)DY (SEQ ID NO: 148).

In certain embodiments, an anti-SARS-CoV-2 binder protein (e.g., Class 3) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of FTLDYYAI (SEQ ID NO: 14) or FTVNSHAI (SEQ ID NO: 70), or a CDR1 comprising the amino acid sequence of F(T/I/A)LD(Y/F/H)YAI (SEQ ID NO: 149);
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of CISSSGGRTN (SEQ ID NO: 15), or a CDR2 comprising the amino acid sequence of CIS(S/I)SGG(R/S)TN (SEQ ID NO: 150); and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of AAWEASRWYCPLQFSADFSS (SEQ ID NO: 16), or a CDR3 comprising the amino acid sequence of AAWE(A/G)S(R/T/S)(W/R/E)YCPLQ(F/T/Y)SADF(S/V/A/D)S (SEQ ID NO: 151).

In certain embodiments, an anti-SARS-CoV-2 binder protein (e.g., Class 3) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 14, 47, 65, 70, or 76;
(b) a CDR2 comprising an amino acid sequence having at least 75% (e.g., 76%, 77%, 78%7, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 15, 66, or 77; and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 16, 48, 67, or 73.

In certain embodiments, an anti-SARS-CoV-2 binder protein (e.g., Class 4 including Nanosota-5) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SIFRFEAV (SEQ ID NO: 90), SIFRMDVV (SEQ ID NO: 95), SIFRMELM (SEQ ID NO: 102), or SIFR(F/M)(E/D)(A/V/L)(V/M) (SEQ ID NO: 160);
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of TVARDGTTN (SEQ ID NO: 91), SITRSGSTN (SEQ ID NO: 96), TINRCGSTN (SEQ ID NO: 103), TITRSGSTN (SEQ ID NO: 163) or (S/T)(V/I)(A/T/N)R(D/S/C)G(T/S)TN (SEQ ID NO: 161); and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of NARWWTNF (SEQ ID NO: 92), HARTWTSY (SEQ ID NO: 97), HARTWTSS (SEQ ID NO: 104), or (H/N)AR(T/W)WT(S/N)(S/Y/F) (SEQ ID NO: 162).

In certain embodiments, an anti-SARS-CoV-2 binder protein (e.g., Class 5 such as Nanosota-6) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 135;
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 136; and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 137.

In certain embodiments, an anti-SARS-CoV-2 binder protein (e.g., Class 6) comprises one or more CDRs selected from the group consisting of:
(a) a CDR1 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 144;
(b) a CDR2 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 145; and
(c) a CDR3 comprising an amino acid sequence having at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to an amino acid sequence of SEQ ID NO: 146.

In certain embodiments, the anti-SARS-CoV-2 binder protein as described herein comprises one or more CDRs selected from the group consisting of:
(a) a CDR1 comprising the amino acid sequence of SEQ ID NO: 2;
(b) a CDR2 comprising the amino acid sequence of SEQ ID NO: 3; and
(c) a CDR3 comprising the amino acid sequence of SEQ ID NO: 4.

In certain embodiments, the anti-SARS-CoV-2 binder protein as described herein comprises one or more CDRs selected from the group consisting of:
(a) a CDR1 comprising the amino acid sequence of SEQ ID NO: 8;
(b) a CDR2 comprising the amino acid sequence of SEQ ID NO: 9 or 157; and
(c) a CDR3 comprising the amino acid sequence of SEQ ID NO: 10; or
(a) a CDR1 comprising the amino acid sequence of SEQ ID NO: 34;
(b) a CDR2 comprising the amino acid sequence of SEQ ID NO: 35; and
(c) a CDR3 comprising the amino acid sequence of SEQ ID NO: 36.

In certain embodiments, the anti-SARS-CoV-2 binder protein as described herein comprises one or more CDRs selected from the group consisting of:
(a) a CDR1 comprising the amino acid sequence of SEQ ID NO: 8;
(b) a CDR2 comprising the amino acid sequence of (V/F)ISSIAST(Q/S) (SEQ ID NO: 159); and
(c) a CDR3 comprising the amino acid sequence of SEQ ID NO: 10.

In certain embodiments, the anti-SARS-CoV-2 binder protein as described herein comprises one or more CDRs selected from the group consisting of:
(a) a CDR1 comprising the amino acid sequence of SEQ ID NO: 8;
(b) a CDR2 comprising the amino acid sequence of FISSIASTS (SEQ ID NO: 157); and
(c) a CDR3 comprising the amino acid sequence of SEQ ID NO: 10.

In certain embodiments, the anti-SARS-CoV-2 binder protein as described herein comprises one or more CDRs selected from the group consisting of:
(a) a CDR1 comprising the amino acid sequence of SEQ ID NO: 14;
(b) a CDR2 comprising the amino acid sequence of SEQ ID NO: 15; and
(c) a CDR3 comprising the amino acid sequence of SEQ ID NO: 16.

In some embodiments, an anti-SARS-CoV-2 binder protein comprises two or three CDRs as described above (e.g., each CDR is selected from one of (a)-(c)).

For example, in certain embodiments, the anti-SARS-CoV-2 binder protein as described herein comprises:
(a) a CDR1 comprising the amino acid sequence of SEQ ID NO: 2;
(b) a CDR2 comprising the amino acid sequence of SEQ ID NO: 3; and
(c) a CDR3 comprising the amino acid sequence of SEQ ID NO: 4 .

In certain embodiments, the anti-SARS-CoV-2 binder protein as described herein comprises:
(a) a CDR1 comprising the amino acid sequence of SEQ ID NO: 8;
(b) a CDR2 comprising the amino acid sequence of SEQ ID NO: 9; and
(c) a CDR3 comprising the amino acid sequence of SEQ ID NO: 10.

In certain embodiments, the anti-SARS-CoV-2 binder protein as described herein comprises:
(a) a CDR1 comprising the amino acid sequence of SEQ ID NO: 14;
(b) a CDR2 comprising the amino acid sequence of SEQ ID NO: 15; and
(c) a CDR3 comprising the amino acid sequence of SEQ ID NO: 16.

In some embodiments, the binder protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of VHH sequences as described herein (e.g., as described in any of Tables 1-6 below).

In some embodiments, an anti-SARS-CoV-2 binder protein comprises a sequence (e.g., a variable domain of a heavy-chain only antibody (VHH)) derived from any of the following sdAbs described herein: Nanosota-2A, Nanosota-3A, Nanosota-3B, Nanosota-3C, Nanosota-4A, and Nanosota-5. The amino acid sequences of these anti-SARS-CoV-2 sdAb clones are set forth in Table A, Table 1, Table 2, Table 3, or Table 4 below. In certain embodiments, an anti-SARS-CoV-2 binder protein comprises a sequence as described in any of the embodiments provided herein.

In certain embodiments, an anti-SARS-CoV-2 binder protein described herein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 1)
QVQLQESGGGAVQPGGSLGLSCTASGFNFETSTVGWFRQAPGKENEGVSC
INKGYEDTNYADSVKGRFTISRDAAKNTVYLQMDSLQPEDTATYYCAAHN
EPYFCDYSGRFRWNEYSYYGQGTQVTVSS;
(b)
(SEQ ID NO: 7)
QVQLQESGGGLVQAGGSLRLSCAASGSIFSPNTMGWFRQALGKQREMVAV
ISSIASTQYANFVKGRFTITRDNTKNTVHLQMNSLIPEDTAVYYCYAVDK
SQDYWGQGTQVTVSS;
(c)
(SEQ ID NO: 156)
QVQLQESGGGLVQAGGSLRLSCAASGSIFSPNTMGWFRQALGKQREMVAF
ISSIASTSYANFVKGRFTITRDNTKNTVHLQMNSLIPEDTAVYYCYAVDK
SQDYWGQGTQVTVSS;
(d)
(SEQ ID NO: 167)
QVQLQESGGGLVQAGGSLRLSCAASGSIFSPNTMGWFRQALGKQREGVAF
ISSIASTSYWLPVKGRFTITRDNTKNTVHLQMNSLIPEDTAVYYCYAVDK
SQDYWGQGTQVTVSS;
(e)
(SEQ ID NO: 168)
QVQLQESGGGLVQAGGSLRLSCAASGSIFSPNTMGWFRQALGKQREGVAF
ISSIASTSYNWYVKGRFTITRDNTKNTVHLQMNSLIPEDTAVYYCYAVDK
SQDYWGQGTQVTVSS;
(f)
(SEQ ID NO: 169)
QVQLQESGGGLVQAGGSLRLSCAASGSIFSPNTMGWFRQALGKQREGVAF
ISSIASTSYNFFVKGRFTITRDNTKNTVHLQMNSLIPEDTAVYYCYAVDK
SQDYWGQGTQVTVSS;
(g)
(SEQ ID NO: 170)
QVQLQESGGGLVQAGGSLRLSCAASGSIFSPNTMGWFRQALGKQREGVAF
ISSIASTSYGYGVKGRFTITRDNTKNTVHLQMNSLIPEDTAVYYCYAVDK
SQDYWGQGTQVTVSS;
(h)
(SEQ ID NO: 13)
QVQLQESGGGLVQPGGSLRLSCAASGFTLDYYAIGWFRQAPGKEREGVSC
ISSSGGRTNYADSVKGRFTISRDNTKNTVYLQMNSLKPEDTAVYYCAAWE
ASRWYCPLQFSADFSSWGQGTQVTVSS;
and
(i)
(SEQ ID NO: 101)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSSWGRGTQVTVSS.

In some embodiments, an anti-SARS-CoV-2 binder protein comprises an amino acid sequence of any one of SEQ ID NOs: 1, 7, 156, 167, 168, 169, 170, and 13. In some embodiments, an anti-SARS-CoV-2 binder protein consists of the amino acid sequence of any one of SEQ ID NOs: 1, 7, 156, 167, 168, 169, 170, and 13.

In some embodiments, an anti-SARS-CoV-2 binder protein comprises an amino acid sequence of any one of SEQ ID NOs: 1, 7, 101, 164, 156, 167, 168, 169, 170, and 13. In some embodiments, an anti-SARS-CoV-2 binder protein consists of the amino acid sequence of any one of SEQ ID NOs: 1, 7, 156, 167, 168, 169, 170, 101, 164, and 13.

### Binder Class 1 (Including Nanosota-2A, Also Referred to as Nanosota-2 or Clone H6)

### Clones H6 and D4

In some embodiments, an anti-SARS-CoV-2 binder protein comprises a CDR1 comprising the amino acid sequence of SEQ ID NO: 2, a CDR2 comprising the amino acid sequence of SEQ ID NO: 3, and a CDR3 comprising the amino acid sequence of SEQ ID NO: 4. In some embodiments, an anti-SARS-CoV-2 binder protein comprises CDRs 1-3 consisting of the amino acid sequences of SEQ ID NOs: 2, 3, and 4, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 1 or 31. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 1 or 31; and 2) comprises SEQ ID NO: 2, SEQ ID NO: 3 and SEQ ID NO: 4. In some embodiments, an anti-SARS-CoV-2 binder protein comprises an amino acid sequence of SEQ ID NO: 1. In some embodiments, an anti-SARS-CoV-2 binder protein consists of the amino acid sequence of SEQ ID NO: 1. In some embodiments, an anti-SARS-CoV-2 binder protein comprises an amino acid sequence of SEQ ID NO: 31. In some embodiments, an anti-SARS-CoV-2 binder protein consists of the amino acid sequence of SEQ ID NO: 31.

### Binder Class 2 (Including Nanosota-3A, Also Referred to as Nanosota-3 or Clone A3)

In some embodiments, an anti-SARS-CoV-2 binder protein comprises a CDR1 as described herein (e.g., in Table 2), a CDR2 as described herein (e.g., in Table 2) or a CDR2 comprising the amino acid sequence of (V/F)ISSIAST(Q/S) (SEQ ID NO: 159), and a CDR3 comprising the amino acid sequence of Y(A/S)VD(K/S)(S/Y)(Q/R)DY (SEQ ID NO: 148). clones A3, engineered A3, E2, D3, E5 and A12

In some embodiments, an anti-SARS-CoV-2 binder protein comprises a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In some embodiments, an anti-SARS-CoV-2 binder protein comprises CDRs1-3 consisting of the amino acid sequences of SEQ ID NOs: 8, 9, and 10, respectively.

In some embodiments, an anti-SARS-CoV-2 binder protein comprises a CDR1 comprising the amino acid sequence of SEQ ID NO: 8, a CDR2 comprising the amino acid sequence of SEQ ID NO: 157, and a CDR3 comprising the amino acid sequence of SEQ ID NO: 10. In some embodiments, an anti-SARS-CoV-2 binder protein comprises CDRs1-3 consisting of the amino acid sequences of SEQ ID NOs: 8, 157, and 10, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 7 or 44. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 7 or 44; and 2) comprises SEQ ID NO: 8, SEQ ID NO: 9 and SEQ ID NO: 10. In some embodiments, an anti-SARS-CoV-2 binder protein comprises an amino acid sequence of SEQ ID NO: 7. In some embodiments, an anti-SARS-CoV-2 binder protein consists of the amino acid sequence of SEQ ID NO: 7. In some embodiments, an anti-SARS-CoV-2 binder protein comprises an amino acid sequence of SEQ ID NO: 44. In some embodiments, an anti-SARS-CoV-2 binder protein consists of the amino acid sequence of SEQ ID NO: 44.

In some embodiments, an anti-SARS-CoV-2 binder protein (e.g., Nanosota-3B) comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 156. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 156; and 2) comprises SEQ ID NO: 8, SEQ ID NO: 157 and SEQ ID NO: 10. In some embodiments, an anti-SARS-CoV-2 binder protein comprises an amino acid sequence of SEQ ID NO: 156. In some embodiments, an anti-SARS-CoV-2 binder protein consists of the amino acid sequence of SEQ ID NO: 156.

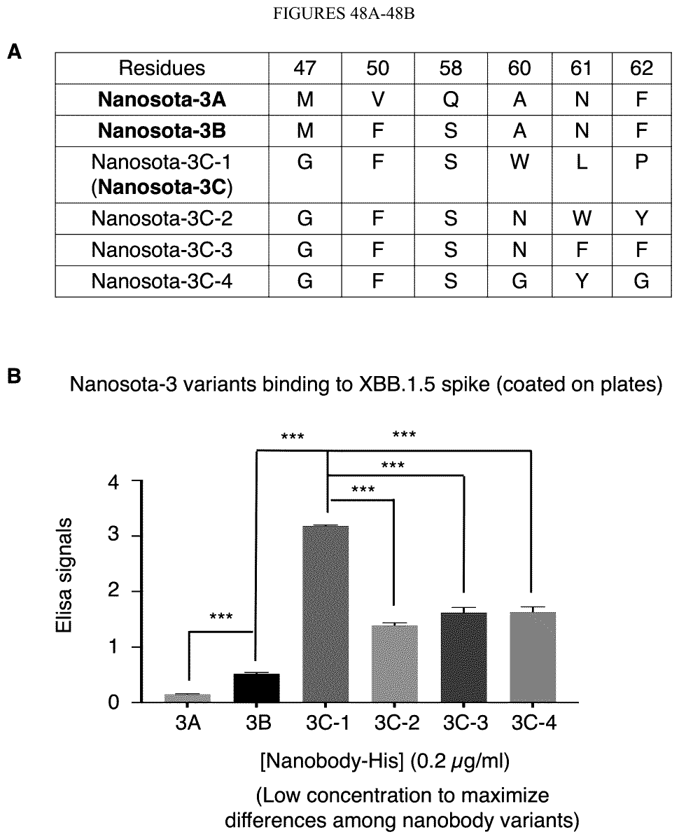

In some embodiments, an anti-SARS-CoV-2 binder protein (e.g., Nanosota-3A, SEQ ID NO: 7) comprises one or more mutations shown in FIG. 48A. In some embodiments, an anti-SARS-CoV-2 binder protein (e.g., SEQ ID NO: 7) comprises one or more mutations selected from the group consisting of M47G, V50F, Q58S, A60W, N61L and F62P. In some embodiments, an anti-SARS-CoV-2 binder protein (e.g., SEQ ID NO: 7) comprises A60W.

In some embodiments, an anti-SARS-CoV-2 binder protein (e.g., Nanosota-3B, SEQ ID NO: 156) comprises one or more mutations shown in FIG. 48A. In some embodiments, an anti-SARS-CoV-2 binder protein (e.g., SEQ ID NO: 156) comprises one or more mutations selected from the group consisting of M47G, A60W, N61L and F62P. In some embodiments, an anti-SARS-CoV-2 binder protein (e.g., SEQ ID NO: 156) comprises A60W.

In some embodiments, an anti-SARS-CoV-2 binder protein (e.g., Nanosota-3C) comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 167, 168, 169, and 170. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 167, 168, 169, and 170; and 2) comprises SEQ ID NO: 8, SEQ ID NO: 157 and SEQ ID NO: 10. In some embodiments, an anti-SARS-CoV-2 binder protein comprises an amino acid sequence of any one of SEQ ID NOs: 167, 168, 169, and 170. In some embodiments, an anti-SARS-CoV-2 binder protein consists of the amino acid sequence of any one of SEQ ID NOs: 167, 168, 169, and 170.

In certain embodiments, a binder protein of the invention (e.g., comprising CDRs or VHH sequence of nanosota-3B, nanosota-3B-Fc, nanosota-3C, or nanosota-3C-Fc) inhibits the viral entry of SARS-CoV-2 Omicron variant (e.g., BA.1, or XBB.1.5) into a cell by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

In certain embodiments, a binder protein of the invention (e.g., comprising CDRs or VHH sequence of nanosota-3B, nanosota-3B-Fc, nanosota-3C, or nanosota-3C-Fc) inhibits the viral entry of SARS-CoV-2 Omicron variant (e.g., BA.1, or XBB.1.5) into a cell with a IC₅₀ potency of about 1-600 ng/mL, 2-300 ng/mL, 3-200 ng/mL, 10-100 ng/mL, 15-35 ng/mL or 16-33 ng/mL. In certain embodiments, a binder protein of the invention (e.g., comprising CDRs or VHH sequence of nanosota-3B, nanosota-3B-Fc, nanosota-3C, or nanosota-3C-Fc) inhibits the viral entry of SARS-CoV-2 Omicron variant (e.g., BA.1, or XBB.1.5) into a cell with a ICso potency of about 1, 5, 10, 11, 12, 13, 14, 15, 16, 20, 21, 22, 23, 24, 25, 30, 31, 32, 33, 34, 35, 40, 50, 60, 70, 80, 90, 100, 110, 120, 130, 140, 150, 160, 180, or 200 ng/mL.

### Clone B3

In some embodiments, an anti-SARS-CoV-2 binder protein comprises a CDR1 comprising the amino acid sequence of SEQ ID NO: 34, a CDR2 comprising the amino acid sequence of SEQ ID NO: 35, and a CDR3 comprising the amino acid sequence of SEQ ID NO: 36. In some embodiments, an anti-SARS-CoV-2 binder protein comprises CDRs1-3 consisting of the amino acid sequences of SEQ ID NOs: 34, 35, and 36, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 33. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 33; and 2) comprises SEQ ID NO: 34, SEQ ID NO: 35 and SEQ ID NO: 36. In some embodiments, an anti-SARS-CoV-2 binder protein comprises an amino acid sequence of SEQ ID NO: 33. In some embodiments, an anti-SARS-CoV-2 binder protein consists of the amino acid sequence of SEQ ID NO: 33.

### Binder Class 3 (Including Nanosota-4A, Also Referred to as Nanosota-4 or Clone E8)

In some embodiments, an anti-SARS-CoV-2 binder protein comprises a CDR1 comprising the amino acid sequence of F(T/I/A)LD(Y/F/H)YAI (SEQ ID NO: 149) or a CDR1 comprising an amino acid sequence having at least 75% sequence identity to an amino acid sequence of FTVNSHAI (SEQ ID NO: 70), a CDR2 comprising an amino acid sequence having at least 75% sequence identity to an amino acid sequence of CISSSGGRTN (SEQ ID NO: 15), and a CDR3 comprising an amino acid sequence having at least 75% sequence identity to an amino acid sequence of AAWEASRWYCPLQFSADFSS (SEQ ID NO: 16).

In some embodiments, an anti-SARS-CoV-2 binder protein comprises a CDR1 comprising the amino acid sequence of F(T/I/A)LD(Y/F/H)YAI (SEQ ID NO: 149) or FTVNSHAI (SEQ ID NO: 70), a CDR2 as described herein (e.g., in Table 3), and a CDR3 as described herein (e.g., in Table 3).

In some embodiments, an anti-SARS-CoV-2 binder protein comprises a CDR1 comprising the amino acid sequence of SEQ ID NO: 14, 47, 65, 70, or 76, a CDR2 as described herein (e.g., in Table 3), and a CDR3 as described herein (e.g., in Table 3).

In some embodiments, an anti-SARS-CoV-2 binder protein comprises a CDR1 comprising an amino acid sequence having at least 75% sequence identity to an amino acid sequence of FTLDYYAI (SEQ ID NO: 14) or FTVNSHAI (SEQ ID NO: 70), a CDR2 comprising the amino acid sequence of CIS(S/I)SGG(R/S)TN (SEQ ID NO: 150), and a CDR3 comprising an amino acid sequence having at least 75% sequence identity to an amino acid sequence of AAWEASRWYCPLQFSADFSS (SEQ ID NO: 16).

In some embodiments, an anti-SARS-CoV-2 binder protein comprises a CDR1 as described herein (e.g., in Table 3), a CDR2 comprising the amino acid sequence of CIS(S/I)SGG(R/S)TN (SEQ ID NO: 150), and a CDR3 as described herein (e.g., in Table 3).

In some embodiments, an anti-SARS-CoV-2 binder protein comprises a CDR1 as described herein (e.g., in Table 3), a CDR2 comprising the amino acid sequence of SEQ ID NO: 15, 66, or 77, and a CDR3 as described herein (e.g., in Table 3).

In some embodiments, an anti-SARS-CoV-2 binder protein comprises a CDR1 comprising an amino acid sequence having at least 75% sequence identity to an amino acid sequence of FTLDYYAI (SEQ ID NO: 14) or FTVNSHAI (SEQ ID NO: 70), a CDR2 comprising an amino acid sequence having at least 75% sequence identity to an amino acid sequence of CISSSGGRTN (SEQ ID NO: 15), and a CDR3 comprising the amino acid sequence of AAWE(A/G)S(R/T/S)(W/R/E)YCPLQ(F/T/Y)SADF(S/V/A/D)S (SEQ ID NO: 151).

In some embodiments, an anti-SARS-CoV-2 binder protein comprises a CDR1 as described herein (e.g., in Table 3), a CDR2 as described herein (e.g., in Table 3), and a CDR3 comprising the amino acid sequence of AAWE(A/G)S(R/T/S)(W/R/E)YCPLQ(F/T/Y)SADF(S/V/A/D)S (SEQ ID NO: 151).

In some embodiments, an anti-SARS-CoV-2 binder protein comprises a CDR1 as described herein (e.g., in Table 3), a CDR2 as described herein (e.g., in Table 3), and a CDR3 comprising the amino acid sequence of SEQ ID NO: 16, 48, 67, or 73.

In some embodiments, an anti-SARS-CoV-2 binder protein comprises a CDR1 comprising the amino acid sequence of F(T/I/A)LD(Y/F/H)YAI (SEQ ID NO: 149) or a CDR1 comprising an amino acid sequence having at least 75% sequence identity to an amino acid sequence of FTVNSHAI (SEQ ID NO: 70), a CDR2 comprising the amino acid sequence of CIS(S/I)SGG(R/S)TN (SEQ ID NO: 150), and a CDR3 comprising the amino acid sequence of AAWE(A/G)S(R/T/S)(W/R/E)YCPLQ(F/T/Y)SADF(S/V/A/D)S (SEQ ID NO: 151).

In some embodiments, an anti-SARS-CoV-2 binder protein comprises a CDR1 comprising the amino acid sequence of SEQ ID NO: 14, 47, 65, 70, or 76; a CDR2 comprising the amino acid sequence of SEQ ID NO: 15, 66, or 77; and a CDR3 comprising the amino acid sequence of SEQ ID NO: 16, 48, 67, or 73.

### Clone E8

In some embodiments, an anti-SARS-CoV-2 binder protein comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16. In some embodiments, an anti-SARS-CoV-2 binder protein comprises CDRs1-3 consisting of the amino acid sequences of SEQ ID NOs: 14, 15, and 16, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 13. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 13; and 2) comprises SEQ ID NO: 14, SEQ ID NO: 15 and SEQ ID NO: 16. In some embodiments, an anti-SARS-CoV-2 binder protein comprises an amino acid sequence of SEQ ID NO: 13. In some embodiments, an anti-SARS-CoV-2 binder protein consists of the amino acid sequence of SEQ ID NO: 13.

### Clones A6, A7, C9, D5, F10, F11, G4, D7

In some embodiments, an anti-SARS-CoV-2 binder protein comprises a CDR1 comprising the amino acid sequence of SEQ ID NO: 47, a CDR2 comprising the amino acid sequence of SEQ ID NO: 15, and a CDR3 comprising the amino acid sequence of SEQ ID NO: 48. In some embodiments, an anti-SARS-CoV-2 binder protein comprises CDRs1-3 consisting of the amino acid sequences of SEQ ID NOs: 47, 15, and 48, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 46 or 62. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 46 or 62; and 2) comprises SEQ ID NO: 47, SEQ ID NO: 15 and SEQ ID NO: 48. In some embodiments, an anti-SARS-CoV-2 binder protein comprises an amino acid sequence of SEQ ID NO: 46. In some embodiments, an anti-SARS-CoV-2 binder protein consists of the amino acid sequence of SEQ ID NO: 46. In some embodiments, an anti-SARS-CoV-2 binder protein comprises an amino acid sequence of SEQ ID NO: 62. In some embodiments, an anti-SARS-CoV-2 binder protein consists of the amino acid sequence of SEQ ID NO: 62.

### Clones G8, G3, F9, C10, C12, H11, C8, F2, and E12

In some embodiments, an anti-SARS-CoV-2 binder protein comprises a CDR1 comprising the amino acid sequence of SEQ ID NO: 14, 65, 70, or 76; a CDR2 comprising the amino acid sequence of SEQ ID NO: 66, or 77; and a CDR3 comprising the amino acid sequence of SEQ ID NO: 67, or 73.

In some embodiments, an anti-SARS-CoV-2 binder protein comprises CDRs 1-3 comprising the amino acid sequences of SEQ ID NOs: 65, 66, and 67, respectively. In some embodiments, an anti-SARS-CoV-2 binder protein comprises CDRs 1-3 consisting of the amino acid sequences of SEQ ID NOs: 65, 66, and 67, respectively.

In some embodiments, an anti-SARS-CoV-2 binder protein comprises CDRs 1-3 comprising the amino acid sequences of SEQ ID NOs: 70, 66, and 67, respectively. In some embodiments, an anti-SARS-CoV-2 binder protein comprises CDRs 1-3 consisting of the amino acid sequences of SEQ ID NOs: 70, 66, and 67, respectively.

In some embodiments, an anti-SARS-CoV-2 binder protein comprises CDRs 1-3 comprising the amino acid sequences of SEQ ID NOs: 76, 77, and 67, respectively. In some embodiments, an anti-SARS-CoV-2 binder protein comprises CDRs 1-3 consisting of the amino acid sequences of SEQ ID NOs: 76, 77, and 67, respectively.

In some embodiments, an anti-SARS-CoV-2 binder protein comprises CDRs 1-3 comprising the amino acid sequences of SEQ ID NOs: 14, 66, and 67, respectively. In some embodiments, an anti-SARS-CoV-2 binder protein comprises CDRs 1-3 consisting of the amino acid sequences of SEQ ID NOs: 14, 66, and 67, respectively.

In some embodiments, an anti-SARS-CoV-2 binder protein comprises CDRs 1-3 comprising the amino acid sequences of SEQ ID NOs: 14, 66, and 73, respectively. In some embodiments, an anti-SARS-CoV-2 binder protein comprises CDRs 1-3 consisting of the amino acid sequences of SEQ ID NOs: 14, 66, and 73, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 64, 69, 72, 75, 79, 81, 83, or 87. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 64, 69, 72, 75, 79, 81, 83, or 87; and 2) comprises a CDR1 comprising the amino acid sequence of SEQ ID NO: 14, 65, 70, or 76; a CDR2 comprising the amino acid sequence of SEQ ID NO: 66 or 77; and a CDR3 comprising the amino acid sequence of SEQ ID NO: 67 or 73. In some embodiments, an anti-SARS-CoV-2 binder protein comprises an amino acid sequence of SEQ ID NO: 64, 69, 72, 75, 79, 81, 83, or 87. In some embodiments, an anti-SARS-CoV-2 binder protein consists of the amino acid sequence of SEQ ID NO: 64, 69, 72, 75, 79, 81, 83, or 87.

Binder Class 4 (including Nanosota-5, also referred to as clone E10)

Certain embodiments of the invention provide an isolated anti-SARS-CoV-2 binder protein (e.g., Class 4, including Nanosota-5, as described in Table 4) that comprises one or more complementarity determining regions (CDRs) selected from the group consisting of:
(a) a CDR1 comprising an amino acid sequence having at least 75% sequence identity to an amino acid sequence of SIFRFEAV (SEQ ID NO: 90), SIFRMDVV (SEQ ID NO: 95), or SIFRMELM (SEQ ID NO: 102);
(b) a CDR2 comprising an amino acid sequence having at least 75% sequence identity to an amino acid sequence of TVARDGTTN (SEQ ID NO: 91), SITRSGSTN (SEQ ID NO: 96), TINRCGSTN (SEQ ID NO: 103) or TITRSGSTN (SEQ ID NO: 163); and
(c) a CDR3 comprising an amino acid sequence having at least 75% sequence identity to an amino acid sequence of NARWWTNF (SEQ ID NO: 92), HARTWTSY (SEQ ID NO: 97), or HARTWTSS (SEQ ID NO: 104).

In some embodiments, an anti-SARS-CoV-2 binder protein comprises one or more CDRs (e.g., 3 CDRs) selected from the group consisting of:
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSS (SEQ ID NO: 104); or
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSY (SEQ ID NO: 97); or
(a) a CDR1 comprising an amino acid sequence of SIFRFEAV (SEQ ID NO: 90);
(b) a CDR2 comprising an amino acid sequence of TVARDGTTN (SEQ ID NO: 91); and
(c) a CDR3 comprising an amino acid sequence of NARWWTNF (SEQ ID NO: 92); or
(a) a CDR1 comprising an amino acid sequence of SIFRMDVV (SEQ ID NO: 95);
(b) a CDR2 comprising an amino acid sequence of SITRSGSTN (SEQ ID NO: 96); and
(c) a CDR3 comprising an amino acid sequence of HARTWTSY (SEQ ID NO: 97); and
(a) a CDR1 comprising an amino acid sequence of SIFRMELM (SEQ ID NO: 102);
(b) a CDR2 comprising an amino acid sequence of TITRSGSTN (SEQ ID NO: 163); and
(c) a CDR3 comprising an amino acid sequence of HARTWTSY (SEQ ID NO: 97).

In some embodiments, an anti-SARS-CoV-2 binder protein comprises one or more CDRs selected from the group consisting of:
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSS (SEQ ID NO: 104); or
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSY (SEQ ID NO: 97); or
(a) a CDR1 comprising an amino acid sequence of SIFRFEAV (SEQ ID NO: 90);
(b) a CDR2 comprising an amino acid sequence of TVARDGTTN (SEQ ID NO: 91); and
(c) a CDR3 comprising an amino acid sequence of NARWWTNF (SEQ ID NO: 92); or
(a) a CDR1 comprising an amino acid sequence of SIFRMDVV (SEQ ID NO: 95);
(b) a CDR2 comprising an amino acid sequence of SITRSGSTN (SEQ ID NO: 96); and
(c) a CDR3 comprising an amino acid sequence of HARTWTSY (SEQ ID NO: 97).

In some embodiments, the anti-SARS-CoV-2 binder protein comprises an amino acid sequence that has at least 85% sequence identity to:

(SEQ ID NO: 101)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSSWGRGTQVTVSS,
(SEQ ID NO: 106)
QVQLQESGGGLVQAGGSLRLSCAASESIFRMELMQWYRQAPGKQRELVAT
ITRSGSTNYSDSVKGRLIISSDNAKNSVYLQMNSLKAEDTAVYLCHARTW
TSYWGQGTQVTVSS,
(SEQ ID NO: 89)
QVQLQESGGGLVQAGGSLRLSCVASGSIFRFEAVGWYRQAPGKQRELVAT
VARDGTTNYADSVKGRFTISTDNAKNSVYLQMNSLKAEDTAVYVCNARWW
TNFWGQGTQVTVSS,
(SEQ ID NO: 94)
QVQLQESGGGLVQAGGSLRLSCAASESIFRMDVVQWYRQAPGKQRELVAS
ITRSGSTNYADSVKGRFIISSDNAKNSVYLQMKSLKVEDTAVYLCHARTW
TSYWGQGTQVTVSS,
(SEQ ID NO: 99)
QVQLQESGGGLVQPGGSLRLSCAASESIFRMDVVQWYRQAPGKQRELVAS
ITRSGSTNYADSVKGRFIISSDNAKNSVYLQMKSLKVEDTAVYLCHARTW
TSYWGQGTQVTVSS;
(SEQ ID NO: 108)
QVQLQESGGGLVQAGGSLRLSCAASESIFRMELMEWYRQAPGKQRELVAT
ITRSGSTNYSDSVKGRFIISSDNAKNSVYLQMNSLKAEDTAVYLCHARTW
TSYWGQGTQVTVSS;
(SEQ ID NO: 118)
QVQLQESGGGLVQAGGSLRLSCAASESIFRMELMQWYRQAPGKQRELVAT
ITRSGSTNYSDSVKGRFIISSDNAKNSVYLQMNSLKAEDTAVYLCHARTW
TSYWGQGTQVTVSS;
or
(SEQ ID NO: 132)
QVQLQESGGGLVQAGGSLRLSCAASESIFRMELMAWYRQAPGKQRELVAT
ITRSGSTNYSDSVKGRFIISSDNAKNSVYLQMNSLKAEDTAVYLCHARTW
TSYWGQGTQVTVSS.

In some embodiments, the isolated anti-SARS-CoV-2 binder protein comprises an amino acid sequence that has at least 90% sequence identity to SEQ ID NO: 89, 94, 99, 101, 106, 108, 118, or 132.

In some embodiments, the isolated anti-SARS-CoV-2 binder protein comprises the amino acid sequence of SEQ ID NO: 89, 94, 99, 101, 106, 108, 118, or 132.

In some embodiments, an anti-SARS-CoV-2 binder protein (e.g., Nanosota-5^{s} or Nanosota-5) comprises one or more CDRs selected from the group consisting of:
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSS (SEQ ID NO: 104); or
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSY (SEQ ID NO: 97)

In some embodiments, the anti-SARS-CoV-2 binder protein (e.g., Nanosota-5^{s} or Nanosota-5) comprises an amino acid sequence that has at least 85% (e.g., 86%, 87%, 88%, 89%, 90%, 91%, 92%, 93%, 94%, 95%, 96%, 97%, 98%, 99% or 100%) sequence identity to:

(SEQ ID NO: 101)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSSWGRGTQVTVSS ,
or
(SEQ ID NO: 164)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSYWGRGTQVTVSS.

In some embodiments, the isolated anti-SARS-CoV-2 binder protein (e.g., Nanosota-5^{s} or Nanosota-5) comprises an amino acid sequence that has at least 90% (e.g., 91%, 92%, 93%, 94%, 95%, 96%, 97%, 98%, 99% or 100%) sequence identity to SEQ ID NO: 101, or 164.

In some embodiments, the isolated anti-SARS-CoV-2 binder protein comprises the amino acid sequence of SEQ ID NO: 101, or 164.

In some embodiments, the isolated anti-SARS-CoV-2 binder protein (e.g., Nanosota-5^{s} or Nanosota-5) further comprises two residues of methionine-alanine (MA) at the N-terminal of the protein (e.g., before the VHH sequence such as SEQ ID NO: 101 or 164).

In some embodiments, an anti-SARS-CoV-2 binder protein comprises a CDR1 comprising the amino acid sequence of SIFRFEAV (SEQ ID NO: 90), SIFRMDVV (SEQ ID NO: 95), or SIFRMELM (SEQ ID NO: 102), a CDR2 comprising the amino acid sequence of TVARDGTTN (SEQ ID NO: 91), SITRSGSTN (SEQ ID NO: 96), or TINRCGSTN (SEQ ID NO: 103), and a CDR3 comprising the amino acid sequence of NARWWTNF (SEQ ID NO: 92), HARTWTSY (SEQ ID NO: 97), or HARTWTSS (SEQ ID NO: 104).

In some embodiments, an anti-SARS-CoV-2 binder protein comprises a CDR1 comprising the amino acid sequence of SIFR(F/M)(E/D)(A/V/L)(V/M) (SEQ ID NO: 160), a CDR2 comprising the amino acid sequence of TINRCGSTN (SEQ ID NO: 103), and a CDR3 comprising the amino acid sequence of HARTWTSS (SEQ ID NO: 104).

In some embodiments, an anti-SARS-CoV-2 binder protein comprises a CDR1 comprising the amino acid sequence of SIFRMELM (SEQ ID NO: 102), a CDR2 comprising the amino acid sequence of (S/T)(V/I)(A/T/N)R(D/S/C)G(T/S)TN (SEQ ID NO: 161), and a CDR3 comprising the amino acid sequence of HARTWTSS (SEQ ID NO: 104).

In some embodiments, an anti-SARS-CoV-2 binder protein comprises a CDR1 comprising the amino acid sequence of SIFRMELM (SEQ ID NO: 102), a CDR2 comprising the amino acid sequence of TINRCGSTN (SEQ ID NO: 103), and a CDR3 comprising the amino acid sequence of (H/N)AR(T/W)WT(S/N)(S/Y/F) (SEQ ID NO: 162).

In some embodiments, an anti-SARS-CoV-2 binder protein comprises a CDR1 comprising the amino acid sequence of SIFRMELM (SEQ ID NO: 102), a CDR2 comprising the amino acid sequence of TINRCGSTN (SEQ ID NO: 103), and a CDR3 comprising the amino acid sequence of HARTWTSY (SEQ ID NO: 97), or HARTWTSS (SEQ ID NO: 104).

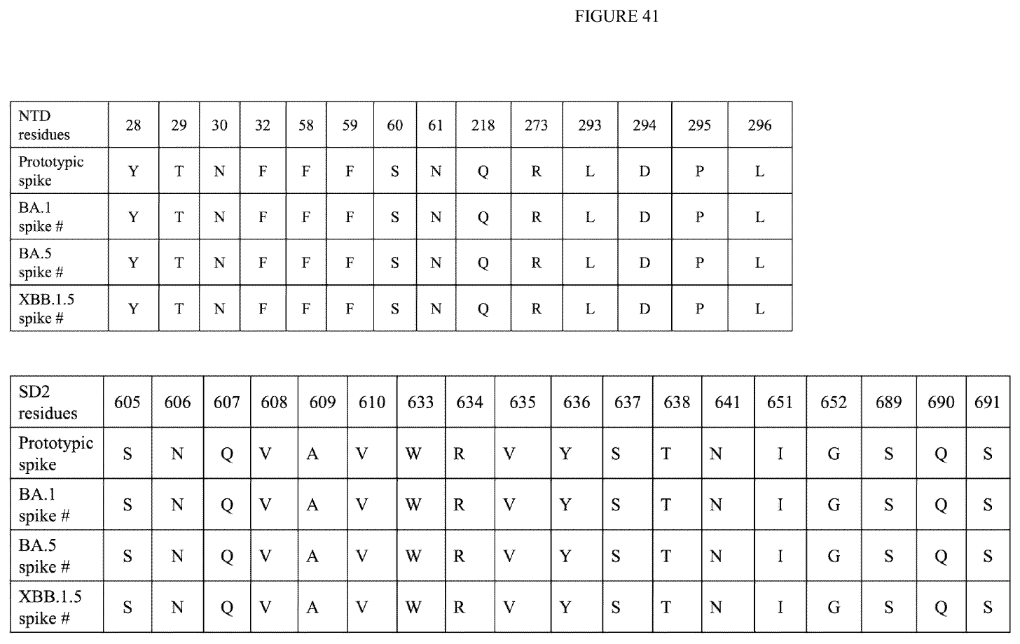

In certain embodiments, an anti-SARS-CoV-2 binder protein (e.g., comprising CDRs or VHH sequence of nanosota-5, or nanosota-5-Fc) binds an epitope comprising ten or more (e.g., 11, 12, 13, or 14 residues) residues selected from the group of NTD residues shown in FIG. 41. In certain embodiments, an anti-SARS-CoV-2 binder protein binds an epitope comprising fourteen or more (e.g., 15, 16, 17, or 18 residues) residues selected from the group of SD2 residues shown in FIG. 41. In certain embodiments, an anti-SARS-CoV-2 binder protein binds an epitope (e.g., NTD and SD2 of the same spike polypeptide chain) comprising or consisting of fourteen NTD residues and eighteen SD2 residues shown in FIG. 41.

In certain embodiments, a binder protein of the invention (e.g., comprising CDRs or VHH sequence of nanosota-5, or nanosota-5-Fc) inhibits the viral entry of SARS-CoV-2 Omicron variant (e.g., BA.1, BA.5 or XBB.1.5) into a cell by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

In certain embodiments, a binder protein of the invention (e.g., comprising CDRs or VHH sequence of nanosota-5, or nanosota-5-Fc) inhibits the viral entry of SARS-CoV-2 Omicron variant (e.g., BA.1, BA.5 or XBB.1.5) into a cell with a IC₅₀ potency of about 1-600 ng/mL, 2-300 ng/mL, 3-200 ng/mL, 5-180 ng/mL, 6-160 ng/mL, or 10-150 ng/mL. In certain embodiments, a binder protein of the invention (e.g., comprising CDRs or VHH sequence of nanosota-5, or nanosota-5-Fc) inhibits the viral entry of SARS-CoV-2 Omicron variant (e.g., BA.1, BA.5 or XBB.1.5) into a cell with a IC₅₀ potency of about 1, 5, 10, 11, 12, 13, 14, 15, 20, 30, 40, 50, 60, 70, 80, 90, 100, 110, 120, 130, 140, 150, 160, 180, or 200 ng/mL.

### Clone E10

In some embodiments, an anti-SARS-CoV-2 binder protein comprises a CDR1 comprising the amino acid sequence of SEQ ID NO: 102, a CDR2 comprising the amino acid sequence of SEQ ID NO: 103, and a CDR3 comprising the amino acid sequence of SEQ ID NO: 97. In some embodiments, an anti-SARS-CoV-2 binder protein comprises CDRs 1-3 consisting of the amino acid sequences of SEQ ID NOs: 102, 103, and 97, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 164. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 164; and 2) comprises SEQ ID NOs: 102, 103 and 97. In some embodiments, an anti-SARS-CoV-2 binder protein comprises an amino acid sequence of SEQ ID NO: 164. In some embodiments, an anti-SARS-CoV-2 binder protein consists of the amino acid sequence of SEQ ID NO: 164.

### Nanosota-5^{s}

In some embodiments, an anti-SARS-CoV-2 binder protein comprises a CDR1 comprising the amino acid sequence of SEQ ID NO: 102, a CDR2 comprising the amino acid sequence of SEQ ID NO: 103, and a CDR3 comprising the amino acid sequence of SEQ ID NO: 104. In some embodiments, an anti-SARS-CoV-2 binder protein comprises CDRs 1-3 consisting of the amino acid sequences of SEQ ID NOs: 102, 103, and 104, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 101. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 101; and 2) comprises SEQ ID NOs: 102, 103 and 104. In some embodiments, an anti-SARS-CoV-2 binder protein comprises an amino acid sequence of SEQ ID NO: 101. In some embodiments, an anti-SARS-CoV-2 binder protein consists of the amino acid sequence of SEQ ID NO: 101.

### Clone A2

In some embodiments, an anti-SARS-CoV-2 binder protein comprises a CDR1 comprising the amino acid sequence of SEQ ID NO: 90, a CDR2 comprising the amino acid sequence of SEQ ID NO: 91, and a CDR3 comprising the amino acid sequence of SEQ ID NO: 92. In some embodiments, an anti-SARS-CoV-2 binder protein comprises CDRs 1-3 consisting of the amino acid sequences of SEQ ID NOs: 90, 91, and 92, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 89. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 89; and 2) comprises SEQ ID NOs: 90, 91 and 92. In some embodiments, an anti-SARS-CoV-2 binder protein comprises an amino acid sequence of SEQ ID NO: 89. In some embodiments, an anti-SARS-CoV-2 binder protein consists of the amino acid sequence of SEQ ID NO: 89.

### Clones E6 and F6

In some embodiments, an anti-SARS-CoV-2 binder protein comprises a CDR1 comprising the amino acid sequence of SEQ ID NO: 95, a CDR2 comprising the amino acid sequence of SEQ ID NO: 96, and a CDR3 comprising the amino acid sequence of SEQ ID NO: 97. In some embodiments, an anti-SARS-CoV-2 binder protein comprises CDRs 1-3 consisting of the amino acid sequences of SEQ ID NOs: 95, 96, and 97, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 94 or 99. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 94 or 99; and 2) comprises SEQ ID NOs: 95, 96 and 97. In some embodiments, an anti-SARS-CoV-2 binder protein comprises an amino acid sequence of SEQ ID NO: 94 or 99. In some embodiments, an anti-SARS-CoV-2 binder protein consists of the amino acid sequence of SEQ ID NO: 94 or 99.

### Clones D6, C2, D1, E1, E4, H4, A5, B1, B6, B7, F1, F3, F4, and H5

In some embodiments, an anti-SARS-CoV-2 binder protein comprises a CDR1 comprising the amino acid sequence of SEQ ID NO: 102, a CDR2 comprising the amino acid sequence of SEQ ID NO: 163, and a CDR3 comprising the amino acid sequence of SEQ ID NO: 97. In some embodiments, an anti-SARS-CoV-2 binder protein comprises CDRs 1-3 consisting of the amino acid sequences of SEQ ID NOs: 102, 163, and 97, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 106, 108, 118, or 132. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 106, 108, 118, or 132; and 2) comprises SEQ ID NOs: 102, 163 and 97. In some embodiments, an anti-SARS-CoV-2 binder protein comprises an amino acid sequence of SEQ ID NO: 106, 108, 118, or 132. In some embodiments, an anti-SARS-CoV-2 binder protein consists of the amino acid sequence of SEQ ID NO: 106, 108, 118, or 132.

### Binder Class 5 (Including Nanosota-6)

### Clones H1, H3, and D2

In some embodiments, an anti-SARS-CoV-2 binder protein comprises a CDR1 comprising the amino acid sequence of SEQ ID NO: 135, a CDR2 comprising the amino acid sequence of SEQ ID NO: 136, and a CDR3 comprising the amino acid sequence of SEQ ID NO: 137. In some embodiments, an anti-SARS-CoV-2 binder protein comprises CDRs 1-3 consisting of the amino acid sequences of SEQ ID NOs: 135, 136, and 137, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 134, 139, or 141. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 134, 139, or 141; and 2) comprises SEQ ID NOs: 135, 136, and 137. In some embodiments, an anti-SARS-CoV-2 binder protein comprises an amino acid sequence of SEQ ID NO: 134, 139, or 141. In some embodiments, an anti-SARS-CoV-2 binder protein consists of the amino acid sequence of SEQ ID NO: 134, 139, or 141.

### Binder Class 6 (Nanosota-7)

### Clone C3

In some embodiments, an anti-SARS-CoV-2 binder protein comprises a CDR1 comprising the amino acid sequence of SEQ ID NO: 144, a CDR2 comprising the amino acid sequence of SEQ ID NO: 145, and a CDR3 comprising the amino acid sequence of SEQ ID NO: 146. In some embodiments, an anti-SARS-CoV-2 binder protein comprises CDRs 1-3 consisting of the amino acid sequences of SEQ ID NOs: 144, 145, and 146, respectively.

In some embodiments, an anti-SARS-CoV-2 binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 143. In some embodiments, an anti-SARS-CoV-2 binder protein comprises an amino acid sequence that 1) has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 143; and 2) comprises SEQ ID NOs: 144, 145, and 146. In some embodiments, an anti-SARS-CoV-2 binder protein comprises an amino acid sequence of SEQ ID NO: 143. In some embodiments, an anti-SARS-CoV-2 binder protein consists of the amino acid sequence of SEQ ID NO: 143.

In certain embodiments, a binder protein described herein is a broad-spectrum SARS-CoV binding protein.

In certain embodiments, the binder protein described herein specifically binds to SARS-CoV-2 (prototypic) or its variant (e.g., alpha, delta, and/or omicron), and SARS-CoV-1. In certain embodiments, the binder protein is capable of binding to a RBD region that is conserved across two or more SARS strains (e.g., SARS-CoV-2 and CoV-1) or variants.

In certain embodiments, the binder protein specifically binds to SARS-CoV-2 (prototypic) and one or more variants (e.g., Alpha, Delta, or Omicron). For example, in certain embodiments, the binder protein binds to SARS-CoV-2 and Omicron. In certain embodiments, the binder protein binds to SARS-CoV-2 and Alpha. In certain embodiments, the binder protein binds to SARS-CoV-2 and Delta. In certain embodiments, the binder protein binds to SARS-CoV-2, Alpha, and Delta. In certain embodiments, the binder protein binds to SARS-CoV-2, Alpha, and Omicron. In certain embodiments, the binder protein binds to SARS-CoV-2, Alpha, Delta, and Omicron. In certain embodiments, the binder protein binds to SARS-CoV-2, Alpha, Delta, and Omicron BA. 1, Omicron BA.5, Omicron XBB.1.5, Omicron EG.5, and Omicron JN.1.

In certain embodiments, the binder protein also binds to bat SARS1 (strain Rs3367) and/or bat SARS2 (strain BANAL236 strain).

“Broad-spectrum” as used herein refers to the ability of a binder protein to bind to two or more (e.g., 3, 4, 5, 6, or more) SARS-CoV strains or variants. For example, in certain embodiments, a binder protein as described herein (e.g., sdAb, or sdAb-Fc) is capable of binding to 4, 5, 6 or more SARS-CoV strains or variants. In certain embodiments, the two or more (e.g., 3, 4, 5, 6, or more) SARS-CoV strains or variants are selected from the group consisting of SARS-CoV-2, Alpha, Delta, Omicron, SARS-CoV-1, bat SARS1, and bat SARS2. The spike protein of SARS-CoV-2, Alpha, Delta, Omicron, SARS-CoV-1, bat SARS1, and bat SARS2 are known in the art and described herein, for example, the NCBI or GISAID accession numbers for S proteins (or gene) of SARS-CoV-2 (prototypic), Alpha, Delta, Omicron BA.1, Omicron BA.5, Omicron XBB.1.5, Omicron EG.5, Omicron JN.1, SARS-CoV-1, bat SARS1, and bat SARS2 are GenBank: QHD43416.1, GISAID: EPI_ISL_6135157, GenBank: UEM53021.1, GISAID: EPI_ISL_6590782.2, GISAID: EPI_ISL_12954165, GISAID: EPI_ISL_17774216, GISAID: EPI_ISL_17524442, GISAID: EPI_ISL_18701931, GenBank: AFR58742.1, GenBank: AGZ48818.1, and GenBank: MZ937003.2, respectively.

In certain embodiments, a binder protein described herein is capable of specifically binding to the receptor binding domain (RBD) of a SARS-CoV spike protein (e.g., prototypic RBD of SARS-CoV-2 or variant). In other embodiments, a binder protein described herein is not capable of specifically binding to the receptor binding domain (RBD) of a SARS-CoV spike protein (e.g., prototypic RBD of SARS-CoV-2 or variant).

In certain embodiments, a binder protein described herein is capable of neutralizing (e.g., neutralize viral entry) one or more SARS-CoV strains or variants. In certain embodiments, a binder protein described herein is capable of neutralizing two or more (e.g., 3, 4, 5, 6, or more) SARS-CoV strains or variants. In certain embodiments, a binder protein described herein is capable of neutralizing one or more SARS-CoV-2 or its variant.

In certain embodiments, the binder protein described herein has higher affinity for the receptor binding domain (RBD) of a SARS-CoV spike protein (e.g., prototypic RBD of SARS-CoV-2 or variant) than that of human angiotensin converting enzyme 2 (ACE2). In certain embodiments, the binder protein prevents ACE2 from binding to the RBD.

In certain embodiments, the binder protein's binding footprint on the RBD overlaps with ACE2's binding region on the RBD. In certain embodiments, the binder protein's binding footprint on the RBD completely overlaps with ACE2's binding region on the RBD. In certain embodiments, the binder protein's binding footprint on the RBD partially overlaps with ACE2's binding region on the RBD. In certain embodiments the binder protein has a footprint as described herein (e.g., binds one or more residues as described herein).

The RBD of SARS-CoV-2 spike protein is capable of adopting both a standing up and lying down configuration. These configurations are known in the art and described herein (e.g., see Example 1). In certain embodiments, the binder protein is capable of binding to the standing up RBD. In certain embodiments, the binder protein is capable of binding to the lying down RBD. In certain embodiments, the binder protein is capable of binding to both the standing up RBD and the lying down RBD.

In certain embodiments, the binder protein is capable of binding to the RBD (e.g., lying down RBD) in a region (e.g., a cavity in the trimeric spike) that is inaccessible to or that cannot accommodate a full-size antibody, or an antigen binding fragment thereof having both a heavy chain and light chain (e.g., a scFv, a Fab, or a full-size IgG antibody).

### Tags

In certain embodiments, a binder protein described herein is operably linked to at least one detectable agent (e.g., a polypeptide tag such as His6 tag (SEQ ID NO: 20)). In certain embodiments, an isolated anti-SARS-CoV-2 binder protein as described herein is operably linked to at least one detectable agent. The location of the detectable agent is not critical, provided that it does not interfere with the function of the binder protein. In certain embodiments, the detectable agent is operably linked to the N-terminus of the binder protein. In certain embodiments, the detectable agent is operably linked to the C-terminus of the binder protein.

In certain embodiments, a binder protein (e.g., class 1, 2, 3, 4, 5, or 6 (e.g., Nanosota-5, 6, or 7)) as described in Tables 1-6 is operably linked to a detectable agent described herein.

In certain embodiments, the at least one detectable agent is a tag, such as an affinity tag or an epitope tag. For example, such a tag may be useful for detecting, isolating and/or purifying the binder protein. In certain embodiments, the tag is a polypeptide tag. Polypeptide tags are known in the art and include, but are not limited to, e.g., a His tag, Myc tag, HA tag or an Fc tag. In certain embodiments, the at least one detectable agent is an Fc tag (e.g., an IgG1, IgG2, IgG3, or IgG4 Fc). For example, as described below, a sdAb may be operably linked to an Fc domain amino acid sequence, to produce a sdAb-Fc fusion protein. In certain embodiments, the at least one detectable agent is a His tag (e.g., His6 tag (SEQ ID NO: 20)). In certain embodiments, the at least one detectable agent is a HA tag. In certain embodiments, multiple tags may be operably linked in tandem either directly or via a linker group. In certain embodiments, the detectable agent(s) comprise a HA tag and/or a His tag (e.g., His6 tag (SEQ ID NO: 20)).

In certain embodiments, the binder protein is directly linked to the detectable agent, such as a polypeptide tag (e.g., through a peptide bond).

In certain other embodiments, the binder protein is linked to the detectable agent, such as a polypeptide tag, via one or more optional linker group(s). The nature of the linker group is not critical, provided that the linker group does not interfere with the function of the binder protein or the detectable agent. In certain embodiments, the linker group is an amino acid sequence (e.g., a sequence described herein). In certain embodiments, the linker group is an amino acid sequence that is at least 1, 2, 3, 4, 5, 6, 7, 8, 9, 10 or more amino acids in length. In certain embodiments, the linker group is an amino acid sequence about 1 to about 25 amino acids in length, or about 1 to about 20 amino acids in length, or about 1 to about 15 amino acids in length, or about 1 to about 10 amino acids in length (e.g., 1, 2, 3, 4, 5, 6, 7, 8, 9 or 10 amino acids in length). In certain embodiments, the linker group is glycine rich linker (e.g., having about more than 60% of the amino acid residues in the linker group is glycine). In certain embodiments, the linker group is glycine-serine linker (e.g., GS, GGS, or GGSGGS (SEQ ID NO: 155)).

Accordingly, in certain embodiments, an anti-SARS-CoV-2 binder protein described herein operably linked to a polypeptide tag, comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 5)
QVQLQESGGGAVQPGGSLGLSCTASGFNFETSTVGWFRQAPGKENEGVSC
INKGYEDTNYADSVKGRFTISRDAAKNTVYLQMDSLQPEDTATYYCAAHN
EPYFCDYSGRFRWNEYSYYGQGTQVTVSSGSHHHHHH;
(b)
(SEQ ID NO: 11)
QVQLQESGGGLVQAGGSLRLSCAASGSIFSPNTMGWFRQALGKQREMVAV
ISSIASTQYANFVKGRFTITRDNTKNTVHLQMNSLIPEDTAVYYCYAVDK
SQDYWGQGTQVTVSSGSHHHHHH;
and
(c)
(SEQ ID NO: 17)
QVQLQESGGGLVQPGGSLRLSCAASGFTLDYYAIGWFRQAPGKEREGVSC
ISSSGGRTNYADSVKGRFTISRDNTKNTVYLQMNSLKPEDTAVYYCAAWE
ASRWYCPLQFSADFSSWGQGTQVTVSSGSHHHHHH.

In some embodiments, an anti-SARS-CoV-2 binder protein comprises an amino acid sequence of any one of SEQ ID NOs: 5, 11, and 17. In some embodiments, an anti-SARS-CoV-2 binder protein consists of the amino acid sequence of any one of SEQ ID NOs: 5, 11, and 17.

Accordingly, in certain embodiments, an anti-SARS-CoV-2 binder protein described herein operably linked to a polypeptide tag, comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of SEQ ID NOs: 32, 37, 39, 45, 49, 63, 68, 71, 74, 78, 80, 82, 84, 88, 93, 98, 100, 105, 107, 109, 119, 133, 138, 140, 142, and 147.

In some embodiments, an anti-SARS-CoV-2 binder protein comprises an amino acid sequence of any one of SEQ ID NOs: 32, 37, 39, 45, 49, 63, 68, 71, 74, 78, 80, 82, 84, 88, 93, 98, 100, 105, 107, 109, 119, 133, 138, 140, 142, and 147. In some embodiments, an anti-SARS-CoV-2 binder protein consists of the amino acid sequence of any one of SEQ ID NOs: 32, 37, 39, 45, 49, 63, 68, 71, 74, 78, 80, 82, 84, 88, 93, 98, 100, 105, 107, 109, 119, 133, 138, 140, 142, and 147.

In certain embodiments, a detectable agent is a small molecule with molecular weight no greater than 1000 g/mol (e.g., biotin or fluorophore).

In certain embodiments, a detectable agent is a nanoparticle (e.g., gold nanoparticle or magnetic particle).

In certain embodiments, a detectable agent is suitable for a lateral flow assay.

In certain embodiments, a detectable agent (e.g., tag) is an enzyme (e.g., horseradish peroxidase (HRP) or an enzyme that is suitable for a chemiluminescent assay, or a colorimetric assay such as ELISA).

In certain embodiments, a detectable agent is suitable for a colorimetric, chemiluminescent, or fluorescent assay.

In certain embodiments, a detectable agent is suitable for an ELISA, chemiluminescence assay, or flow cytometry or fluorescent imaging assay.

In some embodiments, the binder protein is encoded by a polynucleotide comprising a nucleic acid sequence that has at least about 80% (e.g., at least about 80%, 85%, 90%, 91%, 92%, 93%, 94%, 95%, 96%, 97%, 98%, 99% or 100%) sequence identity to any one of SEQ ID NOs: 22, 24, or 26.

In certain embodiments, a sdAb as described herein is a recombinant sdAb. In certain embodiments, a sdAb as described herein is a chimeric sdAb. In certain embodiments, a sdAb as described herein is humanized.

In certain embodiments, a sdAb of the invention is a monoclonal sdAb. In some embodiments, the monoclonal sdAb recognizes an epitope within SARS-CoV-2.

In certain embodiments, an isolated anti-SARS-CoV-2 binder protein described herein is an inhibitor of SARS-CoV-2.

The term “inhibitor of SARS-CoV-2” as used herein refers to a binder protein that is capable of inhibiting the function of SARS-CoV-2 (e.g., inhibits binding to ACE2). For example, in certain embodiments, a binder protein as described herein detectably inhibits the biological activity of SARS-CoV-2 as measured, e.g., using an assay described herein. In certain embodiments, the binder protein inhibits the biological activity of SARS-CoV-2 by at least about 5%, at least about 10%, at least about 20%, at least about 30%, at least about 40%, at least about 50%, at least about 60%, at least about 70%, at least about 80%, or at least about 90%.

In certain embodiments, the binder protein is a selective inhibitor of SARS-CoV-2.

For example, a binder protein of the invention may be at least 5, at least 10, at least 50, at least 100, at least 500, or at least 1,000 fold selective for SARS-CoV-2 over another coronavirus in a selected assay (e.g., an assay described in the Examples herein).

Certain embodiments of the invention provide a binder protein as described herein.

### Certain Proteins of the Invention

As described herein, a binder protein may comprise a sdAb of the invention and may optionally be linked to one or more additional polypeptides. For example, in certain embodiments, an isolated anti-SARS-CoV-2 binder protein described herein is further linked to one or more antibody domain sequences (e.g., heavy or light chain domain sequences, such as variable or constant domain sequences). Accordingly, certain embodiments provide a protein comprising a sdAb of the invention operably linked to one or more antibody domain sequences. Such protein molecules comprising a sdAb of the invention and one or more additional antibody domains may be referenced herein as an antibody or antibody fragment. Additionally, such molecules may be further modified as described herein (e.g., humanized or to alter its affinity, etc.). In certain embodiments, the one or more antibody domain sequences are derived from an antibody class or isotype as defined herein (e.g., IgG (IgG1, IgG2, IgG3, IgG4), IgM, IgA (IgA1 and IgA2), IgD, and IgE).

In certain embodiments, the sdAb is not linked to a light chain domain. In certain embodiments, the sdAb is not linked to a constant domain region. In certain embodiments, the sdAb is not linked to a CH1 region.

In certain embodiments, an isolated anti-SARS-CoV-2 sdAb described herein is linked (e.g., through a linker or a direct bond, such as a peptide bond) to at least one heavy chain constant region (e.g., 1, 2, or 3). In certain embodiments, the sdAb is linked to two heavy chain constant regions (e.g., a CH2 and CH3 region). In certain embodiments, the sdAb is operably linked to an Fc domain amino acid sequence (e.g., an IgG Fc domain such as IgG1, IgG2, IgG3, or IgG4 Fc domain), to produce a sdAb-Fc fusion protein.

Thus, certain embodiments of the invention provide a sdAb-Fc-fusion protein comprising a sdAb of the invention operably linked to a Fc domain amino acid sequence. In certain embodiments, the sdAb and Fc domain amino acid sequence are directly linked, e.g., through a peptide bond. In certain embodiments, the sdAb and Fc domain amino acid sequence are linked through an amino acid linking group. In certain embodiments, the Fc domain amino acid sequence is an IgG4 Fc domain amino acid sequence. In certain embodiments, the Fc domain amino acid sequence is an IgG1 Fc domain amino acid sequence. In certain embodiments, the Fc domain amino acid sequence has at least about has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 21. In certain embodiments, the Fc domain amino acid sequence comprises SEQ ID NO: 21. In certain embodiments, the Fc domain amino acid sequence consists of SEQ ID NO: 21.

In certain embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one sdAb-Fc sequence described in any one of Table A, and Tables 1-6.

In certain embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of:

(a)
(SEQ ID NO: 6)
QVQLQESGGGAVQPGGSLGLSCTASGFNFETSTVGWFRQAPGKENEGVSC
INKGYEDTNYADSVKGRFTISRDAAKNTVYLQMDSLQPEDTATYYCAAHN
EPYFCDYSGRFRWNEYSYYGQGTQVTVSSEPKSCDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(b)
(SEQ ID NO: 12)
QVQLQESGGGLVQAGGSLRLSCAASGSIFSPNTMGWFRQALGKQREMVAV
ISSIASTQYANFVKGRFTITRDNTKNTVHLQMNSLIPEDTAVYYCYAVDK
SQDYWGQGTQVTVSSEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK;
(c)
(SEQ ID NO: 18)
QVQLQESGGGLVQPGGSLRLSCAASGFTLDYYAIGWFRQAPGKEREGVSC
ISSSGGRTNYADSVKGRFTISRDNTKNTVYLQMNSLKPEDTAVYYCAAWE
ASRWYCPLQFSADFSSWGQGTQVTVSSEPKSCDKTHTCPPCPAPELLGGP
SVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQ
KSLSLSPGK;
(d)
(SEQ ID NO: 152)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSSWGRGTQVTVSSEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK;
and
(e)
(SEQ ID NO: 166)
QVQLQESGGGLVQAGGSLRLSCAASESIFRMELMEWYHQAPGKQRELVAT
INRCGSTNYSDSVKGRFIISSDNAKNSVYLQMNSLKDEDTAVYSCHARTW
TSYWGRGTQVTVSSEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK.

In some embodiments, the sdAb-Fc fusion protein comprises an amino acid sequence of any one of SEQ ID NOs: 6, 12, 18. In some embodiments, the sdAb-Fc fusion protein consists of an amino acid sequence of any one of SEQ ID NOs: 6, 12, 18.

In some embodiments, the sdAb-Fc fusion protein comprises an amino acid sequence of any one of SEQ ID NOs: 6, 12, 18, 152, and 166. In some embodiments, the sdAb-Fc fusion protein consists of an amino acid sequence of any one of SEQ ID NOs: 6, 12, 18, 152, and 166.

In some embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of any one of SEQ ID NOs: 152, 153, and 154.

In some embodiments, the sdAb-Fc fusion protein comprises an amino acid sequence that has at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to any one of any one of SEQ ID NOs: 152, and 166.

In some embodiments, the sdAb-Fc fusion protein comprises an amino acid sequence of any one of SEQ ID NOs: 152, 153, and 154. In some embodiments, the sdAb-Fc fusion protein consists of an amino acid sequence of any one of SEQ ID NOs: 152, 153, and 154.

In some embodiments, the sdAb-Fc fusion protein comprises an amino acid sequence of any one of SEQ ID NOs: 152 and 166.

In some embodiments, the sdAb-Fc fusion protein is encoded by a polynucleotide comprising a nucleic acid sequence that has at least about 80% (e.g., at least about 80%, 85%, 90%, 91%, 92%, 93%, 94%, 95%, 96%, 97%, 98%, 99% or 100%) sequence identity to any of SEQ ID NOs: 23, 25, or 27.

Certain embodiments of the invention also provide multivalent sdAbs (e.g., bivalent, trivalent, tetravalent, pentavalent or higher valence multivalent sdAbs). Thus, certain embodiments of the invention provide a binder protein comprising two or more independently selected sdAbs as described herein, wherein the sdAbs are operably linked to each other (e.g., to form a dimer, trimer, tetramer, pentamer or higher valence multimer sdAb). In certain embodiments, a multivalent sdAb or binding protein as described herein is a homo-multimer (e.g., dimer, trimer, tetramer or pentamer). In certain embodiments, a multivalent sdAb or binder protein as described herein is a hetero-multimer (e.g., dimer, trimer, tetramer or pentamer).

In certain embodiments, the two or more sdAbs are operably linked via a linker group (e.g., a peptide linker group), disulfide bond(s) and/or by non-covalent interactions. In certain embodiments, the two or more sdAbs are operably linked via oligomerization of tag polypeptides (e.g., multimerization tags, such as a dimerization tags, trimerization tags, tetramerization tags, etc.).

In certain embodiments, the two or more sdAbs are operably linked via a linker group. The nature of the linker group is not critical, provided that the linker group does not interfere with the function of the sdAbs. In certain embodiments, the linker group is a peptide linker group. In certain embodiments, the peptide linker is a glycine-serine rich linker.

In certain embodiments, two independently selected sdAbs are linked via a linker group (e.g., a peptide linker group) to form dimeric sdAb. In certain embodiments, three independently selected sdAbs are linked via two linker groups (e.g., two peptide linker groups) to form a trimeric sdAb. In certain embodiments, four independently selected sdAbs are linked via three linker groups (e.g., three peptide linker groups) to form a tetrameric sdAb. In certain embodiments, five independently selected sdAbs are linked via four linker groups (e.g., four peptide linker groups) to form a pentameric sdAb.

In certain embodiments, the two or more sdAbs are operably linked via oligomerization of tag polypeptides. For example, a sdAb as described herein may be operably linked to a tag polypeptide to form a sdAb-tag fusion protein, wherein the tag polypeptide is capable of oligomerizing. Accordingly, two or more sdAb-tag fusion proteins may be operably linked to form a dimer, trimer, tetramer, pentamer or a higher valence multimer via polypeptide tag-mediated oligomerization.

In certain embodiments, the sdAb and tag polypeptide are linked through a peptide linker to form the sdAb-tag fusion protein. In certain embodiments, a sdAb and tag polypeptide are directly linked without an intervening peptide linker to form the sdAb-tag fusion protein.

In certain embodiments, the tag polypeptide is a human Fc sequence, a human collagen XVIII trimerization domain or a coiled-coil peptide derived from human cartilage oligomeric matrix protein COMP48, which is capable of forming a multimer, such as a pentamer.

In certain embodiments, the tag polypeptide is a human Fc sequence. Accordingly, certain embodiments of the invention provide a binding protein comprising: two independently selected sdAb-Fc fusion proteins as described herein, wherein the two Fc polypeptides are linked to form a dimer (e.g., linked by a covalent bond, such as a disulfide bond, or by non-covalent interactions such as electrostatic interactions, hydrogen bonding, etc.).

In certain embodiments, the two sdAb-Fc fusion proteins are the same. In certain embodiments, the two sdAb-Fc fusion proteins are different. In certain embodiments, sdAb-Fc fusion proteins as described herein can form homo-dimers. In certain embodiments, sdAb-Fc fusion proteins as described herein can form hetero-dimers. In certain embodiments, sdAb-Fc fusion proteins as described herein can form bispecific hetero-dimers having binding affinities for different SARS-Cov-2 protein(s) and/or epitopes.

In certain other embodiments, a single sdAb of the invention is operably linked to an Fc dimer.

In certain embodiments, a protein molecule as described herein is further operably linked to a detectable agent (e.g., a detectable agent described herein).

Certain embodiments of the invention also provide a protein molecule comprising a sdAb as described herein. The terms “protein”, “protein molecule”, and “polypeptide” are used interchangeably herein. In certain embodiments, the term “protein” or “protein molecule” may refer to a single polypeptide or may refer to two or more polypeptides, wherein the two or more polypeptides may be linked by a covalent (e.g., disulfide bridge) or non-covalent interactions.

As used herein, the term “antibody” includes a single-chain variable fragment (scFv), a dimer of a sdAb, a sdAb-Fc fusion protein or dimer thereof, humanized, fully human or chimeric antibodies, single-chain antibodies, diabodies, and antigen-binding fragments of antibodies that do not contain the Fc region (e.g., Fab fragments). In certain embodiments, the antibody is a camelid antibody, human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “monoclonal sdAb” or “monoclonal antibody” refers to a sdAb/antibody obtained from a group of substantially homogeneous sdAbs/antibodies, that is, a sdAb/antibody group wherein the sdAbs/antibodies constituting the group are homogeneous except for naturally occurring mutants that exist in a small amount. Monoclonal sdAbs/antibodies are highly specific and interact with a single antigenic site. Furthermore, each monoclonal sdAb/antibody targets a single antigenic determinant (epitope) on an antigen, as compared to common polyclonal sdAb/antibody preparations that typically contain various sdAbs/antibodies against diverse antigenic determinants. In addition to their specificity, monoclonal sdAbs/antibodies are advantageous in that they are typically produced from hybridoma cultures not contaminated with other immunoglobulins.

The adjective “monoclonal” indicates a characteristic of antibodies and sdAbs obtained from a substantially homogeneous group of antibodies/sdAbs, and does not specify antibodies/sdAbs produced by a particular method. For example, a monoclonal sdAb to be used in the present invention can be produced by, for example, hybridoma methods (Kohler and Milstein, Nature 256:495, 1975) or recombination methods (U.S. Pat. No. 4,816,567). The monoclonal sdAbs used in the present invention can be also isolated from a phage sdAb library (Clackson et al., Nature 352:624-628, 1991; Marks et al., J. Mol. Biol. 222:581-597, 1991). The monoclonal sdAbs of the present invention may be linked to other antibody domain sequences. Therefore, the resulting polypeptides/protein molecules may be “chimeric” immunoglobulins, wherein a part of the polypeptide is derived from a specific species or a specific antibody class or subclass, and the remaining portion is derived from another species, or another antibody class or subclass. Furthermore, mutant sdAbs, as well as mutant polypeptides/protein molecules comprising a sdAb of the invention, are also comprised in the present invention (U.S. Pat. No. 4,816,567; Morrison et al., Proc. Natl. Acad. Sci. USA 81:6851-6855, 1984).

As used herein, the term “mutant sdAb” or “mutant antibody” refers to a sdAb/antibody comprising a variant amino acid sequence in which one or more amino acid residues have been altered. For example, the variable region of a sdAb/antibody can be modified to improve its biological properties, such as antigen binding. Such modifications can be achieved by site-directed mutagenesis (see Kunkel, Proc. Natl. Acad. Sci. USA 82:488 (1985)), PCR-based mutagenesis, cassette mutagenesis, and the like. Such mutants comprise an amino acid sequence which is at least 70% identical to the amino acid sequence of the heavy chain variable region of the sdAb, more specifically at least 75%, even more specifically at least 80%, still more specifically at least 85%, yet more specifically at least 90%, and most specifically at least 95% identical. Such mutants also comprise an amino acid sequence which is at least 70% identical to the amino acid sequence of a heavy or light chain variable region of the antibody, more specifically at least 75%, even more specifically at least 80%, still more specifically at least 85%, yet more specifically at least 90%, and most specifically at least 95% identical. As used herein, the term “sequence identity” is defined as the percentage of residues identical to those in the sdAb's/antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

Polyclonal and monoclonal sdAbs/antibodies can be prepared by methods known to those skilled in the art.

In another embodiment, antibodies or antibody fragments (e.g., sdAbs) can be isolated from an antibody/sdAb phage library, produced by using the technique reported by McCafferty et al. (Nature 348:552-554 (1990)). Clackson et al. (Nature 352:624-628 (1991)), Marks et al. (J. Mol. Biol. 222:581-597 (1991)) and Muyldermans et al. (Annual Review of Biochemistry Volume 82, pp 775-797(2013)) reported on the respective isolation of mouse, camelid and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal sdAbs/antibodies, instead of using conventional hybridoma technology for monoclonal sdAb/antibody production.

SdAbs/antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant sdAbs/antibodies, produced by gene engineering. The genes encoding the sdAbs/antibodies obtained by a method described above are isolated from B cells or hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The present invention provides the nucleic acids encoding the sdAbs/antibodies of the present invention, and vectors comprising these nucleic acids. Specifically, using a reverse transcriptase, cDNAs encoding the variable region(s) (V region) of the sdAbs/antibodies are synthesized from the mRNAs of B cells or hybridomas. After obtaining the DNAs encoding the variable region(s) of interest, they are optionally ligated with DNAs encoding desired constant regions (C regions), and the resulting DNA constructs are inserted into expression vectors. Alternatively, the DNAs encoding the variable region(s) may be inserted into expression vectors comprising the DNAs of the C regions. These are inserted into expression vectors so that the genes are expressed under the regulation of an expression regulatory region, for example, an enhancer and promoter. Then, host cells are transformed with the expression vectors to express the sdAbs/antibodies. The present invention provides cells expressing sdAbs/antibodies of the present invention. The cells expressing sdAbs/antibodies of the present invention include cells and hybridomas transformed with a gene of such a sdAb/antibody.

The sdAbs/antibodies of the present invention also include sdAbs/antibodies which comprise complementarity-determining regions (CDRs), or regions functionally equivalent to CDRs. The term “functionally equivalent” refers to comprising amino acid sequences similar to the amino acid sequences of CDRs of any of the monoclonal sdAbs isolated in the Examples. The term “CDR” refers to a region in a sdAb/antibody variable region (also called “V region”), and determines the specificity of antigen binding. The H chain and L chain (if present) each have three CDRs, designated from the N terminus as CDR1, CDR2, and CDR3. There are four regions flanking these CDRs: these regions are referred to as “framework,” and their amino acid sequences are highly conserved. The CDRs can be transplanted into other sdAbs/antibodies, and thus a recombinant antibody can be prepared by combining CDRs with the framework of a desired sdAb/antibody. One or more amino acids of a CDR can be modified without losing the ability to bind to its antigen. For example, one or more amino acids in a CDR can be substituted, deleted, and/or added.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and specifically within 35%, and still more specifically within 30% (e.g., within 25%). The identity of amino acid sequences can be determined as described herein.

In the present invention, recombinant sdAbs/antibodies artificially modified to reduce heterologous antigenicity against humans can be used. Examples include chimeric sdAbs/antibodies and humanized sdAbs/antibodies. These modified sdAbs/antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Additionally, a chimeric antibody or polypeptide may be produced by combining a sdAb of the invention with constant regions that are of different species to each other. Such an antibody (e.g., a camelid-human chimeric antibody) can be obtained by (1) ligating a DNA from the different regions; (2) incorporating this into an expression vector; and (3) introducing the vector into a host for production of the antibody.

A humanized sdAb/antibody, which is also called a reshaped human sdAb/antibody, may be obtained by replacing a CDR of a human antibody with an H or L chain CDR of a sdAb/antibody of a nonhuman mammal such as a mouse or camelid. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate a CDR of a mouse/camelid antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of a variable region may be substituted such that the CDR of a reshaped human antibody forms an appropriate antigen-binding site (Sato, K. et al., Cancer Res. (1993) 53, 851-856).

Isotypes of sdAb-fusion proteins or antibodies comprising a sdAb of the present invention, or antibody fragments thereof, are not limited. The isotypes include, for example, IgG (IgG1, IgG2, IgG3, and IgG4), IgM, IgA (IgA1 and IgA2), IgD, and IgE.

As described herein, a sdAb of the present invention may be operably linked to one or more antibody domain sequences (e.g., a sdAb-Fc fusion protein). Therefore, such polypeptides/protein molecules comprising a sdAb of the invention and one or more additional antibody domains may be referenced herein as an antibody or antibody fragment. The term “antibody fragment” refers to a portion of a full-length antibody, and generally to a fragment comprising an antigen-binding domain or a variable region. Such antibody fragments include, for example, single domain antibody (sdAb), Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

The sdAbs/antibodies can be purified to homogeneity. The sdAbs/antibodies can be isolated and purified by a method routinely used to isolate and purify proteins. The sdAbs/antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). SdAbs/antibodies can also be purified by utilizing antigen binding, using carriers on which antigens have been immobilized.

The sdAbs/antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the sdAbs/antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, sdAbs/antibodies of the present invention may be encapsulated in microcapsules (microcapsules made of hydroxycellulose, gelatin, polymethylmethacrylate, and the like), and made into components (encapsulated or as a surface functionalization moiety) of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the sdAbs/antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

### Nucleic Acids, Expression Cassettes, Vectors and Cells

Certain embodiments of the invention provide an isolated nucleic acid encoding a binder protein as described herein (e.g., a sdAb or an antibody, or antibody fragment thereof, comprising a sdAb of the invention).

For example, certain embodiments of the invention provide an isolated nucleic acid comprising one or more CDR sequences (e.g., 1, 2 or 3 CDR sequences), wherein the CDR sequence has at least about 80% (e.g., at least about 80%, 85%, 90%, 91%, 92%, 93%, 94%, 95%, 96%, 97%, 98%, 99% or 100%) sequence identity to a CDR sequence described herein (e.g., identified in any of SEQ ID NOs: 22-27 of Table A). In certain embodiments, the isolated nucleic acid comprises the three CDR sequences shown in any of SEQ ID NOs: 22-27 in Table A. In certain embodiments, the isolated nucleic acid comprises the three CDR sequences shown in any of SEQ ID NOs: 22-27 in Table A. In certain embodiments, the isolated nucleic acid comprises the three CDR sequences shown in any of SEQ ID NOs: 22-27 in Table A.

Certain embodiments of the invention provide an isolated nucleic acid comprising a sequence that has at least about 80% (e.g., at least about 80%, 85%, 90%, 91%, 92%, 93%, 94%, 95%, 96%, 97%, 98%, 99% or 100%) sequence identity to SEQ ID NO: 22 or 23. In certain embodiments, the isolated nucleic acid comprises or consists of SEQ ID NO: 22 or 23. In certain embodiments, the isolated nucleic acid comprises or consists of SEQ ID NO: 22 or 23. In certain embodiments, the isolated nucleic acid comprises of consists of SEQ ID NO: 22 or 23.

Certain embodiments of the invention also provide an isolated nucleic acid comprising a sequence that has at least about 80% (e.g., at least about 80%, 85%, 90%, 91%, 92%, 93%, 94%, 95%, 96%, 97%, 98%, 99% or 100%) sequence identity to SEQ ID NO: 24 or 25. In certain embodiments, the isolated nucleic acid comprises or consists of SEQ ID NO: 24 or 25. In certain embodiments, the isolated nucleic acid comprises or consists of SEQ ID NO: 24 or 25. In certain embodiments, the isolated nucleic acid comprises of consists of SEQ ID NO: 24 or 25.

Certain embodiments of the invention also provide an isolated nucleic acid comprising a sequence that has at least about 80% (e.g., at least about 80%, 85%, 90%, 91%, 92%, 93%, 94%, 95%, 96%, 97%, 98%, 99% or 100%) sequence identity to SEQ ID NO: 26 or 27. In certain embodiments, the isolated nucleic acid comprises or consists of SEQ ID NO: 26 or 27. In certain embodiments, the isolated nucleic acid comprises or consists of SEQ ID NO: 26 or 27. In certain embodiments, the isolated nucleic acid comprises of consists of SEQ ID NO: 26 or 27.

In certain embodiments, the nucleic acid further comprises a promoter.

Certain embodiments of the invention provide an expression cassette comprising a nucleic acid as described herein and a promoter.

Certain embodiments of the invention provide a vector (e.g., a phagemid, Adeno-associated viruses (AAV)) comprising a nucleic acid or an expression cassette as described herein.

Certain embodiments of the invention provide a cell comprising a nucleic acid, expression cassette or vector as described herein. In certain embodiments, the cell is a bacterial cell.

In certain embodiments, the cell is a mammalian cell.

In certain embodiments, the cell is a human mammalian cell. In certain embodiments, the cell is a human embryonic kidney (HEK) 293 cell. In certain embodiments, the cell is a 293F cell. In certain embodiments, the cell is a 293T cell. In certain embodiments, the cell is a human embryonic retinal (PER.C6) cell. In certain embodiments, the cell is a HT-1080 cell. In certain embodiments, the cell is a Huh-7 cell.

In certain embodiments, the cell is a non-human mammalian cell. In certain embodiments, the cell is a Monkey kidney epithelial (Vero) cell. In certain embodiments, the cell is a Chinese Hamster Ovary (CHO) cell. In certain embodiments, the cell is a baby hamster kidney (BHK) cell.

In certain embodiments, the cell is a non-mammalian cell. In certain embodiments, the cell is an insect cell. In certain embodiments, the cell is a yeast cell.

Certain embodiments of the invention provide a phage particle comprising a vector as described herein.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res.,19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more specifically at least 150 nucleotides, and still more specifically at least 400 nucleotides. If not employed for expressing, a “portion” or “fragment” means at least 9, specifically 12, more specifically 15, even more specifically at least 20, consecutive nucleotides, e.g., probes and primers (oligonucleotides), corresponding to the nucleotide sequence of the nucleic acid molecules of the invention.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, culture medium may represent less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., 71%, 72%, 73%, 74%, 75%, 76%, 77%, 78%, to 79%, generally at least 80%, e.g., 81% -84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, viral vector, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. “Operably-linked” also refers to the association two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”
(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.
(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more specifically less than about 0.01, and most specifically less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

### Compositions and Kits

Certain embodiments provide a composition comprising an anti-SARS-CoV-2 binder protein as described herein, or a vector, or a cocktail mixture thereof, as described herein and a carrier. In certain embodiments, the composition is a pharmaceutical composition comprising a pharmaceutically acceptable carrier.

In certain embodiments, the composition is a cocktail mixture comprising two or more anti-SARS-CoV-2 binder proteins that each bind to non-overlapping regions (e.g., different epitopes) on the RBD of SARS-CoV spike protein. In certain embodiments, the composition is a cocktail mixture that is capable of conferring broad spectrum inhibition against SARS-coronaviruses. For example, in certain embodiments, the composition is a cocktail mixture of anti-SARS-CoV-2 binder proteins that collectively are capable of inhibiting or binding to two or more (e.g., 3, 4, 5, 6, or more) SARS-CoV strains or variants selected from the group consisting of SARS-CoV-2, Alpha, Delta, Omicron, SARS-CoV-1, bat SARS1, and bat SARS2. In certain embodiments, the composition is a cocktail mixture of anti-SARS-CoV-2 binder proteins that collectively are capable of inhibiting or binding to SARS-CoV-2, Alpha, Delta, Omicron, SARS-CoV-1, bat SARS1, and bat SARS2. In certain embodiments, the composition is a cocktail mixture comprising two or more (e.g., three) anti-SARS-CoV-2 binder protein (e.g., sdAbs or sdAb-Fc) as described herein.

In certain embodiments, the composition comprises two or more (e.g., three, or four) SARS-CoV-2 binder protein selected from the group consisting of:
(a) an isolated anti-SARS-CoV-2 binder protein comprising an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 1 or 6;
(b) an isolated anti-SARS-CoV-2 binder protein comprising an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 7 or 12;
(c) an isolated anti-SARS-CoV-2 binder protein comprising an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 13 or 18; and
(d) an isolated anti-SARS-CoV-2 binder protein comprising an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 101 or 152; and
(e) an isolated anti-SARS-CoV-2 binder protein comprising an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 164 or 166.

Accordingly, in certain embodiments, the composition comprises three SARS-CoV-2 binder proteins, wherein:
the first binder protein is according to (a),
the second binder protein is according to (b), and
the third binder protein is according to (c).

In certain embodiments, the composition comprises four SARS-CoV-2 binder proteins, wherein:
the first binder protein is according to (a),
the second binder protein is according to (b),
the third binder protein is according to (c), and
the fourth binder protein is according to (d).

As a non-limiting example, in certain embodiments, the composition comprises three SARS-CoV-2 binder protein that are:
(a) Nanosota-2A or Nanosota-2A-Fc as described herein;
(b) Nanosota-3A or Nanosota-3A-Fc as described herein; and
(c) Nanosota-4A or Nanosota-4A-Fc as described herein.

As another non-limiting example, in certain embodiments, the composition comprises two SARS-CoV-2 binder protein that are:
Nanosota-2A or Nanosota-2A-Fc as described herein and
Nanosota-3A or Nanosota-3A-Fc as described herein;
or
Nanosota-2A or Nanosota-2A-Fc as described herein and
Nanosota-4A or Nanosota-4A-Fc as described herein;
or
Nanosota-3A or Nanosota-3A-Fc as described herein and
Nanosota-4A or Nanosota-4A-Fc as described herein.

In certain embodiments, the composition is a liquid composition. In certain embodiments, the composition is a solid composition (e.g., powder or lyophilized formulation). In certain embodiments, the composition is a lyophilized composition that further comprises one or more excipients selected from the group consisting of a cryo-lyoprotectant (e.g., trehalose, sucrose) and a bulking agent (e.g., mannitol, glycine). In certain embodiments, the solid composition may be reconstituted (e.g., with water, saline or Dextrose solution) prior to use.

Certain embodiments also provide a kit comprising an isolated anti-SARS-CoV-2 binder protein as described herein, or a vector, or a cocktail mixture thereof, as described herein, packaging material, and instructions for detecting the presence of SARS-CoV-2 in a biological sample or for administering the binder protein/vector, to a mammal to treat a SARS-CoV (e.g., SARS-CoV-2 or SARS-CoV-1) infection. In certain embodiments, the kit further comprises at least one additional therapeutic agent. In certain embodiments, the at least one additional therapeutic agent is useful for preventing or treating a viral infection or inflammation. In certain embodiments, the at least one additional therapeutic agent is an antibody or a sdAb.

In certain embodiments, the kit further comprises a syringe (e.g., a pre-filled syringe) or a vial comprising the composition as described herein.

In certain embodiments, the kit further comprises an atomizer nozzle for nasal or pulmonary delivery, wherein the atomizer nozzle is or could be fitted with the syringe or vial to produce a spray or mist.

In certain embodiments, the kit further comprises an inhaler device.

In certain embodiments, the kit further comprises a needle that is or could be fitted with the syringe (e.g., to deliver subcutaneous, intradermal, or intramuscular injection).

In certain embodiments, the kit comprises instructions for detecting the presence of SARS-CoV-2 in a biological sample (e.g., saliva, sputum, blood, serum, or a biological sample obtained from swab of nostril or throat). In certain embodiments, the kit is a lateral flow assay kit (e.g., strip or cartridge). In certain embodiments, the kit is suitable for conducting a colorimetric, chemiluminescent, or fluorescent assay. In certain embodiments, the kit is suitable for conducting flow cytometry, or ELISA. In certain embodiments, the kit comprises a binder protein (e.g., from Class 1-6, or Nanosota-2A, -3A, -4A, -5, -6, or -7) as described in Tables 1-6, wherein the binder protein may be operably linked to an optional detectable agent (e.g., tag) described herein. In certain embodiments, the kit comprises a binder protein (e.g., class 4, 5, 6, or Nanosota-5, 6, or 7) as described in Tables 4-6, wherein the binder protein may be operably linked to an optional detectable agent (e.g., tag) described herein. In certain embodiments, the kit comprises a binder protein (e.g., Nanosota-5 from Class4) as described in Table 4, wherein the binder protein may be operably linked to an optional detectable agent (e.g., tag) described herein. In some embodiments, the binder protein comprises CDRs 1-3 consisting of the amino acid sequences of SEQ ID NOs: 102, 103, and 104, respectively. In some embodiments, the binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 101 or 152. In some embodiments, the binder protein comprises CDRs 1-3 consisting of the amino acid sequences of SEQ ID NOs: 102, 103, and 97, respectively. In some embodiments, the binder protein comprises an amino acid sequence that has at least about 80% (e.g., 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO: 164 or 166.

### Methods of Use

Certain embodiments provide a method of inhibiting the activity of SARS-CoV (e.g., SARS-CoV-2 or SARS-CoV-1), comprising contacting SARS-CoV with an isolated anti-SARS-CoV-2 binder protein as described herein, (e.g., under conditions suitable for binding between the binder protein and SARS-CoV, such as between the binder protein and the SARS-CoV RBD). In certain embodiments, binding between SARS-CoV and ACE2 is inhibited. Thus, certain embodiments also provide a method for inhibiting the binding between SARS-CoV (e.g., Omicron variant) and ACE2, comprising contacting SARS-CoV with an isolated anti-SARS-CoV-2 binder protein (e.g., Nanosota-5) as described herein, (e.g., under conditions suitable for binding between the binder protein and SARS-CoV, such as between the binder protein and the SARS-CoV RBD).

In certain embodiments, the SARS-CoV protein (e.g., spike protein) is contacted in vitro. In certain embodiments, the SARS-CoV protein is contacted in vivo. In certain embodiments, the SARS-CoV protein is contacted extracellularly. In certain embodiments, the SARS-CoV protein is contacted intracellularly (e.g., intracellularly delivered or expressed sdAb binds SARS-Cov within an infected cell). Methods for measuring the activity of SARS-CoV (e.g., ability to bind ACE2) are known in the art. For example, in certain embodiments, an assay described herein may be used. In certain embodiments, a binder protein of the invention inhibits the activity of SARS-CoV (e.g., its ability to bind ACE2) by at least about 5%, at least about 10%, at least about 15%, at least about 20%, at least about 25%, at least about 30%, at least about 40%, at least about 50%, at least about 60%, at least about 70%, at least about 80%, at least about 90%, at least about 95%, at least about 99% or at least about 100% as compared to a control.

In certain embodiments, a binder protein of the invention inhibits the activity of a SARS-CoV (live virus or pseudovirus of a SARS-CoV strain or variant) with a IC₅₀ potency of about 0.1, 1, 2, 3, 4, 5, 6, 7, 8, 9, 10, 20, 30, 40, 50, 60, 70, 80, 90, 100, 200, 300, 400, 500, 600, 700, 800, or 900 pM. In certain embodiments, a binder protein of the invention inhibits the activity of a SARS-CoV (live virus or pseudovirus of a SARS-CoV strain or variant) with a IC₅₀ potency of about 0.1 pM to 10 nM, 1 pM to 1 nM, 2 pM to 300 pM, 3 pM to 200 pM, 4 pM to 150 pM, or 5 pM to 100 pM.

Certain embodiments also provide a method for treating or preventing a SARS-CoV (e.g., SARS-CoV-1, or SARS-CoV-2 or variant thereof, such as Omicron) infection in a mammal, comprising administering an effective amount of an isolated anti-SARS-CoV-2 binder protein (e.g., Nanosota-5 or Nanosota-5-Fc), or a vector as described herein to the mammal. In certain embodiments, the SARS-CoV is SARS-CoV-2 Omicron variant (e.g., BA.1, BA. 5, XBB.1.5, EG.5, or JN.1).

In certain embodiments, the method further comprises administering at least one additional therapeutic agent to the mammal. In certain embodiments, the at least one additional therapeutic agent is useful for treating a viral infection or inflammation. In certain embodiments, the at least one additional therapeutic agent is an antibody or a sdAb.

Certain embodiments provide an isolated anti-SARS-CoV-2 binder protein, or vector as described herein for the prophylactic or therapeutic treatment of a SARS-CoV (e.g., SARS-CoV-1, or SARS-CoV-2 or variant thereof, such as Omicron) infection.

Certain embodiments provide the use of an isolated anti-SARS-CoV-2 binder protein or vector as described herein to prepare a medicament for the treatment of a SARS-CoV (e.g., SARS-CoV-1, or SARS-CoV-2 or variant thereof, such as Omicron) infection in a mammal.

Certain embodiments provide an isolated anti-SARS-CoV-2 binder protein or vector as described herein for use in medical therapy.

Certain embodiments provide a method of detecting the presence of SARS-CoV (e.g., SARS-CoV-1, or SARS-CoV-2 or variant thereof, such as Omicron) in a biological sample, the method comprising contacting the biological sample (e.g., saliva, sputum, blood, serum, or a biological sample obtained from swab of nostril or throat) with an isolated anti-SARS-CoV-2 binder protein described herein and detecting whether a complex is formed between the anti-SARS-CoV-2 binder protein and SARS-CoV. In certain embodiments, the binder protein comprises a protein as described in Tables 1-6. In certain embodiments, the binder protein comprises a protein as described in Table 4 (e.g., Nanosota-5 or Nanosota-5-Fc).

### Administration

For in vivo use, a protein molecule as described herein (e.g., a sdAb of the invention, or a polypeptide or protein molecule comprising such a sdAb), or a vector, or a cocktail mixture thereof, as described herein is generally incorporated into a pharmaceutical composition prior to administration. Within such compositions, one or more protein molecules or vectors of the invention may be present as active ingredient(s) (i.e., are present at levels sufficient to provide a statistically significant effect on the symptoms of a relevant disease (e.g., a SARS-CoV-2 infection), as measured using a representative assay). A pharmaceutical composition comprises one or more such protein molecules or vectors in combination with any pharmaceutically acceptable carrier(s) known to those skilled in the art to be suitable for the particular mode of administration. In addition, other pharmaceutically active ingredients (including other therapeutic agents) may, but need not, be present within the composition.

In certain embodiments, a SARS-CoV binder protein or composition described herein could be administered to an animal (e.g., mammal such as human) in need of before or after SARS-CoV infection, for example, for prevention or treatment of SARS-CoV infection. In certain embodiments, a SARS-CoV binder protein or composition described herein could be administered to an animal about 3, 2, 1 week(s) or 6, 5, 4, 3, 2, 1 day(s) or 20, 15, 10, 5, 1 hour(s) before SARS-CoV infection or before potential exposure to SARS-CoV. In certain embodiments, a SARS-CoV binder protein or composition described herein could be administered to an animal about 1, 2, 3, 4, 5, 6, 7, 8, 9, 10, 11, 12, 13, 14, 15, 16, 17, 18, 19, 20, 21, 22, 23 hour(s), 1, 2, 3, 4, 5, 6 day(s), or 1, 2, 3 week(s) after suspected or confirmed SARS-CoV infection.

In certain embodiments, a SARS-CoV binder protein (e.g., a sdAb, a sdAb-Fc, or a protein comprising the sdAb) is administered to an animal (e.g., mammal such as human) in need of at a dosage of about 1, 2, 3, 4, 5, 6, 7, 8, 9, 10, 11, 12, 13, 14, 15, 16, 17, 18, 19, 20, 21, 22, 23, 25, 26, 27, 28, 29, or 30 mg/kg. In certain embodiments, a SARS-CoV binder protein (e.g., a sdAb, a sdAb-Fc, or a protein comprising the sdAb) is administered to an animal (e.g., mammal such as human) in need of at a dosage range of about 0.1 to 50, 0.5 to 40, 1 to 30, 2 to 25, 3 to 20, 4 to 18, or 5 to 16 mg/kg.

The term “therapeutically effective amount,” in reference to treating a disease state/condition, refers to an amount of a protein molecule or vector either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder, such as a SARS-CoV-2 infection. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

In certain embodiments, the present protein molecules/vectors may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the protein molecule/vector may be combined with one or more excipients and used in the form of ingestible tablets, buccal tablets, troches, capsules, elixirs, suspensions, syrups, wafers, and the like. Such compositions and preparations should contain at least 0.1% of a protein molecule/vector of the present invention. The percentage of the compositions and preparations may, of course, be varied and may conveniently be between about 2 to about 60% of the weight of a given unit dosage form. The amount of protein molecule/vector in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the protein molecule/vector, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the protein molecule/vector may be incorporated into sustained-release preparations and devices.

The protein molecule or a vector as described herein may also be administered subcutaneously, intradermally, intranasally, intramuscularly, intravenously or intraperitoneally by infusion or injection. The protein molecule or a vector as described herein may also be administered via intranasal and/or pulmonary delivery (e.g., delivered as a spray or mist). Additionally, the protein molecule or vector may be administered by local injection, such as by intrathecal injection, epidural injection or peri-neural injection using a scope. Solutions of the protein molecule or vector may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the protein molecule or vector that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be useful to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the protein molecule or vector in the required amount in the appropriate solvent with various of the other ingredients enumerated above, as required, followed by filter sterilization. In the case of sterile powders for the preparation of sterile injectable solutions, the methods of preparation are vacuum drying and the freeze drying techniques, which yield a powder of the protein molecule or vector plus any additional desired ingredient present in the previously sterile-filtered solutions.

For topical administration, the present protein molecules/vectors may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present protein molecules/vectors can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the protein molecules/vectors of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the protein molecules or vectors of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of a protein molecule or vector of the present invention required for use in treatment will vary with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Protein molecules or vectors of the invention can also be administered in combination with other therapeutic agents and/or treatments, such as other agents or treatments that are useful for the treatment of a SARS-CoV (e.g., SARS-CoV-2 or SARS-CoV-1) infection. In certain embodiments such an agent is an antibody or a sdAb. Additionally, one or more protein molecules or vectors of the invention, may be administered (e.g., a combination of sdAbs, polypeptides, protein molecules and/or vectors may be administered). Accordingly, one embodiment the invention also provides a composition comprising a protein molecule or vector of the invention, at least one other therapeutic agent, and a pharmaceutically acceptable diluent or carrier. The invention also provides a kit comprising a protein molecule or vector of the invention, at least one other therapeutic agent, packaging material, and instructions for administering a protein molecule or vector of the invention, and the other therapeutic agent or agents to an animal to treat a SARS-CoV (e.g., SARS-CoV-2 or SARS-CoV-1) infection.

As used herein, the term “therapeutic agent” refers to any agent or material that has a beneficial effect on the mammalian recipient.

The invention will now be illustrated by the following non-limiting Examples.

### EXAMPLE 1. Identification of Nanosota-2, -3, and -4 as Super Potent and Wide-Spectrum Therapeutic Candidates Against COVID-19

Nanobodies are antigen-binding domains of heavy-chain only antibodies produced by camelids; they are superior therapeutic candidates due to their small sizes. Here in this Example, we describe the identification of three anti-SARS-CoV-2 nanobodies, named Nanosota-2, -3 and -4, from alpacas immunized with SARS-CoV-2 spike protein. As described below, their potency in vitro and in mice were extensively evaluated. The potency metrics of Nanosota-2 are among the best of known anti-SARS-CoV-2 entry inhibitors. Nanosota-3 is highly potent against the omicron variant. Nanosota-4 is effective against both SARS-CoV-1 and SARS-CoV-2. The combined antiviral spectrum of Nanosota-2, -3 and -4 cover SARS-CoV-2, its major variants and SARS-CoV-1. Cryo-EM data explain the antiviral potency and spectrum of the nanobodies. In addition to their super potency and wide antiviral spectrum, the nanobodies are cost-effective, and have the potential to being administered as inhalers. In sum, the Nanosota series are powerful therapeutic candidates that can help end the COVID-19 pandemic and prepare for possible future coronavirus pandemics.

### Introduction

The COVID-19 pandemic has been complicated by the emergence of SARS-CoV-2 variants and many breakthrough infections of vaccinated people (1, 2). Potent, wide-spectrum, cost effective and easily administered anti-SARS-CoV-2 therapeutics are urgently needed to help end the current pandemic and prepare for possible future coronavirus pandemics. The virus-surface spike protein mediates SARS-CoV-2 entry into host cells, induces most of the host immune responses, and is a prime target for antiviral interventions (3, 4). The receptor-binding domain (RBD) on the tip of the spike attaches SARS-CoV-2 to its host receptor ACE2 and is heavily targeted by host immune responses (5-7). The RBD takes either a standing-up conformation for receptor binding or a lying-down conformation for immune evasion (8-10). The spike protein, particularly the RBD, undergo extensive mutations in SARS-CoV-2 variants (1, 11). The immune evasiveness and rapid evolution of the RBD make the development of potent and wide-spectrum entry inhibitors challenging.

Conventional antibodies have been the main therapeutic tools targeting SARS-CoV-2 entry (Table S1), but the pandemic exposed their limitations. First, conventional antibodies are expensive due to the high costs associated with production, storage, and transportation (12, 13). Second, they have limited potency, working well when administered before or shortly after SARS-CoV-2 infection, but not against actively replicating SARS-CoV-2 (Table S2) (14, 15). Third, they have limited antiviral spectrums as viral mutations almost inevitably abolish their efficacy (9, 10). Finally, their use is severely limited because they can only be administered through injections (16-18). Therefore, combating a global pandemic using conventional antibodies has many drawbacks.

Nanobodies are single domain antibodies derived from heavy chain-only antibodies in camelid animals (e.g., llamas and alpacas) (19, 20). Due to their small size, nanobodies have many advantages over conventional antibodies as antiviral therapeutics, such as their access to cryptic sites on targets, excellent tissue permeability, ease of production, superior pharmacokinetics, strong physical and chemical stabilities, ability to adapt to viral variants using phage display, and potential to being administered as inhalers (21-27). In addition, nanobodies have low immunogenicity in humans (21, 22). A nanobody drug is clinically available to treat a blood clotting disorder (28), confirming the safety of nanobodies as human therapeutics. A number of anti-SARS-CoV-2 nanobodies have been developed, which show similar potencies as conventional antibodies (Table S1), including Nanosota-1 that we identified from a naive nanobody phage display library and then optimized using phage display (24). However, none of these nanobodies have demonstrated extraordinary potency or spectrum against SARS-CoV-2 both in vitro and in animal models (Table S1).

In this Example, we identified three more nanobodies, named Nanosota-2, -3 and -4, from alpacas immunized with SARS-CoV-2 spike. Their anti-SARS-CoV-2 potencies both in vitro and in animal models were evaluated and the structural basis for their anti-SARS-CoV-2 potencies and spectrums were also determined. This Example demonstrated that nanobodies can potentially achieve super potency and wide spectrum against their viral targets and that the Nanosota series can serve as a powerful therapeutics tool in battling the current and possibly future coronavirus pandemics.

### Results

Identification of Three Novel Nanobodies from Immunized Alpacas

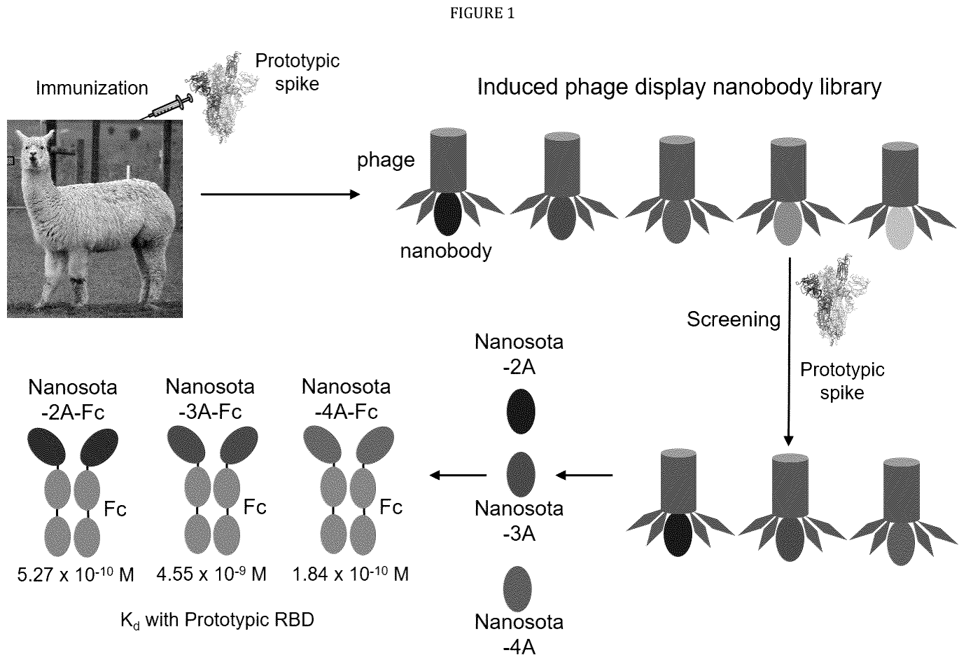

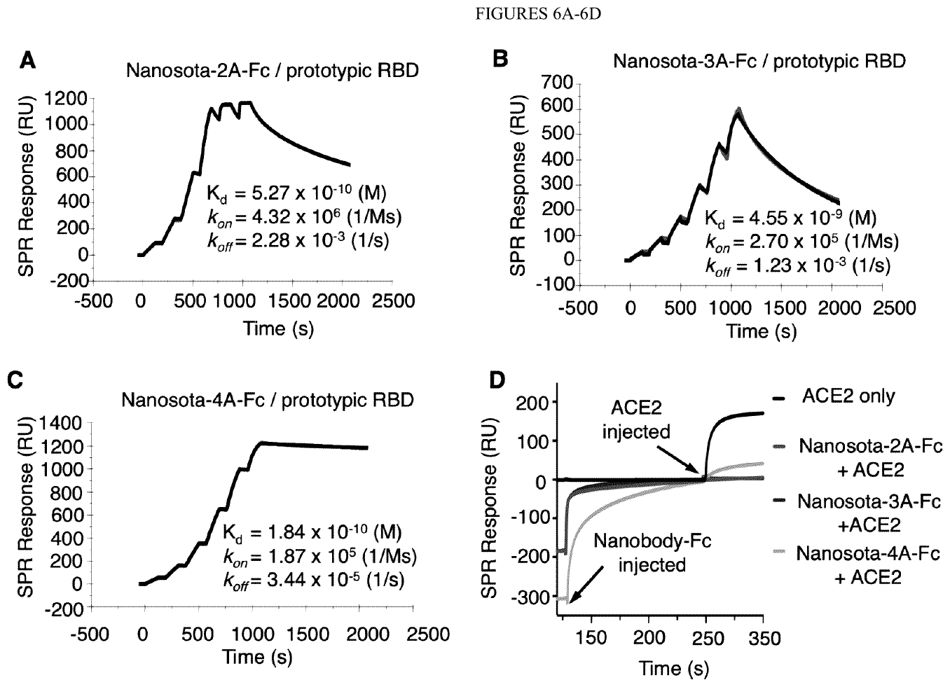

To identify potent anti-SARS-CoV-2 nanobodies, alpacas were immunized with recombinant SARS-CoV-2 spike (prototypic strain), their peripheral blood mononuclear cells (PBMCs) were collected, and an induced nanobody phage display library was established (FIG. 1). This library was screened using recombinant SARS-CoV-2 spike and three spike-binding nanobodies were identified, named Nanosota-2A, Nanosota-3A and Nanosota-4A. Human Fc-tagged nanobodies (nanobody-Fc fusion proteins that are dimerized via Fc domain), named Nanosota-2A-Fc, Nanosota-3A-Fc and Nanosota-4A-Fc, respectively, were then constructed. All three Fc-tagged nanobodies could be expressed in both bacteria and mammalian cells with high production yields (e.g., >40 mg/L cell culture without any optimization). The mammalian cell-expressed proteins were used in this Example. As measured using surface plasmon resonance, Nanosota-2A-Fc, Nanosota-3A-Fc and Nanosota-4A-Fc bind to prototypic SARS-CoV-2 RBD with a K_{d} of 5.27×10⁻¹⁰ M, 4.55×10⁻⁹ M and 1.84×10⁻¹⁰ M, respectively (FIG. 1; FIG. 6A-6C). In comparison, recombinant human ACE2 binds to the same RBD with a K_{d} of 4.4 ×10⁻⁸ M (6). Thus, these Fc-tagged nanobodies all have significantly higher affinity for the prototypic RBD than does ACE2.

The construction of the Fc-tagged nanobodies yielded several desirable properties. Single-domain nanobodies have an in vivo half-life of several hours because their molecular weight (˜14 kDa) is below the kidney clearance threshold (˜60 kDa). In contrast, Fc-tagged nanobodies have an in vivo half-life of >10 days due to their increased molecular weight (˜78 kDa) (24). Fc-tagged dimeric nanobodies are still only about half of the size of conventional antibodies and their single-domain antigen-binding site can still access cryptic epitopes on targets (24). Importantly, compared to conventional antibodies, Fc-tagged nanobodies possess superior therapeutic features (24).

### Evaluation of Anti-SARS-CoV-2 Potency of the Nanobodies In Vitro

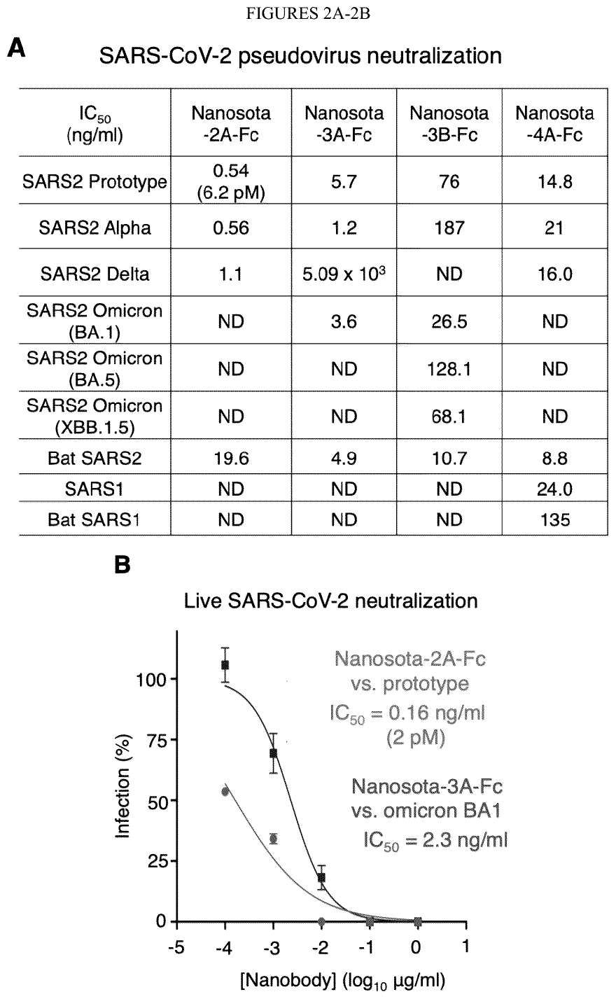

To evaluate the anti-SARS-CoV-2 potency of the nanobodies, we performed SARS-CoV-2 neutralization assays in vitro. Both SARS-CoV-2 pseudoviruses and live SARS-CoV-2 were used. For the former, retroviruses pseudotyped with SARS-CoV-2 spike (i.e., SARS-CoV-2 pseudoviruses) entered human ACE2-expressing cells in the presence of one of the nanobodies. To evaluate antiviral spectrums of the nanobodies, pseudoviruses corresponding to pre-omicron SARS-CoV-2 strains (prototype, alpha and delta), omicron subvariants (early BA.1, later BA.5, and currently circulating XBB1.5), and bat SARS2 were included. Pseudoviruses corresponding to SARS-CoV-1 and bat SARS1 were also included, both of which are related to SARS-CoV-2 and use human ACE2 as their entry receptor (29-31). Both Nanosota-2A-Fc and Nanosota-4A-Fc were potent against all of the SARS-CoV-2 strains except omicron, whereas Nanosota-3A-Fc was potent against the prototype, alpha, omicron BA.1, and bat SARS2, but was weak against delta and ineffective against omicron BA.5 and XBB1.5 (FIG. 2A). Surprisingly, Nanosota-4A-Fc was also effective against SARS-CoV-1 and bat SARS1 (FIG. 2A). For the live SARS-CoV-2 neutralization assay, prototypic SARS-CoV-2 and its omicron variant infected Vero cells in the presence of Nanosota-2A-Fc and Nanosota-3A-Fc, respectively. Nanosota-2A-Fc and Nanosota-3A-Fc were potent against the prototypic strain and omicron strain, respectively (FIG. 2B). Of note, the IC₅₀ values of Nanosota-2A-Fc against the prototypic SARS-CoV-2 pseudoviruses and live prototypic SARS-CoV-2 were 6.2 pM (0.5 ng/ml) and 2 pM (0.16 ng/ml), respectively, both of which are among the best of all known anti-SARS-CoV-2 antibodies and nanobodies (Table S1).

### Evaluation of Anti-SARS-CoV-2 Potency of the Nanobodies in Mouse Models

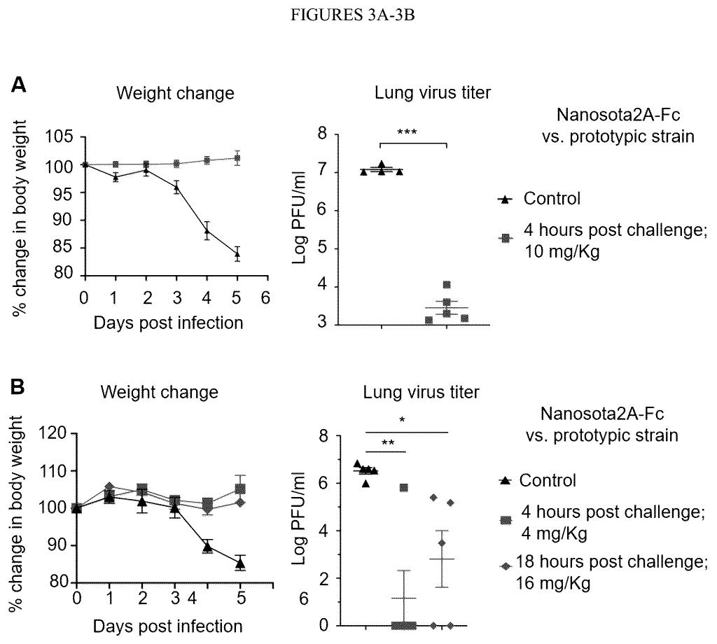

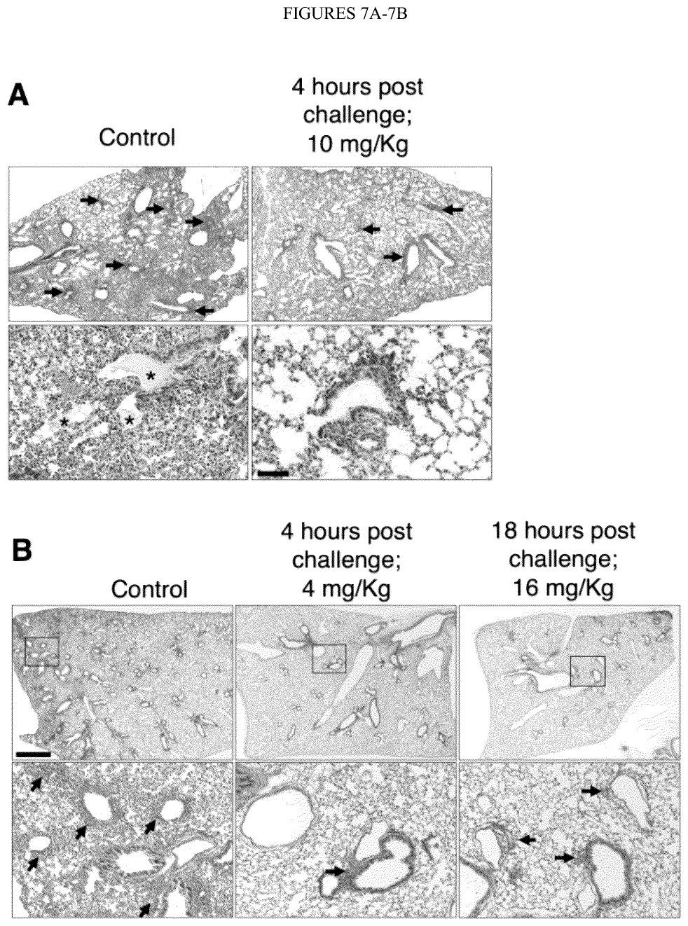

Next, the therapeutic efficacy of Nanosota-2A-Fc in human-ACE2-transgenic mice challenged with prototypic SARS-CoV-2 strain was measured. Specifically, mice were challenged by SARS-CoV-2 via intranasal inoculation; four hours post-challenge, Nanosota-2A-Fc was administered through the intraperitoneal route at the dosage of 10 mg/Kg weight (FIG. 3A). The mice were separated into two groups. One group was monitored for their weight change for five days, whereas the other group was checked for virus titers and pathology in their lungs on day 2 when virus titers peaked. Mice treated with PBS buffer were used as controls. Mice in the control group experienced significant weight loss, contained high-titer viruses in the lungs (˜10⁷ PFU/ml), and developed significant lung pathology (FIG. 3A; FIG. 7A). In comparison, mice treated with Nanosota-2A-Fc experienced no body weight loss, contained low-titer viruses in the lungs (<10⁴ PFU/ml), and developed less significant lung pathology (FIG. 3A; FIG. 7A). Thus Nanosota-2A-Fc effectively treated mice from prototypic SARS-CoV-2 infections when administered 4 hours post-challenge at 10 mg/Kg weight.

To explore the limits of Nanosota-2A-Fc's therapeutic efficacy, the above mouse challenge experiment was repeated with a lowered nanobody dosage or a delayed nanobody administration time (FIG. 3B). For the former condition, Nanosota-2A-Fc at a dosage of 4 mg/Kg weight was administered to mice (at 4 hours post-challenge); for the latter condition, Nanosota-2A-Fc was administered to mice 18 hours post-challenge (at 16 mg/Kg weight). Under these conditions, Nanosota-2A-Fc still effectively treated human-ACE2-transgenic mice from prototypic SARS-CoV-2 challenge (FIG. 3B; FIG. 7B). Hence, Nanosota-2A-Fc treats mice from prototypic SARS-CoV-2 infections at a lowered dosage or at a delayed administration time. Of note, the dosage and the administration time for Nanosota-2A-Fc are among the lowest and latest, respectively, of known anti-SARS-CoV-2 antibodies and nanobodies (Table S2).

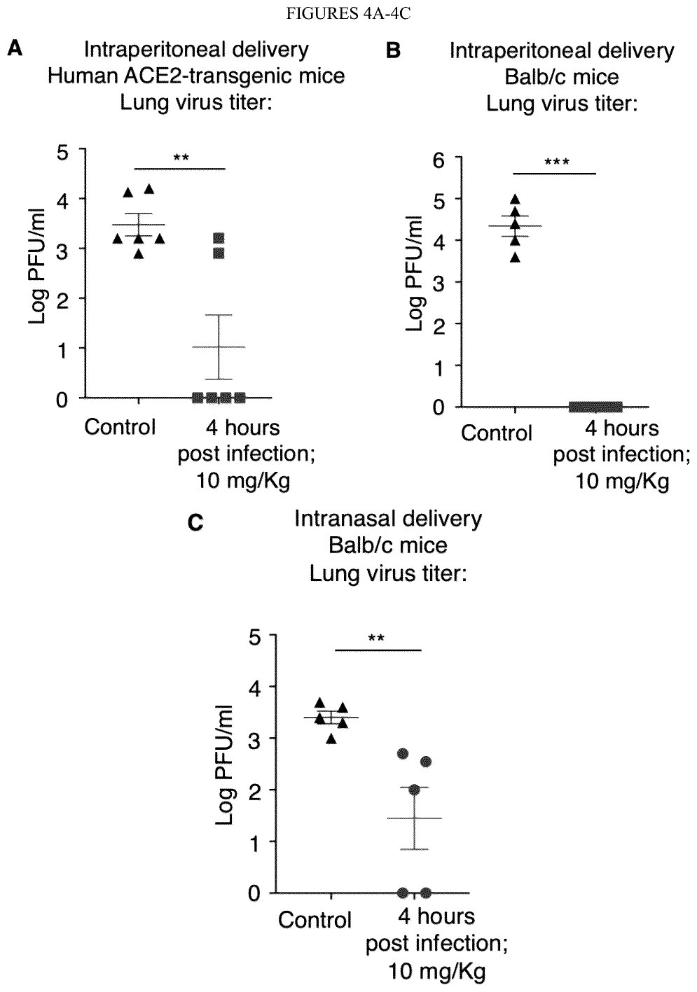

The therapeutic efficacy of Nanosota-3A-Fc was also measured in two different mouse models challenged with the SARS-CoV-2 omicron variant. These two different models are human-ACE2-transgenic mice and wild-type mice, both of which are susceptible to the omicron variant (only the former is susceptible to the prototypic SARS-CoV-2 strain) (32). The result showed that after receiving Nanosota-3A-Fc (10 mg/Kg weight and 4 hours post-challenge), both mouse models experienced insignificant reduction in body weight and significant reduction in lung virus titers (FIG. 4A; 4B). Thus, Nanosota-3A-Fc effectively treats both of the mouse models from the infection of the omicron variant.

To explore the potential of Nanosota-3A-Fc as an inhalation treatment, we conducted a similar experiment using mice, but this time administering the drug through the intranasal route. Balb/c mice were administered Nanosota-3A-Fc as nasal drops (at a dosage of 10 mg/Kg weight and four hours post-challenge). Interestingly, even with this method, Nanosota-3A-Fc still resulted in a significant reduction in lung virus titers in the mouse model (FIG. 4C). Therefore, when administered through the intranasal route, Nanosota-3A-Fc effectively treated the mouse model infected with the omicron BA.1 subvariant.

### Structural Mechanisms for Anti-SARS-CoV-2 Potency of the Nanobodies

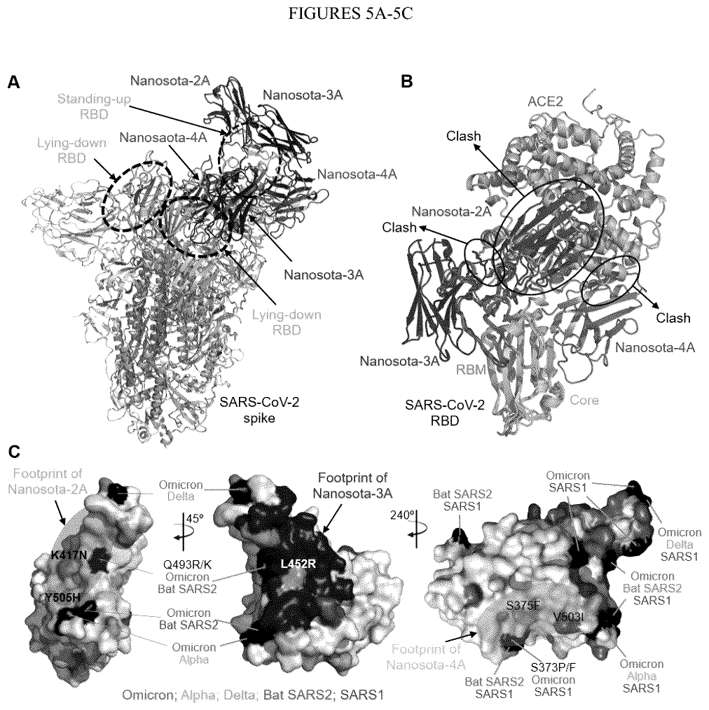

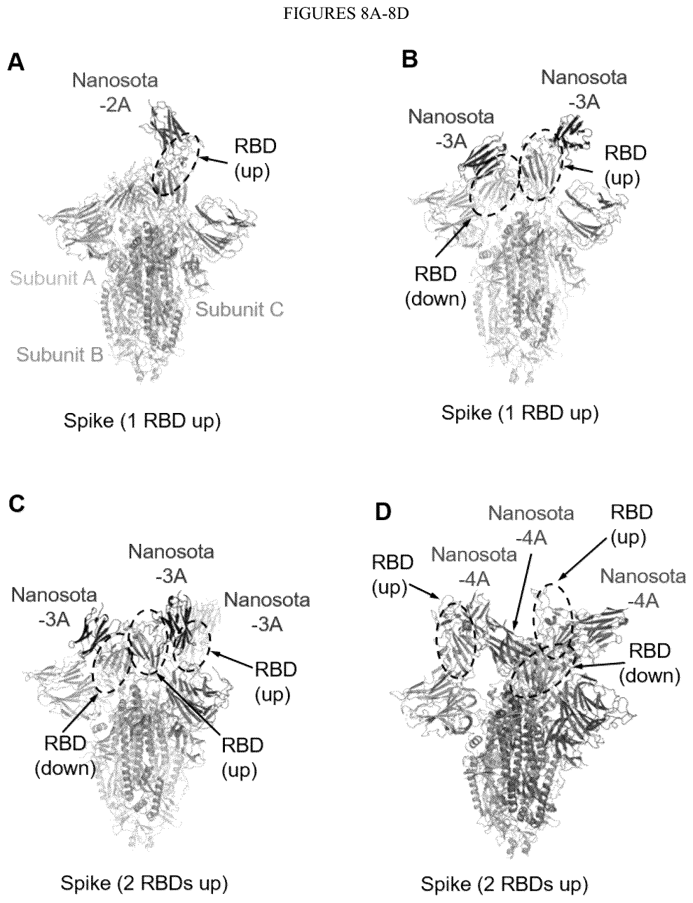

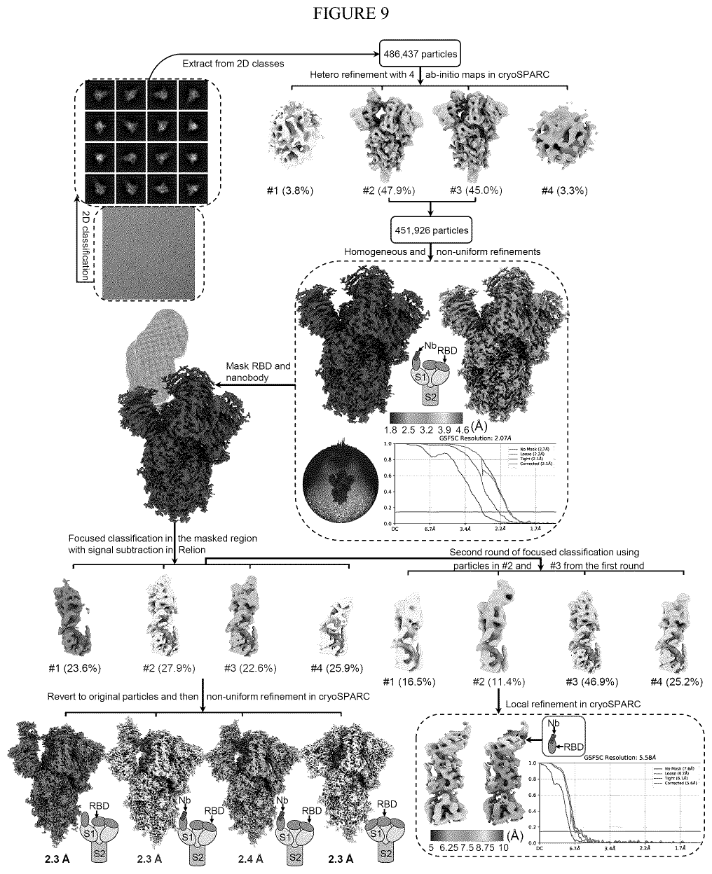

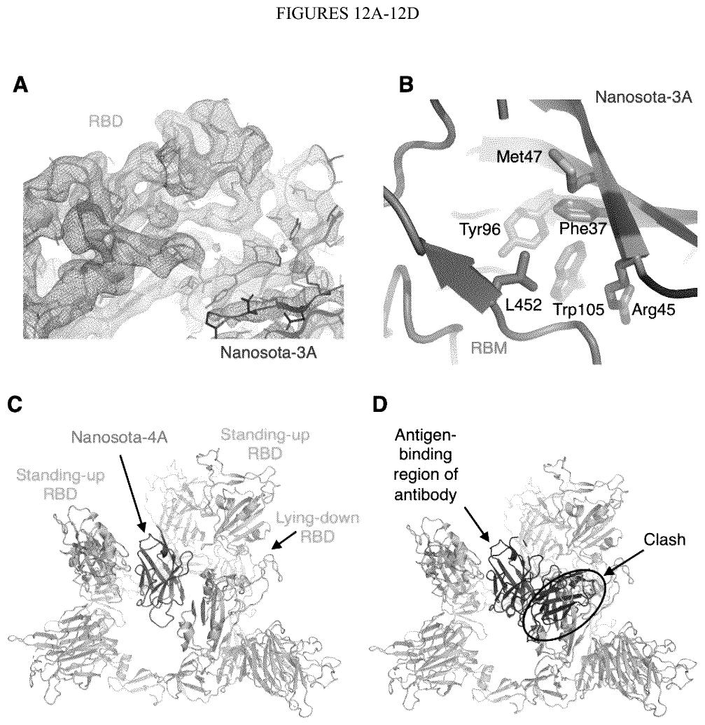

To investigate the structural mechanisms of the three nanobodies in inhibiting SARS-CoV-2, the cryo-EM structures of SARS-CoV-2 spike (prototypic strain) complexed with each of them (Nanosota-2A, Nanosota-3A and Nanosota-4A) was determined (FIG. 5A; FIG. 8). The overall resolutions of the cryo-EM maps were 2.1 Å, 2.5 Å and 3.4 Å, respectively (Table S3; FIG. 9-11). Due to the mobility of the bound nanobodies, the local resolutions of the maps in the nanobody regions were lower. Hence only the atomic model of Nanosota-3A was built based on a 3.2 Å local map (FIG. 12A), whereas docking models of Nanosota-2A and Nanosota-4A were generated. Nanosota-2A only binds to the standing-up RBD, whereas Nanosota-3A and Nanosota-4A bind to both the standing-up and lying-down RBDs. The footprints of all three nanobodies on the RBD were mapped, showing that the three nanobodies bind to non-overlapping regions (FIG. 5B). Specifically, Nanosota-2A binds to the top of the RBD and its footprint completely overlaps with the ACE2-binding region, whereas Nanosota-3A and Nanosota-4A bind to each side of the RBD and both of their footprints only partially overlap with the ACE2-binding region. Thus, binding of each of these nanobodies to the RBD prevents ACE2 from binding to the RBD, explaining their capability in neutralizing SARS-CoV-2 entry.

To validate the above structural information, we conducted competition SPR experiments to see if any of the three nanobodies would compete with ACE2 for RBD binding. Our results indicated that when each of the three Fc-tagged nanobodies bound to the RBD, it either completely blocked ACE2 from binding to the RBD (in the case of Nanosota-2A and Nanosota-3A) or mostly did so (for Nanosota-4A) (FIG. 6D).

To understand the antiviral spectrums of the three nanobodies, we mapped the mutations of different SARS-CoV-2 variants and SARS-CoV-1 to the prototypic SARS-CoV-2 RBD (FIG. 5C). Falling into the footprint of Nanosota-2A are two RBD mutations in the omicron variant, K417N and Y505H, and another RBD mutation shared by omicron and bat SARS2 (FIG. 5C); these RBD mutations may reduce the ability of Nanosota-2A to neutralize the entry of omicron and bat SARS2 (FIG. 2A). In direct contact with Nanosota-3A are one RBD mutation in the delta strain, L452R, and another RBD mutation shared by omicron and bat SARS2 (FIG. 5C). Leu452 in the prototypic RBD forms a hydrophobic patch with several hydrophobic residues in Nanosota-3A (FIG. 12B); the L452R mutation likely disrupts these favorable interactions, leading to the inability of Nanosota-3A to neutralize delta entry (FIG. 2A). Although SARS-CoV-1 and SARS-CoV-2 RBDs differ in many residues, surprisingly only two of them, S473F and V501I, fall into the footprint of Nanosota-4A (FIG. 5C); these mutations may slightly reduce the capability of Nanosota-4A to neutralize SARS1 entry (FIG. 2A). In addition, an omicron mutation, S452F, falls into the footprint of Nanosota-4A (FIG. 5C), which may account for the inability of Nanosota-4A to neutralize omicron entry (FIG. 2A). These structural data are consistent with the antiviral spectrums of the nanobodies.

### Structural-Guided Engineering of Nanosota-3 to Expand its Antiviral Spectrum

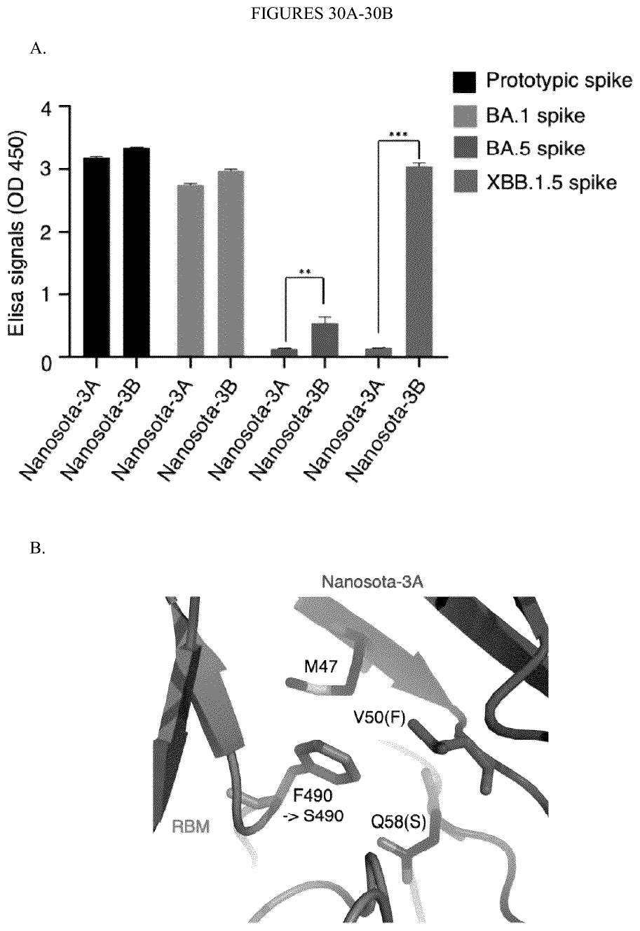

The above structural information provided a basis for the rational engineering of Nanosota-3A, with the goal of creating a highly effective entry inhibitor against the currently circulating omicron XBB.1.5 subvariant. By conducting ELISA between Nanosota-3A and the spikes from different omicron subvariants, we observed that Nanosota-3A binds to the prototypic spike and BA.1 spike, but not XBB.1.5 spike (FIG. 30A); these results align with the pseudovirus neutralization data (FIG. 2A). Examination of the structural interface between Nanosota-3A and the RBD led to the identification of a mutation from BA.1 to XBB.1.5 that potentially disrupted the binding of Nanosota-3A to the XBB.1.5 spike (FIG. 30B). Specifically, while both the prototypic RBD and the BA.1 RBD contained a phenylalanine at position 490, the XBB.1.5 RBD featured a serine at the same position. To overcome the F490S mutation in the XBB.1.5 RBD, random mutations were introduced to three residues (Met47, Val50 and Gln58) surrounding this mutation in Nanosota-3A. Through phage display, a mutant nanobody named Nanosota-3B was successfully selected, which exhibited a high affinity binding to XBB1.5 spike. Compared to Nanosota-3A, Nanosota-3B contained two mutations: V50F and Q58S. Nanosota-3B-Fc (Nanosota-3B with an added C-terminal Fc tag) effectively neutralized the entry of XBB.1.5 pseudoviruses (FIG. 2A). While the engineering of the nanobody did not specifically target the BA.5 spike, Nanosota-3B also displayed a moderate, yet significantly improved, affinity towards the BA.5 spike (FIG. 30A). Moreover, Nanosota-3B-Fc efficiently neutralized the entry of BA.5 pseudoviruses (FIG. 2A). Detailed structural mechanisms underlying the ability of Nanosota-3B to inhibit BA.5 and XBB1.5 pseudoviruses will be examined in future structural studies.

### Discussion

The COVID-19 pandemic has exposed the limitations of conventional antibodies as therapeutics, such as their limited potency, ineffectiveness against new variants, high costs, and injection-only administration route. There are many drawbacks to using conventional antibody drugs in a global pandemic. In addition to SARS-CoV-2, the past 20 years also saw the emergence of two other highly pathogenic coronaviruses, SARS-CoV-1 and MERS-CoV, from animal reservoirs (33, 34). SARS-CoV-1 is genetically related to SARS-CoV-2 and uses the same receptor ACE2 as does SARS-CoV-2 (5, 29). This recent history of coronavirus epidemics causes concerns about the recurrence of new coronavirus pandemics, especially coronaviruses that recognize ACE2 as receptor. Therefore, highly potent, wide-spectrum, cost effective, and easily administered anti-coronavirus therapeutics are urgently needed to help end the current pandemic and prepare for possible future coronavirus pandemics.

Nanobodies are superior therapeutics but have been largely overlooked in the current pandemic. Three features of nanobody therapeutics stand out compared to conventional antibodies. First, both monomeric nanobodies and Fc-tagged dimeric nanobodies may be administered as inhalers (25-27). This feature can greatly broaden the use of nanobody therapeutics. Second, both monomeric nanobodies and Fc-tagged dimeric nanobodies can be produced in large quantities in either bacteria or mammalian cells and are stable even at high temperatures in vitro (Fc-tagged nanobodies also have extended half lives in vivo) (24). This feature makes nanobodies cost-effective. Third, nanobodies can be adapted to new viral variants through in vitro affinity maturation using phage display (24). This feature greatly expands the potential antiviral spectrums of nanobodies. Moreover, nanobodies have low immunogenicity in humans and their safety as human therapeutics is indicated by an FDA's approval of a nanobody drug to treat a blood clotting disorder (24). Notwithstanding these benefits, the main uncertainty about nanobodies is whether they can reach high potency and wide spectrum. To date numerous anti-SARS-CoV-2 nanobodies have been identified, but none demonstrated extraordinary potency and spectrum as evaluated both in vitro and in animal models (Table S1). As described herein, this study identified three super potent and wide-spectrum nanobodies, Nanosota-2, -3 and -4, establishing nanobodies as ideal therapeutics against global viral pandemics.

All of Nanosota-2, -3 and -4 demonstrate high anti-SARS-CoV-2 potency. These nanobodies were identified from alpacas immunized with prototypic SARS-CoV-2 spike. Different from Nanosota-1 that we recently identified from a naive camelid nanobody phage display library and optimized using phage display, Nanosota-2, -3 and -4 took longer to identify, but they demonstrated great potency against their target even before any in vitro affinity maturation. In particular, Nanosota-2 inhibits the infection of prototypic SARS-CoV-2 in vitro at low IC₅₀ (2 pM or 0.16 ng/ml against live virus; 6.2 pM or 0.54 ng/ml against pseudovirus) and in mice at low dosage (4 mg/Kg weight) or late administration time (18 hours post-challenge); these potency metrics are among the best of known anti-SARS-CoV-2 entry inhibitors. Similarly, Nanosota-3 also demonstrates high potency in inhibiting the omicron variant in vitro and in mouse models. Nanosota-4A is effective against both SARS-CoV-1 and SARS-CoV-2. Cryo-EM data revealed that Nanosota-2 binds to the top region of SARS-CoV-2 RBD, whereas Nanosota-3 and Nanosota-4 bind to the two different sides of the RBD. Hence, two factors account for the high anti-SARS-CoV-2 potencies of these nanobodies. First, the nanobodies bind to the RBD with high affinity, overpowering their competitor ACE2. Second, the nanobodies bind to the functionally critical locations on the RBD. The binding site of Nanosota-2 on the RBD completely overlaps with the ACE2-binding site, explaining its super potency. The binding sites of Nanosota-3 and Nanosota-4 on the RBD only partially overlaps with that of ACE2-binding site, but they bind to the RBD in both standing-up and lying-down states, explaining their high potency. Interestingly, to bind the lying-down RBD, Nanosota-4A fits into a cavity in the trimeric spike (FIG. 12A), which would not accommodate conventional antibodies (FIG. 12B). Overall, this Example is the first to systematically show that super therapeutic potency can be achieved by nanobodies using both in vitro and in vivo metrics.

Nanosota-2, -3 and -4 also demonstrate a wide anti-SARS-CoV-2 spectrum. Nanosota-2 and Nanosota-4 potently inhibit all of the major SARS-CoV-2 strains except omicron, whereas Nanosota-3 potently inhibits all of the major SARS-CoV-2 strains except delta. Nanosota-4 even inhibits SARS-CoV-1 and bat SARS1. Cryo-EM data reveal that the wide spectrums of each of the individual nanobodies result from the nanobodies binding to RBD regions relatively conserved for major SARS-CoV-2 variants. Amazingly, although SARS-CoV-1 and SARS-CoV-2 RBDs differ tremendously in sequences, Nanosota-4 binds to an RBD region that is relatively conserved between SARS-CoV-1 and SARS-CoV-2; this region is inaccessible to conventional antibodies when the RBD is in the lying-down state (FIG. 12A, 12B). Because binding to the lying-down RBD benefits the potency of antibodies (8, 24), Nanosota-4 has an advantage over conventional antibodies in achieving high potency against both SARS-CoV-1 and SARS-CoV-2. Importantly, the combined antiviral spectrums of the three nanobodies cover SARS-CoV-2, its major variants and SARS-CoV-1. Because the three nanobodies bind to non-overlapping regions on SARS-CoV-2 RBD, they can be used as a cocktail to battle these viruses and their variants. It is worth nothing that the original version of Nanosota-3 was only effective against the early omicron subvariant BA.1. However, through in vitro affinity maturation, we successfully engineered Nanosota-3 to also become an effective inhibitor of the currently circulating omicron subvariant XBB.1.5. Therefore, our study demonstrates that wide antiviral spectrums potentially targeting all of ACE2-recognizing coronaviruses related to SARS-CoV-2 and SARS-CoV-1 can be achieved by nanobodies.

Because of their single chain structure, nanobodies can be adapted to new mutations in viral RBDs through in vitro affinity maturation using phage display. Hence, even if a nanobody binds to a viral RBD with moderate affinity, in vitro affinity maturation can significantly enhance its affinity for its target (24). In comparison, because of their two chain structures, it is inconvenient for conventional antibodies to be optimized using phage display. Therefore, through optional in vitro affinity maturation, Nanosota-2, -3 and -4 together may cover all of ACE2-recognizing coronaviruses related to SARS-CoV-2 and SARS-CoV-1. Overall, the Nanosota series are powerful therapeutic tools for battling the current and possible future coronavirus pandemics.

### Methods

### Ethics Statement

This study, as described herein in Example 1, was performed in strict accordance with the recommendations in the Guide for the Care and Use of Laboratory Animals of the National Institutes of Health. All of the animals were handled according to approved institutional animal care and use committee (IACUC) protocols of the University of Iowa (protocol number: 9051795).

### Cell Lines, Plasmids and Viruses

HEK293T cells (American Type Culture Collection) were cultured in Dulbecco's modified Eagle medium (DMEM) supplemented with 10% fetal bovine serum, 2 mM L-glutamine, 100 units/mL penicillin, and 100 μg/mL streptomycin (Life Technologies). ss320 E. coli (Lucigen) and TG1 E. coli (Lucigen) were grown in 2YT medium. Vero E6 cells (American Type Culture Collection) were grown in Eagle's minimal essential medium (EMEM) supplemented with penicillin (100 units/ml), streptomycin (100 μg/ml), and 10% fetal bovine serum (FBS). Original SARS-CoV-2 spike gene (Wuhan strain; GenBank: QHD43416.1), bat SARS2 spike gene (strain BANAL-20-236; GenBank: MZ937003.2), SARS-CoV-1 spike gene (strain Tor2; GenBank: AFR58742.1) and bat SARS1 spike gene (strain Rs3367; GenBank: AGZ48818.1) were synthesized (GenScript). Mutations were introduced to the original SARS-CoV-2 spike gene to generate the prototypic SARS-CoV-2 spike gene (encoding the spike protein from Wuhan strain plus D614G mutation) and the spike gene of alpha variant (strain B.1.1.7; GISAID: EPI_ISL_6135157), delta variant (strain B.1.617.2; GenBank: UEM53021.1), and omicron BA.1 variant (strain BA. 1; GISAID: EPI_ISL_6590782.2). Each of the spike gene was cloned into the pcDNA3.1(+) vector.

SARS-CoV-2 spike ectodomain (residues 14-1211) and SARS-CoV-2 RBD (residues 319-529) were subcloned into Lenti-CMV vector (Vigene Biosciences) with an N-terminal tissue plasminogen activator (tPA) signal peptide and a C-terminal His tag. For the spike ectodomain construct, D614G and six proline mutations were introduced to the S2 subunit region to stabilize the spike protein in its prefusion state (9, 35). Nanosota-2A-Fc, Nanosota-3A-Fc and Nanosota-4A-Fc were constructed in the same way as the RBD except that a C-terminal human IgG₁ Fc tag replaced the His tag. Infectious prototypic SARS-CoV-2 (US WA-1 isolate) and omicron variant (B.1.1.529 strain) were obtained from CDC (Atlanta) and Dr. Mehul Suthar (Emory University), respectively. Experiments involving infectious viruses were conducted at the University of Iowa in approved biosafety level 3 laboratories.

### Construction of Induced Nanobody Phage Display Library

Induced nanobody phage display libraries were constructed as previously described (36). Briefly, an alpaca was immunized 6 times with 125 μg of purified SARS-CoV-2 spike ectodomain in Gerbu adjuvant. Following immunization, blood was drawn and peripheral blood mononuclear cells (PBMCs) were isolated by centrifugation from 50 mL of blood using Sepmate centrifugal devices according to the manufacturer's protocol (Stemcell Technologies). A cDNA library was made by reverse transcription using oligo dT primers and Superscript IV reverse transcriptase (Thermo Scientific). A nested PCR strategy was used to amplify coding regions of VHH fragments. The resulting PCR fragments were cloned into a modified pADL22 vector (Antibody Design Labs), and the phage library was produced with a library size of 5×10⁹ following the manufacturer's protocols (Antibody Design Labs).

### Screening of Induced Nanobody Phage Display Library

To identify anti-SARS-CoV-2 nanobodies, the above nanobody phage display library was used in bio-panning as previously described (24). Briefly, 5 g purified prototypic SARS-CoV-2 spike ectodomain was used for one round of panning to obtain the SARS-CoV-2 spike-targeting nanobodies. After washing, the retained phages were eluted using 500 μl 100 mM triethylamine, neutralized with 250 μl 1 M Tris-HCl pH 7.5 and then used to infect ss320 E. coli. Single colonies were picked and expressions of nanobodies were induced by 1 mM IPTG. The supernatants were subjected to ELISA for identification of strong binders. The identified strong binders were expressed, purified (described below) and subjected to SARS-CoV-2 pseudovirus entry assay for selection of neutralizing nanobodies (described below).

### Protein Expression and Purification

Nanosota-2A, Nanosota-3A and Nanosota-4A were purified as previously described (24). Briefly, the nanobodies (all with a C-terminal His tag) were each purified from the periplasm of ss320 E. coli after induction by 1 mM IPTG. The E. coli cells were collected and re-suspended in 15 ml TES buffer (0.2 M Tris pH 8, 0.5 mM EDTA, 0.5 M sucrose), shaken on ice for 1 hour, diluted with 40 ml ¼ TES buffer, and then shaken on ice for another hour. The proteins in the supernatant were sequentially purified using a Ni-NTA column and a Superdex200 gel filtration column (Cytiva).

Prototypic SARS-CoV-2 spike ectodomain (with a His tag), SARS-CoV-2 RBD (with a His tag) and Fc-tagged nanobodies were prepared from 293F mammalian cells as previously described (37). Briefly, lentiviral particles were packaged using the plasmid encoding one of the above proteins and then infected 293F cells for selection of stable cell lines in the presence of Puromycin (Gibco). The proteins were harvested from the supernatants of cell culture medium, purified on Ni-NTA column for His-tagged proteins or on Protein A column for Fc-tagged proteins, and purified further on Superdex200 gel filtration column (Cytiva).

To prepare the complexes of the prototypic SARS-CoV-2 spike ectodomain and one of three nanobodies (Nanosota-2A, Nanosota-3A and Nanosota-4A), the spike ectodomain and one of the nanobodies were incubated (with the nanobody in excess) at room temperature for 30 minutes before the mixture was subjected to Superose 6 increase 10/300 GL gel filtration column (Cytiva).

### ELISA

To detect the binding between His-tagged SARS-CoV-2 spike ectodomain and HA-tagged nanobodies from the supernatant of ss320 E. coli, ELISA was carried out as previously described (24). Briefly, ELISA plates were coated with recombinant SARS-CoV-2 spike ectodomain and were then incubated sequentially with the supernatant of ss320 E. coli containing nanobodies and HRP-conjugated anti-HA antibody (1:5,000) (Sigma). Subsequently ELISA substrate (Invitrogen) was added and the reactions were stopped using 1N H₂SO4. The absorbance at 450 nm (A₄₅₀) was measured using a Synergy LX Multi-Mode Reader (BioTek).

### Surface Plasmon Resonance

To measure the binding affinity between each of the nanobodies and SARS-CoV-2 RBD, surface plasmon resonance assay was performed using a Biacore S200 system (Cytiva) as previously described (24). Briefly, each of the Fc-tagged nanobodies was immobilized on a protein A sensor chip (Cytiva). Serial dilutions of His-tagged SARS-CoV-2 RBD were injected at different concentrations from 2.5 nM to 80 nM. The resulting data were fitted to a 1:1 binding model using Biacore Evaluation Software (Cytiva). The analysis was conducted according to the Biacore handbook's guidelines, specifically for interactions between a coated Fc-tagged antibody and a monomeric analyte that flows through. RU: resonance unit.

To assess the potential competition between human ACE2 and each of the three Fc-tagged nanobodies, competition SPR experiments were carried out. The prototypical SARS-CoV-2 RBD (with a His-tag) was immobilized onto four CM5 sensor chips (Cytiva) (800 RU for each chip). Subsequently, each Fc-tagged nanobody (at 6250 nM), specifically Nanosota-2A-Fc, Nanosota-3A-Fc, or Nanosota-4A-Fc, was injected to the first three sensor chips. As a control, running buffer was injected to the fourth sensor chip. After the first three sensor chips were saturated with their respective nanobodies, a mixture of recombinant human ACE2 (His-tagged, at 6250 nM) and the same individual nanobody (6250 nM) were injected to each of the first three chips. On the control chip, only the ACE2 was injected. The resulting sensorgrams from all four chips were overlaid, setting the point at which ACE2 injection began as the baseline. Finally, the competitive binding between human ACE2 and each Fc-tagged nanobody was evaluated by comparing the SPR binding signals from the mixed nanobody/ACE2 injections to those from the ACE2-only injection.

### Pseudovirus Entry Assay

The neutralizing potencies of Nanosota-2A-Fc, Nanosota-3A-Fc and Nanosota-4A-Fc against SARS-CoV-2 and SARS-CoV-1 pseudoviruses were evaluated using pseudovirus entry assay as previously described (37). Briefly, to prepare the pseudoviruses, HEK293T cells were co-transfected with a pcDNA3.1(+) plasmid encoding one of the coronavirus spike proteins, a helper plasmid psPAX2 and a reporter plasmid plenti-CMV-luc. Pseudoviruses were collected 72 hours post transfection, incubated with nanobodies at different concentrations at 37° C. for 1 hour, and then used to enter HEK293T cells stably expressing human ACE2. After another 60 hours, cells were lysed. Aliquots of cell lysates were transferred to new plates, a luciferase substrate was added, and Relative Light Units (RLUs) were measured using an EnSpire plate reader (PerkinElmer). The efficacy of each nanobody was calculated and expressed as the concentration of the nanobody capable of inhibiting pseudovirus entry by 50% (IC₅₀).

### SARS-CoV-2 Plaque Reduction Neutralization Test

The neutralizing potencies of Nanosota-2A-Fc and Nanosota-3A-Fc against infectious SARS-CoV-2 in vitro were evaluated using a plaque reduction neutralization test (PRNT) assay as previously described (24). Briefly, one of the nanobodies was serially diluted in DMEM and mixed with the virus (prototypic SARS-CoV-2 for Nanosota-2A-Fc and omicron variant for Nanosota-3A-Fc) at a titer of 800 plaque-forming unit (PFU/ml) at 37° C. for 1 hour. The mixture was then incubated with Vero E6 cells at 37° C. for an additional 45 minutes. Subsequently the cell culture medium was removed and the cells were overlaid with 0.6% agarose containing 2% FBS and cultured for 3 days. Plaques were visualized by 0.1% crystal violet staining. The efficacy of each nanobody was calculated and expressed as the concentration capable of reducing the number of virus plaques by 50% (i.e., IC₅₀) compared to control serum-exposed virus.

### SARS-CoV-2 Challenge Experiment in Mouse Models

The neutralizing potencies of Nanosota-2A-Fc and Nanosota-3A-Fc against infectious SARS-CoV-2 in vivo were evaluated using SARS-CoV-2 challenge experiments in mouse models as previously described (24).

The efficacy of Nanosota-2A-Fc against prototypic SARS-CoV-2 was determined in K18-human-ACE2-transgenic mice (K18-Tg mice) (Jackson Laboratory). All mice were challenged via intranasal inoculation of prototypic SARS-CoV-2 (5×10³ PFU/mouse) in a volume of 50 μl DMEM. In the treatment group (n=10), mice received Nanosota-2A-Fc (10 mg/Kg weight) via intraperitoneal injection at 4 hours post-challenge. In the control group (n=8), mice were administered PBS buffer at 4 hours post-challenge. The virus titers in the lungs of the mice were measured using by a virus plaque assay as previously described (24). Briefly, half of the mice from each group were euthanized on day 2 post-challenge and lung tissue homogenate supernatants were collected. 12-well plates of Vero E6 cells were inoculated with serial diluted lung homogenates (in DMEM) and then incubated at 37° C. in 5% CO₂ for 1 hour with gently shaking every 15 minutes. Then the inocula were removed and the plates were overlaid with 0.6% agarose containing 2% FBS. After 3 days, the overlays were removed and the plaques were visualized via staining with 0.1% crystal violet. Virus titers were quantified as PFU per ml tissue. The body weights of the other half of the mice from each group were monitored daily. Mouse lungs were collected on day 5 post-challenge and then were fixed in 10% formalin. Histopathology of the lung was examined by hematoxylin and eosin-staining tissue section.

To further determine the limits of its efficacy, Nanosota-2A-Fc was administered at a lower dosage or at a later treatment time point in the SARS-CoV-2 challenge experiment in K18-Tg mice. More specifically, mice were divided into three groups (n=10 in each group): (i) low dosage treatment group—Nanosota-2A-Fc was administered 4 hours post-challenge at 4 mg/Kg weight; (ii) late treatment group—Nanosota-2A-Fc was administered 18 hours post-challenge at 16 mg/Kg weight; (iii) negative control group −PBS buffer was administered. Lung virus titers, body weights and lung histology data were collected in the same way as above.

The efficacy of Nanosota-3A-Fc against the omicron variant was determined in both K18-Tg mice (n=6) and Balb/c mice (n=5). More specifically, mice were challenged via intranasal with omicron B.1.1.529 (105 PFU/mouse) and were then treated with either Nanosota-3A-Fc (10 mg/Kg weight) or PBS buffer at 4 hours post-challenge. Lung virus titers on day 3 post-challenge were determined in the same way as above.

The efficacy of Nanosota-3A-Fc was further evaluated via intranasal delivery in the mouse model. Balb/c mice (n=5) were challenged intranasally with 10⁵ PFU omicron B.1.1.529 and were then treated with either Nanosota-3A-Fc (10 mg/Kg weight) or PBS buffer at 4 hours post-challenge via intranasal delivery. Pipettes were used for the intranasal delivery of Nanosota-3A-Fc. Viral titers of infected lungs on 2^{rd} day post infection were determined in the same way as above.

We established certain priorities for our animal testing. First, we evaluated Nanosota-2A-Fc against the prototypic SARS-CoV-2 due to its pronounced effectiveness against this strain in pseudovirus entry assay and live SARS-CoV-2 infection assay in vitro. However, since the prototypic SARS-CoV-2 cannot infect Balb/c mice efficiently, we limited our testing of Nanosota-2A-Fc to K18-human-ACE2-transgenic mice. Second, we assessed Nanosota-3A-Fc against the omicron BA.1 variant because of its effectiveness against the omicron variant in vitro. Our interest in further developing Nanosota-3A-Fc was partly driven by the current clinical significance of the omicron variant. Consequently, we experimented with Nanosota-3A-Fc in mice using both intraperitoneal and intranasal delivery methods. Since the omicron BA.1 variant demonstrated a reasonable ability to infect both K18-human-ACE2-transgenic and Balb/c mice, we used both mouse models for the Nanosota-3A-Fc tests.

### Cryo-EMgrid Preparation and Data Acquisition

The complexes of SARS-CoV-2 spike ectodomain and each of the nanobodies (4 μl at ˜1.9 pM for the spike/Nanosota-2A complex, -1.3 pM for the spike/Nanosota-3A complex, and ˜1.1 pM for the spike/Nanosota-4A complex, respectively) were supplemented with 8 mM CHAPSO immediately before grid preparation. Each of the complexes was then applied to freshly glow-discharged Quantifoil R1.2/1.3 300-mesh copper grids (EM Sciences) and blotted for 4 seconds at 22° C. under 100% chamber humidity and plunge-frozen in liquid ethane using a Vitrobot Mark IV (FEI). Cryo-EM data were collected using EPU version 3.0 (ThermoFisher Scientific) on a Titan Krios electron microscope (ThermoFisher Scientific) equipped with a Falcon IV direct electron detector and with a Selectris-X energy filter (ThermoFisher Scientific) or using Latitude-S(Gatan) equipped with a K3 direct electron detector and with a Biocontinuum energy filter (Gatan). For the Falcon IV detector, the movies were collected at a nominal magnification of 165,000×(corresponding to 0.73 Å per pixel), slit width 10 eV, a dose rate of 5.9 e⁻ per Å² per second, and a total dose of 40 e⁻/Å². For the K3 detector, the movies were collected at a nominal magnification of 75,300× (corresponding to 0.664 Å per pixel), slit width 20 eV, a dose rate of 25 e⁻ per Å² per second, and a total dose of 50 e⁻/Å². The statistics of cryo-EM data collection are summarized in Table S3.

### Image Processing

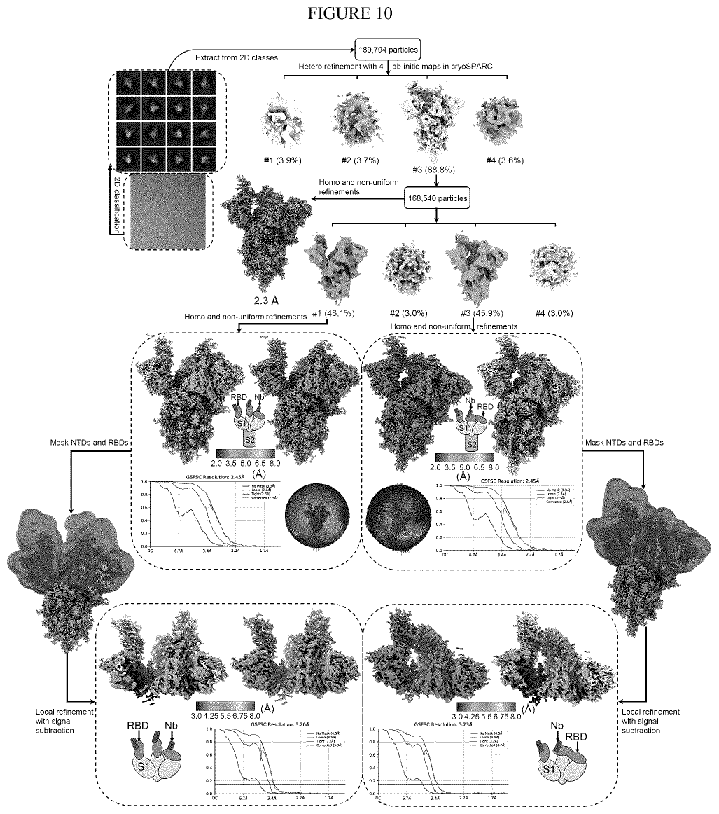

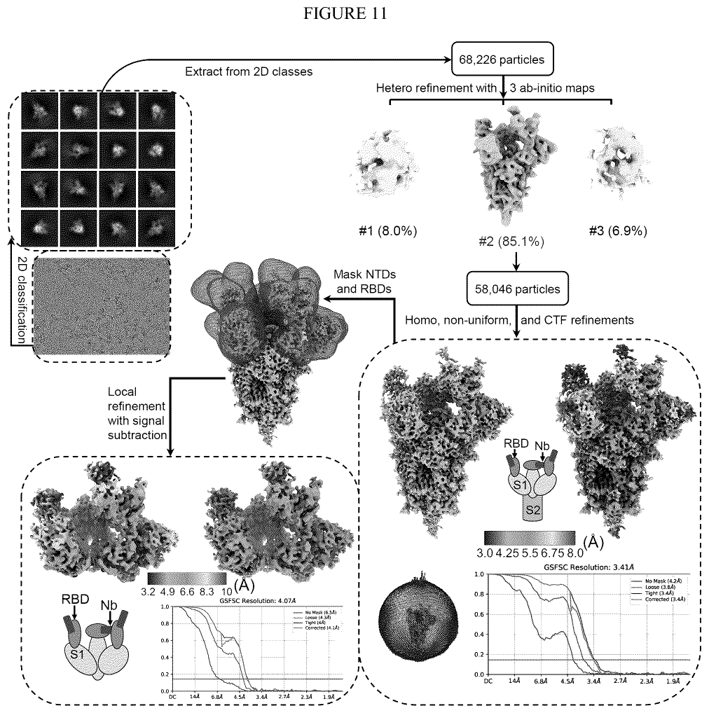

Cryo-EM data were processed using cryoSPARC v3.3.2 (38), and the procedure is outlined in FIG. 9, FIG. 10 and FIG. 11. Briefly, dose-fractionated movies were subjected to Patch motion correction with MotionCor2 (39) and Patch CTF estimation with CTFFIND-4.1.13 (40). Particles were then picked using both Blob picker and Template picker in cryoSPARC v3.3.2 and subjected to the Remove Duplicate Particles Tool. Junk particles were removed through three rounds of 2D classifications. Particles from the good 2D classes were used for Ab-initio Reconstruction of three or four maps. The initial models were set as the starting references for heterogeneous refinement (3D classification). A second round of Ab-initio Reconstruction and heterogeneous refinement was conducted for the spike/Nanosota-3A complex. The selected 3D classes were then subjected to further homogeneous, non-uniform and CTF refinements, generating the final maps. Particles in the good 3D class were then imported into RELION-4.0 (41) using the csparc2star.py module (UCSF pyem v0.5. Zenodo) and subjected to signal subtraction to keep only the receptor-binding subunit of the spike and Nanosota-3A in RELION-4.0, followed by masked 3D classification for the spike/Nanosota-3A complex. Particles with the subtracted signal (spike/Nanosota-3A and spike/Nanosota-4A) and the ones in the selected class from the masked 3D classification (spike/Nanosota-2A) were then subjected to local refinements to improve densities in cryoSPARC v3.3.2. Resolutions of the maps were determined by gold-standard Fourier shell correlation (FSC) at 0.143 between the two half-maps. Local resolution variations were estimated from the two half-maps in cryoSPARC v3.3.2.

### Cryo-EM Model Building and Refinement.

Initial model building of the spike/nanobody complexes was performed in Coot-0.8.9 (42) using PDB 7TGX as the starting model. The initial model of each nanobody was predicted using SWISS-MODEL (world wide web: swissmodel.expasy.org/), and then fitted into the density map. Several rounds of refinement in Phenix-1.16 (43) and manually building in Coot-0.8.9 were performed until the final reliable models were obtained. Standing-up RBDs and spike-bound nanobodies are generally flexible and hence they were fitted into the density as rigid bodies. Specially, in the local map of the receptor-binding subunit from the spike/Nanosota-3A complex, an atomic model was built at the interface between one lying-down RBD and Nanosota-3A. Model and map statistics are summarized in Table S3. Figures were generated using UCSF Chimera X v0.93 (44) and PyMol v2.5.2 (45).

In vitro affinity maturation of Nanosota-3A to generate Nanosota-3B To engineer Nanosota-3 for expansion of its antiviral spectrum, in vitro affinity maturation of Nanosota-3A was performed. Briefly, random mutations were introduced to three residues (Met47, Val50 and Gln58) of Nanosota-3A surrounding Phe490 in the prototypic RBD. Specifically, random mutations were introduced to the PCR primers for generation of mutant Nanosota-3A genes. The mutant Nanosota-3A genes were then inserted into the PADL22c vector and electroporated into the TG1 cells for construction of a mutational library. Subsequently, mutant phages were selected for enhanced binding to XBB.1.5 spike protein as described above. After three rounds of selection, Nanosota-3B with two mutations (V50F and Q58S) was discovered, which showed the highest binding affinity for XBB.1.5 spike.

### Documents Cited in Example 1

1. W. T. Harvey et al., SARS-CoV-2 variants, spike mutations and immune escape. Nat Rev Microbiol 19, 409-424 (2021).
2. M. Lipsitch, F. Krammer, G. Regev-Yochay, Y. Lustig, R. D. Balicer, SARS-CoV-2 breakthrough infections in vaccinated individuals: measurement, causes and impact. Nature reviews. Immunology 22, 57-65 (2022).
3. F. Li, Structure, Function, and Evolution of Coronavirus Spike Proteins. Annual review of virology 3, 237-261 (2016).
4. L. Y. Du et al., The spike protein of SARS-CoV—a target for vaccine and therapeutic development. Nature Reviews Microbiology 7, 226-236 (2009).
5. Y. Wan, J. Shang, R. Graham, R. S. Baric, F. Li, Receptor Recognition by the Novel Coronavirus from Wuhan: an Analysis Based on Decade-Long Structural Studies of SARS Coronavirus. J Virol 94, (2020).
6. J. Shang et al., Structural basis of receptor recognition by SARS-CoV-2. Nature 581, 221-224 (2020).
7. J. Lan et al., Structure of the SARS-CoV-2 spike receptor-binding domain bound to the ACE2 receptor. Nature 581, 215-220 (2020).
8. J. Shang et al., Cell entry mechanisms of SARS-CoV-2. Proc Natl Acad Sci USA 117, 11727-11734 (2020).
9. G. Ye, B. Liu, F. Li, Cryo-EM structure of a SARS-CoV-2 omicron spike protein ectodomain. Nature communications 13, 1214 (2022).
10. Y. Cao et al., Omicron escapes the majority of existing SARS-CoV-2 neutralizing antibodies. Nature 602, 657-663 (2022).
11. S. M. Gobeil et al., Effect of natural mutations of SARS-CoV-2 on spike structure, conformation, and antigenicity. Science 373, (2021).
12. N. Jovanoski, A. Kuznik, U. Becker, M. Hussein, A. Briggs, Cost-effectiveness of casirivimab/imdevimab in patients with COVID-19 in the ambulatory setting. Journal of managed care & specialty pharmacy 28, 555-565 (2022).
13. I. Hernandez et al., Pricing of monoclonal antibody therapies: higher if used for cancer?The American journal of managed care 24, 109-112 (2018).
14. R. Rubin, Monoclonal Antibodies for COVID-19 Preexposure Prophylaxis Can't Come Fast Enough for Some People. Jama 326, 1895-1897 (2021).
15. Tixagevimab and Cilgavimab (Evusheld) for Pre-Exposure Prophylaxis of COVID-19. Jama 327, 384-385 (2022).
16. P. Chames, M. Van Regenmortel, E. Weiss, D. Baty, Therapeutic antibodies: successes, limitations and hopes for the future. British journal of pharmacology 157, 220-233 (2009).
17. F. Paul, G. Cartron, Infusion-related reactions to rituximab: frequency, mechanisms and predictors. Expert review of clinical immunology 15, 383-389 (2019).
18. B. Brobst, J. Borger, in StatPearls. (StatPearls Publishing Copyright © 2022, StatPearls Publishing LLC., Treasure Island (FL), 2022).
19. D. Könning et al., Camelid and shark single domain antibodies: structural features and therapeutic potential. Curr Opin Struct Biol 45, 10-16 (2017).
20. T. De Meyer, S. Muyldermans, A. Depicker, Nanobody-based products as research and diagnostic tools. Trends Biotechnol 32, 263-270 (2014).
21. S. Muyldermans, Nanobodies: natural single-domain antibodies. Annu Rev Biochem 82, 775-797 (2013).
22. S. Steeland, R. E. Vandenbroucke, C. Libert, Nanobodies as therapeutics: big opportunities for small antibodies. Drug discovery today 21, 1076-1113 (2016).
23. E. Romao et al., Identification of Useful Nanobodies by Phage Display of Immune Single Domain Libraries Derived from Camelid Heavy Chain Antibodies. Current pharmaceutical design 22, 6500-6518 (2016).
24. G. Ye et al., The development of Nanosota-1 as anti-SARS-CoV-2 nanobody drug candidates. eLife 10, (2021).
25. X. Wu et al., A potent bispecific nanobody protects hACE2 mice against SARS-CoV-2 infection via intranasal administration. Cell reports 37, 109869 (2021).
26. S. Nambulli et al., Inhalable Nanobody (PiN-21) prevents and treats SARS-CoV-2 infections in Syrian hamsters at ultra-low doses. Science advances 7, (2021).
27. M. Schoof et al., An ultrapotent synthetic nanobody neutralizes SARS-CoV-2 by stabilizing inactive Spike. Science 370, 1473-1479 (2020).
28. M. Scully et al., Caplacizumab Treatment for Acquired Thrombotic Thrombocytopenic Purpura. N Engl J Med 380, 335-346 (2019).
29. J. Cui, F. Li, Z. L. Shi, Origin and evolution of pathogenic coronaviruses. Nat Rev Microbiol 17, 181-192 (2019).
30. F. Li, W. H. Li, M. Farzan, S. C. Harrison, Structure of SARS coronavirus spike receptor-binding domain complexed with receptor. Science 309, 1864-1868 (2005).
31. X. Y. Ge et al., Isolation and characterization of a bat SARS-like coronavirus that uses the ACE2 receptor. Nature 503, 535-538 (2013).
32. W. Zhang et al., Structural basis for mouse receptor recognition by SARS-CoV-2 omicron variant. Proc Natl Acad Sci USA 119, e2206509119 (2022).
33. T. G. Ksiazek et al., A novel coronavirus associated with severe acute respiratory syndrome. New England Journal of Medicine 348, 1953-1966 (2003).
34. A. M. Zaki, S. van Boheemen, T. M. Bestebroer, A. Osterhaus, R. A. M. Fouchier, Isolation of a Novel Coronavirus from a Man with Pneumonia in Saudi Arabia. New England Journal of Medicine 367, 1814-1820 (2012).
35. C. L. Hsieh et al., Structure-based design of prefusion-stabilized SARS-CoV-2 spikes. Science 369, 1501-1505 (2020).
36. E. Pardon et al., A general protocol for the generation of Nanobodies for structural biology. Nature protocols 9, 674-693 (2014).
37. Q. Geng et al., Novel virus-like nanoparticle vaccine effectively protects animal model from SARS-CoV-2 infection. PLoS Pathog 17, e1009897 (2021).
38. A. Punjani, J. L. Rubinstein, D. J. Fleet, M. A. Brubaker, cryoSPARC: algorithms for rapid unsupervised cryo-EM structure determination. Nature methods 14, 290-296 (2017).
39. J. L. Rubinstein, M. A. Brubaker, Alignment of cryo-EM movies of individual particles by optimization of image translations. Journal of structural biology 192, 188-195 (2015).
40. A. Rohou, N. Grigorieff, CTFFIND4: Fast and accurate defocus estimation from electron micrographs. Journal of structural biology 192, 216-221 (2015).
41. J. Zivanov, T. Nakane, S. H. W. Scheres, Estimation of high-order aberrations and anisotropic magnification from cryo-EM data sets in RELION-3.1. IUCrJ7, 253-267 (2020).
42. P. Emsley, K. Cowtan, Coot: model-building tools for molecular graphics. Acta Crystallographica Section D-Biological Crystallography 60, 2126-2132 (2004).
43. P. D. Adams et al., PHTENIX: a comprehensive Python-based system for macromolecular structure solution. Acta Crystallographica Section D-Biological Crystallography 66, 213-221 (2010).
44. T. D. Goddard et al., UCSF ChimeraX: Meeting modern challenges in visualization and analysis. Protein science: a publication of the Protein Society 27, 14-25 (2018).
45. W. L. DeLano, PyMOL: an open-source molecular graphics tool. CCP4Newsl. Protein Crystallogr. 40, 82-92 (2002).

TABLE S1
In vitro anti-SARS-CoV-2 potency of antibodies/nanobodies from the literature

| | | Conventional | | | Animal |
|---|---|---|---|---|---|
| Reference | Nanobody | antibody | Pseudovirus | Live virus | testing? |
| Example | Nanosota- | | 0.50 ng/ml = 6.2 pM (WT) | 0.16 ng/ml = 2 pM (WT) | Yes |
| 1 | 2A-Fc | | 0.56 ng/ml = 6.9 pM | | |
| | | | (Alpha) | | |
| | | | 1.1 ng/ml = 14 pM (Delta) | | |
| Example | Nanosota- | | 5.7 ng/ml = 74 pM (WT) | 2.3 ng/ml = 30 pM | Yes |
| 1 | 3A-Fc | | 1.2 ng/ml = 16 pM (Alpha) | (Omicron) | |
| | | | 3.6 ng/ml = 47 pM | | |
| | | | (Omicron) | | |
| (1) | | A23-58.1, | 0.3-11 ng/ml | 2.1-4.8 ng/ml (WT) | No |
| | | B1.182.1 | (23 variants; no Omicron) | | |
| (2) | | 35B5 | | 1.6 ng/ml (WT) | Yes |
| | | 32C7 | | 8.6 ng/ml (WT) | |
| (3) | | J08, I14 | | 1-10 ng/ml (D614G, Alpha) | Yes |
| (4) | | JMB2002 | 150 ng/ml (omicron) | | No |
| (5) | | 35B5 | 2.4 ng/ml (Delta) | 50 ng/ml (Omicron) | No |
| | | | 6.9 ng/ml (Delta) | | |
| | | | 15 ng/ml (Omicron) | | |
| (6) | | S2E12 | 1.4 ng/ml (WT) | | No |
| | | | 38 ng/ml (Omicron) | | |
| | | LY-CoV1404 | 3.0 ng/ml (WT) | | |
| | | | 5.1 ng/ml (Omicron) | | |
| (7) | | P17 | 165 pM (WT) | 195 pM (WT) | Yes |
| (8) | | Clone2 | 0.54 ng/ml (WT); 37.8 | 7.70 ng/ml (WT); | Yes |
| | | | ng/ml (Beta) | 31.51 ng/ml (Delta) | |
| | | Clone6 | 4.2 ng/ml (WT); 435.8 | 6.14 ng/ml (WT); | |
| | | | ng/ml (Beta) | 111.34 ng/ml (Delta) | |
| | | Clone13A | 6.2 ng/ml (WT) | 59.28 ng/ml (WT); | Yes |
| | | | | 120.10 ng/ml (Delta) | |
| (9) | | STE90-C11 | | 0.56 nM (WT) | Yes |
| (10) | | WRAIR NTD | 6-27 ng/ml (WT) | 32-215 ng/ml (WT) | Yes |
| | | mAbs | | | |
| | | WRAIR RBD | 4-17 ng/ml (WT) | 33-172 ng/ml (WT) | |
| | | mAbs | | | |
| (11) | | 31 RBD | 2-800 ng/ml (WT, Alpha, | | Yes |
| | | mAbs | Beta, Gamma, Delta, | | |
| | | | Lambda); | | |
| | | | <20 ng/ml (Omicron) | | |
| (12) | Nanosota- | | 0.27 ug/ml (WT) | 0.16 ug/ml (WT) | Yes |
| | 1C-Fc | | | | |
| (13) | ANTE- | | 1.32 pM (WT) | 6.04 pM (SARS-CoV-2 | No |
| | CoV2- | | | Munich strain) | |
| | Nab21T_{GS} | | | | |
| (14) | mNb6-tri | | 5.0 ng/ml = 120 pM (WT) | 2.3 ng/ml = 54 pM (WT) | No |
| (15) | C5-Trimer | | | 20 pM (WT) | Yes |
| | | | | 25 pM (Alpha) | |
| (16) | Nb 19 | | 0.7 ng/ml = 9 pM (WT) | | No |
| | trimer | | | | |
| | Nb 56 | | 3.9 ng/ml = 55 pM (WT) | 0.2 ng/ml = 3 pM (WT) | |
| | trimer | | | 0.6 ng/ml = 8 pM (Alpha) | |
| | | | | 1.3 ng/ml = 18 pM (Beta) | |
| | | | | 0.2 ng/ml = 3 pM (Gamma) | |
| (17) | WNb-Fc 36 | | | 100 pM (WT) | Yes |
| | | | | 110 pM (N501Y; D614G) | |
| (18) | aRBD-2-5- | | 20 pM (Omicron) | 83 pM (WT) | Yes |
| | Fc | | | 44 pM (Beta) | |
| | | | | 27 pM (Delta) | |
| | | | | 29 pM (Omicron) | |
| (19) | 8A2 + 7A3 | | 1 nM (WT) | 1 nM (WT) | Yes |
| | | | 400 pM (Alpha) | 6 nM (614G) | |
| | | | 300 pM (Beta) | 2 nM (Alpha) | |
| | | | 200 pM (Gamma) | 870 pM (Beta) | |
| | | | | 140 pM (Gamma) | |
| | | | | 19 nM (Delta) | |
| (20) | 2-67(A) | | 80 pM (WT) | 310 pM (Delta) | No |
| | | | 80 pM (Alpha) | | |
| | | | 180 pM (Delta) | | |
| | | | 110 pM (Omicron) | | |
| (21) | V_{H}-Fc ab8 | | 30 ng/ml (WT) | 40 ng/ml (WT) | Yes |
| (22) | hum VH_ | | | 110 ng/ml (Beta) | Yes |
| | S56A/LA | | | | |
| | L APG- | | | | |
| | Fc/Gen2 | | | | |
| | (hum VHH_ | | | 20 ng/ml (Beta) | |
| | S56A)2/L | | | | |
| | ALAPG- | | | | |
| | Fc/Gen2 | | | | |
| (23) | BP10-Fc | | | 90 ± 20 nM (WT) | Yes |
| | BP19-Fc | | | 112 ± 18 nM (WT) | |
| | BP39-Fc | | | 34 ± 2 nM (WT) | |
| (24) | BiShAb020 | | 24 ng/ml (WT) | | Yes |
| | 1 | | 26 ng/ml (Beta) | | |
| | | | 5 ng/ml (Delta) | | |
| | | | 27 ng/ml (BA.1) | | |
| | | | 35 ng/ml (BA.5) | | |
| | ShAb01H0 | | <23 ng/ml | | |
| | 2K | | (WT) 21 ng/ml | | |
| | | | (Beta) | | |
| | | | 2 ng/ml (Delta) | | |
| | | | 391 ng/ml (BA.1) | | |
| | | | 524 ng/ml (BA.5) | | |
| (25) | MR14 | | 5.3 ± 0.45 ng/ml (WT) | 91 ng/ml (WT) | Yes |
| | | | 5.0 ± 1.7 ng/ml (Delta) | 13 ng/ml (Delta) | |
| | | | 0.16 ± 0.044 ng/ml (BA.1) | 6.6 ng/ml (BA.1) | |
| | | | 0.17 ± 0.082 ng/ml (BA.2) | 3.4 ng/ml (BA.2) | |
| | | | 0.69 ± 0.63 ng/ml (BA.3) | | |
| | | | 18.7 ± 9.7 ng/ml (BA.4/5) | | |
| | MS43 | | 179 ± 7.4 ng/ml (WT) | 1117 ng/ml (WT) | |
| | | | 30 ± 17 ng/ml (Delta) | 20 ng/ml (Delta) | |
| | | | 1.5 ± 0.037 ng/ml (BA.1) | 30 ng/ml (BA.1) | |
| | | | 52520 ± 43208 ng/ml | 2937 ng/ml (BA.2) | |
| | | | (BA.2) | | |
| | | | 50 ± 25 ng/ml (BA.3) | | |
| | | | 4405 ± 2672 ng/ml (BA.4/5) | | |
| (26) | W25-Fc | | | 9.01 nM (WT) | Yes |
| | | | | 0.38 nM (Alpha) | |
| | | | | 1.31 (Beta) | |
| | | | | 0.29 (Gamma) | |
| | | | | 1.45 nM (BA.1) | |
| | | | | 2.07 (BA.2) | |

WT: the original SARS-CoV-2 Wuhan strain isolated in early 2020
D614G: the original SARS-CoV-2 Wuhan strain with the added D614G mutation
Documents in Table S1:
1. L. Wang et al., Ultrapotent antibodies against diverse and highly transmissible SARS-CoV-2 variants. Science 373, (2021).
2. X. Wang et al., A potent human monoclonal antibody with pan-neutralizing activities directly dislocates S trimer of SARS-CoV-2 through binding both up and down forms of RBD. Signal transduction and targeted therapy 7, 114 (2022).
3. E. Andreano et al., Extremely potent human monoclonal antibodies from COVID-19 convalescent patients. Cell 184, 1821-1835.e1816 (2021).
4. W. Yin et al., Structures of the Omicron spike trimer with ACE2 and an anti-Omicron antibody. Science 375, 1048-1053 (2022).
5. X. Wang et al., 35B5 antibody potently neutralizes SARS-CoV-2 Omicron by disrupting the N-glycan switch via a conserved spike epitope. Cell host & microbe 30, 887-895.e884 (2022).
6. T. Zhou et al., Structural basis for potent antibody neutralization of SARS-CoV-2 variants including B.1.1.529. Science 376, eabn8897 (2022).
7. H. Yao et al., Rational development of a human antibody cocktail that deploys multiple functions to confer Pan-SARS-CoVs protection. Cell Res 31, 25-36 (2021).
8. L. Peng et al., Monospecific and bispecific monoclonal SARS-COV-2 neutralizing antibodies that maintain potency against B.1.617. Nature communications 13, 1638 (2022).
9. F. Bertoglio et al., A SARS-CoV-2 neutralizing antibody selected from COVID-19 patients binds to the ACE2-RBD interface and is tolerant to most known RBD mutations. Cell reports 36, 109433 (2021).
10. V. Dussupt et al., Low-dose in vivo protection and neutralization across SARS-CoV-2 variants by monoclonal antibody combinations. Nature immunology 22, 1503-1514 (2021).
11. K. Wang et al., Memory B cell repertoire from triple vaccinees against diverse SARS-CoV-2 variants. Nature 603, 919-925 (2022).
12. G. Ye et al., The development of Nanosota-1 as anti-SARS-CoV-2 nanobody drug candidates. eLife 10, (2021).
13. Y. Xiang et al., Versatile and multivalent nanobodies efficiently neutralize SARS-CoV-2. Science 370, 1479-1484 (2020).
14. M. Schoof et al., An ultrapotent synthetic nanobody neutralizes SARS-CoV-2 by stabilizing inactive Spike. Science 370, 1473-1479 (2020).
15. J. Huo et al., A potent SARS-CoV-2 neutralising nanobody shows therapeutic efficacy in the Syrian golden hamster model of COVID-19. Nature communications 12, 5469 (2021).
16. J. Xu et al., Nanobodies from camelid mice and llamas neutralize SARS-CoV-2 variants. Nature 595, 278-282 (2021).
17. P. Pymm et al., Nanobody cocktails potently neutralize SARS-CoV-2 D614G N501Y variant and protect mice. Proc Natl Acad Sci USA 118, (2021).
18. H. Ma et al., Hetero-bivalent nanobodies provide broad-spectrum protection against SARS-CoV-2 variants of concern including Omicron. Cell Res 32, 831-842 (2022).
19. J. Hong et al., Dromedary camel nanobodies broadly neutralize SARS-CoV-2 variants. Proc Natl Acad Sci USA 119, e2201433119 (2022).
20. Y. Xiang et al., Superimmunity by pan-sarbecovirus nanobodies. Cell reports 39, 111004 (2022).
21. W. Li et al., High Potency of a Bivalent Human V(H) Domain in SARS-CoV-2 Animal Models. Cell 183, 429-441.e416 (2020).
22. Schepens, B. et al. An affinity-enhanced, broadly neutralizing heavy chain-only antibody protects against SARS-CoV-2 infection in animal models. Sci Transl Med 13, eabi7826, doi:10.1126/scitranslmed.abi7826 (2021).
23. Pymm, P. et al. Biparatopic nanobodies targeting the receptor binding domain efficiently neutralize SARS-CoV-2. iScience 25, 105259, doi:10.1016/j.isci.2022.105259 (2022).
24. Chen, W. H. et al. Shark nanobodies with potent SARS-CoV-2 neutralizing activity and broad sarbecovirus reactivity. Nat Commun 14, 580, doi: 10.1038/s41467-023-36106-x (2023).
25. Liu, H. et al. Two pan-SARS-CoV-2 nanobodies and their multivalent derivatives effectively prevent Omicron infections in mice. Cell Rep Med 4, 100918, doi:10.1016/j.xcrm.2023.100918 (2023).
26. Modhiran, N. et al. A nanobody recognizes a unique conserved epitope and potently neutralizes SARS-CoV-2 omicron variants. iScience 26, 107085, doi:10.1016/j.isci.2023.107085 (2023).

TABLE S2
In vivo anti-SARS-CoV-2 potency of antibodies/nanobodies from the literature

| | | | | | Treatment | | |
|---|---|---|---|---|---|---|---|
| | | | | | time | | |
| | | Conventional | | | (hours post- | Dosage | |
| Reference | Nanobody | Antibody | Animal | Route | challenge) | (mg/kg) | Virus |
| Example 1 | Nanosota- | | Mouse | IP | 4 | 4 | WT |
| | 2A-Fc | | | | | 10 | |
| | | | | | 18 | 16 | |
| Example 1 | Nanosota- | | Mouse | IP | 4 | 10 | Omicron |
| | 3A-Fc | | | | | | |
| (1) | | 35B5 | Mouse | IP | 6 | 20 | WT |
| | | 32C7 | | | 4 | 30 | 614G, Beta, Delta |
| (2) | | J08, 114 | Hamster | IP | 24 | 4 | WT |
| (3) | | P17 | Mouse | IP | −12 (pre-) | 20 | WT |
| | | | | | 4 | | |
| (4) | | Clone2 | Mouse | IP | −24 (pre-) | 20 | WT |
| | | Clone6 | | | 18 | | |
| | | Clone13A | | | −24 (pre-) | | Delta |
| (5) | | STE90-C11 | Mouse | IV | 1 | 6 | WT |
| | | | | | | 30 | |
| | | | | | | 60 | |
| | | | | | | 120 | |
| | | | Hamster | IP | 2 | 3.7 | |
| | | | | | | 37 | |
| (6) | | WRAIR NTD | Mouse | IV | −24 (pre-) | 0.25 | WT |
| | | mAbs | | | | | |
| | | WRAIR RBD | | | | 1 | |
| | | mAbs | | | | | |
| | | Cocktail | | | 24 | 0.625 (Partial | |
| | | | | | | protective) | |
| | | | | | | 2.5 | |
| (7) | | 31 RBD mAbs | Mouse | IP | 1 | 5 | Beta |
| | | | | | 2 | 30 | Omicron |
| (8) | Nanosota- | | Mouse | IP | −24 (pre-) | 20 | WT |
| | 1C-Fc | | | | 4 | 10 | |
| | | | | | | 20 | |
| (9) | C5-Trimer | | Hamster | IP | 24 | 4 | WT |
| (10) | WNb-Fc 36 | | Mouse | IP | −24 (pre-) | 0.2 | WT (N501Y D614G) |
| (11) | aRBD-2-5- | | Mouse | IP | −24 (pre-) | 10 | WT |
| | Fc | | Hamster | | −24 (pre-) | | Omicron |
| | | | | | 3 | | |
| (12) | 8A2 + 7A3 | | Mouse | IP | −2 (pre-) | 5 | Beta |
| (13) | V_{H}-Fc ab8 | | Mouse | IP | −12 (pre-) | 8 | WT (Q498T/P499Y) |
| | | | | | | 36 | |
| | | | Hamster | | −24 (pre-) | 10 | WT |
| | | | | | 6 | | |
| (14) | VHH72-Fe | | Mouse | IP | −7 (pre-) | 5 | Muc-IMB-1/2020 |
| | HumVHH_{—} | | | IN | | 1 | |
| | S56A/ | | | | | | |
| | LALAPG- | | | | | | |
| | Fc/Gen2 | | | | | | |
| | (humVHH_{—} | | Hamster | IP | −24 (pre-) | 20 | beta |
| | S56A)₂/ | | | | 4 | 2 | |
| | LALAPG- | | | | | 7 | |
| | Fc/Gen2 | | | | | 20 | |
| | | | | IP | 4 | 2 | |
| | | | | | | 7 | |
| | | | | | | 20 | |
| (15) | ShAb01 | | Mouse | IP | −24 (pre-) | 10 | WT |
| | ShAb02 | | | | | | |
| (16) | MR14 | | Mouse | IP | −6 (pre-) | 5 | BA.2 |
| | | | | IN | | | |
| | | | | IN | 6, 30, 54 | | |
| | | | | (3dose) | | | |
| | | | | IP | 6 | | |
| (17) | W25-Fc | | Mouse | IP | −4 (pre-) | 5 | Beta |
| | | | | | 24 | | |
| | | | | IP | 24 | 5 | BA.1 |
| | | | | IN | | 1 | |

IP: intraperitoneally
Pre-: time refers to hours pre-challenge
IN: intranasally
IV: intravenously
Documents in Table S2:
1. X. Wang et al., A potent human monoclonal antibody with pan-neutralizing activities directly dislocates S trimer of SARS-CoV-2 through binding both up and down forms of RBD. Signal transduction and targeted therapy 7, 114 (2022).
2. E. Andreano et al., Extremely potent human monoclonal antibodies from COVID-19 convalescent patients. Cell 184: 1821-1835.e1816. (2021).
3. H. Yao et al., Rational development of a human antibody cocktail that deploys multiple functions to confer Pan-SARS-CoVs protection. Cell Res 31, 25-36 (2021).
4. L. Peng et al., Monospecific and bispecific monoclonal SARS-CoV-2 neutralizing antibodies that maintain potency against B.1.617. Nature communications 13, 1638 (2022).
5. F. Bertoglio et al., A SARS-CoV-2 neutralizing antibody selected from COVID-19 patients binds to the ACE2-RBD interface and is tolerant to most known RBD mutations. (2021).
6. V. Dussupt, et al., Low-dose in vivo protection and neutralization across SARS-CoV-2 variants by monoclonal antibody combinations. Nat Immunol 22: 1503-1514. (2021).
7. K. Wang et al., Memory B cell repertoire from triple vaccinees against diverse SARS-CoV-2 variants. Nature 603, 919-925 (2022).
8. G. Ye et al., The development of Nanosota-1 as anti-SARS-CoV-2 nanobody drug candidates. eLife 10, (2021).
9. J Huo, et al., A potent SARS-CoV-2 neutralising nanobody shows therapeutic efficacy in the Syrian golden hamster model of COVID-19. Nat Commun 12: 5469. (2021).
10. P. Pymm et al., Nanobody cocktails potently neutralize SARS-CoV-2 D614G N501Y variant and protect mice. Proc Natl Acad Sci USA 118, (2021).
11. H. Ma et al., Hetero-bivalent nanobodies provide broad-spectrum protection against SARS-CoV-2 variants of concern including Omicron. Cell Res 32, 831-842 (2022).
12. J. Hong et al., Dromedary camel nanobodies broadly neutralize SARS-CoV-2 variants. Proc Natl Acad Sci USA 119, e2201433119 (2022).
13. W. Li et al., High Potency of a Bivalent Human V(H) Domain in SARS-CoV-2 Animal Models. Cell 183, 429-441.e416 (2020).
14. B. Schepens, et al., An affinity-enhanced, broadly neutralizing heavy chain-only antibody protects against SARS-CoV-2 infection in animal models. Sci Transl Med 13: eabi7826. (2021).
15. W H Chen, et al., Shark nanobodies with potent SARS-CoV-2 neutralizing activity and broad sarbecovirus reactivity. Nat Commun 14: 580. (2023).
16. H Liu, et al., Two pan-SARS-CoV-2 nanobodies and their multivalent derivatives effectively prevent Omicron infections in mice. Cell Rep Med 4: 100918. (2023).
17. N. Modhiran, et al., A nanobody recognizes a unique conserved epitope and potently neutralizes SARS-CoV-2 omicron variants. iScience 26: 107085. (2023).

TABLE S3
Cryo-EM data collection, refinement and validation statistics.

| | | | | | | | |
|---|---|---|---|---|---|---|---|
| | | | Local | | Local | | Local |
| | | | refinement | | refinement | | refinement |
| | Local | Spike/3A | of Spike/3A | Spike/3A | of Spike/3A | Spike/4A | of spike/4A |
| | refinement | complex | complex | complex | complex | complex | complex |
| Spike/2A | of Spike/2A | with | with 2 | with 1 | with 1 | with 2 | with 2 |
| complex | complex | 2 RBDs up | RBDs up | RBD up | RBD up | RBDs up | RBDs up |
| with 2 | with 2 | and 3 Nbs | and 3 Nbs | and 2 Nbs | and 2 Nbs | and 3 Nbs | and 3 Nbs |
| RBDs up | RBDs up | bound | bound | bound | bound | bound | bound |

Data collection and processing

| | | | | | | | | |
|---|---|---|---|---|---|---|---|---|
| Magnification | 165,000 | | 165,000 | | 165,000 | | 75,300 | |
| Voltage (kV) | 300 | | 300 | | 300 | | 300 | |
| Electron exposure (e−/Å²) | 40.00 | | 40.00 | | 40.00 | | 50.00 | |
| Defocus range (μm) | 0.8-2.4 | | 0.8-2.4 | | 0.8-2.4 | | 0.75-2.5 | |
| Pixel size (Å) | 0.73 | | 0.73 | | 0.73 | | 0.664 | |
| Symmetry imposed | C1 | | C1 | | C1 | | C1 | |
| Initial particle images (no.) | 486,437 | 486,437 | 189,794 | 189,794 | 189,794 | 189,794 | 68,226 | 68,226 |
| Final particle images (no.) | 451,926 | 25124 | 81,068 | 81,068 | 77,360 | 77,360 | 58,046 | 58,046 |
| Map resolution (Å) | 2.1 | 5.6 | 2.5 | 3.3 | 2.5 | 3.2 | 3.4 | 4.1 |
| FSC threshold | 0.143 | 0.143 | 0.143 | 0.143 | 0.143 | 0.143 | 0.143 | 0.143 |
| Map resolution range (Å) | 1.8-4.6 | 5-10 | 2.0-8.0 | 3.0-8.0 | 2.0-8.0 | 3.0-8.0 | 3.0-8.0 | 3.2-10 |

Refinement

| | | | | | | | | |
|---|---|---|---|---|---|---|---|---|
| Initial model used (PDB code) | 7TGX | 7TGX | 7TGX | 7TGX | 7TGX | 7TGX | 7TGX | 7TGX |
| Model resolution (Å) | 2.7 | 9.4 | 2.9 | 4.0 | 2.9 | 3.6 | 3.8 | 7.4 |
| FSC threshold | 0.5 | 0.5 | 0.5 | 0.5 | 0.5 | 0.5 | 0.5 | 0.5 |
| Model resolution range (Å) | 44.3-2.1 | 23.4-5.0 | 51.4-2.4 | 43.3-3.2 | 52.0-2.4 | 40.0-3.2 | 63.0-3.4 | 54.4-4.0 |
| Map sharpening B factor (Å²) | −44.9 | −348.4 | −43.2 | −66.0 | −42.7 | −70.3 | −81.2 | −97.3 |

Model composition

| | | | | | | | | |
|---|---|---|---|---|---|---|---|---|
| Non-hydrogen atoms | 25761 | 3037 | 28420 | 16237 | 27543 | 15406 | 28711 | 17466 |
| Protein residues | 3217 | 338 | 3562 | 2046 | 3447 | 1928 | 3598 | 2193 |
| Ligands | 45 | | 47 | 2 | 47 | 7 | 47 | 12 |

B factors (Å²)

| | | | | | | | | |
|---|---|---|---|---|---|---|---|---|
| Protein | 106.09 | 228.15 | 128.96 | 173.60 | 129.02 | 126.74 | 159.30 | 144.99 |
| Nucleotide | | | | | | | | |
| Ligand | 115.62 | | 118.50 | 172.91 | 129.21 | 150.68 | 144.99 | 126.54 |

R.m.s. deviations

| | | | | | | | | |
|---|---|---|---|---|---|---|---|---|
| Bond lengths (Å) | 0.006 | 0.004 | 0.005 | 0.006 | 0.005 | 0.004 | 0.016 | 0.005 |
| Bond angles (°) | 0.949 | 0.852 | 0.843 | 0.831 | 0.839 | 0.724 | 1.028 | 0.767 |

Validation

| | | | | | | | | |
|---|---|---|---|---|---|---|---|---|
| MolProbity score | 2.33 | 1.78 | 2.17 | 2.18 | 2.09 | 1.96 | 1.81 | 2.04 |
| Clashscore | 32.58 | 15.75 | 21.22 | 24.55 | 20.71 | 14.00 | 14.17 | 17.42 |
| Poor rotamers (%) | 1.24 | 0.00 | 1.26 | 0.61 | 1.16 | 0.29 | 0.54 | 0.05 |

Ramachandran plot

| | | | | | | | | |
|---|---|---|---|---|---|---|---|---|
| Favored (%) | 96.20 | 97.64 | 95.97 | 95.65 | 96.48 | 95.59 | 97.19 | 95.68 |
| Allowed (%) | 3.45 | 2.36 | 3.77 | 4.10 | 3.37 | 4.25 | 2.70 | 4.09 |
| Disallowed (%) | 0.35 | 0.00 | 0.26 | 0.25 | 0.15 | 0.16 | 0.11 | 0.23 |

2A, 3A and 4A represent Nanosota-2A, -3A and -4A, respectively.

TABLE A

| SEQ | | |
|---|---|---|
| ID | | |
| NO: | Sequences | Comment |
| 1 | QVQLQESGGGAVQPGGSLGLSCTASGENFETSTVGWERQA | Nanosota-2A VHH |
| | PGKENEGVSCINKGYEDTNYADSVKGRFTISRDAAKNTVY | sequence |
| | LQMDSLQPEDTATYYCAAHNEPYFCDYSGRFRWNEYSYYG | |
| | QGTQVTVSS | |
| 2 | FNFETSTV | Nanosota-2A: CDR-H1 |
| 3 | CINKGYEDTN | Nanosota-2A: CDR-H2 |
| 4 | AAHNEPYFCDYSGRFRWNEYSY | Nanosota-2A: CDR-H3 |
| 5 | QVQLQESGGGAVQPGGSLGLSCTASGFNFETSTVGWFRQA | Nanosota-2A VHH |
| | PGKENEGVSCINKGYEDTNYADSVKGRFTISRDAAKNTVY | sequence with linker |
| | LQMDSLQPEDTATYYCAAHNEPYFCDYSGRFRWNEYSYYG | (bold) and His₆ tag |
| | QGTQVTVSSGSHHHHHH | (SEQ ID NO: 20) |
| | | (italics) |
| 6 | QVQLQESGGGAVQPGGSLGLSCTASGFNFETSTVGWFRQA | Nanosota-2A VHH |
| | PGKENEGVSCINKGYEDTNYADSVKGRFTISRDAAKNTVY | sequence with IgG1 |
| | LQMDSLQPEDTATYYCAAHNEPYFCDYSGRFRWNEYSYYG | Fc domain |
| | QGTQVTVSSEPKSCDKTHTCPPCPAPELLGGPSVFLFPPK | sequence (bold) |
| | PKDTLMISRTPEVTCVVVDVSHEDPEVKFNWYVDGVEVHN | |
| | AKTKPREEQYNSTYRVVSVLTVLHQDWLNGKEYKCKVSNK | |
| | ALPAPIEKTISKAKGQPREPQVYTLPPSREEMTKNQVSLT | |
| | CLVKGFYPSDIAVEWESNGQPENNYKTTPPVLDSDGSFFL | |
| | YSKLTVDKSRWQQGNVFSCSVMHEALHNHYTQKSLSLSPG | |
| | K | |
| 22 | CAGGTGCAGCTGCAGGAGTCTGGGGGAGGCGCCGTGCAGC | DNA sequence for |
| | CTGGGGGGTCTCTCGGACTCTCCTGTACAGCCTCTGGATT | Nanosota-2A VHH |
| | CAATTTTGAAACTTCAACCGTAGGCTGGTTCCGCCAGGCC | CDR-H1, CDR-H2, CDR- |
| | CCAGGGAAGGAGAATGAGGGGGTCTCATGTATCAATAAAG | H3 in bold |
| | GTTATGAAGATACAAATTATGCAGACTCCGTGAAGGGCCG | |
| | GTTCACCATCTCCAGAGACGCCGCCAAGAACACGGTGTAC | |
| | CTGCAAATGGACAGCCTGCAACCTGAGGACACAGCCACAT | |
| | ATTATTGTGCAGCACATAATGAGCCTTATTTTTGCGACTA | |
| | TAGTGGGCGTTTTCGGTGGAATGAGTACAGCTACTATGGC | |
| | CAGGGGACCCAGGTCACCGTCTCCTCA | |
| 23 | CAGGTGCAGCTGCAGGAGTCTGGGGGAGGCGCCGTGCAGC | DNA sequence for |
| | CTGGGGGGTCTCTCGGACTCTCCTGTACAGCCTCTGGATT | Nanosota-2A-Fc |
| | CAATTTTGAAACTTCAACCGTAGGCTGGTTCCGCCAGGCC | CDR-H1, CDR-H2, CDR- |
| | CCAGGGAAGGAGAATGAGGGGGTCTCATGTATCAATAAAG | H3 in bold, Fc |
| | GTTATGAAGATACAAATTATGCAGACTCCGTGAAGGGCCG | domain in |
| | GTTCACCATCTCCAGAGACGCCGCCAAGAACACGGTGTAC | italic/bold |
| | CTGCAAATGGACAGCCTGCAACCTGAGGACACAGCCACAT | |
| | ATTATTGTGCAGCACATAATGAGCCTTATTTTTGCGACTA | |
| | TAGTGGGCGTTTTCGGTGGAATGAGTACAGCTACTATGGC | |
| | CAGGGGACCCAGGTCACCGTCTCCTCAGAGCCCAAATCTT | |
| | GTGACAAAACTCACACATGCCCACCGTGCCCAGCACCTGA | |
| | ACTCCTGGGGGGACCGTCAGTCTTCCTCTTCCCCCCAAAA | |
| | CCCAAGGACACCCTCATGATCTCCCGGACCCCTGAGGTCA | |
| | CATGCGTGGTGGTGGACGTGAGCCACGAAGACCCTGAGGT | |
| | CAAGTTCAACTGGTACGTGGACGGCGTGGAGGTGCATAAT | |
| | GCCAAGACAAAGCCGCGGGAGGAGCAGTACAACAGCACGT | |
| | ACCGTGTGGTCAGCGTCCTCACCGTCCTGCACCAGGACTG | |
| | GCTGAATGGCAAGGAGTACAAGTGCAAGGTCTCCAACAAA | |
| | GCCCTCCCAGCCCCCATCGAGAAAACCATCTCCAAAGCCA | |
| | AAGGGCAGCCCCGAGAACCACAGGTGTACACCCTGCCCCC | |
| | ATCCCGGGAGGAGATGACCAAGAACCAGGTCAGCCTGACC | |
| | TGCCTGGTCAAAGGCTTCTATCCCAGCGACATCGCCGTGG | |
| | AGTGGGAGAGCAATGGGCAGCCGGAGAACAACTACAAGAC | |
| | CACGCCTCCCGTGCTGGACTCCGACGGCTCCTTCTTCCTC | |
| | TACAGCAAGCTCACCGTGGACAAGAGCAGGTGGCAGCAGG | |
| | GGAACGTCTTCTCATGCTCCGTGATGCATGAGGCTCTGCA | |
| | CAACCACTACACGCAGAAGAGCCTCTCCCTGTCTCCGGGT | |
| | AAA | |
| 7 | QVQLQESGGGLVQAGGSLRLSCAASGSIFSPNTMGWFRQA | Nanosota-3A VHH |
| | LGKQREMVAVISSIASTQYANFVKGRFTITRDNTKNTVHL | sequence |
| | QMNSLIPEDTAVYYCYAVDKSQDYWGQGTQVTVSS | |
| 8 | SIFSPNTM | Nanosota-3A: CDR-H1 |
| 9 | VISSIASTQ | Nanosota-3A: CDR-H2 |
| 10 | YAVDKSQDY | Nanosota-3A: CDR-H3 |
| 11 | QVQLQESGGGLVQAGGSLRLSCAASGSIFSPNTMGWFRQA | Nanosota-3A VHH |
| | LGKQREMVAVISSIASTQYANFVKGRFTITRDNTKNTVHL | sequence with linker |
| | QMNSLIPEDTAVYYCYAVDKSQDYWGQGTQVTVSSGSHHH | (bold) and His₆ tag |
| | HHH | (SEQ ID NO: 20) |
| | | (italics) |
| 12 | QVQLQESGGGLVQAGGSLRLSCAASGSIFSPNTMGWFRQA | Nanosota-3A VHH |
| | LGKQREMVAVISSIASTQYANFVKGRFTITRDNTKNTVHL | sequence with IgG1 |
| | QMNSLIPEDTAVYYCYAVDKSQDYWGQGTQVTVSSEPKSC | Fc domain sequence |
| | DKTHTCPPCPAPELLGGPSVFLFPPKPKDTLMISRTPEVT | (bold) |
| | CVVVDVSHEDPEVKFNWYVDGVEVHNAKTKPREEQYNSTY | |
| | RVVSVLTVLHQDWLNGKEYKCKVSNKALPAPIEKTISKAK | |
| | GQPREPQVYTLPPSREEMTKNQVSLTCLVKGFYPSDIAVE | |
| | WESNGQPENNYKTTPPVLDSDGSFFLYSKLTVDKSRWQQG | |
| | NVFSCSVMHEALHNHYTQKSLSLSPGK | |
| 24 | CAGGTGCAGCTGCAGGAGTCTGGGGGAGGCTTGGTGCAGG | DNA sequence for |
| | CTGGGGGGTCTCTGAGACTCTCCTGTGCAGCCTCTGGAAG | Nanosota-3A VHH |
| | CATCTTCAGTCCTAATACCATGGGCTGGTTTCGCCAGGCT | CDR-H1, CDR-H2, CDR- |
| | CTAGGGAAGCAGCGCGAAATGGTCGCAGTTATTAGTAGTA | H3 in bold |
| | TTGCTAGCACGCAGTATGCAAACTTCGTGAAGGGCCGATT | |
| | CACCATCACCAGAGACAACACCAAGAATACGGTGCATCTC | |
| | CAAATGAACAGCCTGATTCCTGAGGACACAGCCGTCTATT | |
| | ACTGTTATGCCGTCGACAAGTCCCAAGACTACTGGGGCCA | |
| | GGGGACCCAGGTCACCGTCTCCTCA | |
| 25 | CAGGTGCAGCTGCAGGAGTCTGGGGGAGGCTTGGTGCAGG | DNA sequence for |
| | CTGGGGGGTCTCTGAGACTCTCCTGTGCAGCCTCTGGAAG | Nanosota-3A-Fc |
| | CATCTTCAGTCCTAATACCATGGGCTGGTTTCGCCAGGCT | CDR-H1, CDR-H2, CDR- |
| | CTAGGGAAGCAGCGCGAAATGGTCGCAGTTATTAGTAGTA | H3 in bold, Fc |
| | TTGCTAGCACGCAGTATGCAAACTTCGTGAAGGGCCGATT | domain in |
| | CACCATCACCAGAGACAACACCAAGAATACGGTGCATCTC | italic/bold |
| | CAAATGAACAGCCTGATTCCTGAGGACACAGCCGTCTATT | |
| | ACTGTTATGCCGTCGACAAGTCCCAAGACTACTGGGGCCA | |
| | GGGGACCCAGGTCACCGTCTCCTCAGAGCCCAAATCTTGT | |
| | GACAAAACTCACACATGCCCACCGTGCCCAGCACCTGAAC | |
| | TCCTGGGGGGACCGTCAGTCTTCCTCTTCCCCCCAAAACC | |
| | CAAGGACACCCTCATGATCTCCCGGACCCCTGAGGTCACA | |
| | TGCGTGGTGGTGGACGTGAGCCACGAAGACCCTGAGGTCA | |
| | AGTTCAACTGGTACGTGGACGGCGTGGAGGTGCATAATGC | |
| | CAAGACAAAGCCGCGGGAGGAGCAGTACAACAGCACGTAC | |
| | CGTGTGGTCAGCGTCCTCACCGTCCTGCACCAGGACTGGC | |
| | TGAATGGCAAGGAGTACAAGTGCAAGGTCTCCAACAAAGC | |
| | CCTCCCAGCCCCCATCGAGAAAACCATCTCCAAAGCCAAA | |
| | GGGCAGCCCCGAGAACCACAGGTGTACACCCTGCCCCCAT | |
| | CCCGGGAGGAGATGACCAAGAACCAGGTCAGCCTGACCTG | |
| | CCTGGTCAAAGGCTTCTATCCCAGCGACATCGCCGTGGAG | |
| | TGGGAGAGCAATGGGCAGCCGGAGAACAACTACAAGACCA | |
| | CGCCTCCCGTGCTGGACTCCGACGGCTCCTTCTTCCTCTA | |
| | CAGCAAGCTCACCGTGGACAAGAGCAGGTGGCAGCAGGGG | |
| | AACGTCTTCTCATGCTCCGTGATGCATGAGGCTCTGCACA | |
| | ACCACTACACGCAGAAGAGCCTCTCCCTGTCTCCGGGTAA | |
| | A | |
| 13 | QVQLQESGGGLVQPGGSLRLSCAASGFTLDYYAIGWFRQA | Nanosota-4A VHH |
| | PGKEREGVSCISSSGGRTNYADSVKGRFTISRDNTKNTVY | sequence |
| | LQMNSLKPEDTAVYYCAAWEASRWYCPLQFSADFSSWGQG | |
| | TQVTVSS | |
| 14 | FTLDYYAI | Nanosota-4A: CDR-H1 |
| 15 | CISSSGGRTN | Nanosota-4A: CDR-H2 |
| 16 | AAWEASRWYCPLQFSADFSS | Nanosota-4A: CDR-H3 |
| 17 | QVQLQESGGGLVQPGGSLRLSCAASGFTLDYYAIGWFRQA | Nanosota-4A VHH |
| | PGKEREGVSCISSSGGRTNYADSVKGRFTISRDNTKNTVY | sequence with linker |
| | LQMNSLKPEDTAVYYCAAWEASRWYCPLQFSADFSSWGQG | (bold) and His₆ tag |
| | TQVTVSSGSHHHHHH | (SEQ ID NO: 20) |
| | | (italics) |
| 18 | QVQLQESGGGLVQPGGSLRLSCAASGFTLDYYAIGWFRQA | Nanosota-4A VHH |
| | PGKEREGVSCISSSGGRTNYADSVKGRFTISRDNTKNTVY | sequence with IgG1 |
| | LQMNSLKPEDTAVYYCAAWEASRWYCPLQFSADFSSWGQG | Fc domain sequence |
| | TQVTVSSEPKSCDKTHTCPPCPAPELLGGPSVFLFPPKPK | (bold) |
| | DTLMISRTPEVTCVVVDVSHEDPEVKFNWYVDGVEVHNAK | |
| | TKPREEQYNSTYRVVSVLTVLHQDWLNGKEYKCKVSNKAL | |
| | PAPIEKTISKAKGQPREPQVYTLPPSREEMTKNQVSLTCL | |
| | VKGFYPSDIAVEWESNGQPENNYKTTPPVLDSDGSFFLYS | |
| | KLTVDKSRWQQGNVFSCSVMHEALHNHYTQKSLSLSPGK | |
| 26 | CAGGTGCAGCTGCAGGAGTCTGGGGGAGGCTTGGTGCAGC | DNA sequence for |
| | CTGGGGGGTCTCTGAGACTCTCCTGTGCAGCCTCTGGATT | Nanosota-4A VHH |
| | CACTTTGGATTATTATGCCATAGGCTGGTTCCGCCAGGCC | CDR-H1, CDR-H2, CDR- |
| | CCAGGGAAGGAGCGTGAGGGGGTCTCATGTATTAGTAGTA | H3 in bold |
| | GTGGTGGGCGCACAAACTATGCAGACTCCGTGAAGGGCCG | |
| | ATTCACCATCTCCAGAGACAACACCAAGAACACGGTGTAT | |
| | TTGCAGATGAACAGTCTGAAACCTGAGGACACAGCCGTTT | |
| | ATTACTGCGCAGCTTGGGAGGCTAGTAGGTGGTACTGTCC | |
| | ACTCCAATTTTCTGCTGACTTTAGTTCCTGGGGCCAGGGG | |
| | ACCCAGGTCACCGTCTCCTCA | |
| 27 | CAGGTGCAGCTGCAGGAGTCTGGGGGAGGCTTGGTGCAGC | DNA sequence for |
| | CTGGGGGGTCTCTGAGACTCTCCTGTGCAGCCTCTGGATT | Nanosota-4A-Fc |
| | CACTTTGGATTATTATGCCATAGGCTGGTTCCGCCAGGCC | CDR-H1, CDR-H2, CDR- |
| | CCAGGGAAGGAGCGTGAGGGGGTCTCATGTATTAGTAGTA | H3 in bold, Fc |
| | GTGGTGGGCGCACAAACTATGCAGACTCCGTGAAGGGCCG | domain in |
| | ATTCACCATCTCCAGAGACAACACCAAGAACACGGTGTAT | italic/bold |
| | TTGCAGATGAACAGTCTGAAACCTGAGGACACAGCCGTTT | |
| | ATTACTGCGCAGCTTGGGAGGCTAGTAGGTGGTACTGTCC | |
| | ACTCCAATTTTCTGCTGACTTTAGTTCCTGGGGCCAGGGG | |
| | ACCCAGGTCACCGTCTCCTCAGAGCCCAAATCTTGTGACA | |
| | AAACTCACACATGCCCACCGTGCCCAGCACCTGAACTCCT | |
| | GGGGGGACCGTCAGTCTTCCTCTTCCCCCCAAAACCCAAG | |
| | GACACCCTCATGATCTCCCGGACCCCTGAGGTCACATGCG | |
| | TGGTGGTGGACGTGAGCCACGAAGACCCTGAGGTCAAGTT | |
| | CAACTGGTACGTGGACGGCGTGGAGGTGCATAATGCCAAG | |
| | ACAAAGCCGCGGGAGGAGCAGTACAACAGCACGTACCGTG | |
| | TGGTCAGCGTCCTCACCGTCCTGCACCAGGACTGGCTGAA | |
| | TGGCAAGGAGTACAAGTGCAAGGTCTCCAACAAAGCCCTC | |
| | CCAGCCCCCATCGAGAAAACCATCTCCAAAGCCAAAGGGC | |
| | AGCCCCGAGAACCACAGGTGTACACCCTGCCCCCATCCCG | |
| | GGAGGAGATGACCAAGAACCAGGTCAGCCTGACCTGCCTG | |
| | GTCAAAGGCTTCTATCCCAGCGACATCGCCGTGGAGTGGG | |
| | AGAGCAATGGGCAGCCGGAGAACAACTACAAGACCACGCC | |
| | TCCCGTGCTGGACTCCGACGGCTCCTTCTTCCTCTACAGC | |
| | AAGCTCACCGTGGACAAGAGCAGGTGGCAGCAGGGGAACG | |
| | TCTTCTCATGCTCCGTGATGCATGAGGCTCTGCACAACCA | |
| | CTACACGCAGAAGAGCCTCTCCCTGTCTCCGGGTAAA | |
| | GS | Linker sequence |
| 20 | HHHHHH | His6 tag sequence |
| 21 | EPKSCDKTHTCPPCPAPELLGGPSVFLFPPKPKDTLMISR | IgG1 Fc sequence |
| | TPEVTCVVVDVSHEDPEVKFNWYVDGVEVHNAKTKPREEQ | |
| | YNSTYRVVSVLTVLHQDWLNGKEYKCKVSNKALPAPIEKT | |
| | ISKAKGQPREPQVYTLPPSREEMTKNQVSLTCLVKGFYPS | |
| | DIAVEWESNGQPENNYKTTPPVLDSDGSFFLYSKLTVDKS | |
| | RWQQGNVFSCSVMHEALHNHYTQKSLSLSPGK | |

### Example 2. Epitope Mapping of SARS-CoV-2 Spike Protein Using Nanobody Probes

Where are the dominant epitopes on SARS-CoV-2 spike protein and what are their functions? To address these critical questions, we used nanobodies (single-domain antibodies) to probe the epitopes on the spike. We collected spike-induced nanobodies from an alpaca and determined the cryo-EM structure of the spike complexed with all of the spike-bound nanobodies. These nanobodies bind to four unique epitopes on the spike: two recognize epitopes on the receptor-binding domain (RBD) and potently neutralize viral entry, whereas two other recognize epitopes outside the RBD and induce antibody-dependent enhancement (ADE) of viral infection. Depletion assay confirmed that RBD epitopes account for neutralization, while non-RBD epitopes account for ADE. Scenario is possible where ADE epitopes dominate over neutralizing epitopes, enhancing viral infection in COVID-19 patients with pre-existing immunity. For safety and potency, vaccines and therapeutics against COVID-19 may need to selectively target neutralizing epitopes and exclude ADE epitopes on the spike.

### Introduction

A critical gap in virology and immunology is the lack of an overview of the dominant epitopes on viral glycoproteins where high-affinity and prevalent antibodies bind. Three years into the COVID-19 pandemic (1, 2), we still do not know how many dominant epitopes are present on the SARS-CoV-2 spike protein, how they are distributed on the spike, or how they contribute to viral infection. Antibody development has mainly been a trial-and-error practice, where researchers discover virus- or vaccine-induced antibodies and then map their epitopes individually on the spike (3-5). The lack of understanding of the spike's dominant epitopes limits intervention strategies. For example, without knowing the distribution of neutralizing epitopes, it is challenging to design vaccines and therapeutics with high potency (6-8). Moreover, without knowing the distribution of potentially harmful epitopes (such as epitopes that cause antibody-dependent enhancement or ADE of viral infection), it is challenging to guarantee the safety of vaccines and therapeutics (9-12). In this Example, we mapped all of the dominant epitopes on SARS-CoV-2 spike and characterized their functions.

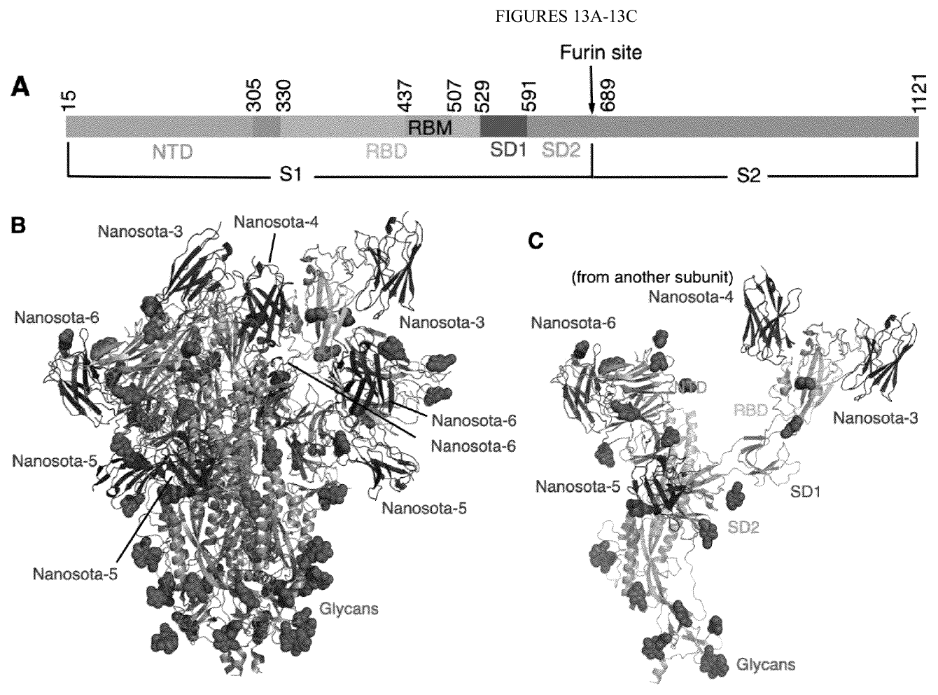

The SARS-CoV-2 spike is a complex multifunctional molecular machine that mediates viral entry into host cells (13-15). Its “pre-fusion structure” on the virus surface is a homotrimer (total molecular weight of >500 kDa), with three receptor-binding S1 subunits sitting on top of a trimeric membrane-fusion S2 stalk. Each S1 contains a receptor-binding domain (RBD), an N-terminal domain (NTD), and two subdomains (SD1 and SD2) (FIG. 13A). The RBD contains a core structure and a receptor-binding motif (RBM) (16-18). The RBD is present in two conformations: standing up for receptor binding or lying down for immune evasion (19, 20). During cell entry, the RBD binds to its host receptor ACE2 (18, 21, 22), whereas the S1/S2 boundary is cleaved by a host protease (19); then S1 dissociates and S2 transitions to its “post-fusion” structure, fusing viral and host membranes (13-15). The surface of the pre-fusion trimeric spike is cover by -66 glycans (23). Among these functional units, RBD is the most divergent and S2 is the most conserved (24). The spike induces most of the host immune responses (13-15). To map all of its dominant epitopes, the spike would need to induce antibodies in a host; subsequently the spike complexed with all of the spike-bound antibodies would need to be prepared and subjected to cryo-EM analysis. However, the above approach is technically challenging because the bivalent IgGs would crosslink the trimeric spike, leading to difficulty in obtaining well-dispersed and homogenous samples for cryo-EM analysis. Nanobodies are the antigen-binding domain of heavy-chain-only antibodies produced by camelid animals (e.g., camels, llamas and alpacas) (25-27). Because of the natural single-domain structure of nanobodies, they are excellent tools for mapping epitopes on the spike.

Here we immunized an alpaca with SARS-CoV-2 spike, collected a mixture of spike-binding nanobodies, purified the complex of the spike and all of the spike-bound nanobodies, and mapped the dominant epitopes on the spike using cryo-EM. We also characterized the functions of these epitopes, identifying both neutralizing epitopes and ADE epitopes. These epitopes potentially play important roles in SARS-CoV-2 infection of patients with pre-existing immunity. This study provides guidance on developing safe and potent vaccines and therapeutics against SARS-CoV-2 spike. This approach may be extended to studying other viral glycoproteins, facilitating vaccine and therapeutics developments against many other viruses.

### Results

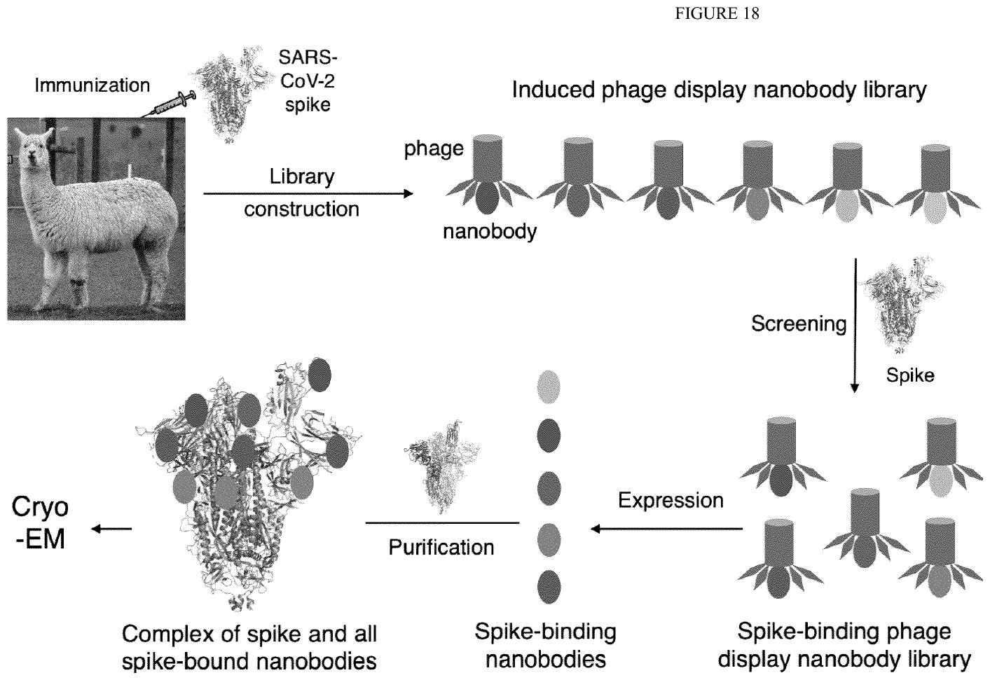

To prepare spike-induced nanobodies, we immunized an alpaca with purified SARS-CoV-2 spike ectodomain, collected its peripheral blood mononuclear cells, made a cDNA library from these cells, amplified the coding regions of the antigen-binding domains (i.e., nanobodies) of the heavy-chain-only antibodies, and generated an induced nanobody phage display library expressing all of the nanobodies. We performed one round of bio-panning using the purified spike, harvested all of the spike-binding phages, and collected a mixture of spike-binding nanobodies from these phages (FIG. 18).

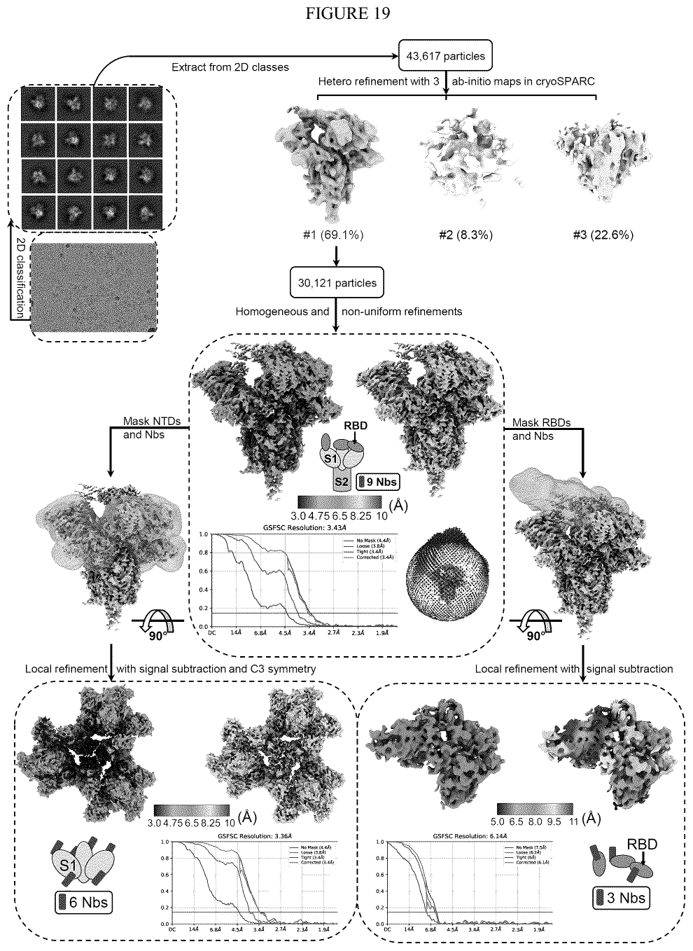

To identify all of the dominant epitopes on the spike, we incubated the spike and the mixture of spike-binding nanobodies, purified the complex of the spike and all of the spike-bound nanobodies, and then determined the cryo-EM structure of this complex (FIG. 13B; FIG. 18). The final cryo-EM map of the complex was calculated at 3.4A resolution (FIG. 19; Table S4). The trimeric spike contains one standing-up RBD and two lying-down RBDs. A total of 57 glycans were built into the trimeric spike (19 for each spike subunit, missing three that are located in the disordered C-terminal region) (23). The densities of nine spike-bound nanobodies were clearly identified (FIG. 13B). Because of the low resolutions of local densities for the nanobodies, the docking models of these nine nanobodies were built initially (eight of them were later replaced by high-resolution atomic models; see below). Due to the asymmetry of the trimeric spike, the nine nanobodies bind to four unique epitopes: two in the RBD (bound by nanobodies named Nanosota-3 and -4) and two outside the RBD (bound by Nanosota-5 and -6) (FIG. 13C).

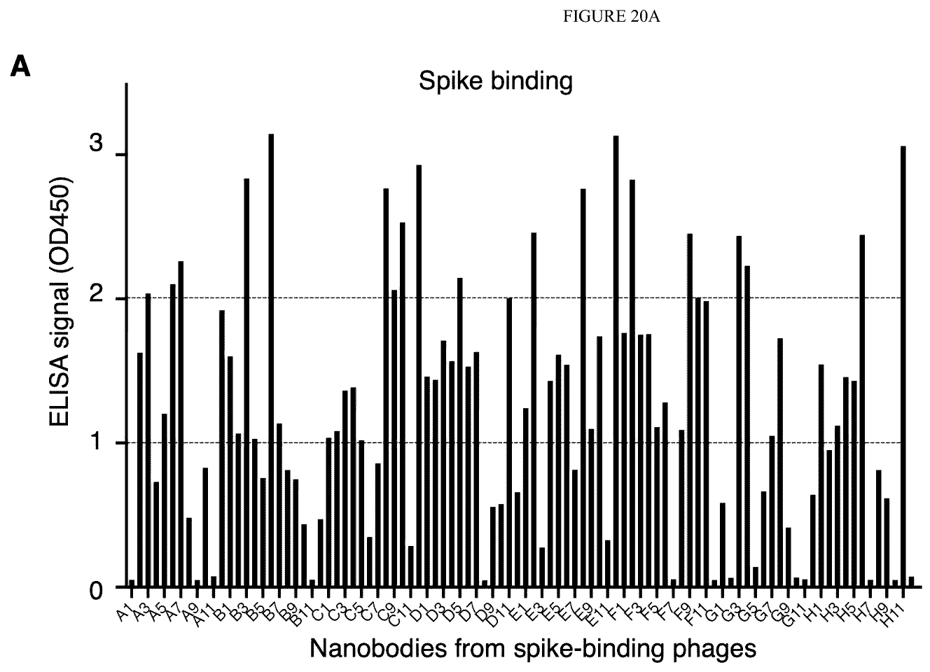

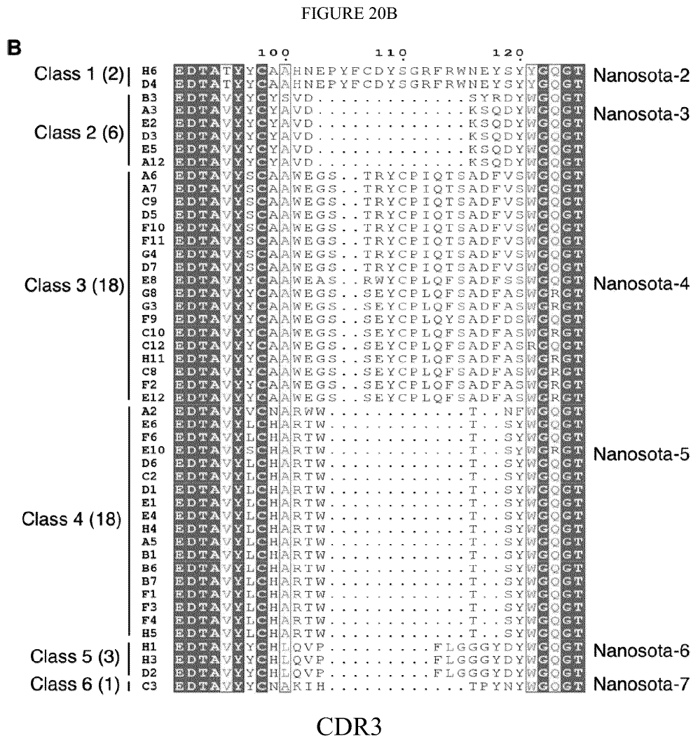

To confirm the epitopes identified in the structure of the spike/nanobody mixture complex, we analyzed individual spike-binding nanobodies. Specifically, we randomly selected 96 spike-binding phages, measured their binding affinity for the spike (FIG. 20A), and chose 48 top spike-binding phages for sequencing of their nanobody gene. These nanobodies fall into six classes (FIG. 20B): the nanobodies in each class are highly similar to each other, suggesting that they originated from the same ancestor but had diverged slightly during immune affinity maturation. The number of nanobodies in each class reflects their relative prevalence in the alpaca's immune response to the spike (FIG. 20B). From each of these six classes of top spike-binding nanobodies, we selected for further analysis one nanobody that bound the spike with the highest affinity.

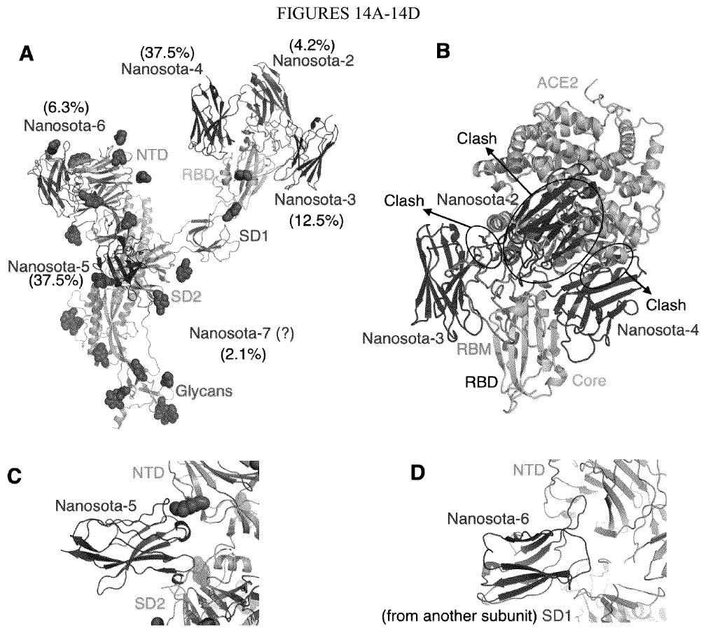

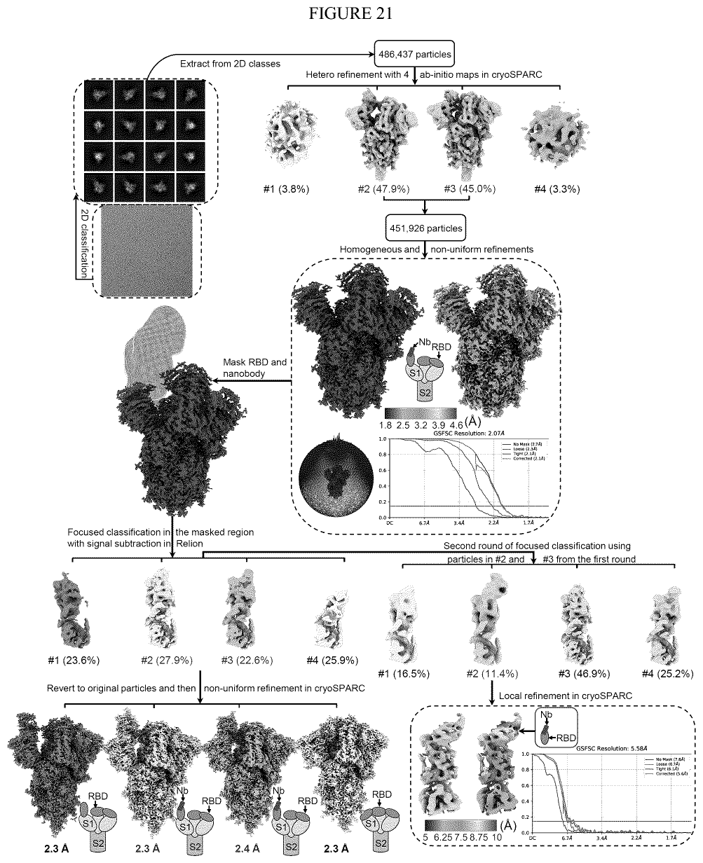

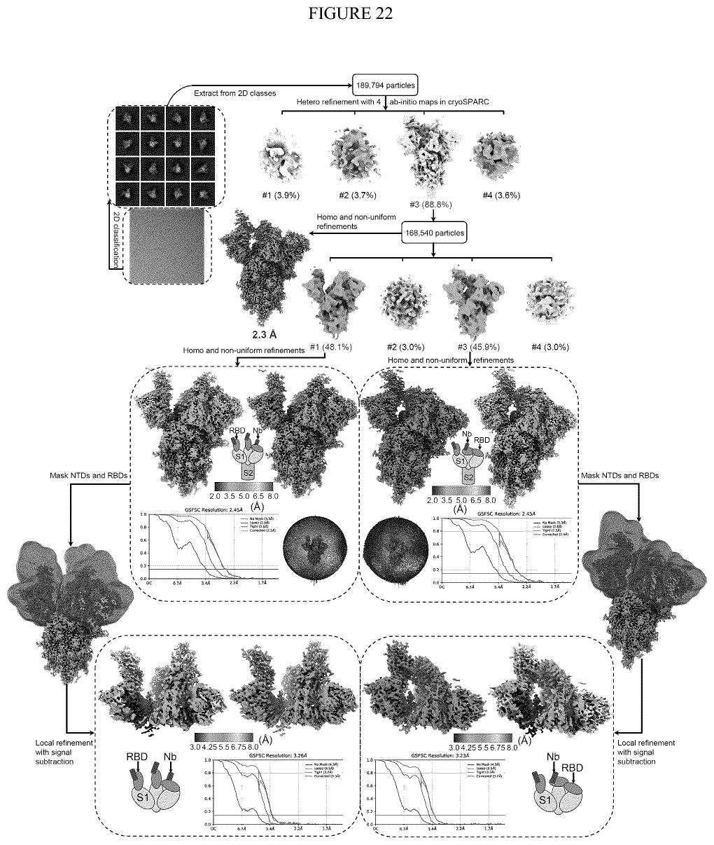

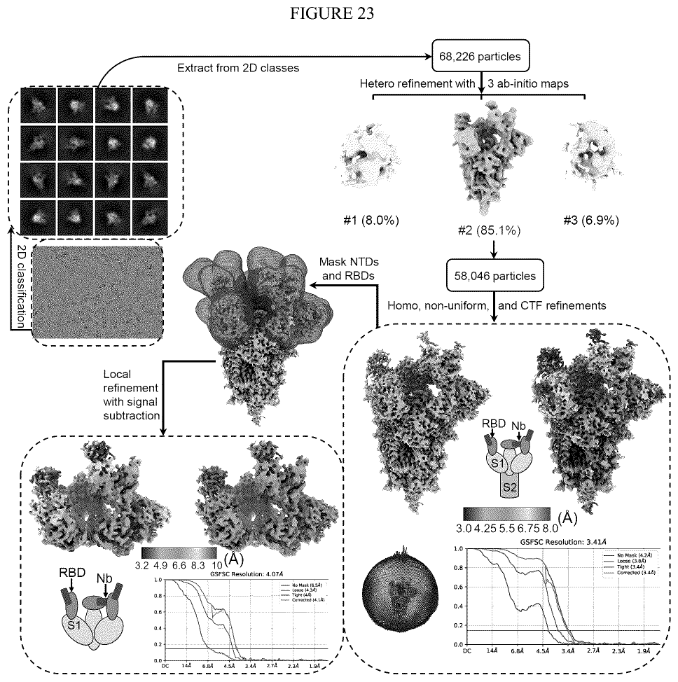

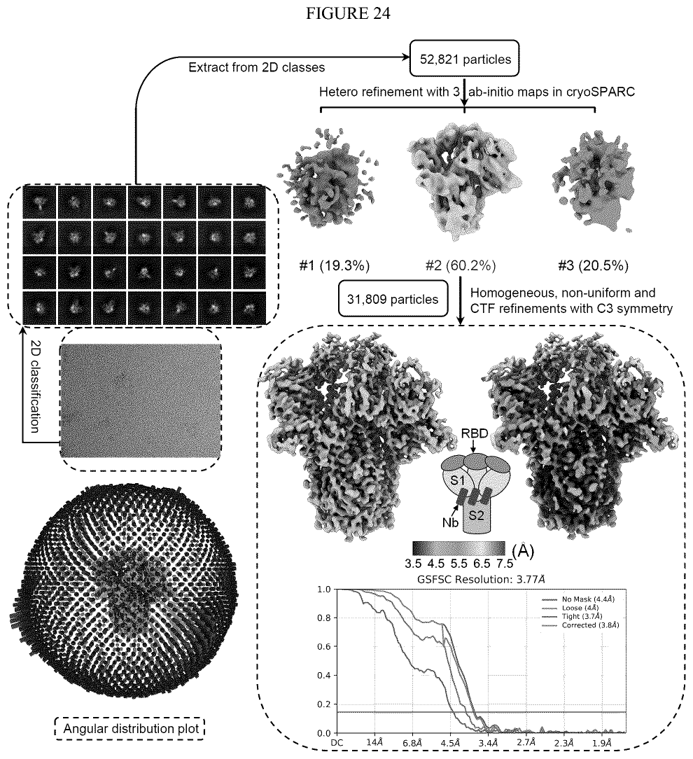

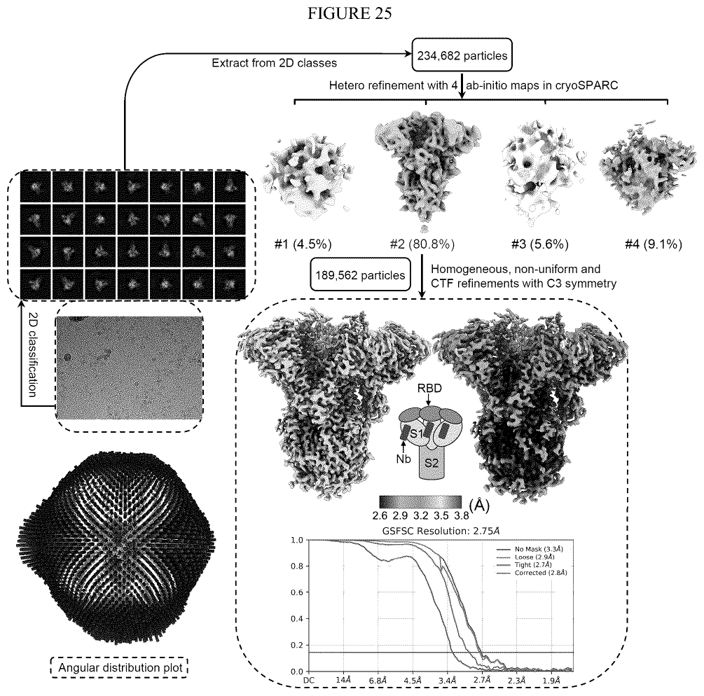

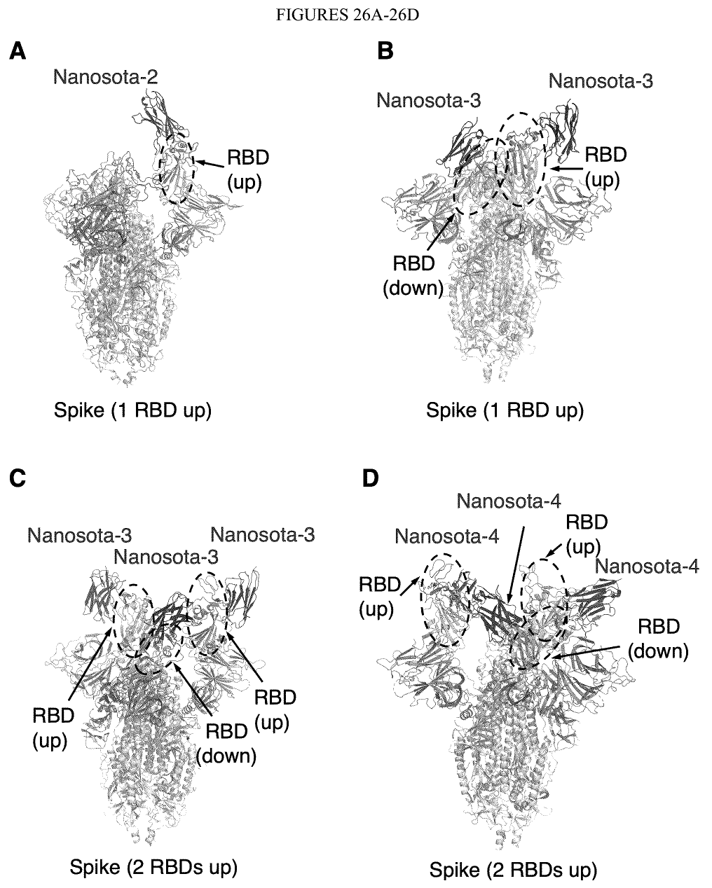

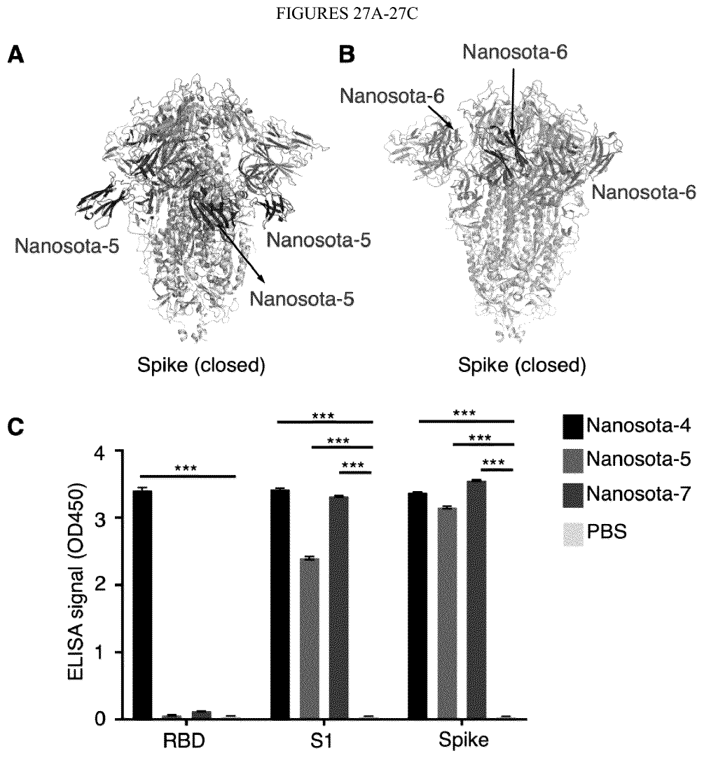

We determined the cryo-EM structures of the spike complexed with each of the six top spike-binding nanobodies (FIG. 14A; FIG. 21-27; Table S5-S6). In addition to Nanosota-3, -4, -5, and -6, we identified one more nanobody (named Nanosota-2), which binds to the RBD with high affinity. We could not identify the binding site for the last nanobody (named Nanosota-7), but ELISA showed that Nanosota-7 binds to a non-RBD region in S1 (FIG. 27C). Based on the respective resolutions of the cryo-EM maps (FIG. 21-27; Table S5-S6), atomic models for Nanosota-3, -5, and -6 were built, whereas docking models for Nanosota-2 and -4 were generated.

The epitopes of the individual nanobodies on the spike indicate their respective function in the immunogenicity of the spike. Nanosota-2, -3, and -4 bind to non-overlapping regions on the RBD (FIG. 14B). Specifically, Nanosota-2 binds to the top of the RBM and its epitope completely overlaps with the ACE2-binding region, whereas Nanosota-3 and -4 bind to each side of the RBD and both of their epitopes partially overlap with the ACE2-binding region. Thus, binding of each of these nanobodies to the RBD would have clash with bound ACE2, hence preventing ACE2 from binding to the RBD. Nanosota-5 binds to the NTD and SD2 from the same spike subunit (FIG. 14C), whereas Nanosota-6 binds to the NTD and SD1 from two different spike subunits (FIG. 14D). The epitopes of Nanosota-5 and -6 do not overlap with the ACE2-binding region. Thus, based on the structural data, the epitopes for Nanosota-2, -3, and -4 are all neutralizing, whereas the epitopes for Nanosota-5 and -6 do not have obvious functions.

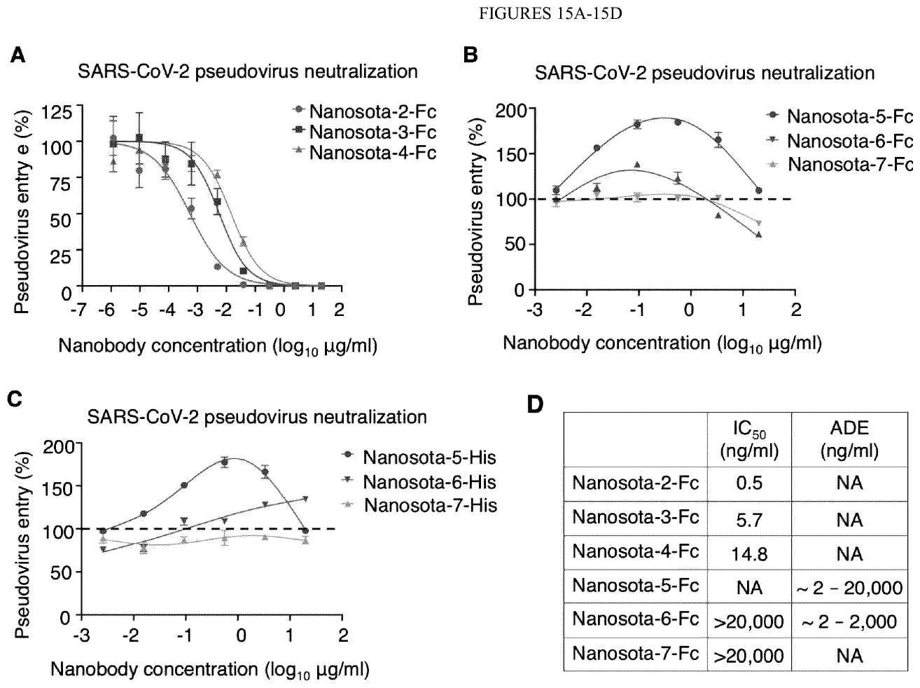

To further understand the functions of the identified epitopes, we investigated how the corresponding nanobodies affect SARS-CoV-2 pseudovirus entry. Since nanobodies function as Fc-tagged dimers in vivo, we introduced a C-terminal Fc tag to Nanosota-2 to -7 (named Nanosota-2-Fc, etc.). Subsequently, retroviruses pseudotyped with SARS-CoV-2 spike (i.e., SARS-CoV-2 pseudoviruses) were used to enter ACE2-expressing HEK293T cells in the presence of each of the six Fc-tagged nanobodies. Nanosota-2-Fc, -3-Fc, and -4-Fc potently inhibited SARS-CoV-2 pseudovirus entry (FIG. 15A). However, Nanosota-6-Fc and -7-Fc only slightly neutralized SARS-CoV-2 pseudovirus entry at high concentrations (FIG. 15B). Curiously, Nanosota-5-Fc at a wide concentration range and Nanosota-6-Fc at low concentrations significantly enhanced SARS-CoV-2 pseudovirus entry (FIG. 15B). This resembled the ADE of coronavirus entry that we observed previously using RBD-targeting neutralizing IgGs (12). We previously showed that RBD-targeting IgGs, which contain Fc, guide coronavirus entry into Fc receptor (FcR)-expressing cells (12). However, Nanosota-5-Fc and -6-Fc enhanced SARS-CoV-2 pseudovirus entry into HEK293T cells that stably express human ACE2, but not FcR. To confirm that FcR did not play a role in the newly identified ADE, we repeated the SARS-CoV-2 pseudovirus entry using His-tagged nanobodies (i.e., Nanosota-5-His, etc.). Both Nanosota-5-His and -6-His caused ADE despite having no Fc tag. The pseudovirus data confirm that the three RBD epitopes are all neutralizing, whereas two of the three non-RBD epitopes cause ADE (FIG. 15D).

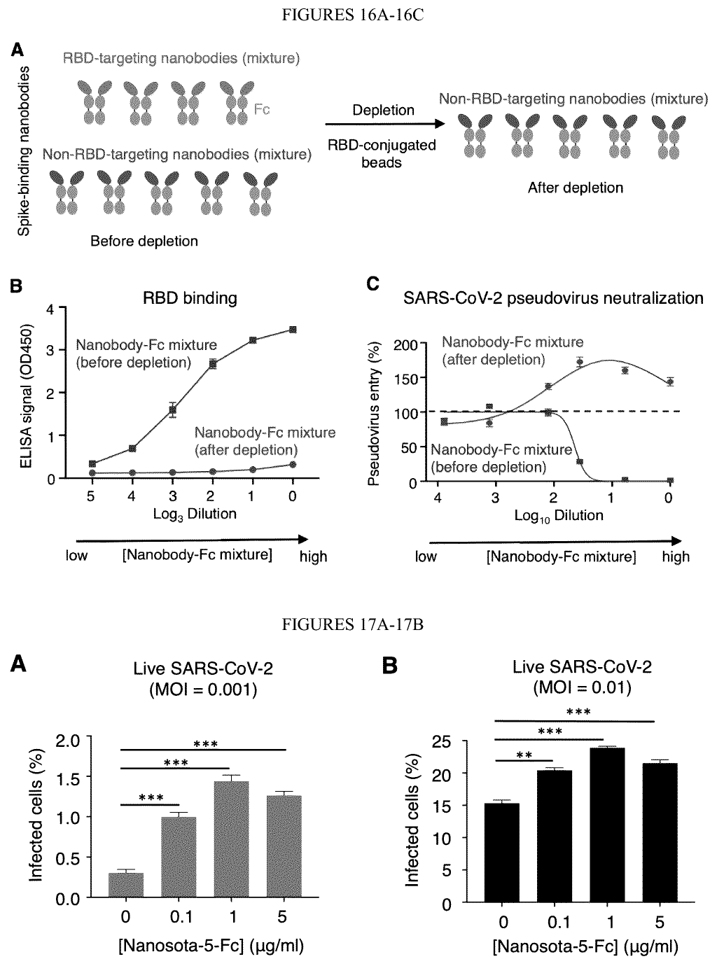

To corroborate the above findings on the functions of the identified epitopes, we performed a depletion assay. Specifically, we inserted a C-terminal Fc tag to all of the spike-binding nanobodies and prepared a mixture of Fc-tagged spike-binding nanobodies (FIG. 16A). From this mixture of Fc-tagged nanobodies, we depleted the RBD-binding ones using RBD-conjugated beads (which was in excess) (FIG. 16A). The mixtures of Fc-tagged spike-binding nanobodies before and after depletion were subjected to SARS-CoV-2 pseudovirus entry assay. The nanobody mixture before depletion inhibited pseudovirus entry efficiently (FIG. 16B), whereas the nanobody mixture after depletion enhanced pseudovirus entry over a wide concentration range (FIG. 16C). This depletion experiment confirmed that RBD epitopes account for neutralization and non-RBD epitopes account for ADE.

To further understand ADE, we examined whether non-RBD-targeting nanobodies can enhance live SARS-CoV-2 infection of cultured cells. Specifically, live SARS-CoV-2 infected ACE2-expressing Vero cells in the presence of Nanosota-5-Fc. Two different virus titers and three different nanobody concentrations were used. At the higher virus titer, significant ADE of viral infections was observed at all nanobody concentrations (FIG. 17A); at the lower virus titer, ADE became even more prominent (viral infection increased by up to -five fold) also at all nanobody concentrations (FIG. 17B). Thus, non-RBD epitopes cause not only ADE of SARS-CoV-2 pseudovirus entry, but also ADE of live SARS-CoV-2 infections of cultured cells; moreover, the ADE effect becomes more prominent at low virus titers.

### Discussion

The monomeric nature of nanobodies allowed us to purify and structurally characterize the complex of SARS-CoV-2 spike and all of the spike-bound nanobodies. The surface of the trimeric spike is heavily protected by glycans, likely a viral mechanism for immune evasion. Among the still exposed regions, four unique epitopes (recognized by Nanosota-3, -4, -5, and -6) were identified on each spike subunit. Because high-affinity and prevalent spike-binding nanobodies were selected during the purification process, these epitopes represent the dominant ones that play important roles in host immune responses against viral infection and vaccination. By analyzing individual top spike-binding nanobodies, we confirmed the above dominant epitopes and also discovered two less dominant epitopes recognized by rare nanobodies: Nanosota-2 is rare probably due to the limited accessibility of its epitope (Nanosota-2 only binds to the standing-up RBD, while Nanosota-3 and -4 bind to both the standing-up RBD and lying-down RBD), whereas the location of the Nanosota-7 epitope is unknown (it is outside the RBD but in S1). Overall, our study developed a novel method to identify dominant epitopes on viral glycoproteins and provided the first overview of the dominant epitopes on SARS-CoV-2 spike.

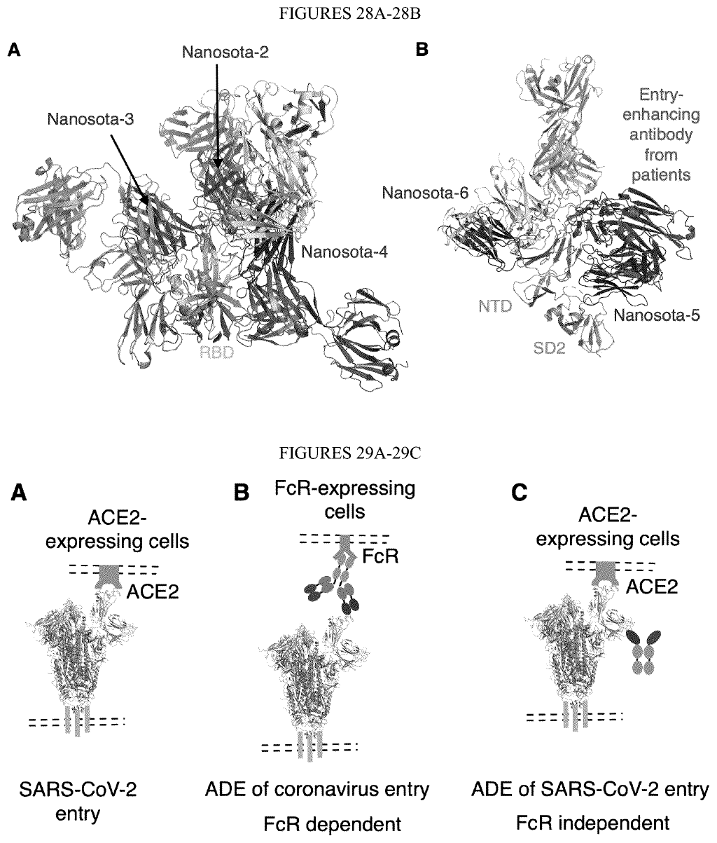

Numerous spike-targeting human antibodies have been discovered individually from COVID-19 patients. They generally recognize eight epitopes on the spike: five on the RBD and three on the NTD (FIG. 28). Among the five RBD epitopes, three overlap with the epitopes recognized by Nanosota-2, -3 and -4; they also overlap with the ACE2-binding site and hence are neutralizing with high potency (28-30). Compared to the above three RBD epitopes, the other two RBD epitopes are far away from the RBM and have limited accessibility; they do not overlap with the ACE2-binding site and are neutralizing with lower potency and unknown mechanism (31, 32). Among the three NTD epitopes, one partially overlaps with the Nanosota-5 epitope (see below for discussion of its function) and two others are both neutralizing with lower potency and unknown mechanism: one overlaps with the Nanosota-6 epitope and whether the other one overlaps with the Nanosota-7 epitope is unclear (33-35). Overall, the identified nanobody epitopes are consistent with previously identified human antibody epitopes. Uniquely, the current study mapped all of the dominant epitopes in one structure and also characterized the prevalence and revealed the distribution of these epitopes on the trimeric spike.

Surprisingly, in addition to the neutralizing RBD epitopes, our study also identified two ADE epitopes outside the RBD. Our depletion assay confirmed that RBD epitopes account for neutralization, while non-RBD epitopes account for ADE. Nanosota-5 induces ADE over a wide concentration range, whereas Nanosota-6 induces ADE at lower concentrations (it causes neutralization at higher concentrations with low potency). The ADE effect becomes more prominent at lower virus titers where up to -5 fold increase in viral infection was observed. We previously discovered a molecular mechanism for ADE of coronavirus entry in which RBD-targeting neutralizing antibodies simultaneously bind to the RBD on the virus and the FcR on cell surfaces, guiding coronaviruses into FcR-expressing cells (e.g., macrophages) (FIG. 29) (12). Different from the above FcR-dependent mechanism for ADE, non-RBD-targeting nanobodies cause ADE using an FcR-independent mechanism (FIG. 29). The Nanosota-5 epitope partially overlaps with an epitope recognized by a group of human antibodies isolated from COVID-19 patients, which enhances SARS-CoV-2 infection by promoting ACE2 binding (36). However, infection-enhancing activity of these human antibodies requires Fc, while the ADE functions of Nanosota-5 and -6 do not. The detailed structural mechanism by which these nanobodies induce ADE will need to be further investigated.

The discovery of ADE epitopes makes the following scenario possible in COVID-19 patients with pre-existing immunity. Previous infection (or vaccination) induces the production of both neutralizing antibodies and ADE antibodies. Initially, neutralizing antibodies dominate over ADE antibodies, protecting the patients from being re-infected by the same viral strain (or infected by the vaccine strain). However, a new strain may emerge containing extensive mutations in the neutralizing RBD epitopes but no mutations in the non-RBD ADE epitopes (e.g., the omicron strain), which would render the pre-existing neutralizing antibodies ineffective and ADE antibodies still effective. Then it is possible that ADE antibodies become dominant over neutralizing antibodies, allowing SARS-CoV-2 to infect previously infected (or vaccinated) people more efficiently than patients with no pre-existing immunity. In addition to the conflicting activities of the two groups of antibodies, the ADE effect may also depend on the concentration of ADE antibodies in a patient when the patient's immune system encounters the new viral strain: different people produce different amounts of antibodies from initial infection (or vaccination) and antibody titers wane over time. Hence, depending on the difference between the initial and new viral strains, ADE may happen to certain people over a certain time window after initial infection (or vaccination). This scenario will need to be confirmed by in vivo studies.

Our study provides guidance on vaccine and therapeutic developments against SARS-CoV-2. There have been a lot of discussions about developing universal coronavirus vaccines and therapeutics that target the more conserved regions outside the RBD (e.g., S2 subunit) (6-8). However, our study revealed a lack of dominant neutralizing epitopes outside the RBD (particularly in S2), suggesting that such efforts can be challenging. Instead, potent vaccines and therapeutics still need to focus on the neutralizing epitopes on the divergent RBD. Importantly, our study identified two ADE epitopes outside the RBD, raising concerns that non-RBD epitopes may cause ADE in some patients under certain conditions. To improve vaccine safety, these ADE epitopes may be knocked out in certain forms of spike-based vaccines (mRNA vaccines, subunit vaccines, viral vector vaccines and inactivated virus particle vaccines), although further assessment may be needed. For potency and safety, both neutralization and potential ADE effects by vaccines and therapeutics may need to be examined in preclinical and clinical testing.

The lack of an overview of the dominant epitopes in other viral glycoproteins is hindering the disease controls against these other viruses. Our approach can be extended to studying other viruses, filling in important gaps for many viruses.

### Methods

### Cell Lines, Plasmids and Virus

HEK293T cells and Calu-3 cells (American Type Culture Collection (ATCC)) were cultured in Dulbecco's modified Eagle medium (DMEM) (containing 10% fetal bovine serum, 2 mM L-glutamine, 100 units/mL penicillin, and 100 μg/mL streptomycin). Vero E6 cells (ATCC) were grown in Eagle's minimal essential medium (EMEM) (containing 100 units/ml penicillin, 100 μg/ml streptomycin, and 10% fetal bovine serum). ss320 E. coli (Lucigen), TG1 E. coli (Lucigen), and SHuffle T7 E. coli (New England Biolabs) were grown in 2YT medium (containing 100 mg/L ampicillin). All mammalian cells were authenticated by ATCC using STR profiling and were also tested for mycoplasma contamination. No commonly misidentified cell lines were used.

Original SARS-CoV-2 spike gene (Wuhan strain; GenBank: QHD43416.1) was synthesized (GenScript). Mutations were introduced to the original SARS-CoV-2 spike gene to generate the prototypic SARS-CoV-2 spike gene (encoding the spike protein from Wuhan strain plus D614G mutation). The prototypic SARS-CoV-2 spike gene was cloned into the pcDNA3.1(+) vector.

Genes encoding SARS-CoV-2 spike ectodomain (residues 14-1211) and RBD (residues 319-529) were each subcloned into Lenti-CMV vector (Vigene Biosciences) with an N-terminal tissue plasminogen activator (tPA) signal peptide and a C-terminal His tag. For the spike ectodomain construct, D614G and six proline mutations were introduced to the S2 subunit region to stabilize the spike protein in its prefusion state (20, 37). Plasmids encoding Fc-tagged Nanosota-2, -3, -4, -5, -6, and -7 as well as SARS-CoV-2 S1 (residues 14-685) were constructed in the same way as above except that a C-terminal human IgG₁ Fc tag replaced the His tag.

Genes encoding monomeric Nanosota-2, -3, -4, -5, -6, and -7 were each cloned into PADL22c vector (Lucigen) with a N-terminal PelB leader sequence and C-terminal His tag and HA tag.

The BAC cDNA clone of recombinant SARS-CoV-2-Venus (rSARS-CoV-2-Venus) was kindly provided by Dr. Luis Enjuanes and constructed as previously described (38, 39). Experiments involving infectious SARS-CoV-2 were conducted at the University of Iowa in approved biosafety level 3 laboratories.

### Construction of Induced Nanobody Phage Display Library

An induced nanobody phage display library was constructed as previously described (40). Briefly, an alpaca was immunized 6 times with 125 μg of purified prototypic SARS-CoV-2 spike ectodomain in Gerbu adjuvant. Following immunization, blood was drawn and peripheral blood mononuclear cells (PBMCs) were isolated through centrifugation from 50 mL of blood. A cDNA library was made through reverse transcription using oligo dT primers and Superscript IV reverse transcriptase (Thermo Scientific). A nested PCR strategy was used to amplify coding regions of antigen-binding domains (i.e., nanobodies) of heavy-chain-only antibodies. The resulting PCR fragments were cloned into a modified pADL22 vector (Antibody Design Labs). The phage library was produced with a size of 5×10⁹ following the manufacturer's protocols (Antibody Design Labs).

### Screening of Induced Nanobody Phage Display Library

To obtain a mixture of SARS-CoV-2 spike-binding nanobodies, the nanobody phage display library encoding monomeric nanobodies was used in bio-panning as previously described (41). Briefly, 5 g purified SARS-CoV-2 spike ectodomain was used for one round of bio-panning. After washing, the retained phages were eluted using 500 μl 100 mM triethylamine, neutralized with 250 μl 1 M Tris-HCl pH 7.5 and then used to infect ss320 E. coli. The mixture of ss320 E. coli were inoculated into 1L TB medium and induced by 1 mM IPTG to express the mixture of spike-binding nanobodies.

To obtain individual SARS-CoV-2 spike-binding nanobodies, single colonies from the above mixture of ss320 E. coli were picked and induced by 1 mM IPTG to express individual nanobodies. The supernatants were subjected to ELISA for identification of strong binders. The identified strong binders were expressed and purified (see below).

### Classification of SARS-CoV-2 Spike-Binding Nanobodies

From the above mixture of ss320 E. coli, 96 single colonies were randomly picked and the nanobody from each colony was assayed for its spike-binding affinity using ELISA. The genes encoding the top 48 spike-binding nanobodies were subjected to sequencing. Sequence alignment of the CDR3 region of these 48 nanobodies was performed using Clustal Omega (ebi.ac.uk/Tools/msa/clustalo/) and ESPript 3.0 (espript.ibcp.fr/ESPript/ESPript/index.php). The nanobodies were classified into six classes based on the variation of their CDR3 region. The top spike-binding nanobody from each of class was named Nanosota-2, -3, -4, -5, -6 and -7, respectively.

### Protein Expression and Purification

Nanosota-2, -3, -4, -5, -6 and -7 (with a His tag) were purified as previously described (41). Briefly, the nanobodies were each purified from the periplasm of ss320 E. coli after induction by 1 mM IPTG. The E. coli cells were collected and re-suspended in 15 ml TES buffer (0.2 M Tris pH 8, 0.5 mM EDTA, 0.5 M sucrose), shaken on ice for 1 hour, diluted with 40 ml ¼ TES buffer, and then shaken on ice for another hour. The proteins in the supernatant were sequentially purified using a Ni-NTA column and a Superdex200 gel filtration column (Cytiva).

The mixture of the spike-binding His-tagged nanobodies were expressed and purified in the same way as the above individual His-tagged nanobodies.

To prepare the mixture of the spike-binding Fc-tagged nanobodies, plasmids containing the nanobody genes were extracted from the above mixture of ss320 E. coli. The nanobody genes were amplified from the plasmids and inserted into pET42b vector (Novagen). The mixture of these newly constructed plasmids were electroporated into Shuffle T7 competent E. coli. The mixture of the Fc-tagged nanobodies were expressed in the cytoplasm of Shuffle T7 competent E. coli after induction by 1 mM IPTG and were purified in the way as the individual Fc-tagged nanobodies.

SARS-CoV-2 spike ectodomain (with a His tag), S1 (with an Fc tag), RBD (with a His tag) and individual Fc-tagged nanobodies were prepared from 293F mammalian cells as previously described (42). Briefly, lentiviral particles were packaged using the plasmid encoding one of the above proteins and then infected 293F cells for selection of stable cell lines in the presence of Puromycin (Gibco). The proteins were harvested from the supernatants of cell culture medium, purified on Ni-NTA column for His-tagged proteins or on Protein A column for Fc-tagged proteins, and purified further on Superdex200 gel filtration column (Cytiva).

To prepare the complex of the spike and all of the spike-bound nanobodies, 1 mg SARS-CoV-2 spike and 20 mg mixture of spike-binding His-tagged nanobodies were incubated at room temperature for 30 minutes. To prepare the complexes of the spike and individual nanobodies, 2 mg SARS-CoV-2 spike and each of the His-tagged nanobodies (with the nanobody in excess) were incubated at room temperature for 30 minutes. The above samples were then subjected to gel filtration using a Superose 6 increase 10/300 GL column (Cytiva).

### ELISA

To detect the binding between purified SARS-CoV-2 spike ectodomain and HA-tagged nanobodies from the supernatant of ss320 E. coli, ELISA was carried out as previously described (41). Briefly, ELISA plates were coated with SARS-CoV-2 spike ectodomain and were then incubated sequentially with the supernatant of ss320 E. coli (containing nanobodies) and HRP-conjugated anti-HA antibody (1:5,000) (Sigma). Subsequently ELISA substrate (Invitrogen) was added and the reactions were stopped using 1N H₂SO4. The absorbance at 450 nm (A₄₅₀) was measured using a Synergy LX Multi-Mode Reader (BioTek).

To detect the binding between purified SARS-CoV-2 spike domains and purified HA-tagged nanobodies, ELISA plates were coated with SARS-CoV-2 spike domains (RBD, S1, or spike ectodomain) and were then incubated sequentially with the HA-tagged nanobodies (Nanosota-2, -5, or -7) and HRP-conjugated anti-HA antibody (1:5,000) (Sigma). The remaining procedure was the same as above.

To detect the binding between purified His-tagged SARS-CoV-2 RBD and Fc-tagged nanobody mixtures (before and after deletion; see below), ELISA plates were coated with SARS-CoV-2 RBD and were then incubated sequentially with the nanobody mixtures (before or after deletion) and HRP-conjugated anti-Fc antibody (1:5,000) (Sigma). The remaining procedure was the same as above.

### Depletion Assay

To deplete RBD-targeting nanobodies from the mixture of spike-binding nanobodies, 100 μg His-tagged SARS-CoV-2 RBD was incubated with 80 ul His-Tag Dynabeads (Thermo Fisher Scientific) for 30 minutes and then the supernatant containing the RBD was removed. The beads were washed twice with PBS buffer (containing 10 mM imidazole) and then were incubated with 50 μg mixture of Fc-tagged spike-binding nanobodies for 30 minutes. The beads were removed and the supernatant containing the nanobodies was subjected to two more rounds of depletion.

### Pseudovirus Entry Assay

The functions of nanobodies in SARS-CoV-2 entry were evaluated using pseudovirus entry assay as previously described (42). Briefly, to prepare the pseudoviruses, HEK293T cells were co-transfected with a pcDNA3.1(+) plasmid encoding SARS-CoV-2 spike, a helper plasmid psPAX2 and a reporter plasmid plenti-CMV-luc. Pseudoviruses were collected 72 hours post transfection, incubated with nanobodies at different concentrations at 37° C. for 1 hour, and then used to enter HEK293T cells stably expressing human ACE2. After another 60 hours, cells were lysed. Aliquots of cell lysates were transferred to new plates, a luciferase substrate was added, and Relative Light Units (RLUs) were measured using an EnSpire plate reader (PerkinElmer). The neutralization potency of each nanobody was calculated and expressed as the concentration of the nanobody capable of inhibiting pseudovirus entry by 50% (IC₅₀).

### Cryo-EM Grid Preparation and Data Acquisition

4 μl purified complexes of SARS-CoV-2 spike ectodomain and nanobodies (˜1.9 μM for spike/Nanosota-2, ˜1.3 μM for spike/Nanosota-3, and ˜1.1 μM for spike/Nanosota-4, ˜2.6 μM for spike/Nanosota-5, ˜3.1 μM for spike/Nanosota-6, and ˜0.5 μM for spike/nanobody mixture) were supplemented with 8 mM CHAPSO immediately before grid preparation. Each of the complexes was then applied to freshly glow-discharged Quantifoil R1.2/1.3 300-mesh copper grids (EM Sciences) and blotted for 4 seconds at 22° C. under 100% chamber humidity and plunge-frozen in liquid ethane using a Vitrobot Mark IV (FEI). Cryo-EM data were collected using EPU version 3.0 (ThermoFisher Scientific) on a Titan Krios electron microscope (ThermoFisher Scientific) equipped with a Falcon IV direct electron detector and with a Selectris-X energy filter (ThermoFisher Scientific) or using Latitude-S(Gatan) equipped with a K3 direct electron detector and with a Biocontinuum energy filter (Gatan). For the Falcon IV detector, the movies were collected at a nominal magnification of 165,000× (corresponding to 0.73 Å per pixel), slit width 10 eV, a dose rate of 5.9 e⁻ per Å² per second, and a total dose of 40 e⁻/Å². For the K3 detector, the movies were collected at a nominal magnification of 75,300× (corresponding to 0.664 Å per pixel), slit width 20 eV, a dose rate of 25 e⁻ per Å² per second, and a total dose of 50 e⁻/Å². The statistics of cryo-EM data collection are summarized in Table S1-S3.

### Image Processing

Cryo-EM data were processed using cryoSPARC v3.3.2 (43), and the procedure is outlined in FIG. 19 and FIG. 21-25. Briefly, dose-fractionated movies were subjected to Patch motion correction with MotionCor2 (44) and Patch CTF estimation with CTFFIND-4.1.13 (45). Particles were then picked using both Blob picker and Template picker in cryoSPARC v3.3.2 and subjected to the Remove Duplicate Particles Tool. Junk particles were removed through three rounds of 2D classifications. Particles from the good 2D classes were used for Ab-initio Reconstruction of three or four maps. The initial models were set as the starting references for heterogeneous refinement (3D classification). Specifically, a second round of Ab-initio Reconstruction and heterogeneous refinement was conducted for the spike/Nanosota-3 complex. The selected 3D classes were then subjected to further homogeneous, non-uniform and CTF refinements, generating the final maps. Particles in the good 3D class were then imported into RELION-4.0 (46) using the csparc2star.py module (UCSF pyem v0.5. Zenodo) and subjected to signal subtraction to keep only the S1 subunit and nanobodies in RELION-4.0, followed by masked 3D classification for the spike/Nanosota-3 complex. Particles with the subtracted signal (spike/Nanosota-3 and spike/Nanosota-4 complexes) and the ones in the selected class from the masked 3D classification (spike/Nanosota-2 complex) were then subjected to local refinements to improve densities in cryoSPARC v3.3.2. For the samples of spike/Nanosota-5 and spike/Nanosota-6 complexes, the good 3D classes were finally subjected to further homogeneous, non-uniform and CTF refinements to generate the final maps with applying C3 symmetry. For the sample of spike/nanobody mixture complex, further local refinements with signal subtraction were performed to improve the densities of the nanobodies through masking the local regions of spike NTD/nanobodies or spike RBD/nanobodies. Map resolutions were determined by gold-standard Fourier shell correlation (FSC) at 0.143 between the two half-maps. Local resolution variation was estimated from the two half-maps in cryoSPARC v3.3.2 or v4.0.3.

### Cryo-EM Model Building and Refinement

Initial model building of the spike/nanobody complexes was performed in Coot-0.8.9 (47) using PDB 7TGX as the starting model. The initial model of each nanobody was predicted using SWISS-MODEL (swissmodel.expasy.org/), and then fitted into the density map. Several rounds of refinement in Phenix-1.16 (48) and manually building in Coot-0.8.9 were performed until the final reliable models were obtained. In general, standing-up RBDs and nanobodies bound to them are flexible, whereas lying-down RBDs, NTDs and nanodies bound to them are more rigid. Moreover, Nanosota-2 and -4 are flexible, whereas Nanosota-3, -5 and -6 are more rigid. In the final models of spike/individual nanobody complexes, atomic models were built for Nanosota-3 (one copy bound to the closed RBD), Nanosota-5 (all three copies) and Nanosota-6 (all three copies), whereas docking models for the other nanobodies were fitted into the densities as rigid bodies. In the final model of spike/nanobody mixture complex, eight atomic models of Nanosota-3, -5, and -6 and one docking model of Nanosota-4 were fitted into the densities as rigid bodies. Model and map statistics are summarized in Table S6. Figures were generated using UCSF Chimera X v0.93 (49) and PyMol v2.5.2 (50).

### Live SARS-CoV-2 Infection

Live SARS-CoV-2 infection in cultured cells was performed as previously described (39). Briefly, confluent monolayers of Vero E6 cells were transfected with 2.0 μg per well of rSARS-CoV-2-Venus BAC cDNA using Lipofectamnine 3000. 72 hours post transfection, rSARS-CoV-2-Venus virus was harvested and stocked. Subsequently, the virus was incubated with different concentration of Nanosota-5-Fc in 200 ml DMEM at 37° C. for 1 hour. Vero E6 cells (106 cells per well, 12-well plate, triplicates) were either mock inoculated or inoculated with the virus/nanobody mixture. After incubation at 37° C. for 1 hour with gentle rocking every 15 min, the inocula were removed and the plates were overlaid with 10% FBS. 24 hours post-inoculation, cells were fixed and then evaluated via flow cytometry. To this end, cells were fixed and permeabilized with Cytofix/Cytoperm (BD Bioscience) and all flow cytometry data were acquired using a BD FACSVerse and analysed with FlowJo software.

TABLE S4
Cryo-EM data collection, refinement and validation statistics of the
spike/nanobody mixture complex

| | Spike/nanobody | Local refinement of | Local refinement of |
|---|---|---|---|
| | mixture complex | spike/nanobody | spike/nanobody |
| | (with 9 | mixture complex | mixture complex |
| | nanobodies) | around RBD regions | around NTD regions |
| Data collection and processing | | | |
| Magnification | 753.00 | | |
| Voltage (kV) | 300 | | |
| Electron exposure (e−/Å²) | 40.00 | | |
| Defocus range (μm) | 0.8-2.4 | | |
| Pixel size (Å) | 0.664 | | |
| Symmetry imposed | C1 | C1 | C3 |
| Initial particle images (no.) | 43,617 | 43,617 | 43,617 |
| Final particle images (no.) | 30,121 | 30,121 | 30,121 |
| Map resolution (Å) | 3.4 | 6.1 | 3.4 |
| FSC threshold | 0.143 | 0.143 | 0.143 |
| Map resolution range (Å) | 3.0-10 | 5.0-11 | 3.0-10 |
| Refinement | | | |
| Initial model used (PDB code) | 7TGX | | |
| Model resolution (Å) | 3.8 | 10.2 | 3.7 |
| FSC threshold | 0.5 | 0.5 | 0.5 |
| Model resolution moge (Å) | 52.6-3.3 | 25.5-6.1 | 34.9-3.3 |
| Map sharpening B factor (Å²) | −55.7 | −393.5 | −87.8 |
| Model composition | | | |
| Non-hydrogen atoms | 34442 | 7459 | 17041 |
| Protein residues | 4307 | 954 | 2125 |
| Ligands | 69 | | 35 |
| B factors (Å²) | | | |
| Protein | 135.68 | 508.96 | 120.68 |
| Nucleotide | | | |
| Ligand | 141.19 | | 157.09 |
| R.m.s. deviations | | | |
| Bond lengths (Å) | 0.005 | 0.025 | 0.006 |
| Bond angles (°) | 0.734 | 1.000 | 0.833 |
| Validation | | | |
| MolProbity score | 1.19 | 2.44 | 2.18 |
| Clashscore | 11.61 | 35.13 | 14.11 |
| Poor rotamers (%) | 0.54 | 0.12 | 0.76 |
| Ramachandran plot | | | |
| Favored (%) | 94.53 | 93.74 | 91.02 |
| Allowed (%) | 5.12 | 5.63 | 8.45 |
| Disallowed (%) | 0.35 | 0.64 | 0.53 |

TABLE S5
Cryo-EM data collection, refinement and validation statistics of the spike/Nanosota-2, spike/Nanosota-3, and spike/Nanosota-4 complexes.

| | | | | | | | |
|---|---|---|---|---|---|---|---|
| | | | Spike/Nb3 | | Spike/Nb3 | | Spike/Nb4 |
| | | | complex | | complex | | complex |
| | Spike/Nb2 | Spike/Nb3 | with 2 | Spike/Nb3 | with 1 | Spike/Nb4 | with 2 |
| | complex | complex | RBDs up | complex | RBD up | complex | RBDs up |
| Spike/Nb2 | with 2 | with 2 | and 3 Nb3 | with 1 | and 2 Nb3 | with 2 | and 3 Nb4 |
| complex | RBDs up | RBDs up | bound | RBD up | bound | RBDs up | bound |
| with 2 | (local | and 3 Nb3 | (local | and 2 Nb3 | (local | and 3 Nb4 | (local |
| RBDs up | refinement) | bound | refinement) | bound | refinement) | bound | refinement) |

Data collection and processing

| | | | | | | | | |
|---|---|---|---|---|---|---|---|---|
| Magnification | 165,000 | | 165,000 | | 165,000 | | 75,300 | |
| Voltage (kV) | 300 | | 300 | | 300 | | 300 | |
| Electron exposure (e−/Å²) | 40.00 | | 40.00 | | 40.00 | | 50.00 | |
| Defocus range (μm) | 0.8-2.4 | | 0.8-2.4 | | 0.8-2.4 | | 0.75-2.5 | |
| Pixel size (Å) | 0.73 | | 0.73 | | 0.73 | | 0.664 | |
| Symmetry imposed | C1 | | C1 | | C1 | | C1 | |
| Initial particle images (no.) | 486,437 | 486,437 | 189,794 | 189,794 | 189,794 | 189,794 | 68,226 | 68,226 |
| Final particle images (no.) | 451,926 | 25124 | 81,068 | 81,068 | 77,360 | 77,360 | 58,046 | 58,046 |
| Map resolution (Å) | 2.1 | 5.6 | 2.5 | 3.3 | 2.5 | 3.2 | 3.4 | 4.1 |
| FSC threshold | 0.143 | 0.143 | 0.143 | 0.143 | 0.143 | 0.143 | 0.143 | 0.143 |
| Map resolution range (Å) | 1.8-4.6 | 5-10 | 2.0-8.0 | 3.0-8.0 | 2.0-8.0 | 3.0-8.0 | 3.0-8.0 | 3.2-10 |

Refinement

| | | | | | | | | |
|---|---|---|---|---|---|---|---|---|
| Initial model used (PDB code) | 7TGX | 7TGX | 7TGX | 7TGX | 7TGX | 7TGX | 7TGX | 7TGX |
| Model resolution (Å) | 2.7 | 9.4 | 2.9 | 4.0 | 2.9 | 3.6 | 3.8 | 7.4 |
| FSC threshold | 0.5 | 0.5 | 0.5 | 0.5 | 0.5 | 0.5 | 0.5 | 0.5 |
| Model resolution range (Å) | 44.3-2.1 | 23.4-5.0 | 51.4-2.4 | 43.3-3.2 | 52.0-2.4 | 40.0-3.2 | 63.0-3.4 | 54.4-4.0 |
| Map sharpening B factor (Å2 ) | −44.9 | −348.4 | −43.2 | −66.8 | −42.7 | −70.3 | −81.2 | −97.3 |

Model composition

| | | | | | | | | |
|---|---|---|---|---|---|---|---|---|
| Non-hydrogen atoms | 25761 | 3037 | 28420 | 16237 | 27543 | 15406 | 28711 | 17466 |
| Protein residues | 3217 | 338 | 3562 | 2046 | 3447 | 1928 | 3598 | 2193 |
| Ligands | 45 | | 47 | 2 | 47 | 7 | 47 | 12 |

B factors (Å²)

| | | | | | | | | |
|---|---|---|---|---|---|---|---|---|
| Protein | 106.09 | 228.15 | 128.96 | 173.60 | 129.02 | 126.74 | 159.30 | 144.99 |
| Nucleotide | | | | | | | | |
| Ligand | 115.62 | | 118.50 | 172.91 | 129.21 | 150.68 | 144.99 | 126.54 |

R.m.s. deviations

| | | | | | | | | |
|---|---|---|---|---|---|---|---|---|
| Bond lengths (Å) | 0.006 | 0.004 | 0.005 | 0.006 | 0.005 | 0.004 | 0.016 | 0.005 |
| Bond angles (°) | 0.949 | 0.852 | 0.843 | 0.831 | 0.839 | 0.724 | 1.028 | 0.767 |

Validation

| | | | | | | | | |
|---|---|---|---|---|---|---|---|---|
| MolProbity score | 2.33 | 1.78 | 2.17 | 2.18 | 2.09 | 1.96 | 1.81 | 2.04 |
| Clashscore | 32.58 | 15.75 | 21.22 | 24.55 | 20.71 | 14.00 | 14.17 | 17.42 |
| Poor rotamers (%) | 1.24 | 0.00 | 1.26 | 0.61 | 1.16 | 0.29 | 0.54 | 0.05 |

Ramachandran plot

| | | | | | | | | |
|---|---|---|---|---|---|---|---|---|
| Favored (%) | 96.20 | 97.64 | 95.97 | 95.65 | 96.48 | 95.59 | 97.19 | 95.68 |
| Allowed (%) | 3.45 | 2.36 | 3.77 | 4.10 | 3.37 | 4.25 | 2.70 | 4.09 |
| Disallowed (%) | 0.35 | 0.00 | 0.26 | 0.25 | 0.15 | 0.16 | 0.11 | 0.23 |

Nb2, Nb3 and Nb4 represent Nanosota-2, -3 and -4, respectively.

TABLE S6
Cryo-EM data collection, refinement and validation statistics of the
spike/Nanosota-5 and spike/Nanosota-6 complexes.

| | Spike/Nanosota-5 | Spike/Nanosota-6 |
|---|---|---|
| | complex with all | complex with all |
| | RBDs down and 3 | RBDs down and 3 |
| | Nanosota-5 bound | Nanosota-6 bound |
| Data collection and processing | | |
| Magnification | 753.00 | 753.00 |
| Voltage (kV) | 300 | 300 |
| Electron exposure (e−/Å²) | 40.00 | 40.00 |
| Defocus range (μm) | 0.8-2.4 | 0.8-2.4 |
| Pixel size (Å) | 0.664 | 0.664 |
| Symmetry imposed | C3 | C3 |
| Initial particle images (no.) | 52,821 | 234,682 |
| Final particle images (no.) | 31,809 | 189,562 |
| Map resolution (Å) | 3.8 | 2.8 |
| FSC threshold | 0.143 | 0.143 |
| Map resolution range (Å) | 3.5-7.5 | 2.6-3.8 |
| Refinement | | |
| Initial model used (PDB code) | 7TGY | 7TGX |
| Model resolution (Å) | 4.0 | 3.0 |
| FSC threshold | 0.5 | 0.5 |
| Model resolution range (Å) | 42.5-3.6 | 54.2-2.7 |
| Map sharpening B factor (Å²) | −124.9 | −99.8 |
| Model composition | | |
| Non-hydrogen atoms | 28572 | 28945 |
| Protein residues | 3566 | 3608 |
| Ligands | 53 | 61 |
| B factors (Å²) | | |
| Protein | 234.87 | 28.98 |
| Nucleotide | | |
| Ligand | 234.03 | 43.62 |
| R.m.s. deviations | | |
| Bond lengths (Å) | 0.004 | 0.005 |
| Bond angles (°) | 0.620 | 0.828 |
| Validation | | |
| MolProbity score | 1.94 | 1.96 |
| Clashscore | 12.65 | 11.70 |
| Poor rotamers (%) | 0.42 | 128 |
| Ramachandran plot | | |
| Favored (%) | 95.29 | 95.77 |
| Allowed (%) | 4.56 | 4.09 |
| Disallowed (%) | 0.14 | 0.14 |

### Documents Cited in Example 2

1. Q. Li et al., Early Transmission Dynamics in Wuhan, China, of Novel Coronavirus-Infected Pneumonia. N Engl J Med, (2020).
2. C. Huang et al., Clinical features of patients infected with 2019 novel coronavirus in Wuhan, China. Lancet, (2020).
3. J. Chen, K. Gao, R. Wang, D. D. Nguyen, G. W. Wei, Review of COVID-19 Antibody Therapies. Annual review of biophysics 50, 1-30 (2021).
4. Y. C. Hwang et al., Monoclonal antibodies for COVID-19 therapy and SARS-CoV-2 detection. Journal of biomedical science 29, 1 (2022).
5. D. Focosi et al., Monoclonal antibody therapies against SARS-CoV-2. Lancet Infect Dis 22, e311-e326 (2022).
6. D. M. Morens, J. K. Taubenberger, A. S. Fauci, Universal Coronavirus Vaccines—An Urgent Need. N Engl J Med 386, 297-299 (2022).
7. V. M. Vashishtha, P. Kumar, Looking to the future: is a universal coronavirus vaccine feasible?Expert review of vaccines 21, 277-280 (2022).
8. E. Dolgin, Pan-coronavirus vaccine pipeline takes form. Nature reviews. Drug discovery 21, 324-326 (2022).
9. W. S. Lee, A. K. Wheatley, S. J. Kent, B. J. DeKosky, Antibody-dependent enhancement and SARS-CoV-2 vaccines and therapies. Nature microbiology 5, 1185-1191 (2020).
10. K. Okuya et al., Multiple Routes of Antibody-Dependent Enhancement of SARS-CoV-2 Infection. Microbiology spectrum 10, e0155321 (2022).
11. X. Yang et al., Antibody-Dependent Enhancement: “Evil” Antibodies Favorable for Viral Infections. Viruses 14, (2022).
12. Y. Wan et al., Molecular Mechanism for Antibody-Dependent Enhancement of Coronavirus Entry. Journal of Virology 94, e02015-02019 (2020).
13. F. Li, Structure, Function, and Evolution of Coronavirus Spike Proteins. Annual review of virology 3, 237-261 (2016).
14. M. Y. Wang et al., SARS-CoV-2: Structure, Biology, and Structure-Based Therapeutics Development. Frontiers in cellular and infection microbiology 10, 587269 (2020).
15. S. Perlman, J. Netland, Coronaviruses post-SARS: update on replication and pathogenesis. Nature Reviews Microbiology 7, 439-450 (2009).
16. F. Li, Receptor recognition mechanisms of coronaviruses: a decade of structural studies. J Virol 89, 1954-1964 (2015).
17. F. Li, W. H. Li, M. Farzan, S. C. Harrison, Structure of SARS coronavirus spike receptor-binding domain complexed with receptor. Science 309, 1864-1868 (2005).
18. J. Shang et al., Structural basis of receptor recognition by SARS-CoV-2. Nature 581, 221-224 (2020).
19. J. Shang et al., Cell entry mechanisms of SARS-CoV-2. Proc Natl Acad Sci USA 117, 11727-11734 (2020).
20. G. Ye, B. Liu, F. Li, Cryo-EM structure of a SARS-CoV-2 omicron spike protein ectodomain. Nature communications 13, 1214 (2022).
21. Y. Wan, J. Shang, R. Graham, R. S. Baric, F. Li, Receptor Recognition by the Novel Coronavirus from Wuhan: an Analysis Based on Decade-Long Structural Studies of SARS Coronavirus. J Virol 94, (2020).
22. J. Lan et al., Structure of the SARS-CoV-2 spike receptor-binding domain bound to the ACE2 receptor. Nature 581, 215-220 (2020).
23. Y. Watanabe, J. D. Allen, D. Wrapp, J. S. McLellan, M. Crispin, Site-specific glycan analysis of the SARS-CoV-2 spike. Science 369, 330-333 (2020).
24. F. Li, Evidence for a Common Evolutionary Origin of Coronavirus Spike Protein Receptor-Binding Subunits. Journal of Virology 86, 2856-2858 (2012).
25. Y. Asaadi, F. F. Jouneghani, S. Janani, F. Rahbarizadeh, A comprehensive comparison between camelid nanobodies and single chain variable fragments. Biomarker research 9, 87 (2021).
26. S. Muyldermans, Nanobodies: natural single-domain antibodies. Annu Rev Biochem 82, 775-797 (2013).
27. T. De Meyer, S. Muyldermans, A. Depicker, Nanobody-based products as research and diagnostic tools. Trends Biotechnol 32, 263-270 (2014).
28. M. Yuan et al., Structural basis of a shared antibody response to SARS-CoV-2. Science 369, 1119-1123 (2020).
29. B. Ju et al., Human neutralizing antibodies elicited by SARS-CoV-2 infection. Nature 584, 115-119 (2020).
30. K. G. Nabel et al., Structural basis for continued antibody evasion by the SARS-CoV-2 receptor binding domain. Science 375, eab16251 (2022).
31. J. Fedry et al., Structural insights into the cross-neutralization of SARS-CoV and SARS-CoV-2 by the human monoclonal antibody 47D11. Science advances 7, (2021).
32. T. Li et al., Cross-neutralizing antibodies bind a SARS-CoV-2 cryptic site and resist circulating variants. Nature communications 12, 5652 (2021).
33. X. Chi et al., A neutralizing human antibody binds to the N-terminal domain of the Spike protein of SARS-CoV-2. Science 369, 650-655 (2020).
34. N. Suryadevara et al., Neutralizing and protective human monoclonal antibodies recognizing the N-terminal domain of the SARS-CoV-2 spike protein. Cell 184, 2316-2331.e2315 (2021).
35. G. Cerutti et al., Neutralizing antibody 5-7 defines a distinct site of vulnerability in SARS-CoV-2 spike N-terminal domain. Cell reports 37, 109928 (2021).
36. Y. Liu et al., An infectivity-enhancing site on the SARS-CoV-2 spike protein targeted by antibodies. Cell 184, 3452-3466.e3418 (2021).
37. C. L. Hsieh et al., Structure-based design of prefusion-stabilized SARS-CoV-2 spikes. Science 369, 1501-1505 (2020).
38. S. Warming, N. Costantino, D. L. Court, N. A. Jenkins, N. G. Copeland, Simple and highly efficient BAC recombineering using galK selection. Nucleic Acids Res 33, e36 (2005).
39. F. Almazin et al., Engineering the largest RNA virus genome as an infectious bacterial artificial chromosome. Proc Natl Acad Sci USA 97, 5516-5521 (2000).
40. E. Pardon et al., A general protocol for the generation of Nanobodies for structural biology. Nature protocols 9, 674-693 (2014).
41. G. Ye et al., The development of Nanosota-1 as anti-SARS-CoV-2 nanobody drug candidates. eLife 10, (2021).
42. Q. Geng et al., Novel virus-like nanoparticle vaccine effectively protects animal model from SARS-CoV-2 infection. PLoS Pathog 17, e1009897 (2021).
43. A. Punjani, J. L. Rubinstein, D. J. Fleet, M. A. Brubaker, cryoSPARC: algorithms for rapid unsupervised cryo-EM structure determination. Nature methods 14, 290-296 (2017).
44. J. L. Rubinstein, M. A. Brubaker, Alignment of cryo-EM movies of individual particles by optimization of image translations. Journal of structural biology 192, 188-195 (2015).
45. A. Rohou, N. Grigorieff, CTFFIND4: Fast and accurate defocus estimation from electron micrographs. Journal of structural biology 192, 216-221 (2015).
46. J. Zivanov, T. Nakane, S. H. W. Scheres, Estimation of high-order aberrations and anisotropic magnification from cryo-EM data sets in RELION-3.1. IUCrJ7, 253-267 (2020).
47. P. Emsley, K. Cowtan, Coot: model-building tools for molecular graphics. Acta Crystallographica Section D-Biological Crystallography 60, 2126-2132 (2004).
48. P. D. Adams et al., PHENIX: a comprehensive Python-based system for macromolecular structure solution. Acta Crystallographica Section D-Biological Crystallography 66, 213-221 (2010).
49. T. D. Goddard et al., UCSF ChimeraX: Meeting modern challenges in visualization and analysis. Protein science: a publication of the Protein Society 27, 14-25 (2018).
50. W. L. DeLano, PyMOL: an open-source molecular graphics tool. CCP4 Newsl. Protein Crystallogr. 40, 82-92 (2002).

### Example 3. Dual-Role Epitope on SARS-CoV-2 Spike Enhances and Neutralizes Viral Entry Across Different Variants

Grasping the roles of epitopes in viral glycoproteins is essential for unraveling the structure and functions of these proteins. Up to now, all identified epitopes have been found to either neutralize, have no effect on, or enhance viral entry into cells. Here, we used nanobodies (single-domain antibodies) as probes to investigate a unique epitope on the SARS-CoV-2 spike protein, located outside the protein's receptor-binding domain. Nanobody binding to this epitope enhances the cell entry of prototypic SARS-CoV-2, while neutralizing the cell entry of SARS-CoV-2 Omicron variant. Moreover, nanobody binding to this epitope enhances both receptor binding activity and post-attachment activity of prototypic spike, explaining the enhanced viral entry. The opposite occurs with Omicron spike, explaining the neutralized viral entry. This study reveals a unique epitope that can both enhance and neutralize viral entry across distinct viral variants, suggesting that epitopes may vary their roles depending on the viral context.

### INTRODUCTION

Antibodies have long been used to investigate the structure and function of viral glycoproteins by targeting specific epitopes^{1, 2}. Typically, antibody binding to these epitopes either neutralizes the virus or has no impact on its ability to enter cells. However, recent research indicates that antibodies can enhance a virus's entry into cells when they bind to certain epitopes, sparking interest in how they modulate the function of viral glycoproteins^{3, 4, 5, 6, 7}. No epitopes have been identified that can both neutralize and enhance entry for different variants of the same virus. Discovering such dual-function epitopes would significantly advance our understanding of viral entry and evolution, underscoring the need for targeted antiviral therapies that consider epitope variability across viral variants.

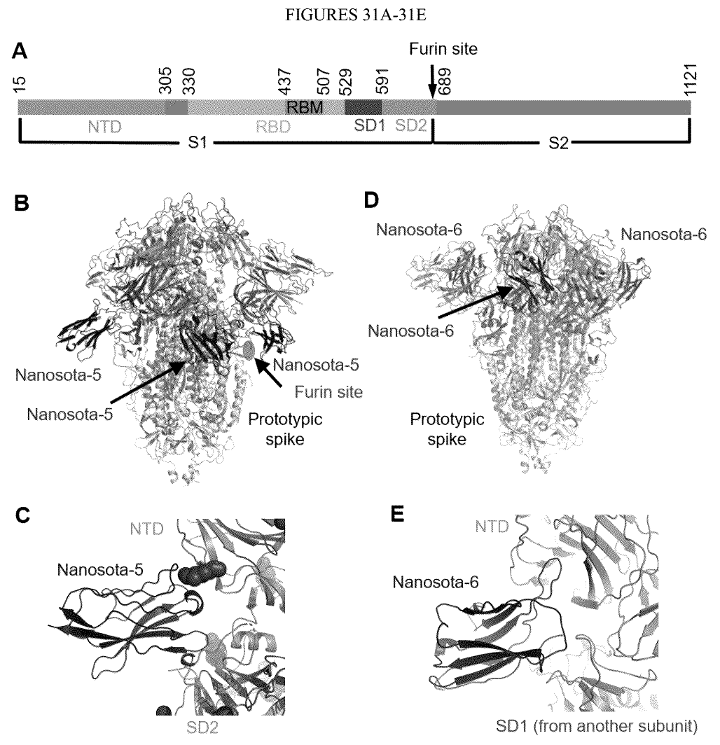

The spike protein of SARS-CoV-2 mediates viral entry into host cells^{8, 9, 10} The spike on the surface of mature virus particles is in the “pre-fusion” state. It is a homotrimer, consisting of three receptor-binding S1 subunits on top of a trimeric membrane-fusion S2 stalk. S1 contains a receptor-binding domain (RBD), an N-terminal domain (NTD), and two subdomains SD1 and SD2 (FIG. 31A). The RBD exists in two conformations within the spike: standing up for receptor binding and lying down for immune evasion^{11, 12} The RBD includes a core subdomain and a receptor-binding motif (RBM). During viral entry, the RBD binds to its host receptor ACE2, and the spike is cleaved at the S1/S2 boundary by a host protease (e.g., proprotein convertase furin, cell-surface TMPRSS2, and lysosomal cathepsins)^{12, 13, 14, 15}. Then S1 dissociates and S2 undergoes a dramatic structural change to fuse viral and host membranes^{8, 9, 10}. After membrane fusion, S2 is in the “post-fusion” state. While the post-fusion spike is the most stable and lowest-energy state, the pre-fusion spike must overcome an initial energy barrier to transition into the post-fusion state¹⁰ .

The spike protein of SARS-CoV-2 is key to triggering antibody responses; it is also the sole antigen in many COVID-19 vaccines and the main target for antibody-based treatments^{10, 16}. Antibodies work by latching onto specific epitopes on the spike protein. Neutralizing antibodies block SARS-CoV-2 from entering cells in two main ways. First, by binding to the RBM epitopes on the spike, they prevent the virus from attaching to the ACE2 receptor^{17, 18, 19, 20}. Second, they can attach to non-RBD epitopes on the spike, keeping the spike in its pre-fusion state and preventing it from fusing membranes^{21, 22, 23}. The ways that antibodies could enhance viral entry are less understood, but they may promote the RBD/ACE2 interaction by maintaining the RBD in the standing up position³. There is ongoing debate about whether such antibodies actually worsen SARS-CoV-2 infections in vivo³. Since neutralizing antibodies and entry-enhancing antibodies act in fundamentally different ways with completely opposite outcomes, it is unconceivable that a single antibody could both neutralize the virus and facilitate its entry into cells across various viral variants.

Nanobodies are the antigen-binding domain of heavy chain only antibodies produced by camelid animals^{24, 25, 26, 27} Here we used nanobodies as probes to investigate the functions of epitopes on the SARS-CoV-2 spike. In this Example 3, we identified an intriguing epitope, located outside the RBD of the spike, that enhances the cell entry of prototypic SARS-CoV-2, but neutralizes the cell entry of Omicron variant. We further examined the molecular mechanisms for such dual functions of this epitope. Our study has important implications for the structure, function and evolution of SARS-CoV-2 spike and for the efficacy and safety of antibody therapeutics.

### Results

### Discovery of Three Non-RBD Epitopes on Prototypic Spike

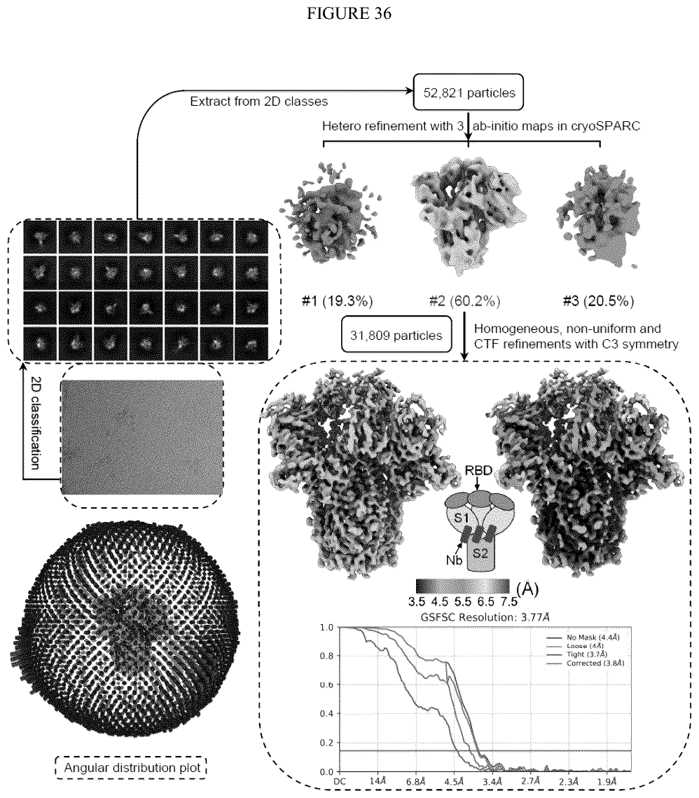

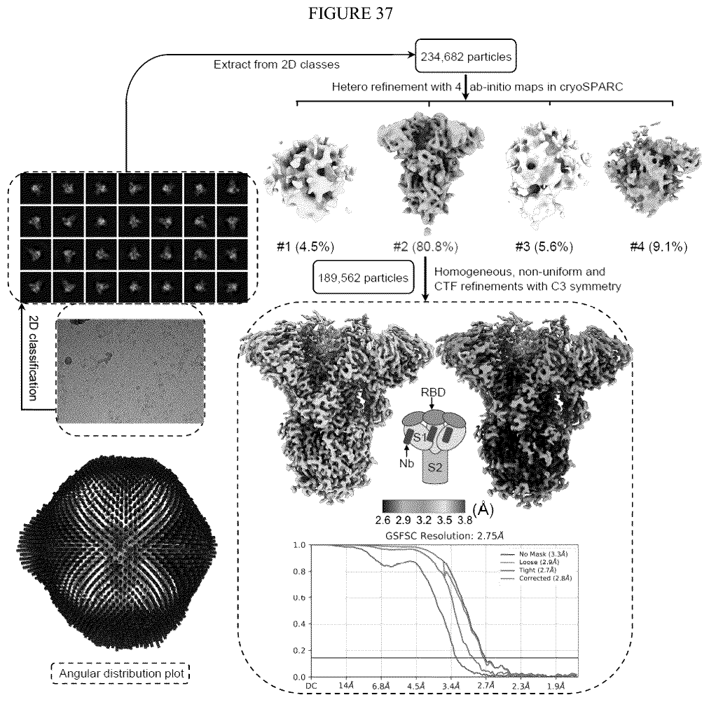

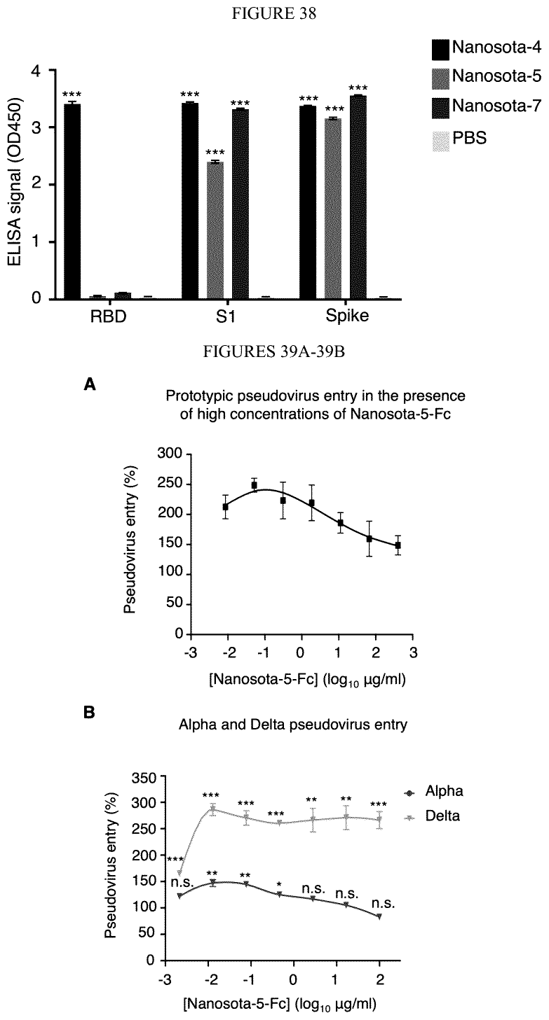

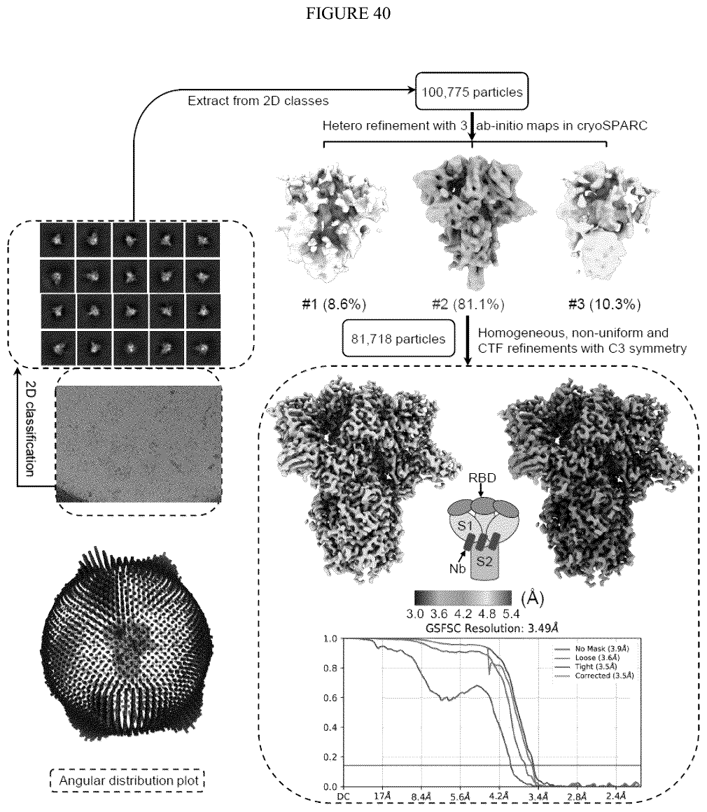

In a recent study, we described the discovery of three RBD-targeting nanobodies, named Nanosota-2, Nanosota-3 and Nanosota-4, from an alpaca immunized with prototypic SARS-CoV-2 spike²⁸. Here prototypic spike refers to the spike protein from the original SARS-CoV-2 variant plus an additional D614G mutation. Each of the three nanobodies blocks ACE2 from binding to the RBD and thereby neutralizes prototypic SARS-CoV-2 entry. In the current study, we discovered three more nanobodies, named Nanosota-5, Nanosota-6 and Nanosota-7, from the same alpaca immunized with prototypic spike. We determined the cryo-EM structures of prototypic spike complexed with either Nanosota-5 or Nanosota-6 (FIG. 31B, 31C, 31D, 31E; FIG. 36, 37; Table S7). The structures revealed that Nanosota-5 binds to the NTD and SD2 from the same spike protomer (FIG. 31B, 31C; FIG. 36), whereas Nanosota-6 binds to the NTD and SD1 from two different spike protomer (FIG. 31D, 31E; FIG. 37). The epitopes of Nanosota-5 and -6 are both located outside the RBD and do not overlap with the ACE2-binding region. The location of the Nanosota-7 epitope could not be identified using cryo-EM probably due to its flexibility, but ELISA indicated that the Nanosota-7 epitope is located outside the RBD but within S1 (FIG. 38). In sum, Nanosota-5, Nanosota-6 and Nanosota-7 all target non-RBD epitopes.

### Non-RBD Epitopes Enhance Cell Entry of Prototypic SARS-CoV-2 Pseudoviruses

To investigate the functions of the newly identified non-RBD epitopes, we studied how nanobodies targeting these epitopes, Nanosota-5, -6 and -7, influence the cell entry of prototypic SARS-CoV-2 pseudoviruses. The three RBD-targeting nanobodies, Nanosota-2, -3 and -4 were used for comparison. To facilitate protein purification and biochemical studies, we introduced a C-terminal Fc tag to each of the nanobodies (named Nanosota-2-Fc, etc.). Subsequently, retroviruses pseudotyped with SARS-CoV-2 spike (i.e., SARS-CoV-2 pseudoviruses, etc.) were used to infect ACE2-expressing HEK293T cells in the presence of each of the Fc-tagged nanobodies. The results revealed that Nanosota-2-Fc, -3-Fc, and -4-Fc all potently neutralized SARS-CoV-2 pseudovirus entry (FIG. 15A). However, Nanosota-6-Fc and -7-Fc only slightly neutralized SARS-CoV-2 pseudovirus entry at high concentrations (FIG. 15B). Interestingly, Nanosota-5-Fc at a wide concentration range and Nanosota-6-Fc at lower concentrations significantly enhanced SARS-CoV-2 pseudovirus entry (FIG. 15B). This resembled the antibody-dependent enhancement (ADE) of coronavirus entry that we observed previously using RBD-targeting neutralizing IgGs⁴. We previously showed that RBD-targeting IgGs, which contain an Fc tag, guide coronavirus entry into FcR-expressing cells⁴. However, Nanosota-5-Fc and -6-Fc enhanced SARS-CoV-2 pseudovirus entry into HEK293T cells that express ACE2, not FcR. To confirm that FcR did not play a role in the enhanced viral entry, we repeated the SARS-CoV-2 pseudovirus entry assay using His-tagged nanobodies (named Nanosota-5-His, etc.). Both Nanosota-5-His and -6-His enhanced SARS-CoV-2 pseudovirus entry despite having no Fc tag (FIG. 15C). Therefore, two of the three non-RBD epitopes on prototypic spike enhance the cell entry of prototypic SARS-CoV-2 pseudoviruses through an FcR-independent mechanism (FIG. 15D).

We selected Nanosota-5 for further investigation because it was the most effective at enhancing prototypic SARS-CoV-2 pseudovirus entry among the non-RBD-targeting nanobodies. Additional pseudovirus entry assays revealed that even at very high concentrations (e.g., 0.4 mg/ml), Nanosota-5-Fc continued to enhance the cell entry of prototypic SARS-CoV-2 pseudoviruses (FIG. 39A). Moreover, Nanosota-5-Fc enhanced the cell entry of both the alpha and delta variants of SARS-CoV-2 pseudoviruses (FIG. 39B). Thus, Nanosota-5 enhances the cell entry of prototypic and other pre-Omicron SARS-CoV-2 variants. Additionally, the distance between two Nanosota-5 epitopes on the prototypic trimeric spike is 143 Å, suggesting that Nanosota-5-Fc cannot bind two spike protomers in the same trimeric spike simultaneously. These results provided additional insights into Nanosota-5's impact on the cell entry of pre-Omicron SARS-CoV-2 variants.

### A Non-RBD Epitope Enhances Cell Infection of Live Prototypic SARS-CoV-2

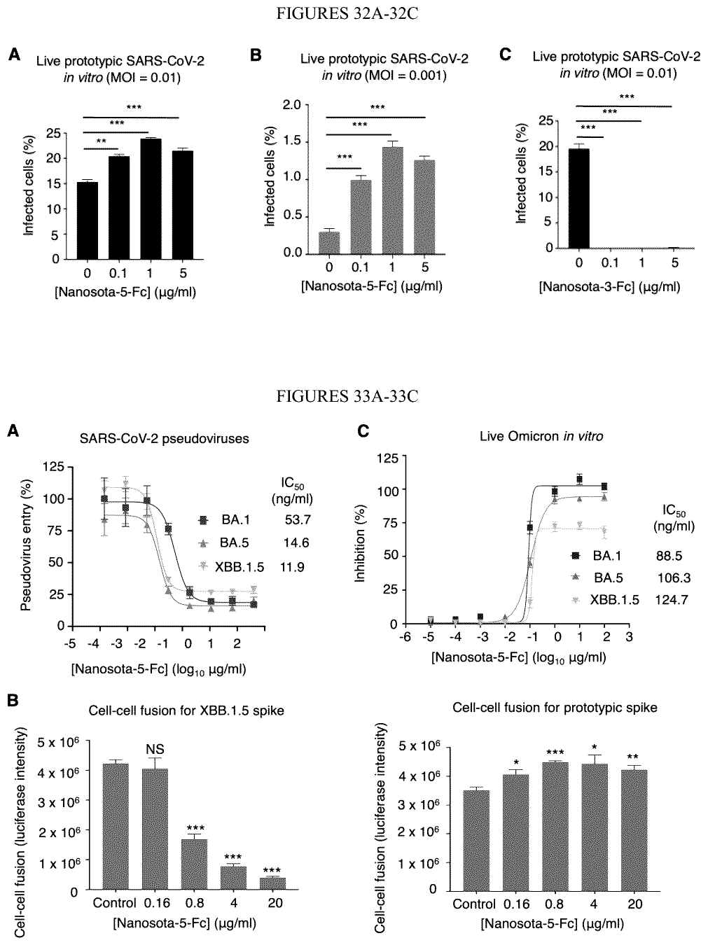

We further explored whether non-RBD epitopes can enhance the infection of live prototypic SARS-CoV-2 in cultured cells. Specifically, we examined the infection efficiency of live SARS-CoV-2 in ACE2-expressing Vero cells in the presence or absence of Nanosota-5-Fc. Two different virus titers and three different nanobody concentrations were tested. At all nanobody concentrations, significantly enhanced viral infection was observed at the high virus titer (FIG. 32A) and became even more prominent at the low virus titer (FIG. 32B). As a comparison, Nanosota-3-Fc, which targets the RBD, potently neutralized the infection of live prototypic SARS-CoV-2 (FIG. 32C). Thus, the Nanosota-5 epitope not only enhances cell entry of prototypic SARS-CoV-2 pseudoviruses, but also enhances cell infection of live prototypic SARS-CoV-2.

### The Same Non-RBD Epitope Neutralizes SARS-CoV-2 Omicron Variant

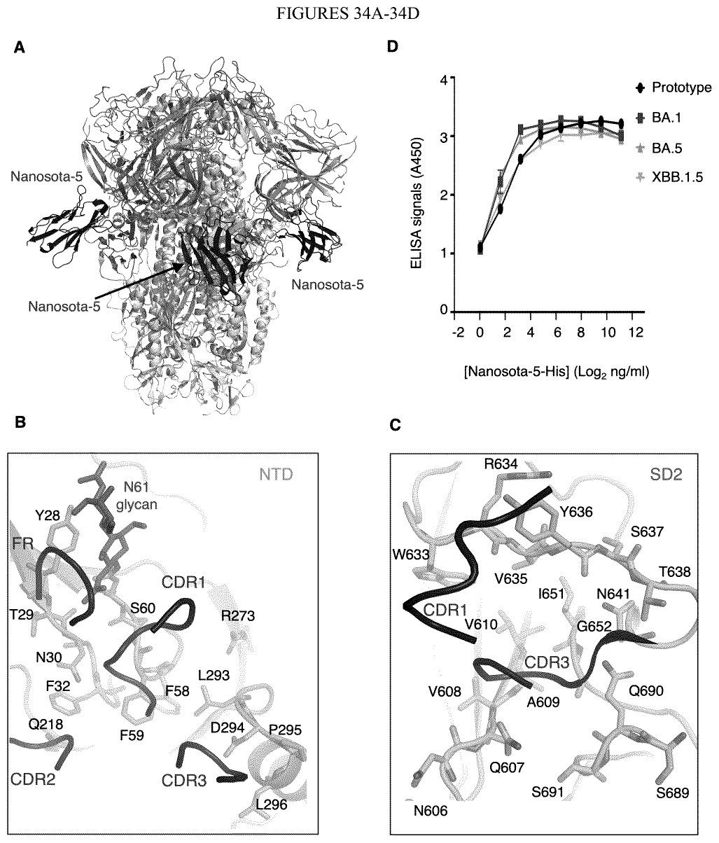

After studying the function of non-RBD epitopes in the cell entry of prototypic SARS-CoV-2, we further investigated their function in the cell entry of SARS-CoV-2 Omicron variant. To this end, SARS-CoV-2 Omicron pseudoviruses were used to enter ACE2-expressing HEK293T cells in the presence of Nanosota-5-Fc (FIG. 33A). The pseudoviruses of three different Omicron subvariants were tested, including the early subvariant BA.1, the later subvariant BA.5 and the recent subvariant XBB.1.5. Surprisingly, Nanosota-5-Fc effectively neutralized the pseudovirus entry of all three Omicron subvariants (FIG. 33A). To validate the above result, we performed a cell-cell fusion assay where cells expressing the spike protein (from either prototypic or XBB.1.5 SARS-CoV-2) and cells expressing ACE2 were incubated together for fusion in the presence of Nanosota-5-Fc. The result revealed that while Nanosota-5 enhanced the cell-cell fusion for prototypic spike, it inhibited the cell-cell fusion for XBB.1.5 spike (FIG. 33B). We also repeated the live SARS-CoV-2 infection assay in the presence of Nanosota-5-Fc, this time using live BA.1, BA.5, and XBB.1.5 instead of prototypic SARS-CoV-2. The result demonstrated that Nanosota-5-Fc neutralized all three live Omicron subvariants in cultured cells (FIG. 33C). Together, all these lines of evidence reveal that the Nanosota-5 epitope has opposing effects on the functions of the spike proteins from prototypic SARS-CoV-2 and Omicron variant: entry-enhancing for the former and neutralizing for the latter.

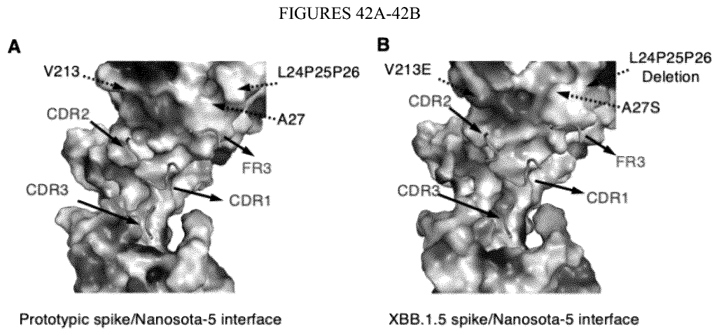

To understand how Nanosota-5 binds to XBB.1.5 spike, we determined the cryo-EM structure of XBB.1.5 spike complexed with Nanosota-5 (FIG. 34A; FIG. 41; Table S7). The result showed that Nanosota-5 binds to the same epitope on XBB.1.5 spike as it does prototypic spike. Again, this epitope is located at the junction of the NTD and SD2 from the same spike protomer. Nanobodies contain three complementarity-determining regions (CDRs) and four framework regions (FRs). All three CDRs and a part of an FR of Nanosota-5 are involved in contacting the NTD (FIG. 34B), while its CDR1 and CDR3 are involved in contacting the SD2 (FIG. 34C). Surprisingly, all of the Nanosota-5-contacting residues are conserved between prototypic and Omicron spikes, including a glycan N-linked to Asn61 in prototypic spike (corresponding to Asn58 in XBB.1.5 spike) (FIG. 34B, FIG. 34C, FIG. 41). Additionally, we examined the residues outside but near the Nanosota-5-binding site and identified a few residue differences between prototypic and XBB.1.5 spikes. However, we found that these differences did not significantly affect the surface electrostatic potential in the neighboring regions of the Nanosota-5-binding site (FIG. 42). Indeed, ELISA results showed that Nanosota-5-His exhibited similar binding affinities for the recombinant spike ectodomains from prototypic SARS-CoV-2 and three Omicron subvariants (BA.1, BA.5, and XBB.1.5) (FIG. 34D). Thus, the Nanosota-5 epitope on the spikes from different SARS-CoV-2 variants is conserved, suggesting that its varying effects on the functions of spikes from different viral variants are due to structural differences between prototypic and Omicron spikes outside of this epitope.

### Molecular Mechanism for the Dual Function of the Non-RBD Epitope

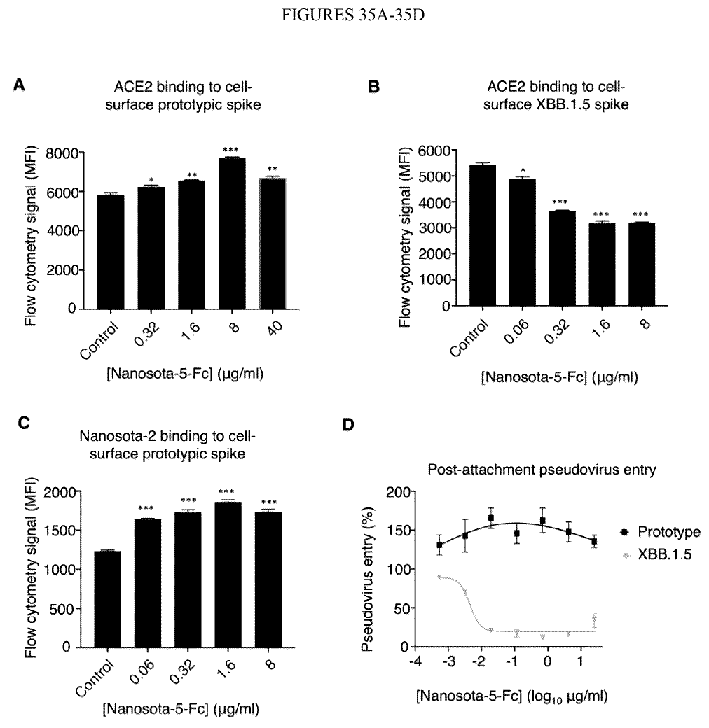

We investigated the molecular mechanism behind the non-RBD epitope having opposing effects on the functions of spikes from different SARS-CoV-2 variants. We examined how Nanosota-5-Fc affects the binding interactions between the SARS-CoV-2 spike and ACE2. To do this, recombinant ACE2 was incubated with cell-surface spikes in the presence or absence of Nanosota-5-Fc, and the interaction between the spikes and ACE2 was measured using flow cytometry. Both prototypic and XBB.1.5 spikes were tested. The results showed that Nanosota-5-Fc increased the binding affinity between prototypic spike and ACE2 (FIG. 35A), while decreasing the binding affinity between XBB.1.5 spike and ACE2 (FIG. 35B). Since only the standing-up RBD can bind to ACE2, these results suggest that Nanosota-5-Fc promotes more RBDs to stand up in prototypic spike, while having the opposite effect on XBB.1.5 spike. To confirm this conclusion, we further investigated how Nanosota-5-Fc affects the binding interaction between prototypic spike and Nanosota-2, which, like ACE2, is only accessible to the standing-up RBD²⁸. Flow cytometry results confirmed that Nanosota-5-Fc increased the binding affinity between prototypic spike and Nanosota-2 (FIG. 35C). Therefore, the Nanosota-5 epitope has opposing effects on the ACE2-binding affinity of prototypic and XBB.1.5 spikes, likely by promoting the RBD to stand up.

We also analyzed whether Nanosota-5 affects post-attachment events during the viral entry process. To do this, we incubated pseudoviruses with cells before adding Nanosota-5-Fc and allowing pseudovirus entry to occur. The results showed that even after viral attachment to cells had occurred, Nanosota-5-Fc still enhanced the cell entry of prototypic pseudoviruses, while neutralizing the cell entry of XBB.1.5 pseudoviruses (FIG. 35D). Therefore, Nanosota-5 has opposing effects not only on ACE2 binding between prototypic and XBB.1.5 spikes but also on post-attachment steps between prototypic and XBB.1.5 pseudoviruses.

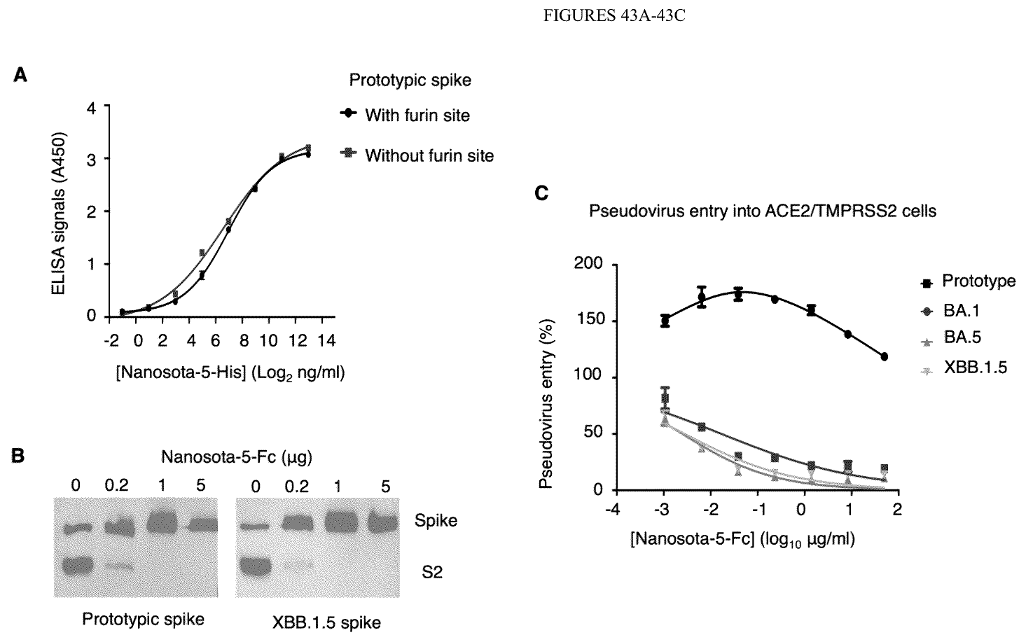

Since the Nanosota-5 epitope is near the furin cleavage site (FIG. 31B), we examined whether the furin cleavage site impacts Nanosota-5's binding to the SARS-CoV-2 spike. ELISA results showed that Nanosota-5-His had similar binding affinities for prototypic spike, regardless of the presence of the furin cleavage site (FIG. 43A). Next, we investigated whether Nanosota-5 affects the furin cleavage of the spikes. To do this, we co-expressed Nanosota-5-Fc with either prototypic or XBB.1.5 spike in cells. The results suggested that Nanosota-5-Fc inhibited furin cleavage of both prototypic and XBB.1.5 spikes (FIG. 43B). It is important to note that during molecular maturation, the majority of the spike molecules had already been cleaved (FIG. 43B). Therefore, even though Nanosota-5 inhibits furin cleavage of the spikes, it may not have a significant impact on viral entry through furin cleavage inhibition. Additionally, since Nanosota-5 inhibits furin cleavage of both prototypic and XBB.1.5 spikes, and this study focuses on the opposing effects of Nanosota-5 on these spikes, Nanosota-5's inhibition of furin cleavage is not a primary focus of the current study.

Since pre-Omicron and Omicron spikes differ in their sensitivity to TMPRSS2²⁹, we analyzed whether TMPRSS2 affects Nanosota-5's impact on SARS-CoV-2 entry. Pseudovirus entry assay results showed that in the presence of TMPRSS2, Nanosota-5-Fc still enhanced the entry of prototypic pseudoviruses while neutralizing the entry of Omicron pseudoviruses into cells overexpressing TMPRSS2 (FIG. 43C). Therefore, the opposing effects of Nanosota-5 on prototypic and Omicron spikes are not due to the different TMPRSS2 sensitivities between the two spikes.

### Efficacy of Nanosota-5-Fc Against Live SARS-CoV-2 in Mice

FIG. 46A showed that Nanosota-5-Fc had no impact on prototypic SARS-CoV-2 in mice. It was administered at dosages of 2 or 10 mg/kg body weight, 4 hours post-challenge. Mice were challenged via intranasal inoculation with the prototypic SARS-CoV-2 (mouse-adapted prototypic variant M15, containing mutations in the RBD but not in the Nanosota-5 epitope). FIG. 46B showed that Nanosota-5-Fc effectively neutralized different Omicron subvariants in mice. It was administered at a dosage of 10 mg/kg body weight, 4 hours post-challenge. Mice were challenged via intranasal inoculation with each of the indicated Omicron subvariants (BA.1, BA.5, XBB.1.5, EG.5, JN.1). For both (FIG. 46A) and (FIG. 46B), the (−) control groups received PBS buffer, and virus titers in the mouse lungs were measured on day 2 post-challenge.

### Discussion

Epitopes on viral glycoproteins are not just tools for deciphering the structure and function of these molecules; they are also fundamental to developing antiviral vaccines and antibody-based treatments. Typically, epitopes are classified as either neutralizing, non-neutralizing, or infection-enhancing. However, by using nanobodies in Example 3, we have identified a SARS-CoV-2 spike epitope that has a dual role: it neutralizes one variant of SARS-CoV-2 and enhances infection in another. This discovery challenges the traditional categorization of epitopes, underscores the complex evolution of SARS-CoV-2 spike, and offers new insights into antiviral antibody therapies.

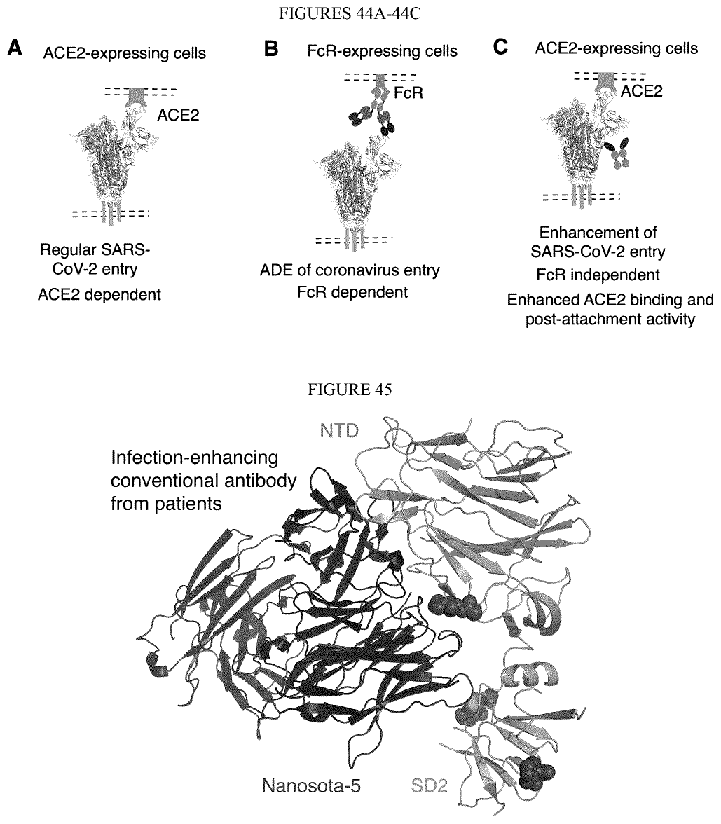

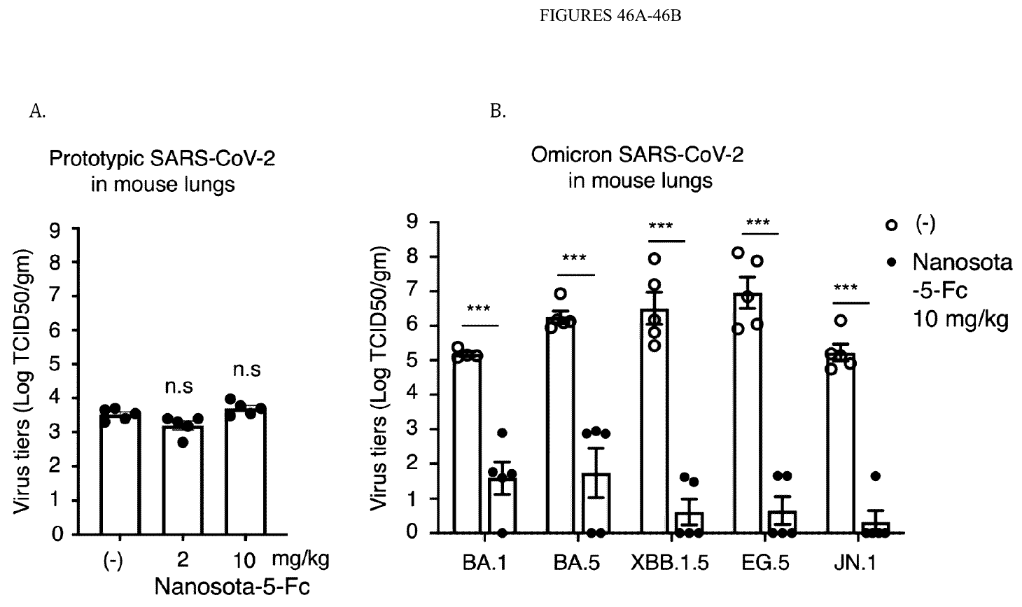

Infection-enhancing epitopes in coronavirus spikes have attracted great scientific interest. The classical pathway for coronavirus entry into cells relies on host receptors, where the viruses attach to their receptor, undergo endocytosis, and subsequently fuse viral and host membranes¹⁰. Previously, we discovered a unique molecular mechanism for ADE of coronavirus entry that is dependent on FcR but independent of host receptors. Specifically, neutralizing antibodies that target the RBD simultaneously bind to the RBD on the virus and FcR on the cell surfaces. This interaction guides coronaviruses into cells expressing FcR, such as macrophages (FIG. 44)⁴. This mechanism has later been validated in vivo^{30, 31, 32, 33}. More recently, a group of human antibodies, targeting a non-RBD epitope, was isolated from COVID-19 patients and found to enhance SARS-CoV-2 infection by promoting the spike's binding to its ACE2 receptor 3. While resembling ADE, this mechanism has not yet been confirmed in vivo. Additionally, the infection-enhancing activity of these human antibodies requires Fc, although the reason for this remains unclear 3. In our current study, we identified two entry-enhancing epitopes outside the RBD of prototypic SARS-CoV-2 spike and delved deeper into one of them, recognized by Nanosota-5. This epitope is situated at the junction between the NTD and SD2. It partially overlaps with the entry-enhancing epitope mentioned earlier, which is recognized by human antibodies (FIG. 45). In contrast to the FcR-dependent ADE observed with the RBD epitope, the Nanosota-5 epitope enhances entry through an FcR-independent mechanism. It is not clear whether the Nanosota-5 epitope causes ADE in vivo, which will be investigated in future studies. Overall, our research offers new perspectives on how non-RBD epitopes can increase prototypic SARS-CoV-2's ability to enter cells in vitro.

Remarkably, the Nanosota-5 epitope exhibits dual functions: it enhances the cell entry of prototypic SARS-CoV-2 but neutralizes the cell entry of Omicron variant, including BA.1, BA.5, and XBB.1.5 subvariants. This discovery challenges conventional epitope paradigms. To decipher the molecular mechanisms underpinning how the same non-RBD epitope can exert opposing effects on different SARS-CoV-2 variants, we conducted comparative studies between prototypic and Omicron spikes. Our investigations revealed that for prototypic spike, Nanosota-5-Fc promotes its ACE2 binding, thereby enhancing viral entry, and also increases its post-attachment activity, likely facilitating its transition to the post-fusion structure. Surprisingly, although our flow cytometry data showed that Nanosota-5 promotes the prototypic spike to bind ACE2, our cryo-EM study of the prototypic spike in complex with Nanosota-5 revealed that all Nanosota-5-bound prototypic spike molecules were in the three-RBD down conformation, which disfavors ACE2 binding. This contrasts with the cryo-EM studies on unliganded prototypic spike, which typically has 40-50% of the molecules with at least one RBD in the standing up position^{11, 12}. The reason for the discrepancy between our flow cytometry and cryo-EM results is unclear but could be due to the cryo-EM procedure (for instance, Nanosota-5-bound open spike might be too unstable to be characterized by cryo-EM). The open and closed populations of Nanosota-5-bound prototypic spike will be further investigated in future cryo-EM studies. Nevertheless, using flow cytometry, we also demonstrated that Nanosota-5 promotes the prototypic spike to bind Nanosota-2, which, like ACE2, only binds to the standing up RBD of the spike²⁸. Overall, our data suggest that Nanosota-5 induces the RBD to stand up in prototypic spike, facilitating ACE2 or Nanosota-2 binding. It has been shown that the standing up RBD destabilizes the pre-fusion spike, allowing it to transition more easily to its post-fusion structure^{10, 34} which aligns with our post-attachment entry data for prototypic pseudoviruses. In contrast, Nanosota-5 reduces ACE2 binding by Omicron spike and inhibits the post-attachment entry of Omicron pseudoviruses, likely by inducing the RBDs to adopt the lying down conformation in Omicron spike. More detailed mechanistic analysis of the opposing effects observed with the Nanosota-5 epitope across different SARS-CoV-2 variants will be conducted in future studies.

In summary, in this Example 3, we used nanobodies as probes and identified a unique epitope on the SARS-CoV-2 spike protein with opposing functions across different SARS-CoV-2 variants. It enhances viral infection in pre-Omicron variants but inhibits it in the Omicron variant. We further investigated the molecular mechanism underlying these opposing effects. Although this epitope is not directly involved in receptor binding, nanobody binding to it modulates receptor binding and post-receptor-binding activities. Our study sheds light on the potential for more targeted antiviral antibody therapies. We have found that certain epitopes on viral glycoproteins can have dual roles: they might help one virus variant enter cells but block another variant. This discovery highlights the complexity of viral evolution; accordingly, our approaches to antiviral antibody therapies need to consider the versatile nature of epitopes on viral glycoproteins.

### Data Availability

The atomic models and corresponding cryo-EM density maps have been deposited into the PDB and the Electron Microscopy Data Bank, respectively, with accession numbers PDB 8G76 and EMDB-29798 (prototypic SARS-CoV-2 spike complexed with Nanosota-5), PDB 8G77 and EMDB-29799 (prototypic SARS-CoV-2 spike complexed with Nanosota-6), and PDB 8UG9 and EMDB-42218 (SARS-CoV-2 Omicron XBB.1.5 spike complexed with Nanosota-5).

### Methods

### Cell Lines, Plasmids and Virus

HEK293T cells and Calu-3 cells (American Type Culture Collection (ATCC)) were grown in Dulbecco's modified Eagle medium (DMEM) (containing 10% fetal bovine serum, 2 mM L-glutamine, 100 units/mL penicillin, and 100 μg/mL streptomycin). 293F cells (ThermoFisher) were grown in FreeStyle 293 Expression Medium (ThermoFisher). Vero E6 cells (ATCC) were cultured in Eagle's minimal essential medium (EMEM) (containing 100 units/ml penicillin, 100 μg/ml streptomycin, and 10% fetal bovine serum). ss320 E. coli (Lucigen), and TG1 E. coli (Lucigen) were grown in 2YT medium. All mammalian cells were authenticated by ATCC using STR profiling and were also tested for mycoplasma contamination. No commonly misidentified cell lines were used.

Original SARS-CoV-2 spike gene (GenBank: QHD43416.1) and human TMPRSS2 gene (UniProt: 015393) were synthesized (GenScript). Mutations were introduced to the original SARS-CoV-2 spike gene to generate the prototypic SARS-CoV-2 spike gene (encoding the spike protein from Wuhan variant plus D614G mutation), alpha variant (GISAID: EPI_ISL_6135157), delta variant (GenBank: UEM53021.1), Omicron BA.1 subvariant (GISAID: EPI_ISL_6590782.2), Omicron BA.5 subvariant (GISAID: EPI_ISL_12954165), and Omicron XBB.1.5 subvariant (GISAID: EPI_ISL_17774216). Each of the spike genes was cloned into the pcDNA3.1(+) vector.

Genes encoding spike ectodomain (residues 14-1211), RBD (residues 319-529), and S1 (residues 14-685) from prototypic SARS-CoV-2 were each subcloned into Lenti-CMV vector (Vigene Biosciences) with an N-terminal tissue plasminogen activator (tPA) signal peptide and a C-terminal His tag. For the spike ectodomain construct, a D614G mutation, two mutations in the furin cleavage site (from RRAR (SEQ ID NO: 177) to AGAR (SEQ ID NO: 178)), and six proline mutations were introduced to the S2 subunit region to stabilize the spike protein in its prefusion state^{35, 36} Plasmids encoding Fc-tagged Nanosota-5, -6, and -7 were constructed in the same way as above except that a C-terminal human IgG₁ Fc tag replaced the His tag.

Genes encoding monomeric Nanosota-5, -6, and -7 were each cloned into PADL22c vector (Antibody Design Labs) with an N-terminal PelB leader sequence and C-terminal His tag and HA tag.

The BAC cDNA clones of recombinant SARS-CoV-2 were kindly provided by Dr. Luis Enjuanes. Recombinant SARS CoV-2-Venus (rSARS-CoV-2-Venus) BAC was constructed as previously described³⁷. The SARS-CoV-2_{MA30} virus was described previously³⁸. The Omicron viruses (hCoV-19/USA/GA-EHC-2811C/2021 for BA.1, hCoV-19/USA/COR-22-063113/2022 for BA.5, and hCoV-19/USA/MD-P40900/2022 for XBB.1.5) were obtained through BEI Resources, NIH. Experiments involving live infectious SARS-CoV-2 were conducted at the University of Iowa and the University of Louisville in approved biosafety level 3 laboratories.

### Screening of Nanobody Phage Display Library

The same nanobody phage display library that was recently used to screen for RBD-targeting nanobodies was also used in the current study to screen for non-RBD-targeting nanobodies²⁸. Briefly, 5 g purified SARS-CoV-2 spike ectodomain coated on an ELISA plate was used for one round of bio-panning. 100 μl phages from the phage library were added to the coated spike and incubated for 1 hour. After washing, the retained phages were eluted and then used to infect ss320 E. coli. The infected ss320 E. coli were spread onto 2YT AG plates, and single colonies were picked and induced by 1 mM IPTG to express individual nanobodies. The supernatants were subjected to ELISA for identification of strong binders against prototypic SARS-CoV-2 spike.

### Protein Expression and Purification

Nanosota-5, -6 and -7 (with a His tag) were purified as previously described 39. Briefly, nanobody expression was induced by 1 mM IPTG and purified from the periplasm of ss320 E. coli. The pellets were collected and re-suspended in 15 ml TES buffer (0.2 M Tris pH 8, 0.5 mM EDTA, 0.5 M sucrose), shaken on ice for 1 hour, diluted with 40 ml ¼ TES buffer, and then shaken on ice for another hour. The proteins in the supernatant were sequentially purified using an Ni-NTA column and a Superdex200 gel filtration column (Cytiva).

SARS-CoV-2 spike ectodomain (with a His tag), RBD (with a His tag) and individual Fc-tagged nanobodies were prepared from 293F cells as previously described⁴⁰. Briefly, lentiviral particles were packaged using the plasmid encoding one of the above proteins and then used to infect 293F cells for selection of stable cell lines in the presence of Puromycin (Gibco). The proteins were harvested from the supernatant of respective cell culture medium, purified on Ni-NTA column for His-tagged proteins or on Protein A column for Fc-tagged proteins, and purified further on Superdex200 gel filtration column (Cytiva).

To prepare the complexes of the spike and individual nanobodies, 2 mg SARS-CoV-2 spike and each of the His-tagged nanobodies (with the nanobody in excess) were incubated at room temperature for 30 minutes. The above samples were then subjected to gel filtration using a Superose 6 increase 10/300 GL column (Cytiva).

### ELISA

To detect the binding between SARS-CoV-2 spike ectodomain and HA-tagged nanobodies from the supernatant of ss320 E. coli, ELISA was conducted as previously described³⁹. Briefly, ELISA plates were coated with purified SARS-CoV-2 spike ectodomain and were then incubated sequentially with the supernatant of ss320 E. coli (containing nanobodies) and HRP-conjugated anti-HA antibody (1:5,000) (Sigma). ELISA substrate (Invitrogen) was added and the reactions were stopped using 1N H₂SO4. The absorbance at 450 nm (A₄₅₀) was measured using a Synergy LX Multi-Mode Reader (BioTek).

To detect the binding between SARS-CoV-2 spike domains and purified HA-tagged nanobodies, ELISA plates were coated with SARS-CoV-2 spike domains and were then incubated sequentially with the HA-tagged nanobodies and HRP-conjugated anti-HA antibody (1:5,000) (Sigma). The remaining procedure was the same as above.

### Pseudovirus Entry Assay

The activities of nanobodies in SARS-CoV-2 entry were evaluated using pseudovirus entry assay as previously described⁴⁰. Briefly, to prepare the pseudoviruses, HEK293T cells were co-transfected with a pcDNA3.1(+) plasmid encoding SARS-CoV-2 spike, a helper plasmid psPAX2 and a reporter plasmid plenti-CMV-luc. Pseudoviruses were collected 72 hours post-transfection, incubated with individual nanobody or nanobody-Fc mixture at different concentrations at 37° C. for 1 hour, and then used to enter HEK293T cells stably expressing human ACE2 (HEK293T/hACE2 cells). After another 60 hours, cells were lysed. Aliquots of cell lysates were transferred to new plates, a luciferase substrate was added, and Relative Light Units (RLUs) were measured using an EnSpire plate reader (PerkinElmer). The neutralization potency of each nanobody was calculated and expressed as the concentration of the nanobody capable of inhibiting pseudovirus entry by 50% (IC₅₀).

To assess the effects of Nanosota-5-Fc on pseudovirus entry into TMPRSS2-expressing cells, HEK293T cells transiently expressing human ACE2 and TMPRSS2 were used instead of HEK293T/hACE2 cells. All other procedures remained the same as described above.

For the post-attachment pseudovirus entry assay, pseudoviruses were adsorbed onto HEK293T/hACE2 cells at 4° C. for 1 hour. Unbound pseudoviruses were removed, and the cells were washed three times with cold DMEM. Serially diluted Nanosota-5-Fc was then added to the cells and incubated at 4° C. for 1 hour. The plates were subsequently transferred to 37° C. to allow viral entry. Pseudovirus entry results were measured in the same manner as described above.

Construction of BAC cDNA Clone of rSARS-CoV-2-Venus and Virus Rescue

Recombinant SARS-CoV-2-Venus (rSARS-CoV-2-Venus) was engineered using a GalK/kanamycin dual marker cassette as previously described⁴¹. The BAC cDNA clone of rSARS-CoV-2-Venus was analyzed using restriction enzyme digestion, PCR, and direct sequencing and shown to be correct. The BAC-SARS-CoV-2 cDNA clone was constructed as previously described⁴¹

Confluent monolayers of Vero E6 cells were transfected with 2.0 g per well of rSARS-CoV-2-Venus BAC cDNA using Lipofectamine 3000. At 72 hours post transfection, cell supernatants were harvested, labeled as P0, and stored at −80° C. The P0 virus was used to infect fresh Calu3 cells to generate P1 stocks. P1 viral stocks were aliquoted, titered and stored at −80° C. until use.

### Live SARS-CoV-2 Infection In Vitro

Cell infection of live prototypic SARS-CoV-2 in cultured cells by Nanosota-5-Fc was detected using flow cytometry. Briefly, rSARS-CoV-2-Venus was incubated with different concentrations of Nanosota-5-Fc in 200 ml DMEM at 37° C. for 1 hour. Vero E6 cells (106 cells per well, 12-well plate, triplicates) were either mock inoculated or inoculated with the virus/nanobody mixture. After incubation at 37° C. for 1 hour with gentle rocking every 15 minutes, the inocula were removed and the plates were overlaid with 10% FBS. 24 hours post-inoculation, cells were treated with 100 ml of trypsin for 2 minutes. Following this, 500 ml 10% FBS was added to terminate the trypsinization, and cell suspension was centrifuged with 1000 rpm/min for 5 minutes. Cell pellets were resuspended using Cytofix (BD Bioscience) and fixed for 30 minutes. Expression of Venus was evaluated via flow cytometry. All flow cytometry data were acquired using a BD FACSVerse and analysed with FlowJo software. A previously discovered RBD-targeting nanobody Nanosota-3-Fc was used as a comparison²⁸.

The neutralization potency of Nanosota-5-Fc against live Omicron infections was carried out as previously described⁴². Briefly, Nanosota-5-Fc was 10-times serially diluted in 50 μl virus growth medium and then was mixed with 50 μl of one of the live Omicron viruses (subvariants BA.1, BA.5 or XBB.1.5) (3000 TCID₅₀/ml) at 37° C. for an hour. The mixture was added to Vero E6 cells, which over express TMPRSS2, in 96-well plates and incubated at 37° C. in 5% CO₂ for 4 days. Cell viability was measured using a neutral red assay (Sigma-Aldrich). The efficacy of Nanosota-5-Fc against the Omicron viruses was calculated and expressed as the concentration capable of maintaining the cell viability by 50% (i.e., IC₅₀) compared to the control virus.

### Cell-Cell Fusion Assay

Cell-cell fusion was performed as described previously⁴³. Briefly, HEK293T cells were co-transfected with the plasmid pFR-Luc (containing a luciferase gene whose expression is controlled by a synthetic promoter) and the plasmid expressing SARS-CoV-2 spike. Besides, HEK293T cells expressing ACE2 were transfected with the plasmid pBD-NF-xB (encoding a fusion protein that can activate the luciferase gene expression of the pFR-Luc). After the cells were cultured for 36 hours, Nanosota-5-Fc at various concentrations were added to the spike-expressing cells and incubated for 30 minutes. Subsequently, ACE2-expressing cells were overlaid onto spike-expressing cells. The luciferase gene expression was activated when cell-cell fusion occurred. After incubation for 6 hours, the cells were lysed, and relative luciferase units were measured using an EnSpire plate reader (PerkinElmer Life Sciences).

### Flow Cytometry Assay

To assess the effects of Nanosota-5-Fc on the binding between cell-surface spike and recombinant ACE2 ectodomain, various concentrations of Nanosota-5-Fc were incubated with spike-expressing HEK293T cells for 20 minutes. Then, 2 μg/ml human ACE2 ectodomain (containing a C-terminal His tag) was added and incubated with the cell and Nanosota-5-Fc mixture for another 20 minutes. Spike-bound ACE2 molecules were stained with a PE-conjugated anti-His-tag antibody and analyzed using flow cytometry. The results were analyzed using FlowJo software (version 10).

To assess the effects of Nanosota-5-Fc on the binding between cell-surface spike and recombinant Nanosota-2-His (which only binds to the standing up RBD in the spike), 0.5 μg/ml Nanosota-2-His replaced 2 μg/ml human ACE2 ectodomain in the above experiment.

### Furin Cleavage Inhibition Assay

HEK293T cells were seeded into a 6-well plate and transfected with 1 μg of prototypic or XBB.1.5 spike-expressing plasmids. Different amounts (0, 0.2, 1, and 5 μg) of plasmids expressing Nanosota-5-Fc were co-transfected with the spike-expressing plasmids. Twenty-four hours post-transfection, cells were collected, and the cell surface spike (containing a C-terminal C9 tag) was detected using an anti-C9 antibody by Western blot.

### Cryo-EM Grid Preparation and Data Acquisition

4 μl purified complexes of SARS-CoV-2 spike ectodomain and nanobodies (˜2.6 μM for prototypic spike/Nanosota-5, -3.1 μM for prototypic spike/Nanosota-6, and ˜2.4 μM for XBB.1.5 spike/Nanosota-5) were supplemented with 8 mM CHAPSO immediately before grid preparation. Each complex was then applied to freshly glow-discharged Quantifoil R1.2/1.3 300-mesh copper grids (EM Sciences) and blotted for 4 seconds at 22° C. under 100% chamber humidity and plunge-frozen in liquid ethane using a Vitrobot Mark IV (FEI). Cryo-EM data were collected using Latitude-S(Gatan) equipped with a K3 direct electron detector and with a Biocontinuum energy filter (Gatan). For the prototypic spike/Nanosota-5 and prototypic spike/Nanosota-6 complexes, the movies were collected at a nominal magnification of 130,000× (corresponding to 0.664 Å per pixel). For the XBB.1.5 spike/Nanosota-5 complex, the movies were collected at a nominal magnification of 81,000× (corresponding to 1.1 Å per pixel). Statistics of cryo-EM data collection are summarized in Table S1.

### Cryo-EM Data Processing, Model Building and Refinement

Cryo-EM data were processed using cryoSPARC v3.3.2⁴⁴, and the procedure is outlined in FIG. S1, S2 and S5. Briefly, dose-fractionated movies were subjected to Patch motion correction with MotionCor2⁴⁵ and Patch CTF estimation with CTFFIND-4.1.13⁴⁶. Particles were then picked using both Blob picker and Template picker in cryoSPARC v3.3.2 and subjected to the Remove Duplicate Particles Tool. Junk particles were removed through multiple rounds of 2D classifications. Particles from the good 2D classes were used for Ab-initio Reconstruction of three or four maps. The initial models were set as the starting references for heterogeneous refinement (3D classification). The good 3D classes were then subjected to further homogeneous, non-uniform and CTF refinements to generate the final maps with applied C3 symmetry. Map resolutions were determined by gold-standard Fourier shell correlation (FSC) at 0.143 between the two half-maps. Local resolution variation was estimated from the two half-maps in cryoSPARC v3.3.2 or v4.0.3.

Initial model building of the spike/nanobody complexes was performed in Coot-0.8.9⁴⁷ using PDB 7TGY and 8IOS as the starting model for prototypic and XBB.1.5 spikes, respectively. The initial model of each nanobody was predicted using SWISS-MODEL (https://swissmodel.expasy.org/), and then fitted into the density map. Several rounds of refinement in Phenix-1.16⁴⁸ and manual building in Coot-0.8.9 were performed until the final reliable models were obtained. Model and map statistics are summarized in Table S1. Figures were generated using UCSF Chimera X v0.93⁴⁹ and PyMol v2.5.2⁵⁰.

TABLE S7
Cryo-EM data collection, refinement and validation statistics of the
prototypic spike/Nanosota-5, prototypic spike/Nanosota-6, and
XBB.1.5 spike/Nanosota-5 complexes.

| | Prototypic | Prototypic | XBB.1.5 |
|---|---|---|---|
| | spike/ | spike/ | spike/ |
| | Nanosota-5 | Nanosota-6 | Nanosota-5 |
| | (EMD-29798) | (EMD-29799) | EMD-42218) |
| | (PDB 8G76) | (PDB 8G77) | (PDB 8UG9) |
| Data collection and | | | |
| processing | | | |
| Magnification | 130,000 | 130.000 | 81,000 |
| Voltage (kV) | 300 | 300 | 300 |
| Electron exposure | 40.00 | 40.00 | 50.00 |
| (e−/Å²) | | | |
| Defocus range (μm) | −0.8~−2.4 | −0.8~−2.4 | −1.0~−2.0 |
| Pixel size (Å) | 0.664 | 0.664 | 1.1 |
| Symmetry imposed | C3 | C3 | C3 |
| Initial particle images | 52,821 | 234,682 | 100,775 |
| (no.) | | | |
| Final particle images | 31,809 | 189,562 | 81,718 |
| (no.) | | | |
| Map resolution (Å) | 3.8 | 2.8 | 3.49 |
| FSC threshold | 0.143 | 0.143 | 0.143 |
| Map resolution range | 3.5-7.5 | 2.6-3.8 | 3.0-5.4 |
| (Å) | | | |
| Refinement | | | |
| Initial model used | 7TGY | 7TGY | 8IOS |
| (PDB code) | | | |
| Model resolution (Å) | 4.0 | 3.0 | 3.6 |
| FSC threshold | 0.5 | 0.5 | 0.5 |
| Model resolution | 42.5-3.6 | 54.2-2.7 | 44.5-3.6 |
| range (Å) | | | |
| Map sharpening B | −124.9 | −99.8 | −106.9 |
| factor (Å²) | | | |
| Model composition | | | |
| Non-hydrogen atoms | 28572 | 28945 | 28386 |
| Protein residues | 3566 | 3608 | 3531 |
| Ligands | 53 | 61 | 57 |
| B factors (Å²) | | | |
| Protein | 234.87 | 28.98 | 32.11 |
| Nucleotide | | | |
| Ligand | 234.03 | 43.62 | 62.73 |
| R.m.s. deviations | | | |
| Bond lengths (Å) | 0.004 | 0.005 | 0.003 |
| Bond angles (°) | 0.620 | 0.828 | 0.553 |
| Validation | | | |
| MolProbity score | 1.94 | 1.96 | 1.57 |
| Clashscore | 12.65 | 11.70 | 5.59 |
| Poor rotamers (%) | 0.42 | 1.28 | 0.23 |
| Ramachandran plot | | | |
| Favored (%) | 95.29 | 95.77 | 96.12 |
| Allowed (%) | 4.56 | 4.09 | 3.82 |
| Disallowed (%) | 0.14 | 0.14 | 0.06 |

### References Cited in Example 3

1. Graham B S, Gilman M S A, McLellan J S. Structure-Based Vaccine Antigen Design. Annual Review of Medicine 70, 91-104 (2019).
2. Shafqat A, et al. SARS-CoV-2 epitopes inform future vaccination strategies. Frontiers in immunology 13, 1041185 (2022).
3. Liu Y, et al. An infectivity-enhancing site on the SARS-CoV-2 spike protein targeted by antibodies. Cell 184, 3452-3466.e3418 (2021).
4. Wan Y, et al. Molecular Mechanism for Antibody-Dependent Enhancement of Coronavirus Entry. Journal of Virology 94, e02015-02019 (2020).
5. Katzelnick L C, et al. Antibody-dependent enhancement of severe dengue disease in humans. Science 358, 929-932 (2017).
6. Takada A, Kawaoka Y. Antibody-dependent enhancement of viral infection: molecular mechanisms and in vivo implications. Reviews in medical virology 13, 387-398 (2003).
7. Lee W S, Wheatley A K, Kent S J, DeKosky B J. Antibody-dependent enhancement and SARS-CoV-2 vaccines and therapies. Nature microbiology 5, 1185-1191 (2020).
8. Wang M Y, Zhao R, Gao L J, Gao X F, Wang D P, Cao J M. SARS-CoV-2: Structure, Biology, and Structure-Based Therapeutics Development. Front Cell Infect Microbiol 10, 587269 (2020).
9. Perlman S, Netland J. Coronaviruses post-SARS: update on replication and pathogenesis. Nature Reviews Microbiology 7, 439-450 (2009).
10. Li F. Structure, function, and evolution of coronavirus spike proteins. Annual review of virology 3, 237-261 (2016).
11. Geng Q, et al. Lys417 acts as a molecular switch that regulates the conformation of SARS-CoV-2 spike protein. eLife 12, e74060 (2023).
12. Shang J, et al. Cell entry mechanisms of SARS-CoV-2. Proceedings of the National Academy of Sciences 117, 11727-11734 (2020).
13. Lan J, et al. Structure of the SARS-CoV-2 spike receptor-binding domain bound to the ACE2 receptor. Nature 581, 215-220 (2020).
14. Shang J, et al. Structural basis of receptor recognition by SARS-CoV-2. Nature 581, 221-224 (2020).
15. Wan Y, Shang J, Graham R, Baric R S, Li F. Receptor Recognition by the Novel Coronavirus from Wuhan: an Analysis Based on Decade-Long Structural Studies of SARS Coronavirus. J Virol 94, (2020).
16. Du LY, He Y X, Zhou Y S, Liu S W, Zheng B J, Jiang S B. The spike protein of SARS-CoV—a target for vaccine and therapeutic development. Nature Reviews Microbiology 7, 226-236 (2009).
17. Ju B, et al. Human neutralizing antibodies elicited by SARS-CoV-2 infection. Nature 584, 115-119 (2020).
18. Barnes C O, et al. SARS-CoV-2 neutralizing antibody structures inform therapeutic strategies. Nature 588, 682-687 (2020).
19. Kreye J, et al. A Therapeutic Non-self-reactive SARS-CoV-2 Antibody Protects from Lung Pathology in a COVID-19 Hamster Model. Cell 183, 1058-1069.e1019 (2020).
20. Zhou B, et al. A broadly neutralizing antibody protects Syrian hamsters against SARS-CoV-2 Omicron challenge. Nature communications 13, 3589 (2022).
21. Li C J, et al. Neutralizing Monoclonal Antibodies Inhibit SARS-CoV-2 Infection through Blocking Membrane Fusion. Microbiol Spectr 10, e0181421 (2022).
22. Li C J, Chang SC. SARS-CoV-2 spike S2-specific neutralizing antibodies. Emerg Microbes Infect 12, 2220582 (2023).
23. Liu L, et al. Antibodies targeting a quaternary site on SARS-CoV-2 spike glycoprotein prevent viral receptor engagement by conformational locking. Immunity 56, 2442-2455.e2448 (2023).
24. Asaadi Y, Jouneghani F F, Janani S, Rahbarizadeh F. A comprehensive comparison between camelid nanobodies and single chain variable fragments. Biomarker research 9, 87 (2021).
25. Muyldermans S. Nanobodies: natural single-domain antibodies. Annu Rev Biochem 82, 775-797 (2013).
26. De Meyer T, Muyldermans S, Depicker A. Nanobody-based products as research and diagnostic tools. Trends Biotechnol 32, 263-270 (2014).
27. Du L, Yang Y, Zhang X, Li F. Recent advances in nanotechnology-based COVID-19 vaccines and therapeutic antibodies. Nanoscale 14, 1054-1074 (2022).
28. Ye G, et al. Discovery of Nanosota-2,-3, and -4 as super potent and broad-spectrum therapeutic nanobody candidates against COVID-19. Journal of Virology, e01448-01423 (2023).
29. Meng B, et al. Altered TMPRSS2 usage by SARS-CoV-2 Omicron impacts infectivity and fusogenicity. Nature 603, 706-714 (2022).
30. Shimizu J, et al. Reevaluation of antibody-dependent enhancement of infection in anti-SARS-CoV-2 therapeutic antibodies and mRNA-vaccine antisera using FcR- and ACE2-positive cells. Scientific reports 12, 15612 (2022).
31. Maemura T, Kuroda M, Armbrust T, Yamayoshi S, Halfmann P J, Kawaoka Y. Antibody-Dependent Enhancement of SARS-CoV-2 Infection Is Mediated by the IgG Receptors Fc γ RIIA and Fc γ RIIIA but Does Not Contribute to Aberrant Cytokine Production by Macrophages. mBio 12, e0198721 (2021).
32. Zhou Y, et al. Enhancement versus neutralization by SARS-CoV-2 antibodies from a convalescent donor associates with distinct epitopes on the RBD. Cell reports 34, 108699 (2021).
33. Li D, et al. In vitro and in vivo functions of SARS-CoV-2 infection-enhancing and neutralizing antibodies. Cell 184, 4203-4219.e4232 (2021).
34. Hu W, et al. Mechanical activation of spike fosters SARS-CoV-2 viral infection. Cell Res 31, 1047-1060 (2021).
35. Hsieh C L, et al. Structure-based design of prefusion-stabilized SARS-CoV-2 spikes. Science 369, 1501-1505 (2020).
36. Ye G, Liu B, Li F. Cryo-EM structure of a SARS-CoV-2 omicron spike protein ectodomain. Nature communications 13, 1214 (2022).
37. Warming S, Costantino N, Court D L, Jenkins N A, Copeland N G. Simple and highly efficient BAC recombineering using galK selection. Nucleic Acids Res 33, e36 (2005).
38. Wong L R, et al. Eicosanoid signalling blockade protects middle-aged mice from severe COVID-19. Nature 605, 146-151 (2022).
39. Ye G, et al. The development of Nanosota-1 as anti-SARS-CoV-2 nanobody drug candidates. eLife 10, (2021).
40. Geng Q, et al. Novel virus-like nanoparticle vaccine effectively protects animal model from SARS-CoV-2 infection. PLoS Pathog 17, e1009897 (2021).
41. Almazin F, et al. Engineering the largest RNA virus genome as an infectious bacterial artificial chromosome. Proc Natl Acad Sci USA 97, 5516-5521 (2000).
42. Repetto G, del Peso A, Zurita J L. Neutral red uptake assay for the estimation of cell viability/cytotoxicity. Nature protocols 3, 1125-1131 (2008).
43. Zheng Y, et al. Lysosomal Proteases Are a Determinant of Coronavirus Tropism. J Virol 92, (2018).

44. Punjani A, Rubinstein J L, Fleet D J, Brubaker M A. cryoSPARC: algorithms for rapid unsupervised cryo-EM structure determination. Nature methods 14, 290-296 (2017).
45. Rubinstein J L, Brubaker M A. Alignment of cryo-EM movies of individual particles by optimization of image translations. Journal of structural biology 192, 188-195 (2015).
46. Rohou A, Grigorieff N. CTFFIND4: Fast and accurate defocus estimation from electron micrographs. Journal of structural biology 192, 216-221 (2015).
47. Emsley P, Cowtan K. Coot: model-building tools for molecular graphics. Acta Crystallographica Section D-Biological Crystallography 60, 2126-2132 (2004).
48. Adams P D, et al. PHENIX: a comprehensive Python-based system for macromolecular structure solution. Acta Crystallographica Section D-Biological Crystallography 66, 213-221 (2010).
49. Goddard T D, et al. UCSF ChimeraX: Meeting modern challenges in visualization and analysis. Protein science: a publication of the Protein Society 27, 14-25 (2018).
50. DeLano W L. PyMOL: an open-source molecular graphics tool. CCP4 Newsl Protein Crystallogr 40, 82-92 (2002).

### Example 4. Structure-guided in vitro evolution of nanobodies targeting new viral variants

A major challenge in antiviral antibody therapy is keeping up with the rapid evolution of viruses. Our research shows herein that nanobodies—single-domain antibodies derived from camelids—can be rapidly re-engineered to combat new viral strains through structure-guided in vitro evolution. Specifically, for viral mutations occurring at nanobody-binding sites, we introduce randomized amino acid sequences into nanobody residues near these mutations. We then select nanobody variants that effectively bind to the mutated viral target from a phage display library. As a proof of concept, we used this approach to adapt Nanosota-3, a nanobody originally identified to target the receptor-binding domain (RBD) of early Omicron subvariants, making it highly effective against recent Omicron subvariants. Remarkably, this adaptation process can be completed in less than two weeks, allowing drug development to keep pace with viral evolution and provide timely protection to humans.

### Introduction

The COVID-19 pandemic has exposed the limitations of conventional antibodies, particularly how viral mutations can swiftly negate the efficacy of existing antibody therapeutics. A striking instance occurred early in the pandemic when several antibody therapeutics effective against earlier variants of SARS-CoV-2 were suddenly rendered obsolete by the emergence of the Omicron variant [1-6]. Antibody treatments target the spike protein on the virus surface, which is crucial for the virus to enter cells by binding to the ACE2 receptor on the cell surface and then fusing the viral and cellular membranes [7-9]. To keep pace with the continuously evolving spike protein, antiviral antibody treatments must either target a universally conserved spike epitope or be swiftly modifiable to address new spike mutations [10-13]. Our research investigates the latter strategy, proposing an innovative method for the rapid adaptation of antibodies to confront newly arising viral mutations.

Nanobodies are single-domain antibodies sourced from camelid animals such as llamas and alpacas [14-16]. Their small size provides multiple advantages over conventional antibodies for antiviral therapy, such as reduced production costs, high thermal stability, and the ability to bind to hidden epitopes on viruses, along with their potential for administration via inhalers [17-19]. Importantly, the simplicity of their single-domain structure makes them ideal for phage display, which is crucial for screening purposes and for in vitro evolution [20]. We have recently identified a series of nanobodies that bind to the receptor-binding domain (RBD) of the SARS-CoV-2 spike protein [21, 22]. By blocking the interaction between the spike protein and the ACE2 receptor on host cells, these nanobodies effectively neutralize the virus's ability to enter and infect cells. Notably, one of these nanobodies, named Nanosota-1, was identified from a naive camelid nanobody library using the prototypic SARS-CoV-2 RBD as bait [21]. To enhance its affinity for the RBD, Nanosota-1 underwent two rounds of random in vitro maturation, receiving random mutations throughout its coding sequence. However, the indiscriminate nature of these random mutations across the entire nanobody led to an inefficient affinity maturation process. A more rational and targeted approach is important to adapt nanobodies more effectively to the fast-evolving viral targets.

Nanosota-3 is another SARS-CoV-2 RBD-targeting nanobody that we recently discovered [22]. Derived from an alpaca immunized with the prototypic SARS-CoV-2 spike, Nanosota-3 has demonstrated high potency against both the prototypic SARS-CoV-2 variant and an early Omicron subvariant BA.1, both in vitro and in mouse models. However, it is ineffective against a more recent Omicron subvariant XBB.1.5. The cryo-EM structure of prototypic spike in complex with Nanosota-3 revealed the detailed interactions between the two proteins [22]. Therefore, Nanosota-3 is an excellent candidate for structure-guided in vitro evolution aimed at countering emerging viral mutations.

In this study, we adapted Nanosota-3 to the XBB.1.5 subvariant using a novel structure-guided in vitro evolution approach. We confirmed the potency of the XBB1.5-adapted Nanosota-3 both in vitro and in a mouse model, and further elucidated the structural basis for its neutralization of XBB.1.5. This study serves as a proof of concept that structure-guided in vitro evolution is a highly effective strategy for equipping nanobodies to match rapid viral evolutions, thereby offering prompt and effective protection for humans.

### Results

### Design and Procedure of a Novel Structure-Guided In Vitro Evolution of Nanobodies

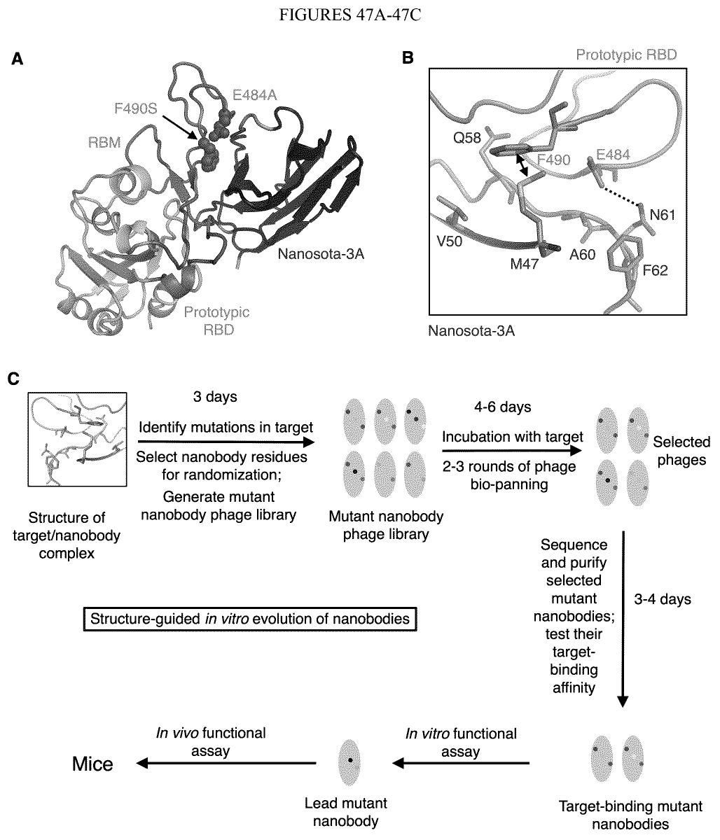

To assess the antiviral spectrum of Nanosota-3, we examined the structural interface between Nanosota-3 and the prototypic SARS-CoV-2 RBD. Compared to the prototypic RBD, XBB.1.5 RBD contains two mutations at this interface: E484A and F490S (FIG. 47A). The E484A mutation is likely to disrupt the hydrogen bond formed between Glu484 of the prototypical RBD and Asn61 of Nanosota-3 (FIG. 47B). Similarly, the F490S mutation is expected to disrupt the hydrophobic interaction between Phe490 of the prototypical RBD and Met47 of Nanosota-3. Consequently, these two mutations combined significantly reduced Nanosota-3's affinity for the XBB.1.5 RBD. Therefore, for Nanosota-3 to neutralize XBB.1.5 effectively, it would require engineering to address these specific mutations.

We developed an innovative, structure-based in vitro evolution strategy to improve the binding of Nanosota-3 to XBB.1.5 spike (FIG. 47C). Our goal was to comprehensively explore all possible combinations of important amino acid residue changes in Nanosota-3 near the two mutation sites of the XBB.1.5 RBD. We identified six residues on Nanosota-3 for this purpose. In addition to Asn61 and Met47, which directly interact with the mutation sites, we chose four additional residues near the mutation sites on the XBB.1.5 RBD: Ala60 and Phe62 in proximity to the E484A mutation, and Val50 and Gln58 near the F490S mutation. We then constructed a phage display library of Nanosota-3 mutants, which included a fully random assortment of these six residues, and screened for variants that bind with high affinity to XBB.1.5 spike. This screening could be performed using either a single library that includes all six randomized residues or two separate libraries, each randomizing a portion of the six residues. For this study, we opted for the two-library approach, though the single-library method is also feasible.

We constructed the first phage display library of nanobody mutants by randomizing two nanobody residues, 50 and 58, surrounding the F490S mutation in the XBB.1.5 RBD. To do this, we introduced mutations into the original Nanosota-3, now referred to as Nanosota-3A, through PCR using primers with fully randomized codons at the intended mutation sites. We then conducted bio-panning of the phages using the XBB.1.5 spike ectodomain as both the target and bait. Phages that showed affinity were sequenced across their nanobody-coding genes, and their respective nanobody variants were produced. These nanobody variants were further assessed through in vitro functional assays, including target-binding assays and SARS-CoV-2 pseudovirus entry assays. Among the 96 sequenced target-binding phages, one contained a nanobody with significantly higher binding affinity to the target than Nanosota-3A (FIG. 48A, 48B). This top-performing nanobody variant, named Nanosota-3B [22], contains two mutations, V50F and Q58S (FIG. 48A), compared to Nanosota-3A. Nanosota-3B was then used as the foundation for the second phage display library, where we randomized four residues, 47, 60, 61, and 62, surrounding the E484A mutation in the XBB.1.5 RBD. After another round of selection, four of the 96 sequenced target-binding phages contained a nanobody that demonstrated significantly improved binding affinity to the target compared to Nanosota-3B (FIG. 48A, 48B). The nanobody with the highest binding affinity for XBB.1.5 spike was identified as the top-performing variant, named Nanosota-3C (FIG. 48A, 48B). Nanosota-3C contains four mutations—M47G, A60W, N61L, and F62P—compared to Nanosota-3B (FIG. 48A).

### Characterization of Nanosota-3C Produced Through Structure-Guided In Vitro Evolution

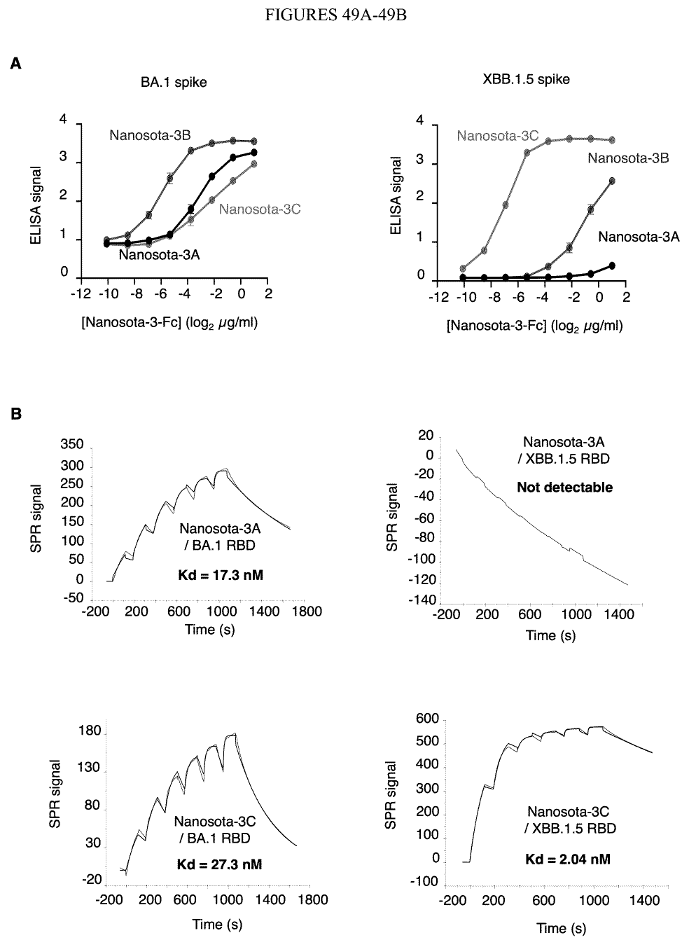

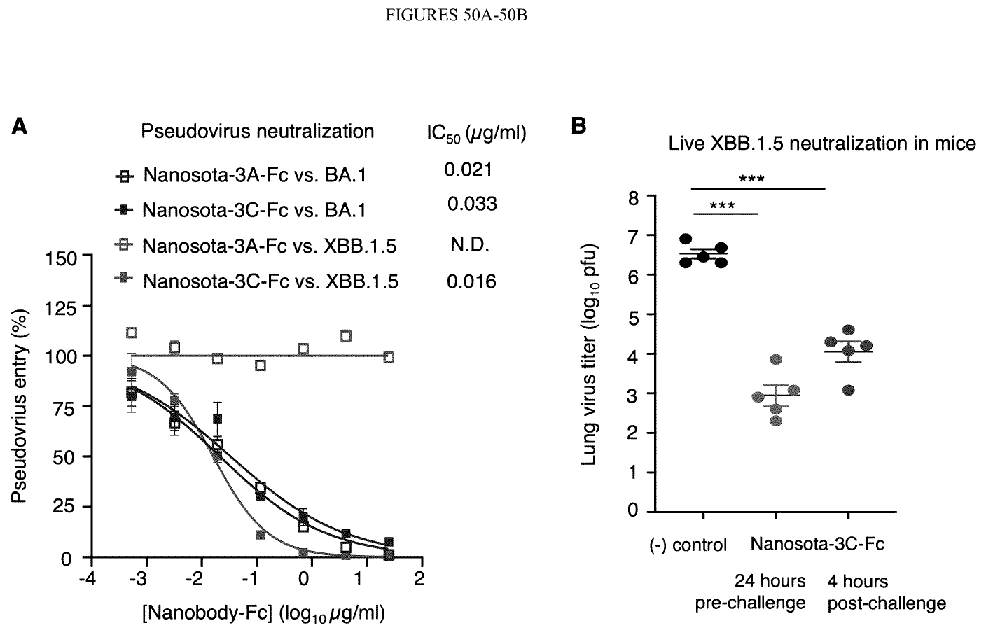

To evaluate the binding affinity of Nanosota-3C with XBB.1.5 spike, we conducted two separate assays. First, we performed an ELISA to assess the binding interactions between Nanosota-3C and both the BA.1 and XBB.1.5 spikes, using Nanosota-3A and -3B as comparisons (FIG. 49A). The results showed that all three Nanosota-3 variants bind to BA.1 spike with significant affinity. However, Nanosota-3A, -3B, and -3C bind to XBB.1.5 spike with low, intermediate, and high affinity, respectively. Second, we used surface plasmon resonance (SPR) to quantify the binding interactions between Nanosota-3C and the BA.1 and XBB.1.5 RBDs, with Nanosota-3A serving as a comparison (FIG. 49B). The results showed that Nanosota-3C binds strongly to both the BA.1 and XBB.1.5 RBDs, with Kds of 27.3 nM and 2.04 nM, respectively. In comparison, Nanosota-3A binds strongly to the BA.1 RBD but does not bind to the XBB.1.5 RBD. Thus, compared to Nanosota-3A, Nanosota-3C demonstrated dramatically enhanced binding affinity for XBB.1.5 spike.

To evaluate the potency of Nanosota-3C in neutralizing XBB.1.5 entry, we conducted two additional assays. For these tests, Nanosota-3C was modified to include a C-terminal Fc tag, creating Nanosota-3C-Fc. The addition of the Fc tag significantly enhances the in vivo half-life of nanobodies by increasing their molecular weight beyond the kidney clearance threshold (˜60 kDa) [21]. Despite this increase in size, Fc-tagged dimeric nanobodies remain about half the size of conventional antibodies, and their single-domain antigen-binding sites still effectively access cryptic epitopes on targets. The first assay measured the neutralizing capacity of Nanosota-3C-Fc against XBB.1.5 pseudoviruses (FIG. 50A). This involved using retroviruses pseudotyped with XBB.1.5 spike to enter ACE2-expressing cells in the presence of Nanosota-3C-Fc. The results showed that Nanosota-3C-Fc effectively neutralized both BA.1 and XBB.1.5 pseudoviruses, with IC₅₀ values of 33 ng/ml and 16 ng/ml, respectively. The second assay assessed the inhibitory potency of Nanosota-3C-Fc against a live XBB.1.5 challenge in a mouse model (FIG. 50B). In this experiment, mice were given Nanosota-3C-Fc either 24 hours before or 4 hours after the XBB.1.5 challenge. Analysis of virus titers in their lung tissues 2 days after the challenge revealed that Nanosota-3C-Fc significantly reduced viral titers at both pre-challenge and post-challenge time points. It is important to note that because Omicron variants cause mild symptoms in mice, virus titers in lung tissues were the only metric available for evaluating Omicron infections. Our results suggest that Nanosota-3C is an effective inhibitor of live XBB.1.5 in mice and can be used both as a preventive measure and as a treatment. In summary, Nanosota-3C has demonstrated strong neutralizing effects against the XBB.1.5 subvariant.

### Structure Basis of Nanosota-3C's Potent Inhibition of XBB.1.5

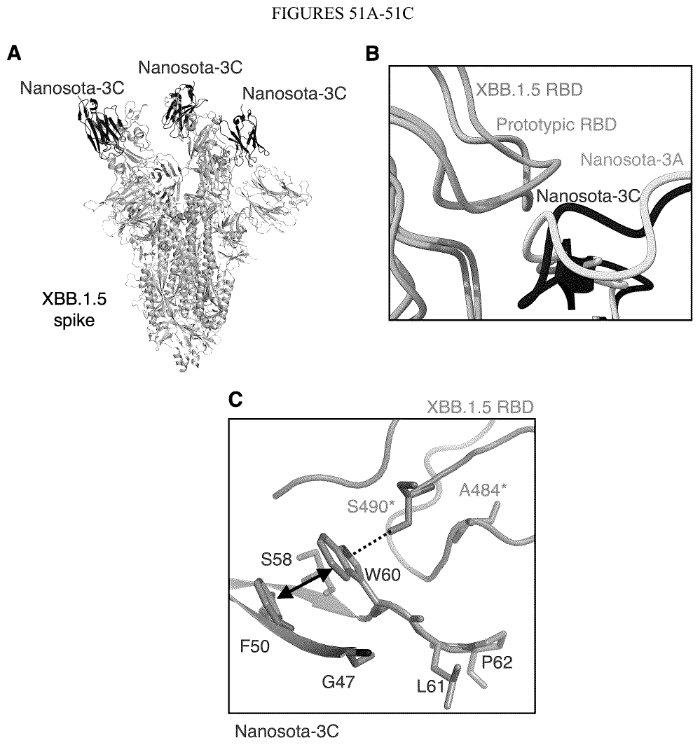

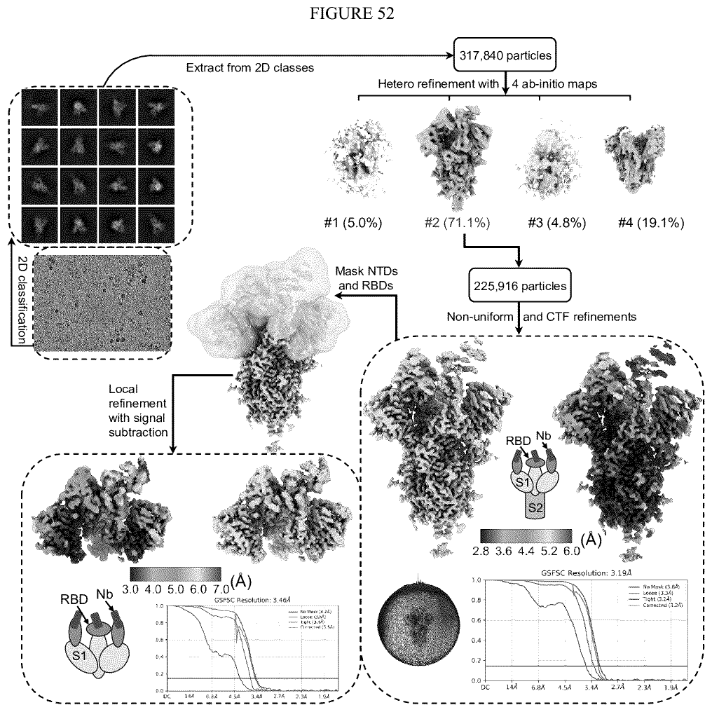

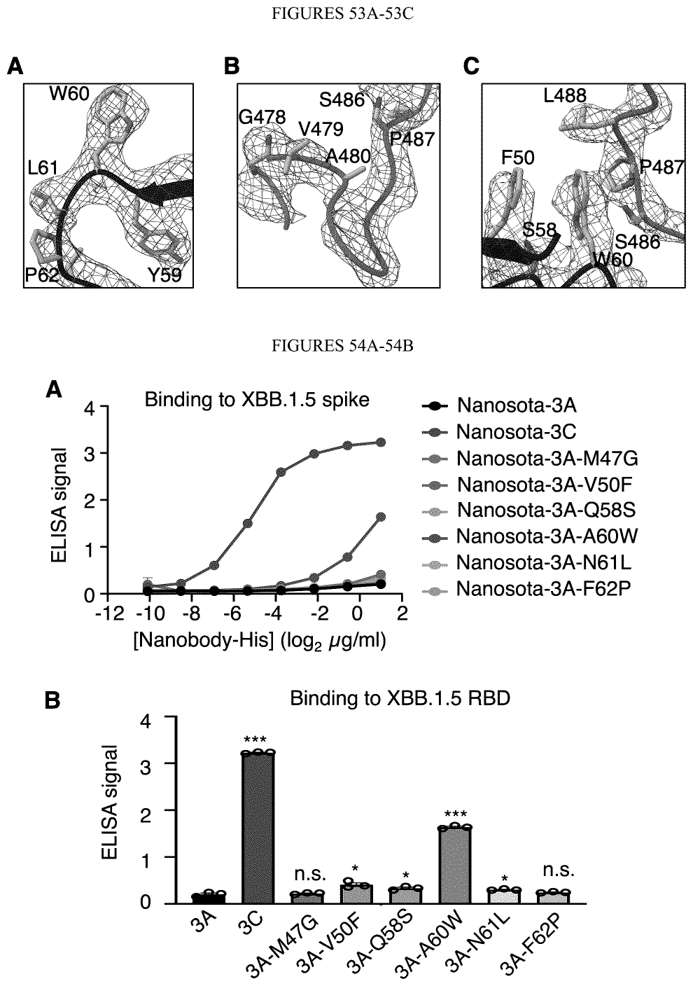

To understand the structural basis for Nanosota-3C's potent inhibition of XBB.1.5, we determined the cryo-EM structure of the XBB.1.5 spike ectodomain in complex with Nanosota-3C at 3.19A resolution (FIG. 51A; FIG. 52; Table S8). Our previous research had shown that the RBD of the SARS-CoV-2 spike adopts two conformations: a standing-up position to facilitate receptor binding and a lying-down position to evade the immune system [23, 24]. Nanosota-3C, like Nanosota-3A, binds to the RBD in both conformations (FIG. 51A; FIG. 52). The density at the interface between XBB.1.5 spike and Nanosota-3C is well-defined in the structure (FIG. 53). Compared to the structure of prototypic spike with Nanosota-3A, both XBB.1.5 spike and Nanosota-3C showed significant conformational shifts in the loop regions where mutations are present (FIG. 51B). Detailed examination of the interfaces revealed substantial structural changes due to these mutations. In the interface between the prototypic RBD and Nanosota-3A, the interaction was stabilized by hydrophobic contact between Phe490 of the RBD and Met47 of the nanobody, along with a hydrogen bond between Glu484 of the RBD and Asn61 of the nanobody (FIG. 47B). However, in the interface between the XBB.1.5 RBD and Nanosota-3C, these interactions were replaced by a rr-hydroxyl hydrogen bond between Trp60 of the nanobody and Ser490 of the RBD, and an aromatic 7L-7L stacking interaction between Trp60 and Phe50 of the nanobody (FIG. 51C). Thus, the mutations in Nanosota-3C align precisely with the mutations in the XBB.1.5 RBD, enabling high-affinity binding between Nanosota-3C and the XBB.1.5 RBD.

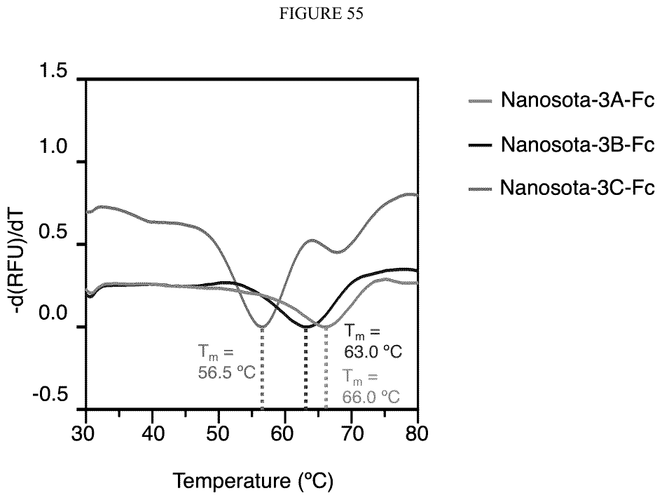

The detailed structural information on the interactions between Nanosota-3C and the XBB.1.5 RBD allowed us to further investigate how each of the in vitro evolved mutations contributes to the target-binding affinity of Nanosota-3C. We assessed the impact of each individual mutation in Nanosota-3C on its binding to the XBB.1.5 RBD by introducing each of the six mutations separately into Nanosota-3A and examining the binding interaction between each mutant Nanosota-3A and the XBB.1.5 RBD using ELISA (FIG. 54). The data revealed that the A60W mutation significantly increased Nanosota-3A's binding affinity for the XBB.1.5 RBD, while the other five mutations either slightly increased or did not significantly affect binding affinity. These results align with the structural data, which showed that while the A60W mutation introduced new hydrophobic stacking interactions at the Nanosota-3C/XBB.1.5 interface, the other mutations did not create direct interactions with the XBB.1.5 RBD. Instead, they primarily influenced the conformation of the RBD-binding loop, with their effects appearing to be synergistic.

Next, we examined how the in vitro evolved mutations affected the stability of Nanosota-3C. A thermostability assay conducted on Nanosota-3A, -3B, and -3C revealed that, compared to Nanosota-3A, Nanosota-3B showed slightly reduced thermostability, while Nanosota-3C exhibited significantly reduced thermostability (FIG. 55). Nanobodies contain four framework regions that act as structural scaffolds and three complementarity-determining regions (CDRs) responsible for antigen binding [21]. Occasionally, however, frameworks can also directly interact with antigens. In Nanosota-3C, all four mutations relative to Nanosota-3B are located within the frameworks. It has been shown that mutations in nanobody frameworks and the resulting structural changes can impact nanobody stability [25]. As a result, the significant conformational change in the RBD-binding loop within the framework likely decreased the stability of Nanosota-3C. However, it is important to note that the stability of Nanosota-3C remains within a reasonable range for nanobodies [26]. Additionally, this study serves as proof of concept that the target-binding affinities of nanobodies can be dramatically increased using a structure-guided in vitro evolution approach. Therefore, future applications of this approach involving the in vitro evolution of CDR regions are less likely to affect the stability of the nanobodies of interest.

While a specific set of residues in Nanosota-3A was chosen for this study, other residues near the two RBD mutation sites could have also been targeted for in vitro evolution. Given the structural flexibility of protein-protein interactions [27, 28], there may be multiple ways for a protein to evolve and adapt to mutations in its partner protein. As a result, if different residues in Nanosota-3A had been selected for in vitro evolution, the nanobody might have developed a different structural mechanism for binding to the XBB.1.5 RBD with high affinity. Therefore, the residue selections in this study and the resulting structural mechanism for RBD/nanobody binding may not represent the only possible evolutionary pathway for Nanosota-3A to adapt to the XBB.1.5 RBD.

### Discussion

The rapid mutation rate of SARS-CoV-2 necessitates two strategies for antibody treatment: broad-spectrum antibody therapeutics or those that can be quickly modified for new viral strains. Our study explores the second strategy, pioneering a structure-guided in vitro evolution of nanobodies to expedite their adaptation to emerging viral variants. From the structural interface between the original nanobody and viral target, we can identify and fully randomize key nanobody residues in close proximity to the viral target's mutations, and then isolate the nanobody variants that bind most strongly to the mutated viral target. This selection is conducted using a phage display library of nanobody variants encompassing all possible permutations of the randomized residues. The selected nanobody variants are then assessed using a series of in vitro and in vivo functional assays. Our method is designed to be fast, efficient, and systematic.

Our method surpasses the traditional random in vitro evolution techniques in tailoring nanobodies to combat new viral variants. The traditional approach scatters mutations throughout either the entire coding sequence or the CDRs of the nanobody, resulting in a limited and unfocused array of mutant nanobodies within the phage display libraries [21, 29-31]. By contrast, our structure-guided in vitro evolution method strategically introduces mutations at key residues of the nanobody that are adjacent to the viral target's mutation sites. This allows for an exhaustive examination of all potential combinations of mutations at these critical sites. Therefore, our structure-guided in vitro evolution represents a significant advancement in the field of in vitro protein evolution using phage display technology.

Our method has advantages over the immune system in terms of speed and is comparable to the immune system in effectiveness when responding to new viral variant outbreaks. The entire in vitro evolution process, from design to selecting the top candidate, can be completed in less than two weeks, which is significantly faster than the rate at which major viral variants emerge [32-34]. In contrast, immunizing animals with antigens from a new virus variant can take several months, not including the additional time required for phage library construction and screening. Moreover, our in vitro method can match the natural immune response in generating potent antiviral nanobodies. For instance, Nanosota-3C, developed through our in vitro evolution process, demonstrates potency comparable to some of the most effective anti-SARS-CoV-2 nanobodies produced by the immune system [35]. The rapid and efficient production of potent antiviral nanobodies positions structure-guided in vitro evolution as a prime strategy for combating emergent viral variants.

Our method confers a distinct advantage to nanobodies in their ability to be rapidly adapted to new viral variants when compared to conventional antibodies. The single-domain structure of nanobodies allows for straightforward cloning into phage vectors and surface display on phages. The selected nanobodies can also be efficiently expressed in bacterial systems for functional characterizations. In contrast, the two-chain structure of conventional antibodies complicates their use in phage display libraries and conventional antibodies also require expression in mammalian cells, creating substantial challenges when attempting to use the same rapid adaptation strategy. Overall, nanobodies are exceptionally well-equipped for this structure-guided in vitro evolution approach, standing out as an excellent solution in the face of rapidly evolving viral threats.

In summary, this study has led to the development of a cutting-edge, structure-guided in vitro evolution technique that efficiently adapts nanobodies to combat new viral variants. Our proof of concept has successfully demonstrated the technique's speed and effectiveness. This approach has the potential to broaden the application of nanobodies in antiviral research. The ongoing fight against viral diseases is made more difficult by the rapid evolution of viruses, which often outpaces current antiviral therapies.

### Data Availability

The atomic models and corresponding cryo-EM density maps is deposited into the PDB and the Electron Microscopy Data Bank, respectively, with accession numbers 9ATO and EMDB-43831 (XBB.1.5 spike complexed with Nanosota-3C), and 9ATP and EMDB-43832 (XBB.1.5 spike complexed with Nanosota-3C; after local refinement).

### Methods

### Ethics Statement

This study was performed in strict accordance with the recommendations in the Guide for the Care and Use of Laboratory Animals of the National Institutes of Health. All of the animals were handled according to approved institutional animal care and use committee (IACUC) protocols of the University of Iowa (protocol number: 9051795).

### Cell Lines, Plasmids and Viruses

HEK293T cells (American Type Culture Collection) were cultured in Dulbecco's modified Eagle medium (DMEM) with 10% fetal bovine serum. TG1 E. coli (Lucigen) and ss320 E. coli (Lucigen) were grown in 2YT medium. Vero E6 cells (American Type Culture Collection) were cultured in Eagle's minimal essential medium (EMEM) supplemented with penicillin (100 units/ml), streptomycin (100 μg/ml), and 10% fetal bovine serum. Prototypic SARS-CoV-2 spike gene (GenBank: QHD43416.1) was synthesized (GenScript) with an introduced D614G mutation. Spike genes of Omicron BA.1 subvariant (GISAID: EPI_ISL_6590782.2) and Omicron XBB.1.5 subvariant (GISAID: EPI_ISL_17774216) were derived from the prototypic SARS-CoV-2 spike gene through mutagenesis. Each of the spike genes was cloned into the pcDNA3.1(+) vector.

SARS-CoV-2 spike ectodomains (residues 14-1211, 14-1207, and 14-1207 for prototypic spike, BA.1 spike and XBB.1.5 spike, respectively) and SARS-CoV-2 RBDs (residues 316-526 and 315-525 for BA.1 RBD and XBB.1.5 RBD, respectively) were subcloned into Lenti-CMV vector (Vigene Biosciences) with an N-terminal tissue plasminogen activator (tPA) signal peptide and a C-terminal His tag. Additionally, in these spike ectodomain constructs, the furin motif RRAR (SEQ ID NO: 177) was replaced with AGAR (SEQ ID NO: 178) and six proline mutations were introduced to the S2 subunit region as previously described [23]. Fc-tagged nanobodies were constructed in the same way as the above RBDs except that a C-terminal human IgG₁ Fc tag replaced the C-terminal His tag.

Infectious XBB.1.5 was purchased from BEI resources. Experiments involving infectious viruses were conducted at the University of Iowa in approved biosafety level 3 laboratories.

### Structure-Guided In Vitro Evolution of Nanosota-3A

To enhance binding to the XBB.1.5 RBD, Nanosota-3A underwent two stages of structure-guided in vitro evolution. The first stage involved random mutations at Val50 and Gln58 through PCR, creating a collection of Nanosota-3A mutant genes. These mutations were completely random within the targeted sites on the PCR primers. This collection of Nanosota-3A mutant genes were then cloned into the PADL22c vector and introduced into TG1 cells via electroporation to construct a mutagenic phage display library. Mutant phages with improved binding to XBB.1.5 spike were then selected through bio-panning. After three rounds of bio-panning, the best binding phages were identified, their nanobody genes sequenced, and their nanobodies tested for affinity to XBB.1.5 spike using ELISA. The best target-binding mutant, named Nanosota-3B, contains two mutations, V50F and Q58S. In the second evolution stage, Nanosota-3B was mutated at four additional sites: Met47, Ala60, Asn61, and Phe62, following the same procedure to produce Nanosota-3C, which contains four additional mutations (M47G, A60W, N61L, and F62P).

Mutagenesis primer for Val50 and Gln58 mutations:

(SEQ ID NO: 174)
GAAGCAGCGCGAAATGGTCGCANNNATTAGTAGTATTGCTAGCACGNNNT
ATGCAAACTTCGTGAAG

Mutagenesis primer for Met47 mutation:

(SEQ ID NO: 175)
GGTTTCGCCAGGCTCTAGGGAAGCAGCGCGAANNNGTCGCATTTATTAGT
AGTATTGCTAGC

Mutagenesis primer for Ala60, Asn61 and Phe62 mutations:

(SEQ ID NO: 176)
CATTTATTAGTAGTATTGCTAGCACGAGTTATNNNNNNNNNGTGAAGGGC
CGATTCACC

### Protein Expression and Purification

Nanosota-3A, Nanosota-3B, and Nanosota-3C were expressed and purified as previously described [22]. Briefly, the His-tagged nanobodies were purified from the periplasm of ss320 E. coli following induction with 1 mM IPTG. The E. coli cells were harvested and re-suspended in 15 ml TES buffer (0.2 M Tris pH 8, 0.5 mM EDTA, 0.5 M sucrose). The proteins in the supernatant underwent sequential purification using a Ni-NTA column and a Superdex200 gel filtration column (Cytiva).

Prototypic, BA. 1, and XBB.1.5 spike ectodomains (all with a C-terminal His tag), BA.1 and XBB.1.5 RBDs (both with a His tag), and Nanosota-3A, -3B, and -3C (all with a C-terminal Fc tag) were produced from 293F mammalian cells as previously described [36]. Briefly, lentiviral particles were packaged using plasmids encoding one of the above proteins and then used to infect 293F cells to establish stable cell lines in the presence of Puromycin (Gibco). The proteins were harvested from the supernatants of the cell culture medium, purified using a Ni-NTA column for His-tagged proteins or a Protein A column for Fc-tagged proteins, and further purified on a Superose6 Increase gel filtration column (Cytiva) for spike ectodomains or a Superdex200 gel filtration column (Cytiva) for other proteins.

To prepare the complex of the XBB.1.5 spike ectodomain and Nanosota-3C, the two proteins were incubated at room temperature for 30 minutes and then subjected to Superose6 increase gel filtration column (Cytiva).

### ELISA

ELISA was performed to evaluate the binding interaction between His-tagged SARS-CoV-2 spike ectodomains and either HA-tagged nanobodies (also containing a His tag) or Fc-tagged nanobodies, as previously described [21]. ELISA plates were coated with one of the recombinant SARS-CoV-2 spike ectodomains and then sequentially incubated with nanobodies (either from the ss320 E. coli supernatant or recombinant nanobodies) and then with either HRP-conjugated anti-HA antibody (1:2,000) (Sigma) for HA-tagged nanobodies or HRP-conjugated anti-Fc antibody (1:3,000) (Jackson ImmunoResearch) for Fc-tagged nanobodies. The ELISA substrate (Invitrogen) was then added, and the reaction was stopped using 1N H₂SO4. Absorbance at 450 nm was measured using a Synergy LX Multi-Mode Reader (BioTek).

### Surface Plasmon Resonance

Surface plasmon resonance (SPR) was conducted to measure the binding affinity between nanobodies and SARS-CoV-2 RBDs using a Biacore S200 system (Cytiva), as previously described [21]. Briefly, one of the recombinant RBDs (with a His tag) was immobilized on a CM5 chip (Cytiva) through chemical cross-linking. Nanobodies (with a His tag) were injected at different concentrations from 32 nM to 1200 nM. The resulting data were fitted to a 1:1 binding model using Biacore Evaluation Software (Cytiva).

### Differential Scanning Fluorimetry (DSF)

To assess the thermostabilities of Nanosota-3A-Fc, -3B-Fc, and -3C-Fc, DSF experiments were performed. Each sample, consisting of 0.02 mg of individual nanobody, was mixed with 1.25 μl of diluted Protein Thermal Shift Dye (Thermo Fisher) in 10 μl of PBS (pH 7.4). The mixture was prepared in a 96-well qPCR plate. Protein stability measurements, indicated by the fluorescence signal during protein denaturation, were conducted in a PCR instrument (Applied Biosystems) using a temperature ramp from 25° C. to 99° C. at a rate of 0.05° C./s. Data collection was performed using real-time PCR software. The negative first derivative of the fluorescence signal was plotted against temperature, with the peak indicating the melting temperature (Tm).

### Pseudovirus Entry Assay

SARS-CoV-2 pseudovirus entry assay was carried out to measure the neutralizing potencies of nanobodies against SARS-CoV-2 pseudoviruses, as previously described [36]. Briefly, pseudoviruses were prepared by co-transfecting HEK293T cells with a pcDNA3.1(+) plasmid encoding the spike protein from one of the SARS-CoV-2 variants, a helper plasmid psPAX2, and a reporter plasmid plenti-CMV-luc. After 72 hours, pseudoviruses were collected, incubated with nanobodies at varying concentrations at 37° C. for 1 hour, and then used to enter HEK293T cells expressing human ACE2. Following an additional 60 hours, cell were lysed. Portions of the cell lysates were transferred to new plates, a luciferase substrate was introduced, and Relative Light Units (RLUs) were measured using an EnSpire plate reader (PerkinElmer). The efficacy of each nanobody was determined and expressed as the concentration capable of inhibiting pseudovirus entry by 50% (IC₅₀).

### Evaluation of Nanobody Potency in Mouse Model

The efficacy of Nanosota-3C-Fc against infectious XBB.1.5 in vivo was evaluated through a SARS-CoV-2 challenge experiment in a mouse model, as previously described [22]. Briefly, C57BL/6 mice (3-4 month old) were randomly separated into 3 groups. All mice were challenged via intranasal inoculation of infectious XBB.1.5 (1×10⁴ PFU/mouse) in a volume of 50 μl DMEM. In the pre-treatment group (n=5), mice received Nanosota-3C-Fc (10 mg/kg weight) via intraperitoneal delivery at 24 hours pre-challenge. In the post-treatment group (n=5), mice received Nanosota-3C-Fc (10 mg/kg weight) via intraperitoneal delivery at 4 hours post-challenge. In the control group (n=5), mice were administered PBS buffer at 4 hours post-challenge. The virus titers in the lungs of the mice were measured using a virus plaque assay, as previously described [22]. Briefly, mice were euthanized on day 2 post-challenge and lung tissue homogenate supernatants were collected. 12-well plates of Vero E6 cells overexpressing ACE2 and TMPRSS2 were inoculated with serially diluted lung homogenates (in DMEM) and then incubated at 37° C. in 5% CO₂ for 1 hour with gently shaking every 15 minutes. Then the inocula were removed and the plates were overlaid with 0.6% agarose containing 2% FBS. After 3 days, the overlays were removed, and the plaques were visualized via staining with 0.1% crystal violet. Virus titers were quantified as PFU per ml tissue.

### Cryo-EM Grid Preparation and Data Acquisition

Cryo-EM data collection was conducted as previously described [22]. Briefly, 4 μl complex (at 2 μM) of the XBB.1.5 spike ectodomain and Nanosota-3C at 0.8 μM was applied to freshly glow-discharged Quantifoil R1.2/1.3 300-mesh copper grids (EM Sciences) and blotted for 4 seconds at 22° C. under 100% chamber humidity and plunge-frozen in liquid ethane using a Vitrobot Mark IV (FEI). Cryo-EM data were collected using EPU (Thermo Fisher) equipped with a K3 direct electron detector and with a Biocontinuum energy filter (Gatan) at a nominal magnification of 130,000× (corresponding to 0.664 Å per pixel) (Table S8).

### Image Processing

Image processing was performed as previously described [22]. Briefly, cryo-EM data were processed using cryoSPARC v3.3.2 [37]. Dose-fractionated movies were subjected to Patch motion correction with MotionCor2 [38] and Patch CTF estimation with CTFFIND-4.1.13 [39]. Particles were then picked using Blob picker and Template picker in cryoSPARC v3.3.2 and subjected to the Remove Duplicate Particles Tool. Junk particles were removed through three rounds of 2D classifications. Particles from the good 2D classes were used for Ab-initio Reconstruction of four maps. The initial models were set as the starting references for heterogeneous refinement (3D classification). The selected 3D classes were then subjected to further non-uniform and CTF refinements, generating the final maps. To improve densities of the RBD and nanobody interface, particles in the good 3D class were imported into RELION-4.0 [40] using the csparc2star.py module (UCSF pyem v0.5. Zenodo) and subjected to signal subtraction to keep only the receptor-binding subunit of the spike and the nanobody in RELION-4.0. This was then followed by local refinements in cryoSPARC v3.3.2. Resolutions of the maps were determined by gold-standard Fourier shell correlation (FSC) at 0.143 between the two half-maps. Local resolution variations were estimated from the two half-maps in cryoSPARC v3.3.2 (FIG. 53; FIG. 55).

### Cryo-EM Model Building and Refinement.

Cryo-EM model building and refinement were carried out as previously described [22]. Briefly, initial model building of the spike/nanobody complex was performed in Coot-0.8.9 [41] using PDB 8VKL as the starting model. Previously published structure of Nanosota-3A was used as the initial model for Nanosota-3C [22]. Several rounds of refinement in Phenix-1.16 [42] and manually building in Coot-0.8.9 were performed until the final reliable model was obtained. In the model, standing-up RBDs and their bound nanobodies are generally flexible and hence they were fitted into the density as rigid bodies. In the local map of the XBB.1.5 spike/Nanosota-3C complex, an atomic model was built at the interface between the lying-down RBD and Nanosota-3C (Table S8). Figures were generated using UCSF Chimera X v0.93 [43] and PyMol v2.5.2 [44].

TABLE S8
Cryo-EM data collection, refinement and validation statistics
of the XBB.1.5 spike/Nanosota-3C complex.

| | | Local refinement of |
|---|---|---|
| | XBB.1.5 | XBB.1.5 |
| | Spike/Nanosota-3C | Spike/Nanosota-3C |
| | (EMDB-43831) | (EMDB-43832) |
| | (PDB 9ATO) | (PDB 9ATP) |
| Data collection and | | |
| processing | | |
| Magnification | 130,000 | |
| Voltage (kV) | 300 | |
| Electron exposure (e−/Å²) | 50.00 | |
| Defocus range (μm) | 1.0-2.0 | |
| Pixel size (Å) | 0.664 | |
| Symmetry imposed | C1 | |
| Initial particle images (no.) | 317,840 | 327,840 |
| Final particle images (no.) | 225,916 | 225,916 |
| Map resolution (Å) | 3.2 | 3.5 |
| FSC threshold | 0.143 | 0.143 |
| Map resolution range (Å) | 2.8-6.0 | 3.0-7.0 |
| Refinement | | |
| Initial model used (PDB | 8VKL | 8VKL |
| code) | | |
| Model resolution (Å) | 3.3 | 3.7 |
| FSC threshold | 0.5 | 0.5 |
| Model resolution range (Å) | 43.3-3.1 | 44.7-3.3 |
| Map sharpening B factor (Å²) | −109.6 | −108.9 |
| Model composition | | |
| Non-hydrogen atoms | 27725 | 13004 |
| Protein residues | 3478 | 1636 |
| Ligands | 40 | 0 |
| B factors (Å²) | | |
| Protein | 162.80 | 176.61 |
| Nucleotide | | |
| Ligand | 130.34 | |
| R.m.s. deviations | | |
| Bond lengths (Å) | 0.003 | 0.005 |
| Bond angles (*) | 0.598 | 0.731 |
| Validation | | |
| MolProbity score | 1.62 | 2.11 |
| Clashscore | 8.31 | 15.12 |
| Poor rotamers (%) | 0.36 | 0.21 |
| Ramachandran plot | | |
| Favored (%) | 97.05 | 93.53 |
| Allowed (%) | 2.86 | 6.16 |
| Disallowed (%) | 0.09 | 0.31 |

### References Cited in Example 4

1. Xiong D, Zhao X, Luo S, Cong Y, Zhang J Z H, Duan L. Immune Escape Mechanisms of SARS-CoV-2 Delta and Omicron Variants against Two Monoclonal Antibodies That Received Emergency Use Authorization. J Phys Chem Lett. 2022; 13(26):6064-73. Epub 20220627. doi: 10.1021/acs.jpclett.2c00912. PubMed PMID: 35758899.
2. Planas D, Bruel T, Staropoli I, Guivel-Benhassine F, Porrot F, Maes P, et al. Resistance of Omicron subvariants B A.2.75.2, B A.4.6, and B Q.1.1 to neutralizing antibodies. Nature communications. 2023; 14(1):824. Epub 20230214. doi: 10.1038/s41467-023-36561-6. PubMed PMID: 36788246; PubMed Central PMCID: PMCPMC9926440.
3. Arora P, Kempf A, Nehlmeier I, Schulz S R, Jack H M, Pöhlmann S, Hoffmann M. Omicron sublineage B Q.1.1 resistance to monoclonal antibodies. Lancet Infect Dis. 2023; 23(1):22-3. Epub 20221118. doi: 10.1016/s1473-3099(22)00733-2. PubMed PMID: 36410372; PubMed Central PMCID: PMCPMC9707647.
4. Hoffmann M, Kruger N, Schulz S, Cossmann A, Rocha C, Kempf A, et al. The Omicron variant is highly resistant against antibody-mediated neutralization: Implications for control of the COVID-19 pandemic. Cell. 2022; 185(3):447-56.el l. Epub 20211224. doi: 10.1016/j.cell.2021.12.032. PubMed PMID: 35026151; PubMed Central PMCID: PMCPMC8702401.
5. Tada T, Zhou H, Dcosta B M, Samanovic M I, Chivukula V, Herati R S, et al. Increased resistance of SARS-CoV-2 Omicron variant to neutralization by vaccine-elicited and therapeutic antibodies. EBioMedicine. 2022; 78:103944. doi: 10.1016/j.ebiom.2022.103944. PubMed PMID: 35465948; PubMed Central PMCID: PMCPMC9021600.
6. Uraki R, Kiso M, Imai M, Yamayoshi S, Ito M, Fujisaki S, et al. Therapeutic efficacy of monoclonal antibodies and antivirals against SARS-CoV-2 Omicron B A.1 in Syrian hamsters. Nature microbiology. 2022; 7(8):1252-8. Epub 20220615. doi: 10.1038/s41564-022-01170-4. PubMed PMID: 35705860.
7. Li F. Structure, Function, and Evolution of Coronavirus Spike Proteins. Annual review of virology. 2016; 3(1):237-61. Epub 2016/09/01. doi: 10.1146/annurev-virology-110615-042301. PubMed PMID: 27578435.
8. Du L Y, He Y X, Zhou Y S, Liu S W, Zheng B J, Jiang S B. The spike protein of SARS-CoV—a target for vaccine and therapeutic development. Nature Reviews Microbiology. 2009; 7(3):226-36. doi: 10.1038/nrmicro2090. PubMed PMID: ISI:000263361000014.
9. Wan Y, Shang J, Graham R, Baric R S, Li F. Receptor recognition by the novel coronavirus from Wuhan: an analysis based on decade-long structural studies of SARS coronavirus. Journal of virology. 2020; 94(7):10.1128/jvi. 00127-20.
10. Bajpai P, Singh V, Chandele A, Kumar S. Broadly Neutralizing Antibodies to SARS-CoV-2 Provide Novel Insights Into the Neutralization of Variants and Other Human Coronaviruses. Front Cell Infect Microbiol. 2022; 12:928279. Epub 20220616. doi: 10.3389/fcimb.2022.928279. PubMed PMID: 35782120; PubMed Central PMCID: PMCPMC9245455.
11. Hao A, Song W, Li C, Zhang X, Tu C, Wang X, et al. Defining a highly conserved cryptic epitope for antibody recognition of SARS-CoV-2 variants. Signal transduction and targeted therapy. 2023; 8(1):269. Epub 20230708. doi: 10.1038/s41392-023-01534-0. PubMed PMID: 37422451; PubMed Central PMCID: PMCPMC10329685.
12. Zhao F, Zai X, Zhang Z, Xu J, Chen W. Challenges and developments in universal vaccine design against SARS-CoV-2 variants. NPJ vaccines. 2022; 7(1):167. Epub 20221219. doi: 10.1038/s41541-022-00597-4. PubMed PMID: 36535982; PubMed Central PMCID: PMCPMC9761649.
13. Huang X, Kon E, Han X, Zhang X, Kong N, Mitchell M J, et al. Nanotechnology-based strategies against SARS-CoV-2 variants. Nat Nanotechnol. 2022; 17(10):1027-37. Epub 20220818. doi: 10.1038/s41565-022-01174-5. PubMed PMID: 35982317.
14. Konning D, Zielonka S, Grzeschik J, Empting M, Valldorf B, Krah S, et al. Camelid and 50 shark single domain antibodies: structural features and therapeutic potential. Curr Opin Struct Biol. 2017; 45:10-6. Epub 2016/11/20. doi: 10.1016/j.sbi.2016.10.019. PubMed PMID: 27865111.
15. De Meyer T, Muyldermans S, Depicker A. Nanobody-based products as research and diagnostic tools. Trends Biotechnol. 2014; 32(5):263-70. Epub 2014/04/05. doi: 10.1016/j.tibtech.2014.03.001. PubMed PMID: 24698358.
16. Du L, Yang Y, Zhang X, Li F. Recent advances in nanotechnology-based COVID-19 vaccines and therapeutic antibodies. Nanoscale. 2022; 14(4):1054-74. Epub 20220127. doi: 10.1039/d1nr03831a. PubMed PMID: 35018939; PubMed Central PMCID: PMCPMC8863106.
17. Muyldermans S. Nanobodies: natural single-domain antibodies. Annu Rev Biochem. 2013; 82:775-97. Epub 2013/03/19. doi: 10.1146/annurev-biochem-063011-092449. PubMed PMID: 23495938.
18. Vanlandschoot P, Stortelers C, Beirnaert E, Ibanez L I, Schepens B, Depla E, Saelens X. Nanobodies: new ammunition to battle viruses. Antiviral Res. 2011; 92(3):389-407. Epub 20110910. doi: 10.1016/j.antiviral.2011.09.002. PubMed PMID: 21939690.
19. Steeland S, Vandenbroucke R E, Libert C. Nanobodies as therapeutics: big opportunities for small antibodies. Drug discovery today. 2016; 21(7):1076-113. Epub 2016/04/16. doi: 10.1016/j.drudis.2016.04.003. PubMed PMID: 27080147.
20. Arbabi Ghahroudi M, Desmyter A, Wyns L, Hamers R, Muyldermans S. Selection and identification of single domain antibody fragments from camel heavy-chain antibodies. FEBS Lett. 1997; 414(3):521-6. doi: 10.1016/s0014-5793(97)01062-4. PubMed PMID: 9323027.
21. Ye G, Gallant J, Zheng J, Massey C, Shi K, Tai W, et al. The development of Nanosota-1 as anti-SARS-CoV-2 nanobody drug candidates. eLife. 2021; 10:e64815.
22. Ye G, Pan R, Bu F, Zheng J, Mendoza A, Wen W, et al. Discovery of Nanosota-2,-3, and -4 as super potent and broad-spectrum therapeutic nanobody candidates against COVID-19. Journal of Virology. 2023:e01448-23.
23. Geng Q, Wan Y, Hsueh F-C, Shang J, Ye G, Bu F, et al. Lys417 acts as a molecular switch that regulates the conformation of SARS-CoV-2 spike protein. eLife. 2023; 12:e74060.
24. Shang J, Wan Y, Luo C, Ye G, Geng Q, Auerbach A, Li F. Cell entry mechanisms of SARS-CoV-2. Proceedings of the National Academy of Sciences. 2020; 117(21):11727-34.
25. Kinoshita S, Nakakido M, Mori C, Kuroda D, Caaveiro JMM, Tsumoto K. Molecular basis for thermal stability and affinity in a VHH: Contribution of the framework region and its influence in the conformation of the CDR3. Protein science: a publication of the Protein Society. 2022; 31 (11):e4450. doi: 10.1002/pro.4450. PubMed PMID: 36153698; PubMed Central PMCID: PMCPMC9601775.
26. Valdes-Tresanco M S, Valdes-Tresanco M E, Molina-Abad E, Moreno E. NbThermo: a new thermostability database for nanobodies. Database (Oxford). 2023; 2023. doi: 10.1093/database/baad021. PubMed PMID: 37042467; PubMed Central PMCID: PMCPMC10091358.
27. Atwell S, Ultsch M, De Vos A M, Wells J A. Structural plasticity in a remodeled protein-protein interface. Science. 1997; 278(5340):1125-8. doi: 10.1126/science.278.5340.1125. PubMed PMID: 9353194.
28. Matthews J M. Plasticity at Protein-Protein Interaction Interfaces. In: Roberts GCK, editor. Encyclopedia of Biophysics. Berlin, Heidelberg: Springer Berlin Heidelberg; 2013. p. 1886-8.
29. Koide A, Koide S. Affinity maturation of single-domain antibodies by yeast surface display. Methods in molecular biology (Clifton, N J). 2012; 911:431-43. doi: 10.1007/978-1-61779-968-6_26. PubMed PMID: 22886267.
30. Schoof M, Faust B, Saunders R A, Sangwan S, Rezelj V, Hoppe N, et al. An ultrapotent synthetic nanobody neutralizes SARS-CoV-2 by stabilizing inactive Spike. Science. 2020; 370(6523):1473-9. Epub 2020/11/07. doi: 10.1126/science.abe3255. PubMed PMID: 33154106; PubMed Central PMCID: PMCPMC7857409.
31. Lipovsek D, Lippow S M, Hackel B J, Gregson M W, Cheng P, Kapila A, Wittrup K D. Evolution of an interloop disulfide bond in high-affinity antibody mimics based on fibronectin type III domain and selected by yeast surface display: molecular convergence with single-domain camelid and shark antibodies. J Mol Biol. 2007; 368(4):1024-41. Epub 20070222. doi: 10.1016/j.jmb.2007.02.029. PubMed PMID: 17382960.
32. Jacobs J L, Haidar G, Mellors J W. COVID-19: Challenges of Viral Variants. Annu Rev Med. 2023; 74:31-53. Epub 20220718. doi: 10.1146/annurev-med-042921-020956. PubMed PMID: 35850493.
33. Vasireddy D, Vanaparthy R, Mohan G, Malayala S V, Atluri P. Review of COVID-19 Variants and COVID-19 Vaccine Efficacy: What the Clinician Should Know?J Clin Med Res. 2021; 13(6):317-25. Epub 20210625. doi: 10.14740/jocmr4518. PubMedPMID: 34267839; PubMed Central PMCID: PMCPMC8256910.
34. Gong W, Parkkila S, Wu X, Aspatwar A. SARS-CoV-2 variants and COVID-19 vaccines: Current challenges and future strategies. Int Rev Immunol. 2023; 42(6):393-414. Epub 20220528. doi: 10.1080/08830185.2022.2079642. PubMed PMID: 35635216.
35. Yang Y, Li F, Du L. Therapeutic nanobodies against SARS-CoV-2 and other pathogenic human coronaviruses. J Nanobiotechnology. 2024; 22(1):304. Epub 20240531. doi: 10.1186/s12951-024-02573-7. PubMed PMID: 38822339; PubMed Central PMCID: PMCPMC 11140877.
36. Geng Q, Tai W, Baxter V K, Shi J, Wan Y, Zhang X, et al. Novel virus-like nanoparticle vaccine effectively protects animal model from SARS-CoV-2 infection. PLoS Pathog. 2021; 17 (9):e1009897.
37. Punjani A, Rubinstein J L, Fleet D J, Brubaker M A. cryoSPARC: algorithms for rapid unsupervised cryo-E M structure determination. Nature methods. 2017; 14(3):290-6. Epub 2017/02/07. doi: 10.1038/nmeth.4169. PubMed PMID: 28165473.
38. Rubinstein J L, Brubaker M A. Alignment of cryo-E M movies of individual particles by optimization of image translations. Journal of structural biology. 2015; 192(2):188-95. Epub 2015/08/25. doi: 10.1016/j.jsb.2015.08.007. PubMed PMID: 26296328.
39. Rohou A, Grigorieff N. CTFFIND4: Fast and accurate defocus estimation from electron micrographs. Journal of structural biology. 2015; 192(2):216-21. Epub 2015/08/19. doi: 10.1016/j.jsb.2015.08.008. PubMed PMID: 26278980; PubMed Central PMCID: PMCPMC6760662.
40. Zivanov J, Nakane T, Scheres SHW. Estimation of high-order aberrations and anisotropic magnification from cryo-E M data sets in RELION-3.1. IUCrJ. 2020; 7 (Pt 2):253-67. Epub 2020/03/10. doi: 10.1107/s2052252520000081. PubMed PMID: 32148853; PubMed Central PMCID: PMCPMC7055373.
41. Emsley P, Cowtan K. Coot: model-building tools for molecular graphics. Acta Crystallographica Section D-Biological Crystallography. 2004; 60:2126-32. doi: 10.1107/s0907444904019158. PubMed PMID: WOS:000225360500002.
42. Adams P D, Afonine P V, Bunkoczi G, Chen V B, Davis I W, Echols N, et al. PHENIX: a comprehensive Python-based system for macromolecular structure solution. Acta Crystallographica Section D-Biological Crystallography. 2010; 66:213-21. doi: 10.1107/s0907444909052925. PubMed PMID: ISI:000273820800013.
43. Goddard T D, Huang C C, Meng E C, Pettersen E F, Couch G S, Morris J H, Ferrin T E. UCSF ChimeraX: Meeting modern challenges in visualization and analysis. Protein science: a publication of the Protein Society. 2018; 27(1):14-25. Epub 2017/07/16. doi: 10.1002/pro.3235. PubMed PMID: 28710774; PubMed Central PMCID: PMCPMC5734306.
44. DeLano W L. PyMOL: an open-source molecular graphics tool. CCP4 Newsl Protein Crystallogr. 2002; 40:82-92.

TABLE 1
Class 1 (2 clones of Nanosota-2 family, such as Nanosota-2 clone H6)

| SEQ | | |
|---|---|---|
| ID | | |
| NO: | Sequences | Comment |
| 1 | QVQLQESGGGAVQPGGSLGLSCTASGFNFETSTVGWFRQA | Nanosota-2A VHH |
| | PGKENEGVSCINKGYEDTNYADSVKGRFTISRDAAKNTVY | sequence (clone H6) |
| | LQMDSLQPEDTATYYCAAHNEPYFCDYSGRFRWNEYSYYG | |
| | QGTQVTVSS | |
| 2 | FNFETSTV | Nanosota-2A: CDR- |
| | | H1 (clone H6) |
| 3 | CINKGYEDTN | Nanosota-2A: CDR- |
| | | H2 (clone H6) |
| 4 | AAHNEPYFCDYSGRFRWNEYSY | Nanosota-2A: CDR- |
| | | H3 (clone H6) |
| 5 | QVQLQESGGGAVQPGGSLGLSCTASGFNFETSTVGWFRQA | Nanosota-2A (clone |
| | PGKENEGVSCINKGYEDTNYADSVKGRFTISRDAAKNTVY | H6) VHH sequence |
| | LQMDSLQPEDTATYYCAAHNEPYFCDYSGRFRWNEYSYYG | with linker |
| | QGTQVTVSSGSHHHHHH | (bold) and His₆ tag |
| | | (SEQ ID NO: 20) |
| | | (italics) |
| 31 | QVQLQESGGGAVQPGGSLGLSCTASGFNFETSTVGWFRQA | (clone D4) |
| | PGKEYEGVSCINKGYEDTNYADSVKGRFTISRDAAKNTVY | |
| | LQMDSLQPEDTATYYCAAHNEPYFCDYSGRFRWNEYSYYG | |
| | QGTQVTVSS | |
| 2 | FNFETSTV | CDR-H1 (clone D4) |
| 3 | CINKGYEDTN | CDR-H2 (clone D4) |
| 4 | AAHNEPYFCDYSGRFRWNEYSY | CDR-H3 (clone D4) |
| 32 | QVQLQESGGGAVQPGGSLGLSCTASGFNFFTSTVGWFRQA | (clone D4) VHH |
| | PGKEYEGVSCINKGYEDTNYADSVKGRFTISRDAAKNTVY | sequence with linker |
| | LQMDSLQPEDTATYYCAAHNEPYFCDYSGRFRWNEYSYYG | (bold) and His₆ |
| | QGTQVTVSSGGQHHHHHHGAYPYDVPDYAS | tag (SEQ ID NO: 20) / |
| | | HA tag (italics) |

TABLE 2
Class 2 (6 clones of Nanosota-3 family, such as Nanosota-3 clone A3)

| SEQ | | |
|---|---|---|
| ID | | |
| NO: | Sequences | Comment |
| 7 | QVQLQESGGGLVQAGGSLRLSCAASGSIFSPNTMGWFRQA | Nanosota-3A VHH |
| | LGKQREMVAVISSIASTQYANFVKGRFTITRDNTKNTVHL | sequence (clone A3) |
| | QMNSLIPEDTAVYYCYAVDKSQDYWGQGTQVTVSS | |
| 8 | SIFSPNTM | Nanosota-3A (clone |
| | | A3): CDR-H1 |
| 9 | VISSIASTQ | Nanosota-3A (clone |
| | | A3): CDR-H2 |
| 10 | YAVDKSQDY | Nanosota-3A (clone |
| | | A3): CDR-H3 |
| 11 | QVQLQESGGGLVQAGGSLRLSCAASGSIFSPNTMGWFRQA | Nanosota-3A (clone |
| | LGKQREMVAVISSIASTQYANFVKGRFTITRDNTKNTVHL | A3) VHH sequence with |
| | QMNSLIPEDTAVYYCYAVDKSQDYWGQGTQVTVSSGSHHH | linker (bold) and |
| | HHH | His₆ tag (SEQ ID |
| | | NO: 20) (italics) |
| 156 | QVQLQESGGGLVQAGGSLRLSCAASGSIFSPNTMGWFRQA | Nanosota-3B VHH |
| | LGKQREMVAFISSIASTSYANFVKGRFTITRDNTKNTVHL | sequence that has |
| | QMNSLIPEDTAVYYCYAVDKSQDYWGQGTQVTVSS | two mutations |
| | | (bold) as compared to |
| | | clone A3 |
| 8 | SIFSPNTM | Nanosota-3B: CDR-H1 |
| 157 | FISSIASTS | Nanosota-3B: CDR-H2 |
| 10 | YAVDKSQDY | Nanosota-3B: CDR-H3 |
| 158 | QVQLQESGGGLVOAGGSLRLSCAASGSIFSPNTMGWFRQA | Nanosota-3B VHH |
| | LGKQREMVAFISSIASTSYANFVKGRFTITRDNTKNTVHL | sequence with linker |
| | QMNSLIPEDTAVYYCYAVDKSQDYWGQGTQVTVSSGSHHH | (bold) and His₆ tag |
| | HHH | (SEQ ID NO: 20) |
| | | (italics) |
| 172 | QVQLQESGGGLVQAGGSLRLSCAASGSIFSPNTMGWFRQA | Nanosota-3B-Fc |
| | LGKQREMVAFISSIASTSYANFVKGRFTITRDNTKNTVHL | Nanosota-3B VHH |
| | QMNSLIPEDTAVYYCYAVDKSQDYWGQGTQVTVSSEPKSC | sequence with IgG1 |
| | DKTHTCPPCPAPELLGGPSVFLFPPKPKDTLMISRTPEVT | Fc domain sequence |
| | CVVVDVSHEDPEVKFNWYVDGVEVHNAKTKPREEQYNSTY | (bold) |
| | RVVSVLTVLHQDWLNGKEYKCKVSNKALPAPIEKTISKAK | |
| | GQPREPQVYTLPPSREEMTKNQVSLTCLVKGFYPSDIAVE | |
| | WESNGQPENNYKTTPPVLDSDGSFFLYSKLTVDKSRWQQG | |
| | NVFSCSVMHEALHNHYTQKSLSLSPGK | |
| 167 | QVQLQESGGGLVQAGGSLRLSCAASGSIFSPNTMGWFRQA | Nanosota-3C VHH |
| | LGKQREGVAFISSIASTSYWLPVKGRFTITRDNTKNTVHL | sequence has the |
| | QMNSLIPEDTAVYYCYAVDKSQDYWGQGTQVTVSS | same CDRs of |
| | | Nanosota-3B but has |
| | | four framework |
| | | region mutations as |
| | | compared to |
| | | Nanosota-3B. |
| | | Nanosota-3C has six |
| | | mutations in total |
| | | (bold) as compared to |
| | | Nanosota-3A (clone |
| | | A3) |
| 8 | SIFSPNTM | Nanosota-3C: CDR-H1 |
| 157 | FISSIASTS | Nanosota-3C: CDR-H2 |
| 10 | YAVDKSQDY | Nanosota-3C: CDR-H3 |
| 171 | QVQLQESGGGLVQAGGSLRLSCAASGSIFSPNTMGWFRQA | Nanosota-3C VHH |
| | LGKQREGVAFISSIASTSYWLPVKGRFTITRDNTKNTVHL | sequence with linker |
| | QMNSLIPEDTAVYYCYAVDKSQDYWGQGTQVTVSSGGQHH | (bold) and His₆ tag |
| | HHHHGAYPYDVPDYAS | (SEQ ID NO: 20) / HA |
| | | tag (italics) |
| 173 | QVQLQESGGGLVQAGGSLRLSCAASGSIFSPNTMGWFRQA | Nanosota-3C-Fc |
| | LGKQREGVAFISSIASTSYWLPVKGRFTITRDNTKNTVHL | Nanosota-3C VHH |
| | QMNSLIPEDTAVYYCYAVDKSQDYWGQGTQVTVSSEPKSC | sequence with IgG1 |
| | DKTHTCPPCPAPELLGGPSVFLFPPKPKDTLMISRTPEVT | Fc domain sequence |
| | CVVVDVSHEDPEVKFNWYVDGVEVHNAKTKPREEQYNSTY | (bold) |
| | RVVSVLTVLHQDWLNGKEYKCKVSNKALPAPIEKTISKAK | |
| | GQPREPQVYTLPPSREEMTKNQVSLTCLVKGFYPSDIAVE | |
| | WESNGQPENNYKTTPPVLDSDGSFFLYSKLTVDKSRWQQG | |
| | NVFSCSVMHEALHNHYTQKSLSLSPGK | |
| 168 | QVQLQESGGGLVQAGGSLRLSCAASGSIFSPNTMGWFRQA | Nanosota-3C-2 VHH |
| | LGKQREGVAFISSIASTSYNWYVKGRFTITRDNTKNTVHL | sequence has same |
| | QMNSLIPEDTAVYYCYAVDKSQDYWGQGTQVTVSS | CDRs of Nanosota-3C |
| | | but has three |
| | | different residues |
| | | at 60, 61, 62 |
| | | positions as |
| | | compared to |
| | | Nanosota-3C |
| 169 | QVQLQESGGGLVQAGGSLRLSCAASGSIFSPNTMGWFRQA | Nanosota-3C-3 VHH |
| | LGKQREGVAFISSIASTSYNFFVKGRFTITRDNTKNTVHL | sequence has same |
| | QMNSLIPEDTAVYYCYAVDKSQDYWGQGTQVTVSS | CDRs of Nanosota-3C |
| | | but has three |
| | | different residues |
| | | at 60, 61, 62 |
| | | positions as |
| | | compared to |
| | | Nanosota-3C |
| 170 | QVQLQESGGGLVQAGGSLRLSCAASGSIFSPNTMGWFRQA | Nanosota-3C-4 VHH |
| | LGKQREGVAFISSIASTSYGYGVKGRFTITRDNTKNTVHL | sequence has same |
| | QMNSLIPEDTAVYYCYAVDKSQDYWGQGTQVTVSS | CDRs of Nanosota-3C |
| | | but has three |
| | | different residues |
| | | at 60, 61, 62 |
| | | positions as |
| | | compared to |
| | | Nanosota-3C |
| 33 | QVQLQESGGGLVQAGGPLRLSCVASASTSASNSMSWERQA | (clone B3) VHH |
| | PGKQREWVATAANGDIRSYANFVKGRFTISRDDAKNTVYL | sequence |
| | QMNGLKPEDTAVYYCYSVDSYRDYWGQGTQVTVSS | |
| 34 | STSASNSM | (clone B3): CDR-H1 |
| 35 | TAANGDIRS | (clone B3): CDR-H2 |
| 36 | YSVDSYRDY | (clone B3): CDR-H3 |
| 37 | QVQLQESGGGLVQAGGPLRLSCVASASTSASNSMSWFRQA | (clone B3) VHH |
| | PGKQREWVATAANGDIRSYANFVKGRFTISRDDAKNTVYL | sequence with linker |
| | QMNGLKPEDTAVYYCYSVDSYRDYWGQGTQVTVSSGGQHH | (bold) and His₆ tag |
| | HHHHGAYPYDVPDYAS | (SEQ ID NO: 20) / HA |
| | | tag (italics) |
| 7 | QVQLQESGGGLVQAGGSLRLSCAASGSIFSPNTMGWFRQA | (clone E2) VHH |
| | LGKQREMVAVISSIASTQYANFVKGRFTITRDNTKNTVHL | sequence |
| | QMNSLIPEDTAVYYCYAVDKSQDYWGQGTQVTVSS | |
| 8 | SIFSPNTM | (clone E2): CDR-H1 |
| 9 | VISSIASTQ | (clone E2): CDR-H2 |
| 10 | YAVDKSQDY | (clone E2): CDR-H3 |
| 39 | QVQLQESGGGLVQAGGSLRLSCAASGSIFSPNTMGWFRQA | (clone E2) VHH |
| | LGKQREMVAVISSIASTQYANFVKGRFTITRDNTKNTVHL | sequence with linker |
| | QMNSLIPEDTAVYYCYAVDKSQDYWGQGTQVTVSSGGQHH | (bold) and His₆ tag |
| | HHHHGAYPYDVPDYAS | (SEQ ID NO: 20) / HA |
| | | tag (italics) |
| 7 | QVQLQESGGGLVQAGGSLRLSCAASGSIFSPNTMGWFRQA | (clone D3) VHH |
| | LGKQREMVAVISSIASTQYANFVKGRFTITRDNTKNTVHL | sequence |
| | QMNSLIPEDTAVYYCYAVDKSQDYWGQGTQVTVSS | |
| 8 | SIFSPNTM | (clone D3): CDR-H1 |
| 9 | VISSIASTO | (clone D3): CDR-H2 |
| 10 | YAVDKSQDY | (clone D3): CDR-H3 |
| 39 | QVQLQESGGGLVQAGGSLRLSCAASGSIFSPNTMGWFRQA | (clone D3) VHH |
| | LGKQREMVAVISSIASTQYANFVKGRFTITRDNTKNTVHL | sequence with linker |
| | QMNSLIPEDTAVYYCYAVDKSQDYWGQGTQVTVSSGGQHH | (bold) and His₆ |
| | HHHHGAYPYDVPDYAS | tag (SEQ ID NO: 20) / |
| | | HA tag (italics) |
| 7 | QVQLQESGGGLVQAGGSLRLSCAASGSIFSPNTMGWFRQA | (clone E5) VHH |
| | LGKQREMVAVISSIASTQYANFVKGRFTITRDNTKNTVHL | sequence |
| | QMNSLIPEDTAVYYCYAVDKSQDYWGQGTQVTVSS | |
| 8 | SIFSPNTM | (clone E5): CDR-H1 |
| 9 | VISSIASTQ | (clone E5): CDR-H2 |
| 10 | YAVDKSQDY | (clone E5): CDR-H3 |
| 39 | QVQLQESGGGLVQAGGSLRLSCAASGSIFSPNTMGWFRQA | (clone E5) VHH |
| | LGKQREMVAVISSIASTQYANFVKGRFTITRDNTKNTVHL | sequence with linker |
| | QMNSLIPEDTAVYYCYAVDKSQDYWGQGTQVTVSSGGQHH | (bold) and His₆ tag |
| | HHHHGAYPYDVPDYAS | (SEQ ID NO: 20) / HA |
| | | tag (italics) |
| 44 | QVQLQESGGGLVQPGGSLRLSCAASGSIFSPNTMGWFRQA | (clone A12) VHH |
| | LGKQREMVAVISSIASTQYANFVKGRFTITRDNTKNTVHL | sequence |
| | QMNSLIPEDTAVYYCYAVDKSQDYWGQGTQVTVSS | |
| 8 | SIFSPNTM | (clone A12): CDR- |
| | | H1 |
| 9 | VISSIAST1 | (clone A12): CDR- |
| | | H2 |
| 10 | YAVDKSQDY | (clone A12): CDR- |
| | | H3 |
| 45 | QVQLQESGGGLVQPGGSLRLSCAASGSIFSPNTMGWFRQA | (clone A12) VHH |
| | LGKQREMVAVISSIASTQYANFVKGRFTITRDNTKNTVHL | sequence with linker |
| | QMNSLIPEDTAVYYCYAVDKSQDYWGQGTQVTVSSGGQHH | (bold) and His₆ tag |
| | HHHHGAYPYDVPDYAS | (SEQ ID NO: 20) / HA |
| | | tag (italics) |
| 159 | (V/F) ISSIAST (Q/S) | CDR-H2 |
| 148 | Y (A/S) VD (K/S) (S/Y) (Q/R) DY | CDR-H3 |

TABLE 3
Class 3 (18 clones of Nanosota-4 family, such as Nanosota-4A clone E8)

| SEQ | | |
|---|---|---|
| ID | | |
| NO: | Sequences | Comment |
| 13 | QVQLQESGGGLVQPGGSLRLSCAASGFTLDYYAIGWFRQA | Nanosota-4A VHH |
| | PGKEREGVSCISSSGGRTNYADSVKGRFTISRDNTKNTVY | sequence (clone E8) |
| | LQMNSLKPEDTAVYYCAAWEASRWYCPLQFSADESSWGQG | |
| | TQVTVSS | |
| 14 | FTLDYYAI | Nanosota-4A (clone |
| | | E8): CDR-H1 |
| 15 | CISSSGGRTN | Nanosota-4A (clone |
| | | E8): CDR-H2 |
| 16 | AAWEASRWYCPLQFSADFSS | Nanosota-4A (clone |
| | | E8): CDR-H3 |
| 17 | QVQLQESGGGLVQPGGSLRLSCAASGFTLDYYAIGWFRQA | Nanosota-4A (clone |
| | PGKEREGVSCISSSGGRTNYADSVKGRFTISRDNTKNTVY | E8) VHH sequence with |
| | LQMNSLKPEDTAVYYCAAWEASRWYCPLQFSADFSSWGQG | linker (bold) and |
| | TQVTVSSGSHHHHHH | His₆ tag (SEQ ID |
| | | NO: 20) (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | A6) |
| | LQMNSLKPEDTAVYSCAAWEGSTRYCPIQTSADFVSWGQG | |
| | TQVTVSS | |
| 47 | FILDFYAI | (clone A6): CDR-H1 |
| 15 | CISSSGGRTN | (clone A6): CDR-H2 |
| 48 | AAWEGSTRYCPIQTSADFVS | (clone A6): CDR-H3 |
| 49 | QVQLQESGGGLVQPGGSLRLSCAASGFILDFYAIGWFRQA | (clone A6) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ |
| | TQVTVSSGGQHHHHHHGAYPYDVPDYAS | tag (SEQ ID NO: 20)/ |
| | | HA tag (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | A7) |
| | LQMNSLKPEDTAVYSCAAWEGSTRYCPIQTSADFVSWGQG | |
| | TQVTVSS | |
| 47 | FILDFYAI | (clone A7): CDR-H1 |
| 15 | CISSSGGRTN | (clone A7): CDR-H2 |
| 48 | AAWEGSTRYCPIQTSADFVS | (clone A7): CDR-H3 |
| 49 | QVQLQESGGGLVQPGGSLRLSCAASGFILDFYAIGWFRQA | (clone A7) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ |
| | TQVTVSSGGQHHHHHHGAYPYDVPDYAS | tag (SEQ ID NO: 20)/ |
| | | HA tag (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | C9) |
| | LQMNSLKPEDTAVYSCAAWEGSTRYCPIQTSADFVSWGQG | |
| | TQVTVSS | |
| 47 | FILDFYAI | (clone C9): CDR-H1 |
| 15 | CISSSGGRTN | (clone C9): CDR-H2 |
| 48 | AAWEGSTRYCPIQTSADFVS | (clone C9): CDR-H3 |
| 49 | QVQLQESGGGLVQPGGSLRLSCAASGFILDFYAIGWFRQA | (clone C9) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ |
| | TQVTVSSGGQHHHHHHGAYPYDVPDYAS | tag (SEQ ID NO: 20)/ |
| | | HA tag (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | D5) |
| | LQMNSLKPEDTAVYSCAAWEGSTRYCPIQTSADFVSWGQG | |
| | TQVTVSS | |
| 47 | FILDFYAI | (clone D5): CDR-H1 |
| 15 | CISSSGGRTN | (clone D5): CDR-H2 |
| 48 | AAWEGSTRYCPIQTSADFVS | (clone D5): CDR-H3 |
| 49 | QVQLQESGGGLVQPGGSLRLSCAASGFILDFYAIGWFRQA | (clone D5) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ tag |
| | TQVTVSSGGQHHHHHHGAYPYDVPDYAS | (SEQ ID NO: 20)/HA |
| | | tag (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | F10) |
| | LQMNSLKPEDTAVYSCAAWEGSTRYCPIQTSADFVSWGQG | |
| | TQVTVSS | |
| 47 | FILDFYAI | (clone F10): CDR- |
| | | H1 |
| 15 | CISSSGGRTN | (clone F10): CDR- |
| | | H2 |
| 48 | AAWEGSTRYCPIQTSADFVS | (clone F10): CDR- |
| | | H3 |
| 49 | QVQLQESGGGLVQPGGSLRLSCAASGFILDFYAIGWFRQA | (clone F10) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ tag |
| | TQVTVSSGGQHHHHHHGAYPYDVPDYAS | (SEQ ID NO: 20)/ |
| | | HA tag (italics) |
| 46 | QVQLQESGGGLVQPGGSLRLSCAASGFILDFYAIGWERQA | VHH sequence (clone |
| | PGKEREGVSCISSSGGRTNYADSVKGRFTISRDNAKNTVY | F11) |
| | LQMNSLKPEDTAVYSCAAWEGSTRYCPIQTSADFVSWGQG | |
| | TQVTVSS | |
| 47 | FILDFYAI | (clone F11): CDR- |
| | | H1 |
| 15 | CISSSGGRTN | (clone F11): CDR- |
| | | H2 |
| 48 | AAWEGSTRYCPIQTSADFVS | (clone F11): CDR- |
| | | H3 |
| 49 | QVQLQESGGGLVQPGGSLRLSCAASGFILDFYAIGWFRQA | (clone F11) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ tag |
| | TQVTVSSGGQHHHHHHGAYPYDVPDYAS | (SEQ ID NO: 20)/ |
| | | HA tag (italics) |
| 46 | QVQLQESGGGLVQPGGSLRLSCAASGFILDFYAIGWFRQA | VHH sequence (clone |
| | PGKEREGVSCISSSGGRTNYADSVKGRFTISRDNAKNTVY | G4) |
| | LQMNSLKPEDTAVYSCAAWEGSTRYCPIQTSADFVSWGQG | |
| | TQVTVSS | |
| 47 | FILDFYAI | (clone G4): CDR-H1 |
| 15 | CISSSGGRTN | (clone G4): CDR-H2 |
| 48 | AAWEGSTRYCPIQTSADFVS | (clone G4): CDR-H3 |
| 49 | QVQLQESGGGLVQPGGSLRLSCAASGFILDFYAIGWFRQA | (clone G4) VHH |
| | PGKEREGVSCISSSGGRTNYADSVKGRFTISRDNAKNTVY | sequence with linker |
| | LQMNSLKPEDTAVYSCAAWEGSTRYCPIQTSADFVSWGQG | (bold) and His₆ tag |
| | TQVTVSSGGQHHHHHHGAYPYDVPDYAS | (SEQ ID NO: 20)/ |
| | | HA tag (italics) |
| 62 | QVQLQESGGGLVQPGGSLRLSCAASGFILDFYAIGWFRQA | VHH sequence (clone |
| | PGKEREGVSCISSSGGRTNYADSVKGRFTISSDNAKNTVY | D7) |
| | LQMNSLKPEDTAVYSCAAWEGSTRYCPIQTSADFVSWGQG | |
| | TQVTVSS | |
| 47 | FILDFYAI | (clone D7): CDR-H1 |
| 15 | CISSSGGRTN | (clone D7): CDR-H2 |
| 48 | AAWEGSTRYCPIQTSADFVS | (clone D7): CDR-H3 |
| 63 | QVQLQESGGGLVQPGGSLRLSCAASGFILDFYAIGWFRQA | (clone D7) VHH |
| | PGKEREGVSCISSSGGRTNYADSVKGRFTISSDNAKNTVY | sequence with linker |
| | LQMNSLKPEDTAVYSCAAWEGSTRYCPIQTSADFVSWGQG | (bold) and His₆ tag |
| | TQVTVSSGGQHHHHHHGAYPYDVPDYAS | (SEQ ID NO: 20)/ |
| | | HA tag (italics) |
| 64 | QVQLQESGGGLVQAGGSLRLTCVASGTTLDHYAIGWLRQA | VHH sequence (clone |
| | PGKEREGVSCISSSGGSTNYADSVKGRFTISRDNAKNTVY | G8) |
| | LQMTGLKPEDTAVYYCAAWEGSSEYCPLQFSADFASWGRG | |
| | TQVTVSS | |
| 65 | TTLDHYAI | (clone G8): CDR-H1 |
| 66 | CISSSGGSTN | (clone G8): CDR-H2 |
| 67 | AAWEGSSEYCPLQFSADFAS | (clone G8): CDR-H3 |
| 68 | QVQLQESGGGLVQAGGSLRLTCVASGTTLDHYAIGWLRQA | (clone G8) VHH |
| | PGKEREGVSCISSSGGSTNYADSVKGRFTISRDNAKNTVY | sequence with linker |
| | LQMTGLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 69 | QVQLQESGGGLVQDGGSLRLSCEASGFTVNSHAIGWFRQS | VHH sequence (clone |
| | PGKEREGVSCISSSGGSTNYADSVKGRFTISRDNAKNTVY | G3) |
| | LQMTSLKPEDTAVYYCAAWEGSSEYCPLQFSADFASWGRG | |
| | TQVTVSS | |
| 70 | FTVNSHAI | (clone G3): CDR-H1 |
| 66 | CISSSGGSTN | (clone G3): CDR-H2 |
| 67 | AAWEGSSEYCPLQFSADFAS | (clone G3): CDR-H3 |
| 71 | QVQLQESGGGLVQDGGSLRLSCEASGFTVNSHAIGWFRQS | (clone G3) VHH |
| | PGKEREGVSCISSSGGSTNYADSVKGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 72 | QVQLQECGGGLVQPGGSLRLSCAASGFTLDYYAIGWFRQA | VHH sequence (clone |
| | PGKEREGVSCISSSGGSTNYADSVKGRFTISRDNAKNTVY | F9) |
| | LQMTSLKPEDTAVYYCAAWEGSSEYCPLQYSADFDSWGQG | |
| | TQVTVSS | |
| 14 | FTLDYYAI | (clone F9): CDR-H1 |
| 66 | CISSSGGSTN | (clone F9): CDR-H2 |
| 73 | AAWEGSSEYCPLQYSADFDS | (clone F9): CDR-H3 |
| 74 | QVQLQECGGGLVQPGGSLRLSCAASGFTLDYYAIGWFRQA | (clone F9) VHH |
| | PGKEREGVSCISSSGGSTNYADSVKGRFTISRDNAKNTVY | sequence with linker |
| | LQMTSLKPEDTAVYYCAAWEGSSEYCPLQYSADFDSWGQG | (bold) and His₆ tag |
| | TQVTVSSGGQHHHHHHGAYPYDVPDYAS | (SEQ ID NO: 20)/ |
| | | HA tag (italics) |
| 75 | QVQLQESGGGLVQPGGSLRLSCAASGFALDYYAIGWERQA | VHH sequence (clone |
| | PKKEREGVSCISISGGSTNYADSVEGRFTISRDNAKNTVY | C10) |
| | LQMTSLKPEDTAVYYCAAWEGSSEYCPLQFSADFASWGRG | |
| | TQVTVSS | |
| 76 | FALDYYAI | (clone C10): CDR- |
| | | H1 |
| 77 | CISISGGSTN | (clone C10): CDR- |
| | | H2 |
| 67 | AAWEGSSEYCPLQFSADFAS | (clone C10): CDR- |
| | | H3 |
| 78 | QVQLQESGGGLVQPGGSLRLSCAASGFALDYYAIGWFRQA | (clone C10) VHH |
| | PKKEREGVSCISISGGSTNYADSVEGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 79 | QVQLQESGGGLVQPGGSLRLSCAASGFTLDYYAIGWFRQA | VHH sequence (clone |
| | PGKEREGVSCISSSGGSTNYADSVKGRFTISRDNAKNTVY | C12) |
| | LQMTSLKPEDTAVYYCAAWEGSSEYCPLQFSADFASRGQG | |
| | TQVTVSS | |
| 14 | FTLDYYAI | (clone C12): CDR- |
| | | H1 |
| 66 | CISSSGGSTN | (clone C12): CDR- |
| | | H2 |
| 67 | AAWEGSSEYCPLQFSADFAS | (clone C12): CDR- |
| | | H3 |
| 80 | QVQLQESGGGLVQPGGSLRLSCAASGFTLDYYAIGWFRQA | (clone C12) VHH |
| | PGKEREGVSCISSSGGSTNYADSVKGRFTISRDNAKNTVY | sequence with linker |
| | LQMTSLKPEDTAVYYCAAWEGSSEYCPLQFSADFASRGQG | (bold) and His₆ tag |
| | TQVTVSSGGQHHHHHHGAYPYDVPDYAS | (SEQ ID NO: 20)/ |
| | | HA tag (italics) |
| 81 | QVQLQESGGGLVQPGGSLRLFCAASGFTLDYYAIGWFRQA | VHH sequence (clone |
| | PGKEREGVSCISSSGGSTNYADSVKGRFTISRDNAKNTVY | H11) |
| | LQMTSLKPEDTAVYYCAAWEGSSEYCPLQFSADFASWGRG | |
| | TQVTVSS | |
| 14 | FTLDYYAI | (clone H11): CDR- |
| | | H1 |
| 66 | CISSSGGSTN | (clone H11): CDR- |
| | | H2 |
| 67 | AAWEGSSEYCPLQFSADFAS | (clone H11): CDR- |
| | | H3 |
| 82 | QVQLQESGGGLVQPGGSLRLFCAASGFTLDYYAIGWFRQA | (clone H11) VHH |
| | PGKEREGVSCISSSGGSTNYADSVKGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 83 | QVQLQESGGGLVQPGGSLRLSCAASGFTLDYYAIGWFRQA | VHH sequence (clone |
| | PGKEREGVSCISSSGGSTNYADSVKGRFTISRDNAKNTVY | C8) |
| | LQMTSLKPEDTAVYYCAAWEGSSEYCPLQFSADFASWGRG | |
| | TQVTVSS | |
| 14 | FTLDYYAI | (clone C8): CDR-H1 |
| 66 | CISSSGGSTN | (clone C8): CDR-H2 |
| 67 | AAWEGSSEYCPLQFSADFAS | (clone C8): CDR-H3 |
| 84 | QVQLQESGGGLVQPGGSLRLSCAASGFTLDYYAIGWFRQA | (clone C8) VHH |
| | PGKEREGVSCISSSGGSTNYADSVKGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 83 | QVQLQESGGGLVQPGGSLRLSCAASGFTLDYYAIGWFRQA | VHH sequence (clone |
| | PGKEREGVSCISSSGGSTNYADSVKGRFTISRDNAKNTVY | F2) |
| | LQMTSLKPEDTAVYYCAAWEGSSEYCPLQFSADFASWGRG | |
| | TQVTVSS | |
| 14 | FTLDYYAI | (clone F2): CDR-H1 |
| 66 | CISSSGGSTN | (clone F2): CDR-H2 |
| 67 | AAWEGSSEYCPLQFSADFAS | (clone F2): CDR-H3 |
| 84 | QVQLQESGGGLVQPGGSLRLSCAASGFTLDYYAIGWFRQA | (clone F2) VHH |
| | PGKEREGVSCISSSGGSTNYADSVKGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 87 | QVQLQESGGGLVQPGGSLRLSCAASGFTLDYYAIGWFRQA | VHH sequence (clone |
| | PGKEREGVSCISSSGGSTNYVDSVKGRFTISRDNAKNTVY | E12) |
| | LQMTSLKPEDTAVYYCAAWEGSSEYCPLQFSADFASWGRG | |
| | TQVTVSS | |
| 14 | FTLDYYAI | (clone E12): CDR- |
| | | H1 |
| 66 | CISSSGGSTN | (clone E12): CDR- |
| | | H2 |
| 67 | AAWEGSSEYCPLQFSADFAS | (clone E12): CDR- |
| | | H3 |
| 88 | QVQLQESGGGLVQPGGSLRLSCAASGFTLDYYAIGWFRQA | (clone E12) VHH |
| | PGKEREGVSCISSSGGSTNYVDSVKGRFTISRDNAKNTVY | sequence with linker |
| | LQMTSLKPEDTAVYYCAAWEGSSEYCPLQFSADFASWGRG | (bold) and His₆ tag |
| | TQVTVSSGGQHHHHHHGAYPYDVPDYAS | (SEQ ID NO: 20)/ |
| | | HA tag (italics) |
| 149 | F(T/I/A)LD(Y/F/H)YAI | CDR-H1 |
| 150 | CIS(S/I)SGG(R/S)TN | CDR-H2 |
| 151 | AAWE(A/G)S(R/T/S)(W/R/E)YCPLQ(F/T/Y)SADF | CDR-H3 |
| | (S/V/A/D)S | |

TABLE 4
Class 4 (18 clones of Nanosota-5 family, such as Nanosota-5 clone E10)

| SEQ | | |
|---|---|---|
| ID | | |
| NO: | Sequences | Comment |
| 89 | QVQLQESGGGLVQAGGSLRLSCVASGSIFRFEAVGWYRQA | (clone A2) |
| | PGKQRELVATVARDGTTNYADSVKGRFTISTDNAKNSVYL | |
| | QMNSLKAEDTAVYVCNARWWTNFWGQGTQVTVSS | |
| 90 | SIFRFEAV | (clone A2): CDR-H1 |
| 91 | TVARDGTTN | (clone A2): CDR-H2 |
| 92 | NARWWTNF | (clone A2): CDR-H3 |
| 93 | QVQLQESGGGLVQAGGSLRLSCVASGSIFRFEAVGWYRQA | (clone A2) VHH |
| | PGKQRELVATVARDGTTNYADSVKGRFTISTDNAKNSVYL | sequence with linker |
| | QMNSLKAEDTAVYVCNARWWTNFWGQGTQVTVSSGGQHHH | (bold) and His₆ tag |
| | HHHGAYPYDVPDYAS | (SEQ ID NO: 20)/HA |
| | | tag (italics) |
| 94 | QVQLQESGGGLVQAGGSLRLSCAASESIFRMDVVQWYRQA | VHH sequence (clone |
| | PGKQRELVASITRSGSTNYADSVKGRFIISSDNAKNSVYL | E6 |
| | QMKSLKVEDTAVYLCHARTWTSYWGQGTQVTVSS | |
| 95 | SIFRMDVV | (clone E6): CDR-H1 |
| 96 | SITRSGSTN | (clone E6): CDR-H2 |
| 97 | HARTWTSY | (clone E6): CDR-H3 |
| 98 | QVQLQESGGGLVQAGGSLRLSCAASESIFRMDVVQWYRQA | (clone E6) VHH |
| | PGKQRELVASITRSGSTNYADSVKGRFIISSDNAKNSVYL | sequence with linker |
| | QMKSLKVEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 99 | QVQLQESGGGLVQPGGSLRLSCAASESIFRMDVVQWYRQA | Nanosota-5 VHH |
| | PGKQRELVASITRSGSTNYADSVKGRFIISSDNAKNSVYL | sequence (clone F6) |
| | QMKSLKVEDTAVYLCHARTWTSYWGQGTQVTVSS | |
| 95 | SIFRMDVV | (clone F6): CDR-H1 |
| 96 | SITRSGSTN | (clone F6): CDR-H2 |
| 97 | HARTWTSY | (clone F6): CDR-H3 |
| 100 | QVQLQESGGGLVQPGGSLRLSCAASESIFRMDVVQWYRQA | (clone F6) VHH |
| | PGKQRELVASITRSGSTNYADSVKGRFIISSDNAKNSVYL | sequence with linker |
| | QMKSLKVEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 101 | QVQLQESGGGLVQAGGSLRLSCAASESIFRMELMEWYHQA | Nanosota-5^{S} VHH |
| | PGKQRELVATINRCGSTNYSDSVKGRFIISSDNAKNSVYL | sequence |
| | QMNSLKDEDTAVYSCHARTWTSSWGRGTQVTVSS | |
| 102 | SIFRMELM | Nanosota-5^{S}: CDR-H1 |
| 103 | TINRCGSTN | Nanosota-5^{S}: CDR-H2 |
| 104 | HARTWTSS | Nanosota-5^{S}: CDR-H3 |
| 105 | QVQLQESGGGLVQAGGSLRLSCAASESIFRMELMEWYHQA | Nanosota-5^{S} VHH |
| | PGKQRELVATINRCGSTNYSDSVKGRFIISSDNAKNSVYL | sequence with linker |
| | QMNSLKDEDTAVYSCHARTWTSSWGRGTQVTVSSGGQHHH | (bold) and His₆ tag |
| | HHHGAYPYDVPDYAS | (SEQ ID NO: 20)/HA |
| | | tag (italics) |
| 152 | QVQLQESGGGLVQAGGSLRLSCAASESIFRMELMEWYHQA | Nanosota-5^{S} VHH |
| | PGKQRELVATINRCGSTNYSDSVKGRFIISSDNAKNSVYL | sequence with IgG1 |
| | QMNSLKDEDTAVYSCHARTWTSSWGRGTQVTVSSEPKSCD | Fc domain |
| | KTHTCPPCPAPELLGGPSVFLFPPKPKDTLMISRTPEVTC | sequence (bold) |
| | VVVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSREEMTKNQVSLTCLVKGFYPSDIAVEW | |
| | ESNGQPENNYKTTPPVLDSDGSFFLYSKLTVDKSRWQQGN | |
| | VFSCSVMHEALHNHYTQKSLSLSPGK | |
| 164 | QVQLQESGGGLVQAGGSLRLSCAASESIFRMELMEWYHQA | Nanosota-5 VHH |
| | PGKQRELVATINRCGSTNYSDSVKGRFIISSDNAKNSVYL | sequence (clone E10) |
| | QMNSLKDEDTAVYSCHARTWTSYWGRGTQVTVSS | |
| 102 | SIFRMELM | Nanosota-5 (clone |
| | | E10): CDR-H1 |
| 103 | TINRCGSTN | Nanosota-5 (clone |
| | | E10): CDR-H2 |
| 97 | HARTWTSY | Nanosota-5 (clone |
| | | E10): CDR-H3 |
| 165 | QVQLQESGGGLVQAGGSLRLSCAASESIFRMELMEWYHQA | Nanosota-5 (clone |
| | PGKQRELVATINRCGSTNYSDSVKGRFIISSDNAKNSVYL | E10) VHH sequence |
| | QMNSLKDEDTAVYSCHARTWTSYWGRGTQVTVSSGGQHHH | with linker (bold) |
| | HHHGAYPYDVPDYAS | and His₆ tag (SEQ ID |
| | | NO: 20)/HA tag |
| | | (italics) |
| 166 | QVQLQESGGGLVQAGGSLRLSCAASESIFRMELMEWYHQA | Nanosota-5 VHH |
| | PGKQRELVATINRCGSTNYSDSVKGRFIISSDNAKNSVYL | sequence with IgG1 |
| | QMNSLKDEDTAVYSCHARTWTSYWGRGTQVTVSSEPKSCD | Fc domain |
| | THTCPPCPAPELLGGPSVFLFPPKPKDTLMISRTPEVTC | sequence (bold) |
| | VVVDVSHEDPEVKFNWYVDGVEVHNAKTKPREEQYNSTYR | |
| | VVSVLTVLHQDWLNGKEYKCKVSNKALPAPIEKTISKAKG | |
| | QPREPQVYTLPPSREEMTKNQVSLTCLVKGFYPSDIAVEW | |
| | ESNGQPENNYKTTPPVLDSDGSFFLYSKLTVDKSRWQQGN | |
| | VFSCSVMHEALHNHYTQKSLSLSPGK | |
| 106 | QVQLQESGGGLVQAGGSLRLSCAASESIFRMELMQWYRQA | VHH sequence (clone |
| | PGKQRELVATITRSGSTNYSDSVKGRLIISSDNAKNSVYL | D6 |
| | QMNSLKAEDTAVYLCHARTWTSYWGQGTQVTVSS | |
| 102 | SIFRMELM | (clone D6): CDR-H1 |
| 163 | TITRSGSTN | (clone D6): CDR-H2 |
| 97 | HARTWTSY | (clone D6): CDR-H3 |
| 107 | QVQLQESGGGLVQAGGSLRLSCAASESIFRMELMQWYRQA | (clone D6) VHH |
| | PGKQRELVATITRSGSTNYSDSVKGRLIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 108 | QVQLQESGGGLVQAGGSLRLSCAASESIFRMELMEWYRQA | VHH sequence (clone |
| | PGKQRELVATITRSGSTNYSDSVKGRFIISSDNAKNSVYL | C2) |
| | QMNSLKAEDTAVYLCHARTWTSYWGQGTQVTVSS | |
| 102 | SIFRMELM | (clone C2): CDR-H1 |
| 163 | TITRSGSTN | (clone C2): CDR-H2 |
| 97 | HARTWTSY | (clone C2): CDR-H3 |
| 109 | QVQLQESGGGLVQAGGSLRLSCAASESIFRMELMEWYRQA | (clone C2) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 108 | QVQLQESGGGLVQAGGSLRLSCAASESIFRMELMEWYRQA | VHH sequence (clone |
| | PGKQRELVATITRSGSTNYSDSVKGRFIISSDNAKNSVYL | D1) |
| | QMNSLKAEDTAVYLCHARTWTSYWGQGTQVTVSS | |
| 102 | SIFRMELM | (clone D1): CDR-H1 |
| 163 | TITRSGSTN | (clone D1): CDR-H2 |
| 97 | HARTWTSY | (clone D1): CDR-H3 |
| 109 | QVQLQESGGGLVQAGGSLRLSCAASESIFRMELMEWYRQA | (clone D1) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 108 | QVQLQESGGGLVQAGGSLRLSCAASESIFRMELMEWYRQA | VHH sequence (clone |
| | PGKQRELVATITRSGSTNYSDSVKGRFIISSDNAKNSVYL | E1) |
| | QMNSLKAEDTAVYLCHARTWTSYWGQGTQVTVSS | |
| 102 | SIFRMELM | (clone E1): CDR-H1 |
| 163 | TITRSGSTN | (clone E1): CDR-H2 |
| 97 | HARTWTSY | (clone E1): CDR-H3 |
| 109 | QVQLQESGGGLVQAGGSLRLSCAASESIFRMELMEWYRQA | (clone E1) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 108 | QVQLQESGGGLVQAGGSLRLSCAASESIFRMELMEWYRQA | VHH sequence (clone |
| | PGKQRELVATITRSGSTNYSDSVKGRFIISSDNAKNSVYL | E4) |
| | QMNSLKAEDTAVYLCHARTWTSYWGQGTQVTVSS | |
| 102 | SIFRMELM | (clone E4): CDR-H1 |
| 163 | TITRSGSTN | (clone E4): CDR-H2 |
| 97 | HARTWTSY | (clone E4): CDR-H3 |
| 109 | QVQLQESGGGLVQAGGSLRLSCAASESIFRMELMEWYRQA | (clone E4) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 108 | QVQLQESGGGLVQAGGSLRLSCAASESIFRMELMEWYRQA | VHH sequence (clone |
| | PGKQRELVATITRSGSTNYSDSVKGRFIISSDNAKNSVYL | H4) |
| | QMNSLKAEDTAVYLCHARTWTSYWGQGTQVTVSS | |
| 102 | SIFRMELM | (clone H4): CDR-H1 |
| 163 | TITRSGSTN | (clone H4): CDR-H2 |
| 97 | HARTWTSY | (clone H4): CDR-H3 |
| 109 | QVQLQESGGGLVQAGGSLRLSCAASESIFRMELMEWYRQA | (clone H4) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | A5) |
| | QMNSLKAEDTAVYLCHARTWTSYWGQGTQVTVSS | |
| 102 | SIFRMELM | (clone A5): CDR-H1 |
| 163 | TITRSGSTN | (clone A5): CDR-H2 |
| 97 | HARTWTSY | (clone A5): CDR-H3 |
| 119 | QVQLQESGGGLVQAGGSLRLSCAASESIFRMELMQWYRQA | (clone A5) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | B1) |
| | QMNSLKAEDTAVYLCHARTWTSYWGQGTQVTVSS | |
| 102 | SIFRMELM | (clone B1): CDR-H1 |
| 163 | TITRSGSTN | (clone B1): CDR-H2 |
| 97 | HARTWTSY | (clone B1): CDR-H3 |
| 119 | QVQLQESGGGLVQAGGSLRLSCAASESIFRMELMQWYRQA | (clone B1) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | B6) |
| | QMNSLKAEDTAVYLCHARTWTSYWGQGTQVTVSS | |
| 102 | SIFRMELM | (clone B6): CDR-H1 |
| 163 | TITRSGSTN | (clone B6): CDR-H2 |
| 97 | HARTWTSY | (clone B6): CDR-H3 |
| 119 | QVQLQESGGGLVQAGGSLRLSCAASESIFRMELMQWYRQA | (clone B6) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | B7) |
| | QMNSLKAEDTAVYLCHARTWTSYWGQGTQVTVSS | |
| 102 | SIFRMELM | (clone B7): CDR-H1 |
| 163 | TITRSGSTN | (clone B7): CDR-H2 |
| 97 | HARTWTSY | (clone B7): CDR-H3 |
| 119 | QVQLQESGGGLVQAGGSLRLSCAASESIFRMELMQWYRQA | (clone B7) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | F1) |
| | QMNSLKAEDTAVYLCHARTWTSYWGQGTQVTVSS | |
| 102 | SIFRMELM | (clone F1): CDR-H1 |
| 163 | TITRSGSTN | (clone F1): CDR-H2 |
| 97 | HARTWTSY | (clone F1): CDR-H3 |
| 119 | QVQLQESGGGLVQAGGSLRLSCAASESIFRMELMQWYRQA | (clone F1) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | F3) |
| | QMNSLKAEDTAVYLCHARTWTSYWGQGTQVTVSS | |
| 102 | SIFRMELM | (clone F3): CDR-H1 |
| 163 | TITRSGSTN | (clone F3): CDR-H2 |
| 97 | HARTWTSY | (clone F3): CDR-H3 |
| 119 | QVQLQESGGGLVQAGGSLRLSCAASESIFRMELMQWYRQA | (clone F3) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 118 | QVQLQESGGGLVQAGGSLRLSCAASESIFRMELMQWYRQA | VHH sequence (clone |
| | PGKQRELVATITRSGSTNYSDSVKGRFIISSDNAKNSVYL | F4) |
| | QMNSLKAEDTAVYLCHARTWTSYWGQGTQVTVSS | |
| 102 | SIFRMELM | (clone F4): CDR-H1 |
| 163 | TITRSGSTN | (clone F4): CDR-H2 |
| 97 | HARTWTSY | (clone F4): CDR-H3 |
| 119 | QVQLQESGGGLVQAGGSLRLSCAASESIFRMELMQWYRQA | (clone F4) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 132 | QVQLQESGGGLVQAGGSLRLSCAASESIFRMELMAWYRQA | VHH sequence (clone |
| | PGKQRELVATITRSGSTNYSDSVKGRFIISSDNAKNSVYL | H5) |
| | QMNSLKAEDTAVYLCHARTWTSYWGQGTQVTVSS | |
| 102 | SIFRMELM | (clone H5): CDR-H1 |
| 163 | TITRSGSTN | (clone H5): CDR-H2 |
| 97 | HARTWTSY | (clone H5): CDR-H3 |
| 133 | QVQLQESGGGLVQAGGSLRLSCAASESIFRMELMAWYRQA | (clone H5) VHH |
| | PGKQRELVATITRSGSTNYSDSVKGRFIISSDNAKNSVYL | sequence with linker |
| | QMNSLKAEDTAVYLCHARTWTSYWGQGTQVTVSSGGQHHH | (bold) and His₆ tag |
| | HHHGAYPYDVPDYAS | (SEQ ID NO: 20)/HA |
| | | tag (italics) |
| 160 | SIFR(F/M)(E/D)(A/V/L)(V/M) | CDR-H1 |
| 161 | (S/T)(V/I)(A/T/N)R(D/S/C)G(T/S)TN | CDR-H2 |
| 162 | (H/N)AR(T/W)WT(S/N)(S/Y/F) | CDR-H3 |

TABLE 5
Class 5 (3 clones of Nanosota-6 family)

| SEQ | | |
|---|---|---|
| ID | | |
| NO: | Sequences | Comment |
| 134 | QVQLQESGGGLVQPGGSLRLSCVASGSVTENSMGWYRQAP | Nanosota-6 VHH |
| | GKQRELVAQITAGGDTHYADSVKGRFTISEHRGKNAVYLE | sequence (clone H1) |
| | MHSLKPEDTAVYYCHLQVPFLGGGYDYWGQGTQVTVSS | |
| 135 | SVTFNSM | Nanosota-6 (clone |
| | | H1): CDR-H1 |
| 136 | QITAGGDTH | Nanosota-6 (clone |
| | | H1): CDR-H2 |
| 137 | HLQVPFLGGGYDY | Nanosota-6 (clone |
| | | H1): CDR-H3 |
| 138 | QVQLQESGGGLVQPGGSLRLSCVASGSVTENSMGWYRQAP | Nanosota-6 (clone |
| | GKQRELVAQITAGGDTHYADSVKGRFTISEHRGKNAVYLE | H1) VHH sequence with |
| | MHSLKPEDTAVYYCHLQVPFLGGGYDYWGQGTQVTVSSGG | linker (bold) and |
| | QHHHHHHGAYPYDVPDYAS | His₆ tag (SEQ ID |
| | | NO: 20)/HA tag |
| | | (italics) |
| 153 | QVQLQESGGGLVQPGGSLRLSCVASGSVTFNSMGWYRQAP | Nanosota-6 VHH |
| | GKQRELVAQITAGGDTHYADSVKGRFTISEHRGKNAVYLE | sequence with IgG1 |
| | MHSLKPEDTAVYYCHLQVPFLGGGYDYWGQGTQVTVSSEP | Fc domain |
| | KSCDKTHTCPPCPAPELLGGPSVFLFPPKPKDTLMISRTP | sequence (bold) |
| | EVTCVVVDVSHEDPEVKFNWYVDGVEVHNAKTKPREEQYN | |
| | STYRVVSVLTVLHQDWLNGKEYKCKVSNKALPAPIEKTIS | |
| | KAKGQPREPQVYTLPPSREEMTKNQVSLTCLVKGFYPSDI | |
| | AVEWESNGQPENNYKTTPPVLDSDGSFFLYSKLTVDKSRW | |
| | QQGNVFSCSVMHEALHNHYTQKSLSLSPGK | |
| 139 | QVQLQESGGGLVQPGGSLRLSCVASGSVTENSMGWYRQAP | (clone H3) |
| | GKQRELVAQITAGGDTHYADSVKGRFTISEHRDKNTVYLE | |
| | MRSLKPEDTAVYYCHLQVPFLGGGYDYWGQGTQVTVSS | |
| 135 | SVTFNSM | (clone H3): CDR-H1 |
| 136 | QITAGGDTH | (clone H3): CDR-H2 |
| 137 | HLQVPFLGGGYDY | (clone H3): CDR-H3 |
| 140 | QVQLQESGGGLVQPGGSLRLSCVASGSVTENSMGWYRQAP | (clone H3) VHH |
| | GKQRELVAQITAGGDTHYADSVKGRFTISEHRDKNTVYLE | sequence with linker |
| | MRSLKPEDTAVYYCHLQVPFLGGGYDYWGQGTQVTVSSGG | (bold) and His₆ tag |
| | QHHHHHHGAYPYDVPDYAS | (SEQ ID NO: 20)/HA |
| | | tag (italics) |
| 141 | QVQLQEWGGGLVQPGGSLRLSCVASGSVTFNSMGWYRQAP | VHH sequence (clone |
| | GKQRELVAQITAGGDTHYADSVKGRFTISEHRGKNAVYLE | D2) |
| | MHSLKPEDTAVYYCHLQVPFLGGGYDYWGQGTQVTVSS | |
| 135 | SVTFNSM | (clone D2): CDR-H1 |
| 136 | QITAGGDTH | (clone D2): CDR-H2 |
| 137 | HLQVPFLGGGYDY | (clone D2): CDR-H3 |
| 142 | QVQLQEWGGGLVQPGGSLRLSCVASGSVTFNSMGWYRQAP | (clone D2) VHH |
| | GKQRELVAQITAGGDTHYADSVKGRFTISEHRGKNAVYLE | sequence with linker |
| | MHSLKPEDTAVYYCHLQVPFLGGGYDYWGQGTQVTVSSGG | (bold) and His₆ tag |
| | QHHHHHHGAYPYDVPDYAS | (SEQ ID NO: 20)/HA |
| | | tag (italics) |

TABLE 6
Class 6 (Nanosota-7: clone C3)

| SEQ | | |
|---|---|---|
| ID | | |
| NO: | Sequences | Comment |
| 143 | QVQLQESGGGLVQAGESLRLSCVASGSIFSINAMGWYRQA | Nanosota-7 VHH |
| | PGKQRELVAGITDDGSTNYTHEVEGRFTISRDNAKNTVYL | sequence (clone C3) |
| | QMNSLKPEDTAVYYCNAKIHTPYNYWGQGTQVTVSS | |
| 144 | SIFSINAM | Nanosota-7 (clone |
| | | C3): CDR-H1 |
| 145 | GITDDGSTN | Nanosota-7 (clone |
| | | C3): CDR-H2 |
| 146 | NAKIHTPYNY | Nanosota-7 (clone |
| | | C3): CDR-H3 |
| 147 | QVQLQESGGGLVQAGESLRLSCVASGSIFSINAMGWYRQA | Nanosota-7 (clone |
| | PGKQRELVAGITDDGSTNYTHEVEGRFTISRDNAKNTVYL | C3) VHH sequence with |
| | QMNSLKPEDTAVYYCNAKIHTPYNYWGQGTQVTVSSGGQH | linker (bold) and |
| | HHHHHGAYPYDVPDYAS | His₆ tag (SEQ ID |
| | | NO: 20) (italics) |
| 154 | QVQLQESGGGLVQAGESLRLSCVASGSIFSINAMGWYRQA | Nanosota-7 VHH |
| | PGKQRELVAGITDDGSTNYTHEVEGRFTISRDNAKNTVYL | sequence with IgG1 |
| | QMNSLKPEDTAVYYCNAKIHTPYNYWGQGTQVTVSSEPKS | Fc domain |
| | CDKTHTCPPCPAPELLGGPSVFLFPPKPKDTLMISRTPEV | sequence (bold) |
| | TCVVVDVSHEDPEVKFNWYVDGVEVHNAKTKPREEQYNST | |
| | YRVVSVLTVLHQDWLNGKEYKCKVSNKALPAPIEKTISKA | |
| | KGQPREPQVYTLPPSREEMTKNQVSLTCLVKGFYPSDIAV | |
| | EWESNGQPENNYKTTPPVLDSDGSFFLYSKLTVDKSRWQQ | |
| | GNVFSCSVMHEALHNHYTQKSLSLSPGK | |

The entire contents of Gang Ye, et al., J Virol. 2023 Nov. 30; 97 (11):e0144823. doi: 10.1128/jvi.01448-23, and Gang Ye, et al., PLoS Pathog. 2024 Sep. 5; 20 (9):e1012493. doi:10.1371/journal.ppat.1012493 are incorporated by reference herein.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. An isolated anti-SARS-CoV-2 binder protein comprising:
(1) one or more CDRs selected from the group consisting of:
(a) a CDR1 comprising an amino acid sequence having at least 75% sequence identity to an amino acid sequence of SIFRMELM (SEQ ID NO: 102), SIFRFEAV (SEQ ID NO: 90), or SIFRMDVV (SEQ ID NO: 95);
(b) a CDR2 comprising an amino acid sequence having at least 75% sequence identity to an amino acid sequence of TINRCGSTN (SEQ ID NO: 103), TVARDGTTN (SEQ ID NO: 91), SITRSGSTN (SEQ ID NO: 96), or TITRSGSTN (SEQ ID NO: 163); and
(c) a CDR3 comprising an amino acid sequence having at least 75% sequence identity to an amino acid sequence of HARTWTSY (SEQ ID NO: 97), NARWWTNF (SEQ ID NO: 92), or HARTWTSS (SEQ ID NO: 104); or
(2) one or more CDRs selected from the group consisting of:
(a) a CDR1 comprising an amino acid sequence having at least 75% sequence identity to an amino acid sequence of SIFSPNTM (SEQ ID NO: 8) or STSASNSM (SEQ ID NO: 34);
(b) a CDR2 comprising an amino acid sequence having at least 75% sequence identity to an amino acid sequence of VISSIASTQ (SEQ ID NO: 9), FISSIASTS (SEQ ID NO: 157), or TAANGDIRS (SEQ ID NO: 35); and
(c) a CDR3 comprising an amino acid sequence having at least 75% sequence identity to an amino acid sequence of YAVDKSQDY (SEQ ID NO: 10) or YSVDSYRDY (SEQ ID NO: 36); or
(3) one or more CDRs selected from the group consisting of:
(a) a CDR1 comprising an amino acid sequence having at least 75% sequence identity to an amino acid sequence of FTLDYYAI (SEQ ID NO: 14), FILDFYAI (SEQ ID NO: 47), TTLDHYAI (SEQ ID NO: 65), FTVNSHAI (SEQ ID NO: 70), or FALDYYAI (SEQ ID NO: 76);
(b) a CDR2 comprising an amino acid sequence having at least 75% sequence identity to an amino acid sequence of CISSSGGRTN (SEQ ID NO: 15), CISSSGGSTN (SEQ ID NO: 66), or CISISGGSTN (SEQ ID NO: 77); and
(c) a CDR3 comprising an amino acid sequence having at least 75% sequence identity to an amino acid sequence of AAWEASRWYCPLQFSADFSS (SEQ ID NO: 16) AAWEGSTRYCPIQTSADFVS (SEQ ID NO: 48), AAWEGSSEYCPLQFSADFAS (SEQ ID NO: 67) or AAWEGSSEYCPLQYSADFDS (SEQ ID NO: 73); or
(4) one or more CDRs selected from the group consisting of:
(a) a CDR1 comprising an amino acid sequence having at least 75% sequence identity to an amino acid sequence of FNFETSTV (SEQ ID NO: 2);
(b) a CDR2 comprising an amino acid sequence having at least 75% sequence identity to an amino acid sequence of CINKGYEDTN (SEQ ID NO: 3); and
(c) a CDR3 comprising an amino acid sequence having at least 75% sequence identity to an amino acid sequence of AAHNEPYFCDYSGRFRWNEYSY (SEQ ID NO: 4); or
(5) one or more CDRs selected from the group consisting of:
(a) a CDR1 comprising an amino acid sequence having at least 75% sequence identity to an amino acid sequence of SVTFNSM (SEQ ID NO: 135);
(b) a CDR2 comprising an amino acid sequence having at least 75% sequence identity to an amino acid sequence of QITAGGDTH (SEQ ID NO: 136); and
(c) a CDR3 comprising an amino acid sequence having at least 75% sequence identity to an amino acid sequence of HLQVPFLGGGYDY (SEQ ID NO: 137); or
(6) one or more CDRs selected from the group consisting of:
(a) a CDR1 comprising an amino acid sequence having at least 75% sequence identity to an amino acid sequence of SIFSINAM (SEQ ID NO: 144);
(b) a CDR2 comprising an amino acid sequence having at least 75% sequence identity to an amino acid sequence of GITDDGSTN (SEQ ID NO: 145); and
(c) a CDR3 comprising an amino acid sequence having at least 75% sequence identity to an amino acid sequence of NAKIHTPYNY (SEQ ID NO: 146).

2. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of SIFRMELM (SEQ ID NO: 102), SIFRFEAV (SEQ ID NO: 90), or SIFRMDVV (SEQ ID NO: 95);
(b) a CDR2 comprising an amino acid sequence having at least 80% sequence identity to an amino acid sequence of TINRCGSTN (SEQ ID NO: 103), TVARDGTTN (SEQ ID NO: 91), SITRSGSTN (SEQ ID NO: 96), or TITRSGSTN (SEQ ID NO: 163); and
(c) a CDR3 comprising an amino acid sequence having at least 80% sequence identity to an amino acid sequence of HARTWTSY (SEQ ID NO: 97), NARWWTNF (SEQ ID NO: 92), or HARTWTSS (SEQ ID NO: 104).

3. The isolated anti-SARS-CoV-2 binder protein of claim 2, comprising one or more CDRs selected from the group consisting of:
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSY (SEQ ID NO: 97); or
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSS (SEQ ID NO: 104); or
(a) a CDR1 comprising an amino acid sequence of SIFRFEAV (SEQ ID NO: 90);
(b) a CDR2 comprising an amino acid sequence of TVARDGTTN (SEQ ID NO: 91); and
(c) a CDR3 comprising an amino acid sequence of NARWWTNF (SEQ ID NO: 92); or
(a) a CDR1 comprising an amino acid sequence of SIFRMDVV (SEQ ID NO: 95);
(b) a CDR2 comprising an amino acid sequence of SITRSGSTN (SEQ ID NO: 96); and
(c) a CDR3 comprising an amino acid sequence of HARTWTSY (SEQ ID NO: 97); or
(a) a CDR1 comprising an amino acid sequence of SIFRMELM (SEQ ID NO: 102);
(b) a CDR2 comprising an amino acid sequence of TITRSGSTN (SEQ ID NO: 163); and
(c) a CDR3 comprising an amino acid sequence of HARTWTSY (SEQ ID NO: 97).

4. The isolated anti-SARS-CoV-2 binder protein of claim 2, comprising:
(a) a CDR1 comprising an amino acid sequence of SIFRMELM (SEQ ID NO: 102);
(b) a CDR2 comprising an amino acid sequence of TINRCGSTN (SEQ ID NO: 103); and
(c) a CDR3 comprising an amino acid sequence of HARTWTSY (SEQ ID NO: 97).

5. The isolated anti-SARS-CoV-2 binder protein of claim 2, comprising an amino acid sequence that has at least 80% sequence identity to:
(SEQ ID NO: 164)
QVQLQESGGGLVQAGGSLRLSCAASESIFRMELMEWYHQAPGKQRELVA
TINRCGSTNYSDSVKGRFIISSDNAKNSVYLQMNSLKDEDTAVYSCHAR
TWTSYWGRGTQVTVSS,
(SEQ ID NO: 101)
QVQLQESGGGLVQAGGSLRLSCAASESIFRMELMEWYHQAPGKQRELVA
TINRCGSTNYSDSVKGRFIISSDNAKNSVYLQMNSLKDEDTAVYSCHAR
TWTSSWGRGTQVTVSS,
(SEQ ID NO: 106)
QVQLQESGGGLVQAGGSLRLSCAASESIFRMELMQWYRQAPGKQRELVA
TITRSGSTNYSDSVKGRLIISSDNAKNSVYLQMNSLKAEDTAVYLCHAR
TWTSYWGQGTQVTVSS,
(SEQ ID NO: 89)
QVQLQESGGGLVQAGGSLRLSCVASGSIFRFEAVGWYRQAPGKQRELVA
TVARDGTTNYADSVKGRFTISTDNAKNSVYLQMNSLKAEDTAVYVCNAR
WWTNFWGQGTQVTVSS,
(SEQ ID NO: 94)
QVQLQESGGGLVQAGGSLRLSCAASESIFRMDVVQWYRQAPGKQRELVA
SITRSGSTNYADSVKGRFIISSDNAKNSVYLQMKSLKVEDTAVYLCHAR
TWTSYWGQGTQVTVSS,
(SEQ ID NO: 99)
QVQLQESGGGLVQPGGSLRLSCAASESIFRMDVVQWYRQAPGKQRELVA
SITRSGSTNYADSVKGRFIISSDNAKNSVYLQMKSLKVEDTAVYLCHAR
TWTSYWGQGTQVTVSS;
(SEQ ID NO: 108)
QVQLQESGGGLVQAGGSLRLSCAASESIFRMELMEWYRQAPGKQRELVA
TITRSGSTNYSDSVKGRFIISSDNAKNSVYLQMNSLKAEDTAVYLCHAR
TWTSYWGQGTQVTVSS;
(SEQ ID NO: 118)
QVQLQESGGGLVQAGGSLRLSCAASESIFRMELMQWYRQAPGKQRELVA
TITRSGSTNYSDSVKGRFIISSDNAKNSVYLQMNSLKAEDTAVYLCHAR
TWTSYWGQGTQVTVSS;
or
(SEQ ID NO: 132)
QVQLQESGGGLVQAGGSLRLSCAASESIFRMELMAWYRQAPGKQRELVA
TITRSGSTNYSDSVKGRFIISSDNAKNSVYLQMNSLKAEDTAVYLCHAR
TWTSYWGQGTQVTVSS.

6-8. (canceled)

9. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of SIFSPNTM (SEQ ID NO: 8) or STSASNSM (SEQ ID NO: 34);
(b) a CDR2 comprising an amino acid sequence having at least 80% sequence identity to an amino acid sequence of VISSIASTQ (SEQ ID NO: 9), FISSIASTS (SEQ ID NO: 157), or TAANGDIRS (SEQ ID NO: 35); and
(c) a CDR3 comprising an amino acid sequence having at least 80% sequence identity to an amino acid sequence of YAVDKSQDY (SEQ ID NO: 10) or YSVDSYRDY (SEQ ID NO: 36).

10. (canceled)

11. The isolated anti-SARS-CoV-2 binder protein of claim 9, comprising:
(a) a CDR1 comprising the amino acid sequence of SIFSPNTM (SEQ ID NO: 8);
(b) a CDR2 comprising the amino acid sequence of VISSIASTQ (SEQ ID NO: 9) or FISSIASTS (SEQ ID NO: 157); and
(c) a CDR3 comprising the amino acid sequence of YAVDKSQDY (SEQ ID NO: 10); or
(a) a CDR1 comprising the amino acid sequence of STSASNSM (SEQ ID NO: 34);
(b) a CDR2 comprising the amino acid sequence of TAANGDIRS (SEQ ID NO: 35); and
(c) a CDR3 comprising the amino acid sequence of YSVDSYRDY (SEQ ID NO: 36).

12. The isolated anti-SARS-CoV-2 binder protein of claim 9, comprising an amino acid sequence that has at least 80% sequence identity to:
(SEQ ID NO: 7)
QVQLQESGGGLVQAGGSLRLSCAASGSIFSPNTMGWFRQALGKQREMVA
VISSIASTQYANFVKGRFTITRDNTKNTVHLQMNSLIPEDTAVYYCYAV
DKSQDYWGQGTQVTVSS;
(SEQ ID NO: 156)
QVQLQESGGGLVQAGGSLRLSCAASGSIFSPNTMGWFRQALGKQREMVA
FISSIASTSYANFVKGRFTITRDNTKNTVHLQMNSLIPEDTAVYYCYAV
DKSQDYWGQGTQVTVSS;
(SEQ ID NO: 167)
QVQLQESGGGLVQAGGSLRLSCAASGSIFSPNTMGWFRQALGKQREGVA
FISSIASTSYWLPVKGRFTITRDNTKNTVHLQMNSLIPEDTAVYYCYAV
DKSQDYWGQGTQVTVSS;
(SEQ ID NO: 168)
QVQLQESGGGLVQAGGSLRLSCAASGSIFSPNTMGWFRQALGKQREGVA
FISSIASTSYNWYVKGRFTITRDNTKNTVHLQMNSLIPEDTAVYYCYAV
DKSQDYWGQGTQVTVSS;
(SEQ ID NO: 169)
QVQLQESGGGLVQAGGSLRLSCAASGSIFSPNTMGWFRQALGKQREGVA
FISSIASTSYNFFVKGRFTITRDNTKNTVHLQMNSLIPEDTAVYYCYAV
DKSQDYWGQGTQVTVSS;
(SEQ ID NO: 170)
QVQLQESGGGLVQAGGSLRLSCAASGSIFSPNTMGWFRQALGKQREGVA
FISSIASTSYGYGVKGRFTITRDNTKNTVHLQMNSLIPEDTAVYYCYAV
DKSQDYWGQGTQVTVSS;
(SEQ ID NO: 33)
QVQLQESGGGLVQAGGPLRLSCVASASTSASNSMSWFRQAPGKQREWVA
TAANGDIRSYANFVKGRFTISRDDAKNTVYLQMNGLKPEDTAVYYCYSV
DSYRDYWGQGTQVTVSS;
or
(SEQ ID NO: 44)
QVQLQESGGGLVQPGGSLRLSCAASGSIFSPNTMGWFRQALGKQREMVA
VISSIASTQYANFVKGRFTITRDNTKNTVHLQMNSLIPEDTAVYYCYAV
DKSQDYWGQGTQVTVSS.

13-15. (canceled)

16. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of FTLDYYAI (SEQ ID NO: 14), FILDFYAI (SEQ ID NO: 47), TTLDHYAI (SEQ ID NO: 65), FTVNSHAI (SEQ ID NO: 70), or FALDYYAI (SEQ ID NO: 76);
(b) a CDR2 comprising an amino acid sequence having at least 80% sequence identity to an amino acid sequence of CISSSGGRTN (SEQ ID NO: 15), CISSSGGSTN (SEQ ID NO: 66), or CISISGGSTN (SEQ ID NO: 77); and
(c) a CDR3 comprising an amino acid sequence having at least 80% sequence identity to an amino acid sequence of AAWEASRWYCPLQFSADFSS (SEQ ID NO: 16), AAWEGSTRYCPIQTSADFVS (SEQ ID NO: 48), AAWEGSSEYCPLQFSADFAS (SEQ ID NO: 67) or AAWEGSSEYCPLQYSADFDS (SEQ ID NO: 73).

17-18. (canceled)

19. The isolated anti-SARS-CoV-2 binder protein of claim 16, comprising:
a) CDRs 1-3 comprising the amino acid sequences of SEQ ID NOs: 14, 15, and 16, respectively;
b) CDRs 1-3 comprising the amino acid sequences of SEQ ID NOs: 47, 15, and 48, respectively;
c) CDRs 1-3 comprising the amino acid sequences of SEQ ID NOs: 65, 66, and 67, respectively;
d) CDRs 1-3 comprising the amino acid sequences of SEQ ID NOs: 70, 66, and 67, respectively;
e) CDRs 1-3 comprising the amino acid sequences of SEQ ID NOs: 76, 77, and 67, respectively;
f) CDRs 1-3 comprising the amino acid sequences of SEQ ID NOs: 14, 66, and 67, respectively; or
g) CDRs 1-3 comprising the amino acid sequences of SEQ ID NOs: 14, 66, and 73, respectively.

20. (canceled)

21. The isolated anti-SARS-CoV-2 binder protein of claim 16, comprising an amino acid sequence that has at least 85% sequence identity to:
(SEQ ID NO: 13)
QVQLQESGGGLVQPGGSLRLSCAASGFTLDYYAIGWFRQAPGKEREGVS
CISSSGGRTNYADSVKGRFTISRDNTKNTVYLQMNSLKPEDTAVYYCAA
WEASRWYCPLQFSADFSSWGQGTQVTVSS;
(SEQ ID NO: 46)
QVQLQESGGGLVQPGGSLRLSCAASGFILDFYAIGWFRQAPGKEREGVS
CISSSGGRTNYADSVKGRFTISRDNAKNTVYLQMNSLKPEDTAVYSCAA
WEGSTRYCPIQTSADFVSWGQGTQVTVSS;
(SEQ ID NO: 62)
QVQLQESGGGLVQPGGSLRLSCAASGFILDFYAIGWFRQAPGKEREGVS
CISSSGGRTNYADSVKGRFTISSDNAKNTVYLQMNSLKPEDTAVYSCAA
WEGSTRYCPIQTSADFVSWGQGTQVTVSS;
(SEQ ID NO: 64)
QVQLQESGGGLVQAGGSLRLTCVASGTTLDHYAIGWLRQAPGKEREGVS
CISSSGGSTNYADSVKGRFTISRDNAKNTVYLQMTGLKPEDTAVYYCAA
WEGSSEYCPLQFSADFASWGRGTQVTVSS;
(SEQ ID NO: 69)
QVQLQESGGGLVQDGGSLRLSCEASGFTVNSHAIGWFRQSPGKEREGVS
CISSSGGSTNYADSVKGRFTISRDNAKNTVYLQMTSLKPEDTAVYYCAA
WEGSSEYCPLQFSADFASWGRGTQVTVSS;
(SEQ ID NO: 72)
QVQLQECGGGLVQPGGSLRLSCAASGFTLDYYAIGWFRQAPGKEREGVS
CISSSGGSTNYADSVKGRFTISRDNAKNTVYLQMTSLKPEDTAVYYCAA
WEGSSEYCPLQYSADFDSWGQGTQVTVSS;
(SEQ ID NO: 75)
QVQLQESGGGLVQPGGSLRLSCAASGFALDYYAIGWFRQAPKKEREGVS
CISISGGSTNYADSVEGRFTISRDNAKNTVYLQMTSLKPEDTAVYYCAA
WEGSSEYCPLQFSADFASWGRGTQVTVSS;
(SEQ ID NO: 79)
QVQLQESGGGLVQPGGSLRLSCAASGFTLDYYAIGWFRQAPGKEREGVS
CISSSGGSTNYADSVKGRFTISRDNAKNTVYLQMTSLKPEDTAVYYCAA
WEGSSEYCPLQFSADFASRGQGTQVTVSS;
(SEQ ID NO: 81)
QVQLQESGGGLVQPGGSLRLFCAASGFTLDYYAIGWFRQAPGKEREGVS
CISSSGGSTNYADSVKGRFTISRDNAKNTVYLQMTSLKPEDTAVYYCAA
WEGSSEYCPLQFSADFASWGRGTQVTVSS;
(SEQ ID NO: 83)
QVQLQESGGGLVQPGGSLRLSCAASGFTLDYYAIGWFRQAPGKEREGVS
CISSSGGSTNYADSVKGRFTISRDNAKNTVYLQMTSLKPEDTAVYYCAA
WEGSSEYCPLQFSADFASWGRGTQVTVSS;
or
(SEQ ID NO: 87)
QVQLQESGGGLVQPGGSLRLSCAASGFTLDYYAIGWFRQAPGKEREGVS
CISSSGGSTNYVDSVKGRFTISRDNAKNTVYLQMTSLKPEDTAVYYCAA
WEGSSEYCPLQFSADFASWGRGTQVTVSS.

22. (canceled)

23. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of FNFETSTV (SEQ ID NO: 2);
(b) a CDR2 comprising an amino acid sequence having at least 80% sequence identity to an amino acid sequence of CINKGYEDTN (SEQ ID NO: 3); and
(c) a CDR3 comprising an amino acid sequence having at least 80% sequence identity to an amino acid sequence of AAHNEPYFCDYSGRFRWNEYSY (SEQ ID NO: 4).

24. (canceled)

25. The isolated anti-SARS-CoV-2 binder protein of claim 23, comprising an amino acid sequence that has at least 80% sequence identity to
(SEQ ID NO: 1)
QVQLQESGGGAVQPGGSLGLSCTASGFNFETSTVGWFRQAPGKENEGVS
CINKGYEDTNYADSVKGRFTISRDAAKNTVYLQMDSLQPEDTATYYCAA
HNEPYFCDYSGRFRWNEYSYYGQGTQVTVSS;
or
(SEQ ID NO: 31)
QVQLQESGGGAVQPGGSLGLSCTASGFNFETSTVGWFRQAPGKEYEGVS
CINKGYEDTNYADSVKGRFTISRDAAKNTVYLQMDSLQPEDTATYYCAA
HNEPYFCDYSGRFRWNEYSYYGQGTQVTVSS.

26-27. (canceled)

28. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of SVTFNSM (SEQ ID NO: 135);
(b) a CDR2 comprising an amino acid sequence having at least 80% sequence identity to an amino acid sequence of QITAGGDTH (SEQ ID NO: 136); and
(c) a CDR3 comprising an amino acid sequence having at least 80% sequence identity to an amino acid sequence of HLQVPFLGGGYDY (SEQ ID NO: 137).

29-30. (canceled)

31. The isolated anti-SARS-CoV-2 binder protein of claim 28, comprising an amino acid sequence that has at least 80% sequence identity to:
(SEQ ID NO: 134)
QVQLQESGGGLVQPGGSLRLSCVASGSVTFNSMGWYRQAPGKQRELVAQ
ITAGGDTHYADSVKGRFTISEHRGKNAVYLEMHSLKPEDTAVYYCHLQV
PFLGGGYDYWGQGTQVTVSS;
(SEQ ID NO: 139)
QVQLQESGGGLVQPGGSLRLSCVASGSVTFNSMGWYRQAPGKQRELVAQ
ITAGGDTHYADSVKGRFTISEHRDKNTVYLEMRSLKPEDTAVYYCHLQV
PFLGGGYDYWGQGTQVTVSS;
or
(SEQ ID NO: 141)
QVQLQEWGGGLVQPGGSLRLSCVASGSVTFNSMGWYRQAPGKQRELVAQ
ITAGGDTHYADSVKGRFTISEHRGKNAVYLEMHSLKPEDTAVYYCHLQV
PFLGGGYDYWGQGTQVTVSS.

32-34. (canceled)

35. The isolated anti-SARS-CoV-2 binder protein of claim 1, comprising one or more CDRs selected from the group consisting of:
(a) a CDR1 comprising an amino acid sequence having at least 80% sequence identity to an amino acid sequence of SIFSINAM (SEQ ID NO: 144);
(b) a CDR2 comprising an amino acid sequence having at least 80% sequence identity to an amino acid sequence of GITDDGSTN (SEQ ID NO: 145); and
(c) a CDR3 comprising an amino acid sequence having at least 80% sequence identity to an amino acid sequence of NAKIHTPYNY (SEQ ID NO: 146).

36-37. (canceled)

38. The isolated anti-SARS-CoV-2 binder protein of claim 35, comprising an amino acid sequence that has at least 80% sequence identity to:
(SEQ ID NO: 143)
QVQLQESGGGLVQAGESLRLSCVASGSIFSINAMGWYRQAPGKQRELVA
GITDDGSTNYTHFVEGRFTISRDNAKNTVYLQMNSLKPEDTAVYYCNAK
IHTPYNYWGQGTQVTVSS.

39-41. (canceled)

42. The isolated anti-SARS-CoV-2 binder protein according to claim 1, wherein the binder protein comprises an anti-SARS-CoV-2 single-domain antibody (sdAb) that is linked to at least one polypeptide tag through a peptide bond or a polypeptide linker.

43-46. (canceled)

47. The isolated anti-SARS-CoV-2 binder protein of claim 42, wherein the at least one polypeptide tag comprises a Fc tag.

48-49. (canceled)

50. The isolated anti-SARS-CoV-2 binder protein of claim 47, comprising an amino acid sequence that has at least 90% sequence identity to any one of:
(a)
(SEQ ID NO: 166)
QVQLQESGGGLVQAGGSLRLSCAASESIFRMELMEWYHQAPGKQRELVA
TINRCGSTNYSDSVKGRFIISSDNAKNSVYLQMNSLKDEDTAVYSCHAR
TWTSYWGRGTQVTVSSEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGK;
(b)
(SEQ ID NO: 12)
QVQLQESGGGLVQAGGSLRLSCAASGSIFSPNTMGWFRQALGKQREMVA
VISSIASTQYANFVKGRFTITRDNTKNTVHLQMNSLIPEDTAVYYCYAV
DKSQDYWGQGTQVTVSSEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPGK;
(c)
(SEQ ID NO: 172)
QVQLQESGGGLVQAGGSLRLSCAASGSIFSPNTMGWFRQALGKQREMVA
FISSIASTSYANFVKGRFTITRDNTKNTVHLQMNSLIPEDTAVYYCYAV
DKSQDYWGQGTQVTVSSEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPGK;
(d)
(SEQ ID NO: 173)
QVQLQESGGGLVQAGGSLRLSCAASGSIFSPNTMGWFRQALGKQREGVA
FISSIASTSYWLPVKGRFTITRDNTKNTVHLQMNSLIPEDTAVYYCYAV
DKSQDYWGQGTQVTVSSEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSL
SPGK;
(e)
(SEQ ID NO: 18)
QVQLQESGGGLVQPGGSLRLSCAASGFTLDYYAIGWFRQAPGKEREGVS
CISSSGGRTNYADSVKGRFTISRDNTKNTVYLQMNSLKPEDTAVYYCAA
WEASRWYCPLQFSADFSSWGQGTQVTVSSEPKSCDKTHTCPPCPAPELL
GGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEA
LHNHYTQKSLSLSPGK,
(f)
(SEQ ID NO: 152)
QVQLQESGGGLVQAGGSLRLSCAASESIFRMELMEWYHQAPGKQRELVA
TINRCGSTNYSDSVKGRFIISSDNAKNSVYLQMNSLKDEDTAVYSCHAR
TWTSSWGRGTQVTVSSEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLS
PGK,
and
(g)
(SEQ ID NO: 6)
QVQLQESGGGAVQPGGSLGLSCTASGFNFETSTVGWFRQAPGKENEGVS
CINKGYEDTNYADSVKGRFTISRDAAKNTVYLQMDSLQPEDTATYYCAA
HNEPYFCDYSGRFRWNEYSYYGQGTQVTVSSEPKSCDKTHTCPPCPAPE
LLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMH
EALHNHYTQKSLSLSPGK.

51-60. (canceled)

61. A pharmaceutical composition comprising the isolated anti-SARS-CoV-2 binder protein according to claim 1, and a pharmaceutically acceptable carrier.

62-64. (canceled)

65. An isolated polynucleotide comprising a nucleotide sequence encoding an isolated anti-SARS-CoV-2 binder protein of claim 1.

66. A vector comprising the polynucleotide of claim 65.

67. A cell comprising the polynucleotide of claim 65.

68. (canceled)

69. A method of inhibiting the activity of SARS-CoV, comprising contacting SARS-CoV with an isolated anti-SARS-CoV-2 binder protein of claim 1.

70-75. (canceled)

76. A method for treating or preventing a SARS-CoV infection in a mammal, comprising administering an effective amount of an isolated anti-SARS-CoV-2 binder protein of claim 1 to the mammal.

77. (canceled)

78. A method of detecting the presence of SARS-CoV-2 in a biological sample, the method comprising contacting the biological sample with the anti-SARS-CoV-2 binder protein of claim 1, and detecting whether a complex is formed between 1) the binder protein; and 2) SARS-CoV-2.

79-85. (canceled)


SEQUENCE LISTING (232 sequences)

SEQ ID NO: 1 - protein, 129 aa, synthetic construct
QVQLQESGGGAVQPGGSLGLSCTASGFNFETSTVGWFRQAPGKENEGVSCINKGYEDTNYADSVKGRFTISRDAAKNTVYLQMDSLQPEDTATYYCAAHNEPYFCDYSGRFRWNEYSYYGQGTQVTVSS

SEQ ID NO: 2 - protein, 8 aa, synthetic construct
FNFETSTV

SEQ ID NO: 3 - protein, 10 aa, synthetic construct
CINKGYEDTN

SEQ ID NO: 4 - protein, 22 aa, synthetic construct
AAHNEPYFCDYSGRFRWNEYSY

SEQ ID NO: 5 - protein, 137 aa, synthetic construct
QVQLQESGGGAVQPGGSLGLSCTASGFNFETSTVGWFRQAPGKENEGVSCINKGYEDTNYADSVKGRFTISRDAAKNTVYLQMDSLQPEDTATYYCAAHNEPYFCDYSGRFRWNEYSYYGQGTQVTVSSGSHHHHHH

SEQ ID NO: 6 - protein, 361 aa, synthetic construct
QVQLQESGGGAVQPGGSLGLSCTASGFNFETSTVGWFRQAPGKENEGVSCINKGYEDTNYADSVKGRFTISRDAAKNTVYLQMDSLQPEDTATYYCAAHNEPYFCDYSGRFRWNEYSYYGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 115 aa, synthetic construct
QVQLQESGGGLVQAGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSS

SEQ ID NO: 8 - protein, 8 aa, synthetic construct
SIFSPNTM

SEQ ID NO: 9 - protein, 9 aa, synthetic construct
VISSIASTQ

SEQ ID NO: 10 - protein, 9 aa, synthetic construct
YAVDKSQDY

SEQ ID NO: 11 - protein, 123 aa, synthetic construct
QVQLQESGGGLVQAGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSSGSHHHHHH

SEQ ID NO: 12 - protein, 347 aa, synthetic construct
QVQLQESGGGLVQAGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 127 aa, synthetic construct
QVQLQESGGGLVQPGGSLRLSCAASGFTLDYYAIGWFRQAPGKEREGVSCISSSGGRTNYADSVKGRFTISRDNTKNTVYLQMNSLKPEDTAVYYCAAWEASRWYCPLQFSADFSSWGQGTQVTVSS

SEQ ID NO: 14 - protein, 8 aa, synthetic construct
FTLDYYAI

SEQ ID NO: 15 - protein, 10 aa, synthetic construct
CISSSGGRTN

SEQ ID NO: 16 - protein, 20 aa, synthetic construct
AAWEASRWYCPLQFSADFSS

SEQ ID NO: 17 - protein, 135 aa, synthetic construct
QVQLQESGGGLVQPGGSLRLSCAASGFTLDYYAIGWFRQAPGKEREGVSCISSSGGRTNYADSVKGRFTISRDNTKNTVYLQMNSLKPEDTAVYYCAAWEASRWYCPLQFSADFSSWGQGTQVTVSSGSHHHHHH

SEQ ID NO: 18 - protein, 359 aa, synthetic construct
QVQLQESGGGLVQPGGSLRLSCAASGFTLDYYAIGWFRQAPGKEREGVSCISSSGGRTNYADSVKGRFTISRDNTKNTVYLQMNSLKPEDTAVYYCAAWEASRWYCPLQFSADFSSWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - skipped

SEQ ID NO: 20 - protein, 6 aa, synthetic construct
HHHHHH

SEQ ID NO: 21 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 387 bp, synthetic construct
  source 1..387: mol_type = other DNA
caggtgcagctgcaggagtctgggggaggcgccgtgcagcctggggggtctctcggactctcctgtacagcctctggattcaattttgaaacttcaaccgtaggctggttccgccaggccccagggaaggagaatgagggggtctcatgtatcaataaaggttatgaagatacaaattatgcagactccgtgaagggccggttcaccatctccagagacgccgccaagaacacggtgtacctgcaaatggacagcctgcaacctgaggacacagccacatattattgtgcagcacataatgagccttatttttgcgactatagtgggcgttttcggtggaatgagtacagctactatggccaggggacccaggtcaccgtctcctca

SEQ ID NO: 23 - DNA, 1083 bp, synthetic construct
  source 1..1083: mol_type = other DNA
caggtgcagctgcaggagtctgggggaggcgccgtgcagcctggggggtctctcggactctcctgtacagcctctggattcaattttgaaacttcaaccgtaggctggttccgccaggccccagggaaggagaatgagggggtctcatgtatcaataaaggttatgaagatacaaattatgcagactccgtgaagggccggttcaccatctccagagacgccgccaagaacacggtgtacctgcaaatggacagcctgcaacctgaggacacagccacatattattgtgcagcacataatgagccttatttttgcgactatagtgggcgttttcggtggaatgagtacagctactatggccaggggacccaggtcaccgtctcctcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - DNA, 345 bp, synthetic construct
  source 1..345: mol_type = other DNA
caggtgcagctgcaggagtctgggggaggcttggtgcaggctggggggtctctgagactctcctgtgcagcctctggaagcatcttcagtcctaataccatgggctggtttcgccaggctctagggaagcagcgcgaaatggtcgcagttattagtagtattgctagcacgcagtatgcaaacttcgtgaagggccgattcaccatcaccagagacaacaccaagaatacggtgcatctccaaatgaacagcctgattcctgaggacacagccgtctattactgttatgccgtcgacaagtcccaagactactggggccaggggacccaggtcaccgtctcctca

SEQ ID NO: 25 - DNA, 1041 bp, synthetic construct
  source 1..1041: mol_type = other DNA
caggtgcagctgcaggagtctgggggaggcttggtgcaggctggggggtctctgagactctcctgtgcagcctctggaagcatcttcagtcctaataccatgggctggtttcgccaggctctagggaagcagcgcgaaatggtcgcagttattagtagtattgctagcacgcagtatgcaaacttcgtgaagggccgattcaccatcaccagagacaacaccaagaatacggtgcatctccaaatgaacagcctgattcctgaggacacagccgtctattactgttatgccgtcgacaagtcccaagactactggggccaggggacccaggtcaccgtctcctcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - DNA, 381 bp, synthetic construct
  source 1..381: mol_type = other DNA
caggtgcagctgcaggagtctgggggaggcttggtgcagcctggggggtctctgagactctcctgtgcagcctctggattcactttggattattatgccataggctggttccgccaggccccagggaaggagcgtgagggggtctcatgtattagtagtagtggtgggcgcacaaactatgcagactccgtgaagggccgattcaccatctccagagacaacaccaagaacacggtgtatttgcagatgaacagtctgaaacctgaggacacagccgtttattactgcgcagcttgggaggctagtaggtggtactgtccactccaattttctgctgactttagttcctggggccaggggacccaggtcaccgtctcctca

SEQ ID NO: 27 - DNA, 1077 bp, synthetic construct
  source 1..1077: mol_type = other DNA
caggtgcagctgcaggagtctgggggaggcttggtgcagcctggggggtctctgagactctcctgtgcagcctctggattcactttggattattatgccataggctggttccgccaggccccagggaaggagcgtgagggggtctcatgtattagtagtagtggtgggcgcacaaactatgcagactccgtgaagggccgattcaccatctccagagacaacaccaagaacacggtgtatttgcagatgaacagtctgaaacctgaggacacagccgtttattactgcgcagcttgggaggctagtaggtggtactgtccactccaattttctgctgactttagttcctggggccaggggacccaggtcaccgtctcctcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 28-30 - skipped (3 entries)

SEQ ID NO: 31 - protein, 129 aa, synthetic construct
QVQLQESGGGAVQPGGSLGLSCTASGFNFETSTVGWFRQAPGKEYEGVSCINKGYEDTNYADSVKGRFTISRDAAKNTVYLQMDSLQPEDTATYYCAAHNEPYFCDYSGRFRWNEYSYYGQGTQVTVSS

SEQ ID NO: 32 - protein, 150 aa, synthetic construct
QVQLQESGGGAVQPGGSLGLSCTASGFNFETSTVGWFRQAPGKEYEGVSCINKGYEDTNYADSVKGRFTISRDAAKNTVYLQMDSLQPEDTATYYCAAHNEPYFCDYSGRFRWNEYSYYGQGTQVTVSSGGQHHHHHHGAYPYDVPDYAS

SEQ ID NO: 33 - protein, 115 aa, synthetic construct
QVQLQESGGGLVQAGGPLRLSCVASASTSASNSMSWFRQAPGKQREWVATAANGDIRSYANFVKGRFTISRDDAKNTVYLQMNGLKPEDTAVYYCYSVDSYRDYWGQGTQVTVSS

SEQ ID NO: 34 - protein, 8 aa, synthetic construct
STSASNSM

SEQ ID NO: 35 - protein, 9 aa, synthetic construct
TAANGDIRS

SEQ ID NO: 36 - protein, 9 aa, synthetic construct
YSVDSYRDY

SEQ ID NO: 37 - protein, 136 aa, synthetic construct
QVQLQESGGGLVQAGGPLRLSCVASASTSASNSMSWFRQAPGKQREWVATAANGDIRSYANFVKGRFTISRDDAKNTVYLQMNGLKPEDTAVYYCYSVDSYRDYWGQGTQVTVSSGGQHHHHHHGAYPYDVPDYAS

SEQ ID NO: 38 - skipped

SEQ ID NO: 39 - protein, 136 aa, synthetic construct
QVQLQESGGGLVQAGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSSGGQHHHHHHGAYPYDVPDYAS

SEQ ID NO: 40-43 - skipped (4 entries)

SEQ ID NO: 44 - protein, 115 aa, synthetic construct
QVQLQESGGGLVQPGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSS

SEQ ID NO: 45 - protein, 136 aa, synthetic construct
QVQLQESGGGLVQPGGSLRLSCAASGSIFSPNTMGWFRQALGKQREMVAVISSIASTQYANFVKGRFTITRDNTKNTVHLQMNSLIPEDTAVYYCYAVDKSQDYWGQGTQVTVSSGGQHHHHHHGAYPYDVPDYAS

SEQ ID NO: 46 - protein, 127 aa, synthetic construct
QVQLQESGGGLVQPGGSLRLSCAASGFILDFYAIGWFRQAPGKEREGVSCISSSGGRTNYADSVKGRFTISRDNAKNTVYLQMNSLKPEDTAVYSCAAWEGSTRYCPIQTSADFVSWGQGTQVTVSS

SEQ ID NO: 47 - protein, 8 aa, synthetic construct
FILDFYAI

SEQ ID NO: 48 - protein, 20 aa, synthetic construct
AAWEGSTRYCPIQTSADFVS

SEQ ID NO: 49 - protein, 148 aa, synthetic construct
QVQLQESGGGLVQPGGSLRLSCAASGFILDFYAIGWFRQAPGKEREGVSCISSSGGRTNYADSVKGRFTISRDNAKNTVYLQMNSLKPEDTAVYSCAAWEGSTRYCPIQTSADFVSWGQGTQVTVSSGGQHHHHHHGAYPYDVPDYAS

SEQ ID NO: 50-61 - skipped (12 entries)

SEQ ID NO: 62 - protein, 127 aa, synthetic construct
QVQLQESGGGLVQPGGSLRLSCAASGFILDFYAIGWFRQAPGKEREGVSCISSSGGRTNYADSVKGRFTISSDNAKNTVYLQMNSLKPEDTAVYSCAAWEGSTRYCPIQTSADFVSWGQGTQVTVSS

SEQ ID NO: 63 - protein, 148 aa, synthetic construct
QVQLQESGGGLVQPGGSLRLSCAASGFILDFYAIGWFRQAPGKEREGVSCISSSGGRTNYADSVKGRFTISSDNAKNTVYLQMNSLKPEDTAVYSCAAWEGSTRYCPIQTSADFVSWGQGTQVTVSSGGQHHHHHHGAYPYDVPDYAS

SEQ ID NO: 64 - protein, 127 aa, synthetic construct
QVQLQESGGGLVQAGGSLRLTCVASGTTLDHYAIGWLRQAPGKEREGVSCISSSGGSTNYADSVKGRFTISRDNAKNTVYLQMTGLKPEDTAVYYCAAWEGSSEYCPLQFSADFASWGRGTQVTVSS

SEQ ID NO: 65 - protein, 8 aa, synthetic construct
TTLDHYAI

SEQ ID NO: 66 - protein, 10 aa, synthetic construct
CISSSGGSTN

SEQ ID NO: 67 - protein, 20 aa, synthetic construct
AAWEGSSEYCPLQFSADFAS

SEQ ID NO: 68 - protein, 148 aa, synthetic construct
QVQLQESGGGLVQAGGSLRLTCVASGTTLDHYAIGWLRQAPGKEREGVSCISSSGGSTNYADSVKGRFTISRDNAKNTVYLQMTGLKPEDTAVYYCAAWEGSSEYCPLQFSADFASWGRGTQVTVSSGGQHHHHHHGAYPYDVPDYAS

SEQ ID NO: 69 - protein, 127 aa, synthetic construct
QVQLQESGGGLVQDGGSLRLSCEASGFTVNSHAIGWFRQSPGKEREGVSCISSSGGSTNYADSVKGRFTISRDNAKNTVYLQMTSLKPEDTAVYYCAAWEGSSEYCPLQFSADFASWGRGTQVTVSS

SEQ ID NO: 70 - protein, 8 aa, synthetic construct
FTVNSHAI

SEQ ID NO: 71 - protein, 148 aa, synthetic construct
QVQLQESGGGLVQDGGSLRLSCEASGFTVNSHAIGWFRQSPGKEREGVSCISSSGGSTNYADSVKGRFTISRDNAKNTVYLQMTSLKPEDTAVYYCAAWEGSSEYCPLQFSADFASWGRGTQVTVSSGGQHHHHHHGAYPYDVPDYAS

SEQ ID NO: 72 - protein, 127 aa, synthetic construct
QVQLQECGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQYSADFDSWGQGTQVTVSS

SEQ ID NO: 73 - protein, 20 aa, synthetic construct
AAWEGSSEYCPLQYSADFDS

SEQ ID NO: 74 - protein, 148 aa, synthetic construct
QVQLQECGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQYSADFDSWGQGTQVTVSSGGQHHHHHHGAYPYDVPDYAS

SEQ ID NO: 75 - protein, 127 aa, synthetic construct
QVQLQESGGGLVQPGGSLRLSCAASGFALDYYAIGWFRQAPKKEREGVSCISISGGSTNYADSVEGRFTISRDNAKNTVYLQMTSLKPEDTAVYYCAAWEGSSEYCPLQFSADFASWGRGTQVTVSS

SEQ ID NO: 76 - protein, 8 aa, synthetic construct
FALDYYAI

SEQ ID NO: 77 - protein, 10 aa, synthetic construct
CISISGGSTN

SEQ ID NO: 78 - protein, 148 aa, synthetic construct
QVQLQESGGGLVQPGGSLRLSCAASGFALDYYAIGWFRQAPKKEREGVSCISISGGSTNYADSVEGRFTISRDNAKNTVYLQMTSLKPEDTAVYYCAAWEGSSEYCPLQFSADFASWGRGTQVTVSSGGQHHHHHHGAYPYDVPDYAS

SEQ ID NO: 79 - protein, 127 aa, synthetic construct
QVQLQESGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQFSADFASRGQGTQVTVSS

SEQ ID NO: 80 - protein, 148 aa, synthetic construct
QVQLQESGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQFSADFASRGQGTQVTVSSGGQHHHHHHGAYPYDVPDYAS

SEQ ID NO: 81 - protein, 127 aa, synthetic construct
QVQLQESGGGLVQPGGSLRLFCAASGFTLDYYAIGWFRQAPGKEREGVSCISSSGGSTNYADSVKGRFTISRDNAKNTVYLQMTSLKPEDTAVYYCAAWEGSSEYCPLQFSADFASWGRGTQVTVSS

SEQ ID NO: 82 - protein, 148 aa, synthetic construct
QVQLQESGGGLVQPGGSLRLFCAASGFTLDYYAIGWFRQAPGKEREGVSCISSSGGSTNYADSVKGRFTISRDNAKNTVYLQMTSLKPEDTAVYYCAAWEGSSEYCPLQFSADFASWGRGTQVTVSSGGQHHHHHHGAYPYDVPDYAS

SEQ ID NO: 83 - protein, 127 aa, synthetic construct
QVQLQESGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQFSADFASWGRGTQVTVSS

SEQ ID NO: 84 - protein, 148 aa, synthetic construct
QVQLQESGGGLVQPGGSLRLSCAASGFTLDYYAIGWFRQAPGKEREGVSCISSSGGSTNYADSVKGRFTISRDNAKNTVYLQMTSLKPEDTAVYYCAAWEGSSEYCPLQFSADFASWGRGTQVTVSSGGQHHHHHHGAYPYDVPDYAS

SEQ ID NO: 85-86 - skipped (2 entries)

SEQ ID NO: 87 - protein, 127 aa, synthetic construct
QVQLQESGGGLVQPGGSLRLSCAASGFTLDYYAIGWFRQAPGKEREGVSCISSSGGSTNYVDSVKGRFTISRDNAKNTVYLQMTSLKPEDTAVYYCAAWEGSSEYCPLQFSADFASWGRGTQVTVSS

SEQ ID NO: 88 - protein, 148 aa, synthetic construct
QVQLQESGGGLVQPGGSLRLSCAASGFTLDYYAIGWFRQAPGKEREGVSCISSSGGSTNYVDSVKGRFTISRDNAKNTVYLQMTSLKPEDTAVYYCAAWEGSSEYCPLQFSADFASWGRGTQVTVSSGGQHHHHHHGAYPYDVPDYAS

SEQ ID NO: 89 - protein, 114 aa, synthetic construct
QVQLQESGGGLVQAGGSLRLSCVASGSIFRFEAVGWYRQAPGKQRELVATVARDGTTNYADSVKGRFTISTDNAKNSVYLQMNSLKAEDTAVYVCNARWWTNFWGQGTQVTVSS

SEQ ID NO: 90 - protein, 8 aa, synthetic construct
SIFRFEAV

SEQ ID NO: 91 - protein, 9 aa, synthetic construct
TVARDGTTN

SEQ ID NO: 92 - protein, 8 aa, synthetic construct
NARWWTNF

SEQ ID NO: 93 - protein, 135 aa, synthetic construct
QVQLQESGGGLVQAGGSLRLSCVASGSIFRFEAVGWYRQAPGKQRELVATVARDGTTNYADSVKGRFTISTDNAKNSVYLQMNSLKAEDTAVYVCNARWWTNFWGQGTQVTVSSGGQHHHHHHGAYPYDVPDYAS

SEQ ID NO: 94 - protein, 114 aa, synthetic construct
QVQLQESGGGLVQAGGSLRLSCAASESIFRMDVVQWYRQAPGKQRELVASITRSGSTNYADSVKGRFIISSDNAKNSVYLQMKSLKVEDTAVYLCHARTWTSYWGQGTQVTVSS

SEQ ID NO: 95 - protein, 8 aa, synthetic construct
SIFRMDVV

SEQ ID NO: 96 - protein, 9 aa, synthetic construct
SITRSGSTN

SEQ ID NO: 97 - protein, 8 aa, synthetic construct
HARTWTSY

SEQ ID NO: 98 - protein, 135 aa, synthetic construct
QVQLQESGGGLVQAGGSLRLSCAASESIFRMDVVQWYRQAPGKQRELVASITRSGSTNYADSVKGRFIISSDNAKNSVYLQMKSLKVEDTAVYLCHARTWTSYWGQGTQVTVSSGGQHHHHHHGAYPYDVPDYAS

SEQ ID NO: 99 - protein, 114 aa, synthetic construct
QVQLQESGGGLVQPGGSLRLSCAASESIFRMDVVQWYRQAPGKQRELVASITRSGSTNYADSVKGRFIISSDNAKNSVYLQMKSLKVEDTAVYLCHARTWTSYWGQGTQVTVSS

SEQ ID NO: 100 - protein, 135 aa, synthetic construct
QVQLQESGGGLVQPGGSLRLSCAASESIFRMDVVQWYRQAPGKQRELVASITRSGSTNYADSVKGRFIISSDNAKNSVYLQMKSLKVEDTAVYLCHARTWTSYWGQGTQVTVSSGGQHHHHHHGAYPYDVPDYAS

SEQ ID NO: 101 - protein, 114 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSSWGRGTQVTVSS

SEQ ID NO: 102 - protein, 8 aa, synthetic construct
SIFRMELM

SEQ ID NO: 103 - protein, 9 aa, synthetic construct
TINRCGSTN

SEQ ID NO: 104 - protein, 8 aa, synthetic construct
HARTWTSS

SEQ ID NO: 105 - protein, 135 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSSWGRGTQVTVSSGGQHHHHHHGAYPYDVPDYAS

SEQ ID NO: 106 - protein, 114 aa, synthetic construct
QVQLQESGGGLVQAGGSLRLSCAASESIFRMELMQWYRQAPGKQRELVATITRSGSTNYSDSVKGRLIISSDNAKNSVYLQMNSLKAEDTAVYLCHARTWTSYWGQGTQVTVSS

SEQ ID NO: 107 - protein, 135 aa, synthetic construct
QVQLQESGGGLVQAGGSLRLSCAASESIFRMELMQWYRQAPGKQRELVATITRSGSTNYSDSVKGRLIISSDNAKNSVYLQMNSLKAEDTAVYLCHARTWTSYWGQGTQVTVSSGGQHHHHHHGAYPYDVPDYAS

SEQ ID NO: 108 - protein, 114 aa, synthetic construct
QVQLQESGGGLVQAGGSLRLSCAASESIFRMELMEWYRQAPGKQRELVATITRSGSTNYSDSVKGRFIISSDNAKNSVYLQMNSLKAEDTAVYLCHARTWTSYWGQGTQVTVSS

SEQ ID NO: 109 - protein, 135 aa, synthetic construct
QVQLQESGGGLVQAGGSLRLSCAASESIFRMELMEWYRQAPGKQRELVATITRSGSTNYSDSVKGRFIISSDNAKNSVYLQMNSLKAEDTAVYLCHARTWTSYWGQGTQVTVSSGGQHHHHHHGAYPYDVPDYAS

SEQ ID NO: 110-117 - skipped (8 entries)

SEQ ID NO: 118 - protein, 114 aa, synthetic construct
QVQLQESGGGLVQAGGSLRLSCAASESIFRMELMQWYRQAPGKQRELVATITRSGSTNYSDSVKGRFIISSDNAKNSVYLQMNSLKAEDTAVYLCHARTWTSYWGQGTQVTVSS

SEQ ID NO: 119 - protein, 135 aa, synthetic construct
QVQLQESGGGLVQAGGSLRLSCAASESIFRMELMQWYRQAPGKQRELVATITRSGSTNYSDSVKGRFIISSDNAKNSVYLQMNSLKAEDTAVYLCHARTWTSYWGQGTQVTVSSGGQHHHHHHGAYPYDVPDYAS

SEQ ID NO: 120-131 - skipped (12 entries)

SEQ ID NO: 132 - protein, 114 aa, synthetic construct
QVQLQESGGGLVQAGGSLRLSCAASESIFRMELMAWYRQAPGKQRELVATITRSGSTNYSDSVKGRFIISSDNAKNSVYLQMNSLKAEDTAVYLCHARTWTSYWGQGTQVTVSS

SEQ ID NO: 133 - protein, 135 aa, synthetic construct
QVQLQESGGGLVQAGGSLRLSCAASESIFRMELMAWYRQAPGKQRELVATITRSGSTNYSDSVKGRFIISSDNAKNSVYLQMNSLKAEDTAVYLCHARTWTSYWGQGTQVTVSSGGQHHHHHHGAYPYDVPDYAS

SEQ ID NO: 134 - protein, 118 aa, synthetic construct
QVQLQESGGGLVQPGGSLRLSCVASGSVTFNSMGWYRQAPGKQRELVAQITAGGDTHYADSVKGRFTISEHRGKNAVYLEMHSLKPEDTAVYYCHLQVPFLGGGYDYWGQGTQVTVSS

SEQ ID NO: 135 - protein, 7 aa, synthetic construct
SVTFNSM

SEQ ID NO: 136 - protein, 9 aa, synthetic construct
QITAGGDTH

SEQ ID NO: 137 - protein, 13 aa, synthetic construct
HLQVPFLGGGYDY

SEQ ID NO: 138 - protein, 139 aa, synthetic construct
QVQLQESGGGLVQPGGSLRLSCVASGSVTFNSMGWYRQAPGKQRELVAQITAGGDTHYADSVKGRFTISEHRGKNAVYLEMHSLKPEDTAVYYCHLQVPFLGGGYDYWGQGTQVTVSSGGQHHHHHHGAYPYDVPDYAS

SEQ ID NO: 139 - protein, 118 aa, synthetic construct
QVQLQESGGGLVQPGGSLRLSCVASGSVTFNSMGWYRQAPGKQRELVAQITAGGDTHYADSVKGRFTISEHRDKNTVYLEMRSLKPEDTAVYYCHLQVPFLGGGYDYWGQGTQVTVSS

SEQ ID NO: 140 - protein, 139 aa, synthetic construct
QVQLQESGGGLVQPGGSLRLSCVASGSVTFNSMGWYRQAPGKQRELVAQITAGGDTHYADSVKGRFTISEHRDKNTVYLEMRSLKPEDTAVYYCHLQVPFLGGGYDYWGQGTQVTVSSGGQHHHHHHGAYPYDVPDYAS

SEQ ID NO: 141 - protein, 118 aa, synthetic construct
QVQLQEWGGGLVQPGGSLRLSCVASGSVTFNSMGWYRQAPGKQRELVAQITAGGDTHYADSVKGRFTISEHRGKNAVYLEMHSLKPEDTAVYYCHLQVPFLGGGYDYWGQGTQVTVSS

SEQ ID NO: 142 - protein, 139 aa, synthetic construct
QVQLQEWGGGLVQPGGSLRLSCVASGSVTFNSMGWYRQAPGKQRELVAQITAGGDTHYADSVKGRFTISEHRGKNAVYLEMHSLKPEDTAVYYCHLQVPFLGGGYDYWGQGTQVTVSSGGQHHHHHHGAYPYDVPDYAS

SEQ ID NO: 143 - protein, 116 aa, synthetic construct
QVQLQESGGGLVQAGESLRLSCVASGSIFSINAMGWYRQAPGKQRELVAGITDDGSTNYTHFVEGRFTISRDNAKNTVYLQMNSLKPEDTAVYYCNAKIHTPYNYWGQGTQVTVSS

SEQ ID NO: 144 - protein, 8 aa, synthetic construct
SIFSINAM

SEQ ID NO: 145 - protein, 9 aa, synthetic construct
GITDDGSTN

SEQ ID NO: 146 - protein, 10 aa, synthetic construct
NAKIHTPYNY

SEQ ID NO: 147 - protein, 137 aa, synthetic construct
QVQLQESGGGLVQAGESLRLSCVASGSIFSINAMGWYRQAPGKQRELVAGITDDGSTNYTHFVEGRFTISRDNAKNTVYLQMNSLKPEDTAVYYCNAKIHTPYNYWGQGTQVTVSSGGQHHHHHHGAYPYDVPDYAS

SEQ ID NO: 148 - protein, 9 aa, synthetic construct
  VARIANT 2: note = A or S
  VARIANT 5: note = K or S
  VARIANT 6: note = S or Y
  VARIANT 7: note = Q or R
YXVDXXXDY

SEQ ID NO: 149 - protein, 8 aa, synthetic construct
  VARIANT 2: note = T, I, or A
  VARIANT 5: note = Y, F, or H
FXLDXYAI

SEQ ID NO: 150 - protein, 10 aa, synthetic construct
  VARIANT 4: note = S or I
  VARIANT 8: note = R or S
CISXSGGXTN

SEQ ID NO: 151 - protein, 20 aa, synthetic construct
  VARIANT 5: note = A or G
  VARIANT 7: note = R, T, or S
  VARIANT 8: note = W, R, or E
  VARIANT 14: note = F, T, or Y
  VARIANT 19: note = S, V, A, or D
AAWEXSXXYCPLQXSADFXS

SEQ ID NO: 152 - protein, 346 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSSWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 350 aa, synthetic construct
QVQLQESGGGLVQPGGSLRLSCVASGSVTFNSMGWYRQAPGKQRELVAQITAGGDTHYADSVKGRFTISEHRGKNAVYLEMHSLKPEDTAVYYCHLQVPFLGGGYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 348 aa, synthetic construct
QVQLQESGGGLVQAGESLRLSCVASGSIFSINAMGWYRQAPGKQRELVAGITDDGSTNYTHFVEGRFTISRDNAKNTVYLQMNSLKPEDTAVYYCNAKIHTPYNYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 6 aa, synthetic construct
GGSGGS

SEQ ID NO: 156 - protein, 115 aa, synthetic construct
QVQLQESGGGLVQAGGSLRLSCAASGSIFSPNTMGWFRQALGKQREMVAFISSIASTSYANFVKGRFTITRDNTKNTVHLQMNSLIPEDTAVYYCYAVDKSQDYWGQGTQVTVSS

SEQ ID NO: 157 - protein, 9 aa, synthetic construct
FISSIASTS

SEQ ID NO: 158 - protein, 123 aa, synthetic construct
QVQLQESGGGLVQAGGSLRLSCAASGSIFSPNTMGWFRQALGKQREMVAFISSIASTSYANFVKGRFTITRDNTKNTVHLQMNSLIPEDTAVYYCYAVDKSQDYWGQGTQVTVSSGSHHHHHH

SEQ ID NO: 159 - protein, 9 aa, synthetic construct
  VARIANT 1: note = V or F
  VARIANT 9: note = Q or S
XISSIASTX

SEQ ID NO: 160 - protein, 8 aa, synthetic construct
  VARIANT 5: note = F or M
  VARIANT 6: note = E or D
  VARIANT 7: note = A, V, or L
  VARIANT 8: note = V or M
SIFRXXXX

SEQ ID NO: 161 - protein, 9 aa, synthetic construct
  VARIANT 1: note = S or T
  VARIANT 2: note = V or I
  VARIANT 3: note = A, T, or N
  VARIANT 5: note = D, S, or C
  VARIANT 7: note = T or S
XXXRXGXTN

SEQ ID NO: 162 - protein, 8 aa, synthetic construct
  VARIANT 1: note = H or N
  VARIANT 4: note = T or W
  VARIANT 7: note = S or N
  VARIANT 8: note = S, Y, or F
XARXWTXX

SEQ ID NO: 163 - protein, 9 aa, synthetic construct
TITRSGSTN

SEQ ID NO: 164 - protein, 114 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSYWGRGTQVTVSS

SEQ ID NO: 165 - protein, 135 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSYWGRGTQVTVSSGGQHHHHHHGAYPYDVPDYAS

SEQ ID NO: 166 - protein, 346 aa, synthetic construct
QVQLQESGGGLVQAGGSLRLSCAASESIFRMELMEWYHQAPGKQRELVATINRCGSTNYSDSVKGRFIISSDNAKNSVYLQMNSLKDEDTAVYSCHARTWTSYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 115 aa, synthetic construct
QVQLQESGGGLVQAGGSLRLSCAASGSIFSPNTMGWFRQALGKQREGVAFISSIASTSYWLPVKGRFTITRDNTKNTVHLQMNSLIPEDTAVYYCYAVDKSQDYWGQGTQVTVSS

SEQ ID NO: 168 - protein, 115 aa, synthetic construct
QVQLQESGGGLVQAGGSLRLSCAASGSIFSPNTMGWFRQALGKQREGVAFISSIASTSYNWYVKGRFTITRDNTKNTVHLQMNSLIPEDTAVYYCYAVDKSQDYWGQGTQVTVSS

SEQ ID NO: 169 - protein, 115 aa, synthetic construct
QVQLQESGGGLVQAGGSLRLSCAASGSIFSPNTMGWFRQALGKQREGVAFISSIASTSYNFFVKGRFTITRDNTKNTVHLQMNSLIPEDTAVYYCYAVDKSQDYWGQGTQVTVSS

SEQ ID NO: 170 - protein, 115 aa, synthetic construct
QVQLQESGGGLVQAGGSLRLSCAASGSIFSPNTMGWFRQALGKQREGVAFISSIASTSYGYGVKGRFTITRDNTKNTVHLQMNSLIPEDTAVYYCYAVDKSQDYWGQGTQVTVSS

SEQ ID NO: 171 - protein, 136 aa, synthetic construct
QVQLQESGGGLVQAGGSLRLSCAASGSIFSPNTMGWFRQALGKQREGVAFISSIASTSYWLPVKGRFTITRDNTKNTVHLQMNSLIPEDTAVYYCYAVDKSQDYWGQGTQVTVSSGGQHHHHHHGAYPYDVPDYAS

SEQ ID NO: 172 - protein, 347 aa, synthetic construct
QVQLQESGGGLVQAGGSLRLSCAASGSIFSPNTMGWFRQALGKQREMVAFISSIASTSYANFVKGRFTITRDNTKNTVHLQMNSLIPEDTAVYYCYAVDKSQDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 347 aa, synthetic construct
QVQLQESGGGLVQAGGSLRLSCAASGSIFSPNTMGWFRQALGKQREGVAFISSIASTSYWLPVKGRFTITRDNTKNTVHLQMNSLIPEDTAVYYCYAVDKSQDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - DNA, 67 bp, synthetic construct
  source 1..67: mol_type = other DNA
  misc_difference 23: note = a, c, t, g, unknown or other
  misc_difference 24: note = a, c, t, g, unknown or other
  misc_difference 25: note = a, c, t, g, unknown or other
  misc_difference 47: note = a, c, t, g, unknown or other
  misc_difference 48: note = a, c, t, g, unknown or other
  misc_difference 49: note = a, c, t, g, unknown or other
gaagcagcgcgaaatggtcgcannnattagtagtattgctagcacgnnntatgcaaacttcgtgaag

SEQ ID NO: 175 - DNA, 62 bp, synthetic construct
  source 1..62: mol_type = other DNA
  misc_difference 33: note = a, c, t, g, unknown or other
  misc_difference 34: note = a, c, t, g, unknown or other
  misc_difference 35: note = a, c, t, g, unknown or other
ggtttcgccaggctctagggaagcagcgcgaannngtcgcatttattagtagtattgctagc

SEQ ID NO: 176 - DNA, 59 bp, synthetic construct
  source 1..59: mol_type = other DNA
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
catttattagtagtattgctagcacgagttatnnnnnnnnngtgaagggccgattcacc

SEQ ID NO: 177 - protein, 4 aa, synthetic construct
RRAR

SEQ ID NO: 178 - protein, 4 aa, synthetic construct
AGAR

SEQ ID NO: 179 - protein, 35 aa, synthetic construct
EDTATYYCAAHNEPYFCDYSGRFRWNEYSYYGQGT

SEQ ID NO: 180 - protein, 35 aa, synthetic construct; identical to SEQ ID NO: 179

SEQ ID NO: 181 - protein, 22 aa, synthetic construct
EDTAVYYCYSVDSYRDYWGQGT

SEQ ID NO: 182 - protein, 22 aa, synthetic construct
EDTAVYYCYAVDKSQDYWGQGT

SEQ ID NO: 183 - protein, 22 aa, synthetic construct
EDTAVYYCYAVDKSQDYWGQGT

SEQ ID NO: 184 - protein, 22 aa, synthetic construct
EDTAVYYCYAVDKSQDYWGQGT

SEQ ID NO: 185 - protein, 22 aa, synthetic construct
EDTAVYYCYAVDKSQDYWGQGT

SEQ ID NO: 186 - protein, 22 aa, synthetic construct
EDTAVYYCYAVDKSQDYWGQGT

SEQ ID NO: 187 - protein, 33 aa, synthetic construct
EDTAVYSCAAWEGSTRYCPIQTSADFVSWGQGT

SEQ ID NO: 188 - protein, 33 aa, synthetic construct; identical to SEQ ID NO: 187

SEQ ID NO: 189 - protein, 33 aa, synthetic construct; identical to SEQ ID NO: 187

SEQ ID NO: 190 - protein, 33 aa, synthetic construct; identical to SEQ ID NO: 187

SEQ ID NO: 191 - protein, 33 aa, synthetic construct; identical to SEQ ID NO: 187

SEQ ID NO: 192 - protein, 33 aa, synthetic construct; identical to SEQ ID NO: 187

SEQ ID NO: 193 - protein, 33 aa, synthetic construct; identical to SEQ ID NO: 187

SEQ ID NO: 194 - protein, 33 aa, synthetic construct; identical to SEQ ID NO: 187

SEQ ID NO: 195 - protein, 33 aa, synthetic construct
EDTAVYYCAAWEASRWYCPLQFSADFSSWGQGT

SEQ ID NO: 196 - protein, 33 aa, synthetic construct
EDTAVYYCAAWEGSSEYCPLQFSADFASWGRGT

SEQ ID NO: 197 - protein, 33 aa, synthetic construct; identical to SEQ ID NO: 196

SEQ ID NO: 198 - protein, 33 aa, synthetic construct
EDTAVYYCAAWEGSSEYCPLQYSADFDSWGQGT

SEQ ID NO: 199 - protein, 33 aa, synthetic construct; identical to SEQ ID NO: 196

SEQ ID NO: 200 - protein, 33 aa, synthetic construct
EDTAVYYCAAWEGSSEYCPLQFSADFASRGQGT

SEQ ID NO: 201 - protein, 33 aa, synthetic construct; identical to SEQ ID NO: 196

SEQ ID NO: 202 - protein, 33 aa, synthetic construct; identical to SEQ ID NO: 196

SEQ ID NO: 203 - protein, 33 aa, synthetic construct; identical to SEQ ID NO: 196

SEQ ID NO: 204 - protein, 33 aa, synthetic construct; identical to SEQ ID NO: 196

SEQ ID NO: 205 - protein, 21 aa, synthetic construct
EDTAVYVCNARWWTNFWGQGT

SEQ ID NO: 206 - protein, 21 aa, synthetic construct
EDTAVYLCHARTWTSYWGQGT

SEQ ID NO: 207 - protein, 21 aa, synthetic construct
EDTAVYLCHARTWTSYWGQGT

SEQ ID NO: 208 - protein, 21 aa, synthetic construct
EDTAVYSCHARTWTSYWGRGT

SEQ ID NO: 209 - protein, 21 aa, synthetic construct
EDTAVYLCHARTWTSYWGQGT

SEQ ID NO: 210 - protein, 21 aa, synthetic construct
EDTAVYLCHARTWTSYWGQGT

SEQ ID NO: 211 - protein, 21 aa, synthetic construct
EDTAVYLCHARTWTSYWGQGT

SEQ ID NO: 212 - protein, 21 aa, synthetic construct
EDTAVYLCHARTWTSYWGQGT

SEQ ID NO: 213 - protein, 21 aa, synthetic construct
EDTAVYLCHARTWTSYWGQGT

SEQ ID NO: 214 - protein, 21 aa, synthetic construct
EDTAVYLCHARTWTSYWGQGT

SEQ ID NO: 215 - protein, 21 aa, synthetic construct
EDTAVYLCHARTWTSYWGQGT

SEQ ID NO: 216 - protein, 21 aa, synthetic construct
EDTAVYLCHARTWTSYWGQGT

SEQ ID NO: 217 - protein, 21 aa, synthetic construct
EDTAVYLCHARTWTSYWGQGT

SEQ ID NO: 218 - protein, 21 aa, synthetic construct
EDTAVYLCHARTWTSYWGQGT

SEQ ID NO: 219 - protein, 21 aa, synthetic construct
EDTAVYLCHARTWTSYWGQGT

SEQ ID NO: 220 - protein, 21 aa, synthetic construct
EDTAVYLCHARTWTSYWGQGT

SEQ ID NO: 221 - protein, 21 aa, synthetic construct
EDTAVYLCHARTWTSYWGQGT

SEQ ID NO: 222 - protein, 21 aa, synthetic construct
EDTAVYLCHARTWTSYWGQGT

SEQ ID NO: 223 - protein, 26 aa, synthetic construct
EDTAVYYCHLQVPFLGGGYDYWGQGT

SEQ ID NO: 224 - protein, 26 aa, synthetic construct
EDTAVYYCHLQVPFLGGGYDYWGQGT

SEQ ID NO: 225 - protein, 26 aa, synthetic construct
EDTAVYYCHLQVPFLGGGYDYWGQGT

SEQ ID NO: 226 - protein, 23 aa, synthetic construct
EDTAVYYCNAKIHTPYNYWGQGT

SEQ ID NO: 227 - protein, 4 aa, synthetic construct
  SITE 4: note = N-Glycan modified residue
FFSN

SEQ ID NO: 228 - protein, 4 aa, synthetic construct
LDPL

SEQ ID NO: 229 - protein, 6 aa, synthetic construct
WRVYST

SEQ ID NO: 230 - protein, 5 aa, synthetic construct
NQVAV

SEQ ID NO: 231 - protein, 4 aa, synthetic construct
FFSN

SEQ ID NO: 232 - protein, 6 aa, synthetic construct
SNQVAV